(19) United States
(12) Patent Application Publication
(10) Pub. No.: US 2024/0400650 A1
(43) Pub. Date: Dec. 5, 2024

(54) Anti-HBsAg antibody for treatment of chronic hepatitis D

(71) Applicant: Bluejay Therapeutics, Inc., San Mateo, CA (US)

(72) Inventor: Hassan Javanbakht, San Francisco, CA (US)

(21) Appl. No.: 18/679,267

(22) Filed: May 30, 2024

Related U.S. Application Data
(60) Provisional application No. 63/505,425, filed on May 31, 2023.

(51) Int. Cl. C07K 16/08; A61K 39/00; A61P 31/20; G01N 33/569

(52) U.S. Cl. CPC C07K 16/082; A61P 31/20; G01N 33/56983; A61K 2039/505; C07K 2317/41; C07K 2317/565; C07K 2317/76

(57) ABSTRACT

The present invention relates to anti-HBsAg antibodies, antibody fragments, and their uses for the prevention and treatment of hepatitis D virus infection and associated diseases. A method of neutralizing a hepatitis D virus infection comprising administering via injection or infusion to a patient in need an effective amount of the antibody, wherein said antibody or antigen binding fragment, is provided.

### INCORPORATION OF SEQUENCE LISTING

This application includes a Sequence Listing which has been submitted in XML format via Patent Center, named “Bluej003. XML” which is 678 KB in size and created on May 28, 2024. The contents of the Sequence Listing are incorporated herein by reference in their entirety.

### FIELD OF THE INVENTION

The present disclosure is directed to anti-hepatitis B surface antigen antibodies (anti-HBsAg), antibody fragments, and their uses for the reducing the likelihood or treatment of hepatitis D viral infection. HBsAg is the key viral protein responsible for hepatitis D virus infection.

Anti-HBsAg antibodies, antibody fragments are potent, selective and fully human immunoglobulin G1 (IgG1)-neutralizing monoclonal antibody that targets the antigenic loop present in all forms of HBsAg proteins, L-/M-/S-HBsAg. HBsAg is the key viral protein responsible for recognition, binding, and entry of HDV virions to hepatocytes. Therefore provides anti HDV benefits by binding to HBsAg on HDV and neutralizing and clearing HDV virions.

### BACKGROUND OF THE INVENTION

Hepatitis delta virus (HDV) is the causative agent of chronic hepatitis delta (CHD), the most severe form of viral hepatitis. HDV infection can occur either simultaneously with HBV or more commonly, as a superinfection in patients already chronically infected with HBV. Relative to chronic hepatitis B (CHB) infection alone, CHD co-infection is associated with more severe liver disease, causing faster progression to cirrhosis, hepatocellular carcinoma, and liver failure.

Although Nucleoside inhibitors are effective in suppressing HBV replication, they have no impact on HDV replication. Antiviral treatments are urgently needed to prevent its progressive course leading to the development of cirrhosis, end-stage liver disease, and HCC.

Pegylated interferon (PegIFN) has been used as an off-label treatment against HDV despite associated side effects, low response rates, and high relapse rates (Bahcecioglu, Ispiroglu et al. 2015; Rizzetto and Smedile 2015). Among the new CHD treatments, bulevirtide is a myristoylated peptide given by daily subcutaneously injection, that blocks HBV and HDV entry by targeting host NTCP receptor and has obtained conditional marketing authorization in Europe (Masetti and Aghemo 2021; Lampertico, Roulot et al. 2022). Lonafarnib, currently being evaluated in late-stage clinical studies, is an inhibitor of farnesyl transferase and thus inhibits HDV release (Yurdaydin, Keskin et al. 2022).

The current approved antiviral therapeutics Bulevirtide works by attaching to and blocking a receptor (target) through which the hepatitis delta and hepatitis B viruses enter liver cells. By blocking the entry of the virus into the cells, it limits the ability of HDV to replicate and its effects in the body, reducing symptoms of the disease. Therefore, better therapeutics to inhibit Hepatitis D infection are needed.

### SUMMARY OF THE INVENTION

The present invention is directed to neutralizing antibodies to hepatitis D virus and/or fragments thereof, and antibodies that reduce the amounts of hepatitis B surface antigen (HBsAg).

The present invention is directed to an antibody, wherein said antibody or antigen binding fragment thereof specifically binds HBsAg as disclosed in U.S. Pat. No. 21,210,221871A1, WO2019229699.

The antibody wherein said antibody or antigen binding fragment thereof specifically binds HBsAg. In one embodiment, the antibody or antigen binding fragment thereof binds to HBsAg and mutations thereof.

The present invention is directed to an antibody wherein said antibody or antigen binding fragment specifically binds to and neutralizes hepatitis D virus. In one embodiment, the antibody or antigen binding fragment thereof neutralizes hepatitis D virus and hepatitis D containing mutations in HBsAg. In another embodiment, the antibody or antigen binding fragment thereof reduces the amount of HBsAg. In another embodiment, the antibody or antigen binding fragment thereof reduces the amount of circulating HBsAg in the blood.

The present invention is directed to an isolated antibody, wherein said antibody or antigen binding fragment thereof comprises:
(i) a heavy chain variable region that comprises (a) a HCDR1 (CDR-Complementarity Determining Region) of SEQ ID NO:9, (b) a HCDR2 of SEQ ID NO: 10, (c) a HCDR3 of SEQ ID NO:11 and a light chain variable region that comprises: (d) a LCDR1 of SEQ ID NO: 25, (e) a LCDR2 of SEQ ID NO: 26, and (f) a LCDR3 of SEQ ID NO: 27;
(ii) a heavy chain variable region that comprises (a) a HCDR1 of SEQ ID NO: 41, (b) a HCDR2 of SEQ ID NO:42, (c) a HCDR3 of SEQ ID NO: 43; and a light chain variable region that comprises: (d) a LCDR1 of SEQ ID NO: 57, (e) a LCDR2 of SEQ ID NO: 58, and (f) a LCDR3 of SEQ ID NO:59;
(iii) a heavy chain variable region that comprises (a) a HCDR1 of SEQ ID NO:73, (b) a HCDR2 of SEQ ID NO:74, (c) a HCDR3 of SEQ ID NO:75; and a light chain variable region that comprises: (d) a LCDR1 of SEQ ID NO:89, (e) a LCDR2 of SEQ ID NO:90, and (f) a LCDR3 of SEQ ID NO:91;
(iv) a heavy chain variable region that comprises: (a) a HCDR1 of SEQ ID NO: 105, (b) a HCDR2 of SEQ ID NO: 106, (c) a HCDR3 of SEQ ID NO:107; and a light chain variable region that comprises: (d) a LCDR1 of SEQ ID NO: 121, (e) a LCDR2 of SEQ ID NO:122, and (f) a LCDR3 of SEQ ID NO:123;
(v) a heavy chain variable region that comprises: (a) a HCDR1 of SEQ ID NO: 137, (b) a HCDR2 of SEQ ID NO:138, (c) a HCDR3 of SEQ ID NO: 139; and a light chain variable region that comprises: (d) a LCDR1 of SEQ ID NO: 153, (e) a LCDR2 of SEQ ID NO: 154, and (f) a LCDR3 of SEQ ID NO:155;
(vi) a heavy chain variable region that comprises: (a) a HCDR1 of SEQ ID NO: 169, (b) a HCDR2 of SEQ ID NO: 170, (c) a HCDR3 of SEQ ID NO:171; and a light chain variable region that comprises: (d) a LCDR1 of SEQ ID NO: 185, (c) a LCDR2 of SEQ ID NO: 186, and (f) a LCDR3 of SEQ ID NO: 187;
(vii) a heavy chain variable region that comprises: (a) a HCDR1 of SEQ ID NO: 201, (b) a HCDR2 of SEQ ID NO: 202, (c) a HCDR3 of SEQ ID NO: 203; and a light chain variable region that comprises: (d) a LCDR1 of SEQ ID NO: 217, (c) a LCDR2 of SEQ ID NO: 218, and (f) a LCDR3 of SEQ ID NO: 219;
(viii) a heavy chain variable region that comprises: (a) a HCDR1 of SEQ ID NO: 233, (b) a HCDR2 of SEQ ID NO: 234, (c) a HCDR3 of SEQ ID NO: 235; and a light chain variable region that comprises: (d) a LCDR1 of SEQ ID NO: 249, (c) a LCDR2 of SEQ ID NO: 250, and (f) a LCDR3 of SEQ ID NO: 251;
(ix) a heavy chain variable region that comprises: (a) a HCDR1 of SEQ ID NO: 265, (b) a HCDR2 of SEQ ID NO:266, (c) a HCDR3 of SEQ ID NO: 267; and a light chain variable region that comprises: (d) a LCDR1 of SEQ ID NO: 281, (c) a LCDR2 of SEQ ID NO: 282, and (f) a LCDR3 of SEQ ID NO: 283;
(x) a heavy chain variable region that comprises: (a) a HCDR1 of SEQ ID NO: 297, (b) a HCDR2 of SEQ ID NO:298, (c) a HCDR3 of SEQ ID NO:299; and a light chain variable region that comprises: (d) a LCDR1 of SEQ ID NO: 313, (e) a LCDR2 of SEQ ID NO: 314, and (f) a LCDR3 of SEQ ID NO:315;
(xi) a heavy chain variable region that comprises: (a) a HCDR1 of SEQ ID NO:329, (b) a HCDR2 of SEQ ID NO: 330, (c) a HCDR3 of SEQ ID NO:331; and a light chain variable region that comprises: (d) a LCDR1 of SEQ ID NO: 345, (e) a LCDR2 of SEQ ID NO: 346, and (f) a LCDR3 of SEQ ID NO: 347;
(xii) a heavy chain variable region that comprises: (a) a HCDR1 of SEQ ID NO: 361, (b) a HCDR2 of SEQ ID NO: 362, (c) a HCDR3 of SEQ ID NO: 363; and a light chain variable region that comprises: (d) a LCDR1 of SEQ ID NO: 377, (e) a LCDR2 of SEQ ID NO:378, and (f) a LCDR3 of SEQ ID NO: 379;
(xiii) a heavy chain variable region that comprises: (a) a HCDR1 of SEQ ID NO: 393, (b) a HCDR2 of SEQ ID NO: 394, (c) a HCDR3 of SEQ ID NO: 395; and a light chain variable region that comprises: (d) a LCDR1 of SEQ ID NO: 409, (e) a LCDR2 of SEQ ID NO: 410; and (f) a LCDR3 of SEQ ID NO: 411;
a heavy chain variable region that comprises: (a) a HCDR1 of SEQ ID NO: 425, (xiv) (b) a HCDR2 of SEQ ID NO: 426, (c) a HCDR3 of SEQ ID NO: 427; and a light chain variable region that comprises: (d) a LCDR1 of SEQ ID NO: 441, (e) a LCDR2 of SEQ ID NO: 442, and (f) a LCDR3 of SEQ ID NO: 443;
(xv) a heavy chain variable region that comprises: (a) a HCDR1 of SEQ ID NO: 457, (b) a HCDR2 of SEQ ID NO: 458, (c) a HCDR3 of SEQ ID NO: 459; and a light chain variable region that comprises: (d) a LCDR1 of SEQ ID NO: 473, (e) a LCDR2 of SEQ ID NO:474, and (f) a LCDR3 of SEQ ID NO: 475; or
(xvi) a heavy chain variable region that comprises: (a) a HCDR1 of SEQ ID NO: 489, (b) a HCDR2 of SEQ ID NO: 490, (c) a HCDR3 of SEQ ID NO:491; and a light chain variable region that comprises: (d) a LCDR1 of SEQ ID NO: 505, (e) a LCDR2 of SEQ ID NO: 506, and (f) a LCDR3 of SEQ ID NO:507.

The antibody wherein one or two amino acids within a CDR have been modified, deleted or substituted.

The present invention of the antibody retains at least 90, 91, 92, 93, 94, 95, 96, 97, 98 or 99% identity over either the variable heavy chain region or the variable light chain region.

The antibody y is a monoclonal antibody, a chimeric antibody, a humanized antibody, a human engineered antibody, a human antibody, a single chain antibody (scFv) or an antibody fragment.

The present invention is directed to an isolated antibody or antigen binding fragment thereof, wherein said antibody or antigen binding fragment thereof comprises:
(i) a heavy chain variable region (vH) that comprises SEQ ID NO: 18, and a light chain variable region (vL) that comprises SEQ ID NO: 34;
(ii) a heavy chain variable region (vH) that comprises SEQ ID NO: 50, and a light chain variable region (vL) that comprises SEQ ID NO: 66;
(iii) a heavy chain variable region (vH) that comprises SEQ ID NO: 82, and a light chain variable region (vL) that comprises SEQ ID NO:98;
(iv) a heavy chain variable region (vH) that comprises SEQ ID NO: 114, and a light chain variable region (vL) that comprises SEQ ID NO:130;
(v) a heavy chain variable region (vH) that comprises SEQ ID NO: 146, and a light chain variable region (vL) that comprises SEQ ID NO:162;
(vi) a heavy chain variable region (vH) that comprises SEQ ID NO:178, and a light chain variable region (vL) that comprises SEQ ID NO:194;
(vii) a heavy chain variable region (vH) that comprises SEQ ID NO:210, and a light chain variable region (vL) that comprises SEQ ID NO:226;
(viii) a heavy chain variable region (vH) that comprises SEQ ID NO:242, and a light chain variable region (vL) that comprises SEQ ID NO:258;
(ix) a heavy chain variable region (vH) that comprises SEQ ID NO:274, and a light chain variable region (vL) that comprises SEQ ID NO:290;
(x) a heavy chain variable region (vH) that comprises SEQ ID NO:306, and a light chain variable region (vL) that comprises SEQ ID NO:322;
(xi) a heavy chain variable region (vH) that comprises SEQ ID NO:338, and a light chain variable region (vL) that comprises SEQ ID NO:354;
(xii) a heavy chain variable region (vH) that comprises SEQ ID NO:370, and a light chain variable region (vL) that comprises SEQ ID NO:386;
(xiii) a heavy chain variable region (vH) that comprises SEQ ID NO:402, and a light chain variable region (vL) that comprises SEQ ID NO:418;
(xiv) a heavy chain variable region (vH) that comprises SEQ ID NO:434, and a light chain variable region (vL) that comprises SEQ ID NO:450;
(xv) a heavy chain variable region (vH) that comprises SEQ ID NO:466, and a light chain variable region (vL) that comprises SEQ ID NO:482; or
(xvi) a heavy chain variable region (vH) that comprises SEQ ID NO:498, and a light chain variable region (vL) that comprises SEQ ID NO: 514.

The antibody or fragment thereof, retains at least 90, 91, 92, 93, 94, 95, 96, 97, 98 or 99% identity over either the variable light or variable heavy region.

The antibody has one, two, three, four or five, but less than 10 amino acids within the variable light or variable heavy region have been modified, deleted or substituted.

The antibody is a monoclonal antibody, a chimeric antibody, a humanized antibody, a human engineered antibody, a human antibody, a single chain antibody (scFv) or an antibody fragment.

The antibody or fragment thereof has reduced glycosylation or no glycosylation or is hypofucosylated.

The present invention is directed to a pharmaceutical composition comprising the antibody or fragment thereof, further comprising a pharmaceutically acceptable carrier.

The pharmaceutical composition comprises the pharmaceutically acceptable carrier contains histidine or a sugar.

The sugar in pharmaceutical composition is sucrose.

The present invention is directed to a pharmaceutical composition comprising a plurality of an antibody or antigen binding fragment wherein at least 0.05%, 0.1%, 0.5%, 1%, 2%, 3%, 5% or more or more of the antibodies in the composition have an .alpha.2,3-linked sialic acid residue.

The present invention is directed to a pharmaceutical composition comprising a plurality of an antibody or antigen binding fragment, wherein none of the antibodies comprise a bisecting GlcNAc.

The pharmaceutical composition comprises the antibody or fragment thereof, wherein the composition is prepared as a lyophilisate.

A method of neutralizing a hepatitis D virus infection comprises administering the composition or pharmaceutical composition via injection or infusion to a patient in need an effective amount of the antibody.

The method comprises the patient in need is diagnosed with hepatitis D viruria or hepatitis D viremia.

The method comprises the patient in need is diagnosed with hepatitis D surface antigen (HBsAg) in the blood or serum.

The present invention is directed to a method of treating or reducing the likelihood of hepatitis D virus associated disorder, comprising administering via injection or infusion to a patient in need an effective amount of the antibody and wherein the disorder is: liver failure, cirrhosis, or hepatocellular carcinoma.

The antibody or composition is further reconstituted prior to injection or infusion.

The antibody or the pharmaceutical composition is administered in combination with another therapeutic agent.

The therapeutic agent is an anti-viral agent.

The anti-viral agent comprises lamivudine, entecavir and tenofovir or alpha-interferon.

The therapeutic agent is an antagonist of immune checkpoint inhibitor.

The antagonist of the immune checkpoint inhibitor is further selected from the group consisting of: PD-1, PD-L1, PD-L2, TIM3, CTLA-4, LAG-3, CEACAM-1, CEACAM-5, VISTA, BTLA, TIGIT, LAIR1, CD160, 2B4 and TGFR.

The antagonist of the immune checkpoint inhibitor is an anti-PD-L1 antibody.

The therapeutic agent is an additional anti-HBsAg antibody.

The antibody or fragment thereof will be for use as a medicament.

The antibody or fragment thereof, will be for use in the neutralization hepatitis D virus infection.

The antibody or fragment thereof, will be for use in the treatment or reducing the likelihood of: liver failure, cirrhosis, and/or hepatocellular carcinoma.

The use according to any of the above, is administered in combination with another therapeutic agent.

The use according to any of the above, comprises that the therapeutic agent is an anti-viral agent including siRNA, antisense, gene editing methods directed against hepatitis B or Hepatitis D targets.

The use according to any of the above, comprises that the anti-viral agent is: lamivudine, entecavir and tenofovir or alpha-interferon.

The use according to any of the above, comprises that the therapeutic agent is an antagonist of immune checkpoint inhibitor.

The use according to any of the above, comprises that the antagonist of the immune checkpoint inhibitor is selected from the group consisting of: PD-1, PD-L1, PD-L2, TIM3, CTLA-4, LAG-3, CEACAM-1, CEACAM-5, VISTA, BTLA, TIGIT, LAIR1, CD160, 2B4 and TGFR.

The use according to any of the above, comprises that the therapeutic agent is an agonist of immunomodulators, wherein the agonist including but not limited to TLR2, TLR3, TLR4, TLR7, TLR8, TLR9 or combination thereof.

The use according to the above, comprises that the antagonist of the immune checkpoint inhibitor is an anti-PD-L1 antibody.

The use according to any of the above, comprises that the therapeutic agent is an additional anti-HBsAg antibody.

The present invention is directed to a nucleic acid that encodes the antibody or antigen binding fragment, avector comprising the nucleic acid, and a host cell comprising the vector.

The present invention is directed to a diagnostic reagent comprising the antibody or antigen binding fragment thereof, which is labeled, wherein the label is selected from the group consisting of a radiolabel, a fluorophore, a chromophore, an imaging agent, and a metal ion.

### Definitions

Unless stated otherwise, the following terms and phrases as used herein are intended to have the following meanings:

The term “antibody” as used herein refers to a polypeptide of the immunoglobulin family that is capable of binding a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antibody” includes, but is not limited to, monoclonal antibodies, human antibodies, humanized antibodies, camelid antibodies, chimeric antibodies, and anti-idiotypic (anti-Id) antibodies (including, e.g., anti-Id antibodies to antibodies of the present disclosure). The antibodies can be of any isotype/class (e.g., IgG, IgE, IgM, IgD, IgA and IgY), or subclass (e.g., IgG1, IgG2, IgG3, IgG4, IgA1 and IgA2).

“Complementarity-determining domains” or “complementarity-determining regions” (“CDRs”) interchangeably refer to the hypervariable regions of VL and VH. The CDRs are the target protein-binding site of the antibody chains that harbors specificity for such target protein. There are three CDRs (CDR1-3, numbered sequentially from the N-terminus) in each human VL or VH, constituting in total about 15-20% of the variable domains. CDRs can be referred to by their region and order. For example, “VHCDR1” or “HCDR1” both refer to the first CDR of the heavy chain variable region. The CDRs are structurally complementary to the epitope of the target protein and are thus directly responsible for the binding specificity. The remaining stretches of the VL or VH, the so-called framework regions, exhibit less variation in amino acid sequence (Kuby, Immunology, 4th ed., Chapter 4. W. H. Freeman & Co., New York, 2000).

The positions of the CDRs and framework regions can be determined using various well known definitions in the art, e.g., Kabat, Chothia, IMGT, and AbM (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Lefranc, M. P., Nucleic Acids Res., 29:207-209 (2001); Al-Lazikani et al., J. Mol. Biol., 273:927-748 (1997)). Definitions of antigen combining sites are also described in the following: Ruiz et al., Nucleic Acids Res., 28:219-221 (2000); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HC CDR1), 50-65 (HC CDR2), and 95-102 (HC CDR3) in a VH, e.g., a mammalian VH, e.g., a human VH; and amino acid residues 24-34 (LC CDR1), 50-56 (LC CDR2), and 89-97 (LC CDR3) in a VL, e.g., a mammalian VL, e.g., a human VL. Under IMGT the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/Domain Gap Align.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2, or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminal domains of the heavy and light chain, respectively.

The term “antigen binding fragment,” as used herein, refers to one or more portions of an antibody that retain the ability to specifically interact with (e.g., by binding, steric hindrance, stabilizing/destabilizing, spatial distribution) an epitope of an antigen. Examples of binding fragments include, but are not limited to, single-chain Fvs (scFv), disulfide-linked Fvs (sdFv), Fab fragments, F(ab′) fragments, a monovalent fragment consisting of the VL, VH, CL and CH1 domains; a F(ab) 2 fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a dAb fragment (Ward et al., Nature 341:544-546, 1989), which consists of a VH domain; and an isolated complementarity determining region (CDR), or other epitope-binding fragments of an antibody.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (“scFv”); see, e.g., Bird et al., Science 242:423-426, 1988; and Huston et al., Proc. Natl. Acad. Sci. 85:5879-5883, 1988). Such single chain antibodies are also intended to be encompassed within the term “antigen binding fragment.” These antigen binding fragments are obtained using conventional techniques known to those of skill in the art, and the fragments are screened for utility in the same manner as are intact antibodies.

Antigen binding fragments can also be incorporated into single domain antibodies, maxibodies, minibodies, nanobodies, intrabodies, diabodies, triabodies, tetrabodies, v-NAR, and bis-scFv (see, e.g., Hollinger and Hudson, Nature Biotechnology 23:1126-1136, 2005). Antigen binding fragments can be grafted into scaffolds based on polypeptides such as fibronectin type III (Fn3) (see U.S. Pat. No. 6,703,199, which describes fibronectin polypeptide monobodies).

Antigen binding fragments can be incorporated into single chain molecules comprising a pair of tandem Fv segments (VH—CH1-VH—CH1) which, together with complementary light chain polypeptides, form a pair of antigen binding regions (Zapata et al., Protein Eng. 8:1057-1062, 1995; and U.S. Pat. No. 5,641,870).

The term “monoclonal antibody” or “monoclonal antibody composition” as used herein refers to polypeptides, including antibodies and antigen binding fragments that have substantially identical amino acid sequence or are derived from the same genetic source. This term also includes preparations of antibody molecules of single molecular composition. A monoclonal antibody composition displays a single binding specificity and affinity for a particular epitope.

The term “human antibody,” as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000).

The human antibodies of the present disclosure can include amino acid residues not encoded by human sequences (e.g., mutations introduced by random or site-specific mutagenesis in vitro or by somatic mutation in vivo, or a conservative substitution to promote stability or manufacturing).

The term “recognize” as used herein refers to an antibody or antigen binding fragment thereof that finds and interacts (e.g., binds) with its epitope, whether that epitope is linear or conformational. The term “epitope” refers to a site on an antigen to which an antibody or antigen binding fragment of the disclosure specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art, for example, x-ray crystallography and 2-dimensional nuclear magnetic resonance (see, e.g., Epitope Mapping Protocols in Methods in Molecular Biology, Vol. 66, G. E. Morris, Ed. (1996)), or electron microscopy. A “paratope” is the part of the antibody which recognizes the epitope of the antigen.

The phrase “specifically binds” or “selectively binds,” when used in the context of describing the interaction between an antigen (e.g., a protein) and an antibody, antibody fragment, or antibody-derived binding agent, refers to a binding reaction that is determinative of the presence of the antigen in a heterogeneous population of proteins and other biologics, e.g., in a biological sample, e.g., a blood, serum, plasma or tissue sample. Thus, under certain designated immunoassay conditions, the antibodies or binding agents with a particular binding specificity bind to a particular antigen at least two times the background and do not substantially bind in a significant amount to other antigens present in the sample. In one aspect, under designated immunoassay conditions, the antibody or binding agent with a particular binding specificity binds to a particular antigen at least ten (10) times the background and does not substantially bind in a significant amount to other antigens present in the sample. Specific binding to an antibody or binding agent under such conditions may require the antibody or agent to have been selected for its specificity for a particular protein. As desired or appropriate, this selection may be achieved by subtracting out antibodies that cross-react with molecules from other species (e.g., mouse or rat) or other subtypes.

Alternatively, in some aspects, antibodies or antibody fragments are selected that cross-react with certain desired molecules.

The term “affinity” as used herein refers to the strength of interaction between antibody and antigen at single antigenic sites. Within each antigenic site, the variable region of the antibody “arm” interacts through weak non-covalent forces with antigen at numerous sites; the more interactions, the stronger the affinity.

The term “isolated antibody” refers to an antibody that is substantially free of other antibodies having different antigenic specificities. An isolated antibody that specifically binds to one antigen may, however, have cross-reactivity to other antigens. Moreover, an isolated antibody may be substantially free of other cellular material and/or chemicals.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other all other known or inferred variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91% 92%, 93%, 94%, 95%, 96%, 97%, 98%, 99%, or 100% sequence identity with the reference variable region nucleic acid or amino acid sequence.

A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically, a specific or selective binding reaction will produce a signal at least twice over the background signal and, more typically, at least 10 to 100 times over the background.

The term “equilibrium dissociation constant (KD, M)” refers to the dissociation rate constant (kd, time-1) divided by the association rate constant (ka, time-1, M-1). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The term “bioavailability” refers to the systemic availability (i.e., blood/plasma levels) of a given amount of drug administered to a patient. Bioavailability is an absolute term that indicates measurement of both the time (rate) and total amount (extent) of drug that reaches the general circulation from an administered dosage form.

As used herein, the phrase “consisting essentially of” refers to the genera or species of active pharmaceutical agents included in a method or composition, as well as any excipients inactive for the intended purpose of the methods or compositions. In some aspects, the phrase “consisting essentially of” expressly excludes the inclusion of one or more additional active agents other than an anti-HBsAg antibody of the present disclosure. In some aspects, the phrase “consisting essentially of” expressly excludes the inclusion of one or more additional active agents other than an anti-HBsAg antibody of the present disclosure and a second co-administered agent.

The term “amino acid” refers to naturally occurring, synthetic, and unnatural amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine Amino acid analogs refer to compounds that have the same basic chemical structure as a naturally occurring amino acid, i.e., an .alpha.-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For example, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)). In some aspects, the term “conservative sequence modifications” are used to refer to amino acid modifications that do not significantly affect or alter the binding characteristics of the antibody containing the amino acid sequence.

The term “optimized” as used herein refers to a nucleotide sequence that has been altered to encode an amino acid sequence using codons that are preferred in the production cell or organism, generally a eukaryotic cell, for example, a yeast cell, a Pichia cell, a fungal cell, a Trichoderma cell, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence.

The terms “percent identical” or “percent identity,” in the context of two or more nucleic acids or polypeptide sequences, refers to the extent to which two or more sequences or subsequences that are the same. Two sequences are “identical” if they have the same sequence of amino acids or nucleotides over the region being compared. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 30 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math. 2:482c (1970), by the homology alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, 2003).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as a basis for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993).

One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, (Comput. Appl. Biosci. 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, (J. Mol. Biol. 48:444-453, 1970), algorithm which has been incorporated into the GAP program in the GCG software package (available from University of South Florida), using either a BLOSUM 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “subject” includes human and non-human animals Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “hepatitis,” “hepatitis B virus” or “HBV” refer to a member of the family Hepadnaviridae, genus Orthohepadnavirus. HBV is a double stranded DNA virus.

### HBsAgHBsAg

The terms “hepatitis D,” “hepatitis D virus” or “HDV” refer to a member of the family Hepadnaviridae, genus Orthohepadnavirus. It is a viral pathogen that requires the presence of the Hepatitis B Virus (HBV) to replicate and cause infection. HDV is considered a defective virus because it cannot produce its own envelope protein and therefore relies on the envelope proteins of HBV to form complete viral particles. It is primarily transmitted through blood-to-blood contact or through sexual contact. Infection with HDV can lead to more severe liver disease compared to HBV infection alone. HDV, is an unusual virus that possesses a single-stranded circular RNA genome. This RNA genome is negative-sense, meaning it serves as a template for the production of complementary positive-sense RNA strands.

“EC50” (half-maximal effective concentration) refers to the concentration of a particular antibody which induces a response halfway (50%) between the baseline control and the maximum possible effect after a specific exposure or treatment time. For example, the EC50 is the concentration of antibody at which virus infection is neutralized by 50%.

“Neutralization” refers to the inhibition of viral infection of a host cell, as demonstrated by the absence of viral gene expression. Without being held to any one theory, mechanisms of neutralization by a particular antibody could include blocking the interaction of viral capsid proteins with cell surface receptors or disruption of any stage of the entry and trafficking process prior to delivery of the viral genome to the nucleus of the host cell.

As used herein, the terms “treat,” “treating,” or “treatment” of any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both.

The phrase “reducing the likelihood” refers to delaying the onset or development or progression of the disease, infection or disorder.

The term “therapeutically acceptable amount” or “therapeutically effective dose” interchangeably refers to an amount sufficient to effect the desired result (i.e., a reduction in tumor size, inhibition of tumor growth, prevention of metastasis, inhibition or prevention of viral, bacterial, fungal or parasitic infection). In some aspects, a therapeutically acceptable amount does not induce or cause undesirable side effects. A therapeutically acceptable amount can be determined by first administering a low dose, and then incrementally increasing that dose until the desired effect is achieved. A “prophylactically effective dosage,” and a “therapeutically effective dosage,” of the molecules of the present disclosure can prevent the onset of, or result in a decrease in severity of, respectively, disease symptoms, including symptoms associated polyoma viral infection.

The term “co-administer” refers to the simultaneous presence of two active agents in the blood of an individual. Active agents that are co-administered can be concurrently or sequentially delivered.

### DESCRIPTIONS OF FIGURES

FIG. 1A: Log 10 HDV RNA over Time of 10 subjects in Cohort F1, Treated with HBsAgBJ 300 mg Once Weekly.

FIG. 1B: Change from baseline of HDV RNA for the 10 subjects in Cohort F1 at different treatment weeks.

FIG. 2A: ALT over Time of 10 subjects in Cohort F1, Treated with HBsAgBJ 300 mg Once Weekly.

FIG. 2B: Change from baseline of ALT for the 10 subjects Cohort F1 at different treatment weeks.

### DETAILED DESCRIPTION

The present disclosure provides for antibodies, antibody fragments (e.g., antigen binding fragments), that bind and neutralize hepatitis D. Furthermore, the present disclosure provides antibodies that have desirable pharmacokinetic characteristics and other desirable attributes, and thus can be used for reducing the likelihood of or treating hepatitis D associate liver failure, liver cirrhosis or hepatocellular cancer. The present disclosure further provides pharmaceutical compositions comprising the antibodies and methods of making and using such pharmaceutical compositions for the prevention and treatment of hepatitis D infection and associated disorders.

### Anti-HBsAg Antibodies

The present disclosure provides for antibodies or antibody fragments (e.g., antigen binding fragments) that specifically bind to HBsAg. Antibodies or antibody fragments (e.g., antigen binding fragments) of the present disclosure include, but are not limited to, the human monoclonal antibodies or fragments thereof, isolated as described, in the Examples below.

The present disclosure in certain aspects provides antibodies or antibody fragments (e.g., antigen binding fragments) that specifically bind to HBsAg, said antibodies or antibody fragments (e.g., antigen binding fragments) comprise a VH domain having an amino acid sequence of SEQ ID NO: 18, 50, 82, 114, 146, 178, 210, 242, 274, 306, 338, 370, 402, 434, 466, or 498 (Table 1). The present disclosure also provides antibodies or antibody fragments (e.g., antigen binding fragments) that specifically bind to HBsAg, said antibodies or antibody fragments (e.g., antigen binding fragments) comprise a VH CDR having an amino acid sequence of any one of the VH CDRs listed in Table 1. In particular aspects, the present disclosure provides antibodies or antibody fragments (e.g., antigen binding fragments) that specifically bind to HBsAg, said antibodies comprising (or alternatively, consist of) one, two, three, or more VH CDRs having an amino acid sequence of any of the VH CDRs listed in Table 1.

The present disclosure provides antibodies or antibody fragments (e.g., antigen binding fragments) that specifically bind to HBsAg, said antibodies or antibody fragments (e.g., antigen binding fragments) comprise a VL domain having an amino acid sequence of SEQ ID NO: 34, 66, 98, 130, 162, 194, 226, 258, 290, 322, 354, 386, 418, 450, 482 or 514 (Table 1). The present disclosure also provides antibodies or antibody fragments (e.g., antigen binding fragments) that specifically bind to HBsAg, said antibodies or antibody fragments (e.g., antigen binding fragments) comprise a VL CDR having an amino acid sequence of any one of the VL CDRs listed in Table 1. In particular, the disclosure provides antibodies or antibody fragments (e.g., antigen binding fragments) that specifically bind to HBsAg, said antibodies or antibody fragments (e.g., antigen binding fragments) comprise (or alternatively, consist of) one, two, three or more VL CDRs having an amino acid sequence of any of the VL CDRs listed in Table 1.

Other antibodies or antibody fragments (e.g., antigen binding fragments) of the present disclosure include amino acids that have been mutated, yet have at least 60, 70, 80, 90 or 95 percent identity in the CDR regions with the CDR regions depicted in the sequences described in Table 1. In some aspects, it includes mutant amino acid sequences wherein no more than 1, 2, 3, 4 or 5 amino acids have been mutated in the CDR regions when compared with the CDR regions depicted in the sequence described in Table 1.

The present disclosure also provides nucleic acid sequences that encode VH, VL, the full length heavy chain, and the full length light chain of the antibodies that specifically bind to HBsAg. Such nucleic acid sequences can be optimized for expression in mammalian cells.

TABLE 1
anti-HBV antibodies

| | | |
|---|---|---|
| SEQ ID NO: 6 | HCDR1 | GFTFDNYAMS |
| (Combined) | | |
| SEQ ID NO: 7 | HCDR2 | SISGSGGSTYYADSVKG |
| (Combined) | | |
| SEQ ID NO: 8 | HCDR3 | SSILSGGHARVYGIDV |
| (Combined) | | |
| SEQ ID NO: 9 (Kabat) | HCDR1 | NYAMS |
| SEQ ID NO: 10 (Kabat) | HCDR2 | SISGSGGSTYYADSVKG |
| SEQ ID NO: 11 (Kabat) | HCDR3 | SSILSGGHARYYGIDV |
| SEQ ID NO: 12 | HCDR1 | GFTFDNY |
| (Chothia) | | |
| SEQ ID NO: 13 | HCDR2 | SGSGGS |
| (Chothia) | | |
| SEQ ID NO: 14 | HCDR3 | SSILSGGHARVYGIDV |
| (Chothia) | | |
| SEQ ID NO: 15 (IMGT) | HCDR1 | GFTFDNYA |
| SEQ ID NO: 16 (IMGT) | HCDR2 | ISGSGGST |
| SEQ ID NO: 17 (IMGT) | HCDR3 | AKSSILSGGHARVYGIDV |
| SEQ ID NO: 18 | VH | EMQVLESGGGLVQPGGSLRLSCAASGFTFDNYAMSW |
| | | VRQYPGKGLEWVSSISGSGGSTYYADSVKGQFTISRD |
| | | NSKNTLYLQMNSLRAEDTAVYYCAKSSILSGGHARV |
| | | YGIDVWGQGTTVTVSS |
| SEQ ID NO: 19 | DNA VH | GAGATGCAGGTCTTGGAATCTGGCGGAGGACTGGT |
| | | TCAACCTGGCGGCTCTCTGAGACTGTCTTGTGCCGC |
| | | CAGCGGCTTCACCTTCGATAACTACCGCCATGTCCTG |
| | | GGTCCGACAGGTGCCAGGCAAAGGACTGGAATGG |
| | | GTGTCCTCTATCAGCGGCTCTGGCGGCAGCACATAT |
| | | TACGCCGATAGCGTGAAGGGCCAGTTCACCATCAG |
| | | CCGGGACAACAGCAAGAACACCCTGTACCTCCAGA |
| | | TGAACAGCCTGAGAGCCGAGGATACCGCCGTGTAC |
| | | TACTGTGCCAAGAGCAGCATTCTGTCTGGCGGCCA |
| | | CGCCAGAGTGTATGGCATTGATGTTTGGGGCCAGG |
| | | GAACCACCGTGACCGTTAGTTCT |
| SEQ ID NO: 20 | Heavy Chain | EMQVLESGGGLVQPGGSLRLSCAASGFTFDNYAMSW |
| | | VRQVPKGLEWVSSISGSGGSTYYADSVKGQFTISRD |
| | | NSKNTLYLQMNSLRAEDTAVYYCAKSSILSGGHARV |
| | | YGIDVWGQGTTVTVSSASTKGPSVFPLAPSSKSTSGG |
| | | TAALGCLVKDYFPEPVTVSWNSGALTSGVHTPPAVL |
| | | QSSGLYSLSSVVTVPSSSLGTQTYICNVNHKPSNTKVD |
| | | KRVEPKSCDKTHTCPPCPAPELLGGPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKC |
| | | KVSNKALPAPIEKTISKAKGQPREPQVYTLPPSREEMT |
| | | KNQVSLTCLVKGFYPSDIAVEWESNGQPENNYKTTPP |
| | | VLDSDGSFFLYSKLTVDKSRWQQGNVFSCSVMHEAL |
| | | HNHYTQKSLSLSPGK |
| SEQ ID NO: 21 | DNA Heavy | GAGATGCAGGTCTTGGAATCTGGCGGAGGACTGGT |
| | Chain | TCAACCTGGCGGCTCTCTGAGACTGTCTTGTGCCGC |
| | | CAGCGGCTTCACCTTCGATAACTACGCCATGTCCTG |
| | | GGTCCGACAGGTGCCAGGCAAAGGACTGGAATGG |
| | | GTGTCCTCTATCAGCGGCTCTGGCGGCAGCACATAT |
| | | TACGCCGATAGCGTGAAGGGCCAGTTCACCATCAG |
| | | CCGGGACAACAGCAAGAACACCCTGTACCTCCAGA |
| | | TGAACAGCCTGAGAGCCGAGGATACCGCCGTGTAC |
| | | TACTGTGCCAAGAGCAGCATTCTGTCTGGCGGCCA |
| | | CGCCAGAGTGTATGGCATTGATGTTTGGGGCCAGG |
| | | GAACCACCGTGACCGTTAGTTCTGCTAGCACCAAG |
| | | GGCCCCAGCGTGTTCCCCCTGGCCCCCAGCAGCAA |
| | | GAGCACCAGCGGCGGCACAGCCGCCCTGGGCTGCC |
| | | TGGTGAAGGACTACTTCCCCGAGCCCGTGACCGTG |
| | | TCCTGGAACAGCGGAGCCCTGACCTCCGGCGTGCA |
| | | CACCTTCCCCGCCGTGCTGCAGAGCAGGCGGCCTGT |
| | | ACAGCCTGTCCAGCGTGGTGACAGTGCCCAGCAGC |
| | | AGCCTGGGCACCCAGACCTACATCTGCAACGTGAA |
| | | CCACAAGCCCAGCAACACCAAGGTGGACAAGAGA |
| | | GTGGAGCCCAAGAGCTGCGACAAGACCCACACATG |
| | | CCCCCCCTGCCCGGCGCCAGAGCTGCTGGGCGGAC |
| | | CCTCCGTGTTCCTGTTCCCCCCCAAGCCCAAGGACA |
| | | CCCTGATGATCAGCAGGACCCCCGAGGTGACCTGC |
| | | GTGGTGGTGGACGTGAGCCACGAGGACCCAGAGGT |
| | | GAAGTTCAACTGGTACGTGGACGGCGTGGAGGTGC |
| | | ACAACGCCAAGACCAAGCCCAGAGAGGAGCAGTA |
| | | CAACAGCACCTACAGGGTGGTGTCCGTGCTGACCG |
| | | TGCTGCACCAGGACTGGCTGAACGGCAAGGAATAC |
| | | AAGTGCAAGGTCTCCAACAAGGCCCTGCCAGCCCC |
| | | CATCGAAAAGACCATCAGCAAGGCCAAGGGCCAG |
| | | CCACGGGAGCCCCAGGTGTACACCCTGCCCCCCTC |
| | | CCGGGAGGAGATGACCAAGAACCAGGTGTCCCTGA |
| | | CCTGTCTGGTGAAGGGCTTCTACCCCAGCGACATC |
| | | GCCGTGGAGTGGGAGAGCAACGGCCAGCCCGAGA |
| | | ACAACTACAAGACCACCCCCCCAGTGCTGGACAGC |
| | | GACGGCAGCTTCTTCCTGTACAGCAAGCTGACCGT |
| | | GGACAAGTCCAGGTGGCAGCAGGGCAACGTGTTCA |
| | | GCTGCAGCGTGATGCACGAGGCCCTGCACAACCAC |
| | | TACACCCAGAAGAGCCTGAGCTTAAGCCCCGGCAA |
| | | G |
| SEQ ID NO: 22 | LCDR1 | GGNNIGSQSVH |
| (Combined) | | |
| SEQ ID NO: 23 | LCDR2 | DDTDRPS |
| (Combined) | | |
| SEQ ID NO: 24 | LCDR3 | QVWDSSSDHVV |
| (Combined) | | |
| SEQ ID NO: 25 (Kabat) | LCDR1 | GGNNIGSQSVH |
| SEQ ID NO: 26 (Kabat) | LCDR2 | DDTDRPS |
| SEQ ID NO: 27 (Kabat) | LCDR3 | QVWDSSSDHVV |
| SEQ ID NO: 28 | LCDR1 | NNIGSQS |
| (Chothia) | | |
| SEQ ID NO: 29 | LCDR2 | DDT |
| (Chothia) | | |
| SEQ ID NO: 30 | LCDR3 | WDSSSDHV |
| (Chothia) | | |
| SEQ ID NO: 31 (IMGT) | LCDR1 | NIGSQS |
| SEQ ID NO: 32 (IMGT) | LCDR2 | DDT |
| SEQ ID NO: 33 (IMGT) | LCDR3 | QVWDSSSDHVV |
| SEQ ID NO: 34 | VL | QSALTQPPSVSVAPGQTARITCGGNNIGSQSVHWYQQ |
| | | KPGQAPILVVYDDTDRPSGIPARFSGSSSGSTATLTIGR |
| | | VEAGDEADYYCQVWDSSSDHVVFGGGTKLTVL |
| SEQ ID NO: 35 | DNA VL | CAGTCTGCCCTGACTCAGCCACCCTCGGTGTCAGTG |
| | | GCCCCAGGACAGACGGCCAGGATTACCTGTGGGGG |
| | | AAACAACATTGGAAGTCAAAGTGTGCACTGGTACC |
| | | AGCAGAAGCCAGGCCAGGCCCCTATACTGGTCGTC |
| | | TATGATGATACCGACCGGCCCTCAGGGATCCCTGC |
| | | GCGATTCTCTGGCTCCAGCTCTGGGAGCACGGCCA |
| | | CCCTGACCATCGGCAGGGTCGAAGCCGGGGATGAG |
| | | GCCGACTATTACTGTCAGGTGTGGGATAGTAGTAG |
| | | TGATCATGTGGTATTCGGCGGAGGGACCAAGCTGA |
| | | CCGTCTTA |
| SEQ ID NO: 36 | Light Chain | QSALTQPPSVSVAPGQTARTTCGGNNIGSQSVHWYQQ |
| | | KPGQAPILVVYDDTDRPSGIPARFSGSSSGSTATLTIGR |
| | | VEAGDEADYYCQVWDSSSDHVVFGGGTKLTVLSQP |
| | | KAAPSVTLFPPSSEELQANKATLVCLISDFYPGAVTVA |
| | | WKADSSPVKAGVETTTPSKQSNNKYAASSYLSLTPEQ |
| | | WKSHRSYSCQVTHEGSTVEKTVAPTECS |
| SEQ ID NO: 37 | DNA Light | CAGTCTGCCCTGACTCAGCCACCCTCGGTGTCAGTG |
| | Chain | GCCCCAGGACAGACGGCCAGGATTACCTGTGGGGG |
| | | AAACAACATTGGAAGTCAAAGTGTGCACTGGTACC |
| | | AGCAGAAGCCAGGCCAGGCCCCTATACTGGTCGTC |
| | | TATGATGATACCGACCGGCCCTCAGGGATCCCTGC |
| | | GCGATTCTCTGGCTCCAGCTCTGGGAGCACGGCCA |
| | | CCCTGACCATCGGCAGGGTCGAAGCCGGGGATGAG |
| | | GCCGACTATTACTGTCAGGTGTGGGATAGTAGTAG |
| | | TGATCATGTGGTATTCGGCGGAGGGACCAAGCTGA |
| | | CCGTCTTAAGTCAGCCCAAGGCTGCCCCCTCGGTCA |
| | | CTCTGTTCCCGCCCTCCTCTGAGGAGCTTCAAGCCA |
| | | ACAAGGCCACACTGGTGTGTCTCATAAGTGACTTCT |
| | | ACCCGGGAGCCGTGACAGTGGCCTGGAAGGCAGAT |
| | | AGCAGCCCCGTCAAGGCGGGAGTGGAGACCACCAC |
| | | ACCCTCCAAACAAAGCAACAACAAGTACGCCGCCA |
| | | GCAGCTACCTGAGCCTGACCCCCGAGCAGTGGAAG |
| | | AGCCACAGAAGCTACAGCTGCCAGGTCACCCACGA |
| | | GGGCAGCACCGTGGAGAAAACCGTGGCCCCCACCG |
| | | AGTGCAGC |
| SEQ ID NO: 38 | HCDR1 | GFTFHNYAMS |
| (Combined) | | |
| SEQ ID NO: 39 | HCDR2 | SISGSGGSTYYADSVKG |
| (Combined) | | |
| SEQ ID NO: 40 | HCDR3 | SSILSGGHARVYGIDV |
| (Combined) | | |
| SEQ ID NO: 41 (Kabat) | HCDR1 | NYAMS |
| SEQ ID NO: 42 (Kabat) | HCDR2 | SISGSGGSTYYADSVKG |
| SEQ ID NO: 43 (Kabat) | HCDR3 | SSILSGGHARVYGIDV |
| SEQ ID NO: 44 | HCDR1 | GFTFHNY |
| (Chothia) | | |
| SEQ ID NO: 45 | HCDR2 | SGSGGS |
| (Chothia) | | |
| SEQ ID NO: 46 | HCDR3 | SSILSGGHARVYGIDV |
| (Chothia) | | |
| SEQ ID NO: 47 (IMGT) | HCDR1 | GFTFHNYA |
| SEQ ID NO: 48 (IMGT) | HCDR2 | ISGSGGST |
| SEQ ID NO: 49 (IMGT) | HCDR3 | AKSSILSGGHARVYGIDV |
| SEQ ID NO: 50 | VH | EMQVLESGGGLVQPGGSLRLSCAASGFTFHNYAMSW |
| | | VRQVPGKGLEWVSSISGSGGSTYYADSVKGQFTISRD |
| | | NSKNTLYLQMNSLRAEDTAVYYCAKSSILSGGHARV |
| | | YGIDVWGQGTTVTVSS |
| SEQ ID NO: 51 | DNA VH | GAGATGCAGGTCTTGGAATCTGGCGGAGGACTGGT |
| | | TCAACCTGGCGGCTCTCTGAGACTGTCTTGTGCCGC |
| | | CAGCGGCTTCACCTTCCATAACTACGCCATGTCCTG |
| | | GGTCCGACAGGTGCCAGGCAAAGGACTGGAATGG |
| | | GTGTCCTCTATCAGCGGCTCTGGCGGCAGCACATAT |
| | | TACGCCGATAGCGTGAAGGGCCAGTTCACCATCAG |
| | | CCGGGACAACAGCAAGAACACCCTGTACCTCCAGA |
| | | TGAACAGCCTGAGAGCCGAGGATACCGCCGTGTAC |
| | | TACTGTGCCAAGAGCAGCATTCTGTCTGGCGGCCA |
| | | CGCCAGAGTGTATGGCATTGATGTTTGGGGCCAGG |
| | | GAACCACCGTGACCGTTAGTTCT |
| SEQ ID NO: 52 | Heavy Chain | EMQVLESGGGLVQPGGSLRLSCAASGFTFHNYAMSW |
| | | VRQVPGKGLEWVSSISGSGGSTYYADSVKGQFTISRD |
| | | NSKNTLYLQMNSLRAEDTAVYYCAKSSILSGGHARV |
| | | YGIDVWGQGTTVTVSSASTKGPSVFPLAPSSKSTSGG |
| | | TAALGGCLVKDYFPEPVTVSWNSGALTSGVHTFPAVL |
| | | QSSGLYSLSSVVTVPSSSLGTQTYICNVNHKPSNTKVD |
| | | KRVEPKSCDKTHTCPPCPAPELLGGPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKC |
| | | KVSNKALPAPIEKTISKAKGQPREPQVYTLPPSREEMT |
| | | KNQVSLTCLVKGFYPSDIAVEWESNGQPENNYKTTPP |
| | | VLDSDGSFPLYSKLTVDKSRWQQGNVFSCSVMHEAL |
| | | HNHYTQKSLSLSPGK |
| SEQ ID NO: 53 | DNA Heavy | GAGATGCAGGTCTTGGAATCTGGCGGAGGACTGGT |
| | Chain | TCAACCTGGCGGCTCTCTGAGACTGTCTTGTGCCGC |
| | | CAGCGGCTTCACCTTCCATAACTACGCCATGTCCTG |
| | | GGTCCGACAGGTGCCAGGCAAAGGACTGGAATGG |
| | | GTGTCCTCTATCAGCGGCTCTGGCGGCAGCACATAT |
| | | TACGCCGATAGCGTGAAGGGCCAGTTCACCATCAG |
| | | CCGGGACAACAGCAAGAACACCCTGTACCTCCAGA |
| | | TGAACAGCCTGAGAGCCGAGGATACCGCCGTGTAC |
| | | TACTGTGCCAAGAGCAGCATTCTGTCTGGCGGCCA |
| | | CGCCAGAGTGTATGGCATTGATGTTTGGGGCCAGG |
| | | GAACCACCGTGACCGTTAGTTCTGCTAGCACCAAG |
| | | GGCCCCAGCGTGTTCCCCCTGGCCCCCAGCAGCAA |
| | | GAGCACCAGCGGCGGCACAGCCGCCCTGGGCTGCC |
| | | TGGTGAAGGACTACTTCCCCGAGCCCGTGACCGTG |
| | | TCCTGGAACAGCGGAGCCCTGACCTCCGGCGTGCA |
| | | CACCTTCCCCGCCGTGCTGCAGAGCAGCGGCCTGT |
| | | ACAGCCTGTCCAGCGTGGTGACAGTGCCCAGCAGC |
| | | AGCCTGGGCACCCAGACCTACATCTGCAACGTGAA |
| | | CCACAAGCCCAGCAACACCAAGGTGGACAAGAGA |
| | | GTGGAGCCCAAGAGCTGCGACAAGACCCACACATG |
| | | CCCCCCCTGCCCGGCGCCAGAGCTGCTGGGCGGAC |
| | | CCTCCGTGTTCCTGTTCCCCCCCAAGCCCAAGGACA |
| | | CCCTGATGATCAGCAGGACCCCCGAGGTGACCTGC |
| | | GTGGTGGTGGACGTGAGCCACGAGGACCCAGAGGT |
| | | GAAGTTCAACTGGTACGTGGACGGCGTGGAGGTGC |
| | | ACAACGCCAAGACCAAGCCCAGAGAGGAGCAGTA |
| | | CAACAGCACCTACAGGGTGGTGTCCGTGCTGACCG |
| | | TGCTGCACCAGGACTGGCTGAACGGCAAGGAATAC |
| | | AAGTGCAAGGTCTCCAACAAGGCCCTGCCAGCCCC |
| | | CATCGAAAAGACCATCAGCAAGGCCAAGGGCCAG |
| | | CCACGGGAGCCCCAGGTGTACACCCTGCCCCCCTC |
| | | CCGGGAGGAGATGACCAAGAACCAGGTGTCCCTGA |
| | | CCTGTCTGGTGAAGGGCTTCTACCCCAGCGACATC |
| | | GCCGTGGAGTGGGAGAGCAACGGCCAGCCCGAGA |
| | | ACAACTACAAGACCACCCCCCCAGTGCTGGACAGC |
| | | GACGGCAGCTTCTTCCTGTACAGCAAGCTGACCGT |
| | | GGACAAGTCCAGGTGGCAGCAGGGCAACGTGTTCA |
| | | GCTGCAGCGTGATGCACGAGGCCCTGCACAACCAC |
| | | TACACCCAGAAGAGCCTGAGCTTAAGCCCCGGCAA |
| | | G |
| SEQ ID NO: 54 | LCDR1 | GGNNIGSQSVH |
| (Combined) | | |
| SEQ ID NO: 55 | LCDR2 | DDTDRPS |
| (Combined) | | |
| SEQ ID NO: 56 | LCDR3 | QVWDSSSDHVV |
| (Combined) | | |
| SEQ ID NO: 57 (Kabat) | LCDR1 | GGNNIGSQSVH |
| SEQ ID NO: 58 (Kabat) | LCDR2 | DDTDRPS |
| SEQ ID NO: 59 (Kabat) | LCDR3 | QVWDSSSDHVV |
| SEQ ID NO: 60 | LCDR1 | NNIGSQS |
| (Chothia) | | |
| SEQ ID NO: 61 | LCDR2 | DDT |
| (Chothia) | | |
| SEQ ID NO: 62 | LCDR3 | WDSSSDHV |
| (Chothia) | | |
| SEQ ID NO: 63 (IMGT) | LCDR1 | NIGSQS |
| SEQ ID NO: 64 (IMGT) | LCDR2 | DDT |
| SEQ ID NO: 65 (IMGT) | LCDR3 | QVWDSSSDHVV |
| SEQ ID NO: 66 | VL | QSALTQPPSVSVAPGQTARITCGGNNIGSQSVHWYQQ |
| | | KPGQAPILVVYDDTDRPSGHPARFSGSSSGSTATLTIGR |
| | | VEAGDEADYYCQVWDSSSDHVVFGGGTKLTVL |
| SEQ ID NO: 67 | DNA VL | CAGTCTGCCCTGACTCAGCCACCCTCGGTGTCAGTG |
| | | GCCCCAGGACAGACGGCCAGGATTACCTGTGGGGG |
| | | AAACAACATTGGAAGTCAAAGTGTGCACTGGTACC |
| | | AGCAGAAGCCAGGCCAGGCCCCTATACTGGTCGTC |
| | | TATGATGATACCGACCGGCCCTCAGGGATCCCTGC |
| | | GCGATTCTCTGGCTCCAGCTCTGGGAGCACGGCCA |
| | | CCCTGACCATCGGCAGGGTCGAAGCCGGGGATGAG |
| | | GCCGACTATTACTGTCAGGTGTGGGATAGTAGTAG |
| | | TGATCATGTGGTATTCGGCGGAGGGACCAAGCTGA |
| | | CCGTCTTA |
| SEQ ID NO: 68 | Light Chain | QSALTQPPSVSVAPGQTARITCGGNNIGSQSVHWYQQ |
| | | KPGQAPILVVYDDTDRPSGHPARFSGSSSGSTATLTIGR |
| | | VEAGDEADYYCQVWDSSSDHVVFGGGTKLTVLSQP |
| | | KAAPSVTLFPPSEELQANKATLVCLISDFYPGAVTVA |
| | | WKADSSPYKAGVETTTPSKQSNNKYAASSYLSLTPEQ |
| | | WKSHRSYSCQVTHEGSTVEKTVAPTECS |
| SEQ ID NO: 69 | DNA Light | CAGTCTGCCCTGACTCAGCCACCCTCGGTGTCAGTG |
| | Chain | GCCCCAGGACAGACGGCCAGGATTACCTGTGGGGG |
| | | AAACAACATTGGAAGTCAAAGTGTGCACTGGTACC |
| | | AGCAGAAGCCAGGCCAGGCCCCTATACTGGTCGTC |
| | | TATGATGATACCGACCGGCCCTCAGGGATCCCTGC |
| | | GCGATTCTCTGGCTCCAGCTCTGGGAGCACGGCCA |
| | | CCCTGACCATCGGCAGGGTCGAAGCCGGGGATGAG |
| | | GCCGACTATTACTGTCAGGTGTGGGATAGTAGTAG |
| | | TGATCATGTGGTATTCGGCGGAGGGACCAAGCTGA |
| | | CCGTCTTAAGTCAGCCCAAGGCTGCCCCCTCGGTCA |
| | | CTCTGTTCCCGCCCTCCTCTGAGGAGCTTCAAGCCA |
| | | ACAAGGCCACACTGGTGTGTCTCATAAGTGACTTCT |
| | | ACCCGGGAGCCGTGACAGTGGCCTGGAAGGCAGAT |
| | | AGCAGCCCCGTCAAGGCGGGAGTGGAGACCACCAC |
| | | ACCCTCCAAACAAAGCAACAACAAGTACGCCGCCA |
| | | GCAGCTACCTGAGCCTGACCCCCGAGCAGTGGAAG |
| | | AGCCACAGAAGCTACAGCTGCCAGGTCACCCACGA |
| | | GGGCAGCACCGTGGAGAAAACCGTGGCCCCCACCG |
| | | AGTGCAGC |
| SEQ ID NO: 70 | HCDR1 | GFTFNNYAMS |
| (Combined) | | |
| SEQ ID NO: 71 | HCDR2 | SISGSGGSTYYADSVKG |
| (Combined) | | |
| SEQ ID NO: 72 | HCDR3 | SSILSGGHARVYGIDV |
| (Combined) | | |
| SEQ ID NO: 73 (Kabat) | HCDR1 | NYAMS |
| SEQ ID NO: 74 (Kabat) | HCDR2 | SISQSGGSTYYADSVKG |
| SEQ ID NO: 75 (Kabat) | HCDR3 | SSILSGGHARVYGIDV |
| SEQ ID NO: 76 | HCDR1 | GFTFNNY |
| (Chothia) | | |
| SEQ ID NO: 77 | HCDR2 | SGSGGS |
| (Chothia) | | |
| SEQ ID NO: 78 | HCDR3 | SSILSGGHARVYGIDV |
| (Chothia) | | |
| SEQ ID NO: 79 (IMGT) | HCDR1 | GFTFNNYA |
| SEQ ID NO: 80 (IMGT) | HCDR2 | ISGSGGST |
| SEQ ID NO: 81 (IMGT) | HCDR3 | AKSSILSGGHARVYGIDV |
| SEQ ID NO: 82 | VH | EMQVLESGGGLVQPGGSLRLSCAASGFTGNNYAMSW |
| | | VRQVPGKGLEWVSSISGSGGSTYYADSVKGQFTISRD |
| | | NSKNTLYLQMNSLRAEDTAVYYCAKSSILSGGHARV |
| | | YGIDVWGQGTTVTVSS |
| SEQ ID NO: 83 | DNA VH | GAAATGCAGGTGTTGGAGTCTGGGGGAGGCCTGGT |
| | | ACAGCCTGGGGGGTCCCTGAGACTCTCCTGTGCAG |
| | | CCTCTGGATTCACCTTTAACAACTATGCCATGAGCT |
| | | GGGTCCGCCAGGTTCCAGGGAAGGGGCTGGAGTGG |
| | | GTCTCAAGTATTAGTGGTAGTGGAGGTAGCACGTA |
| | | CTACGCAGACTCCGTGAAGGGCCAGTTCACCATCT |
| | | CCAGAGACAATTCCAAGAATACGCTGTATCTGCAA |
| | | ATGAACAGCCTGAGAGCCGAGGACACGGCCGTATA |
| | | TTACTGTGCGAAATCCTCTATCTTGAGTGGTGGTCA |
| | | CGCGCGGGTCTACGGCATAGACGTCTGGGGCCAAG |
| | | GGACCACGGTCACCGTCTCCTCA |
| SEQ ID NO: 84 | Heavy Chain | EMQVLESGGGLVQFGGSLRLSCAASGFTGNNYAMSW |
| | | VRQVPGKGLEWVSSISGSGGSTYYADSVKGQFTISRD |
| | | NSKNTLYLQMNSLRAEDTAVYYCAKSSILSGGHARV |
| | | YGIDVWGQGTTVTVSSASTKGPSVFPLAPSSKSTSGG |
| | | TAALGCLVKDYFPEPVTVSWNSGALTSGVHTFPAVL |
| | | QSSGLYSLSSVVTVPSSSLGTQTYICNVNHKPSNTKVD |
| | | KRVEPKSCDKTHTCPPCPAPELLGGPSVFLFPPKPKDT |
| | | LMISRTPEVTCVVVDVSHEDPEVKFNWYVDGVEVHN |
| | | ARTKPREEQYNSTYRVVSVLTVLHQDWLNGKEYKC |
| | | KVSNKALPAPIEKTISKAKGQPREPQVYTLPPSREEMT |
| | | KNQVSLTCLVKGFYPSDIAVEWESNGQPENNYKTTPP |
| | | VLDSDGSFFLYSKLTVDKSRWQQGNVFSCSVMHEAL |
| | | HNHYTQKSLSLSPGK |
| SEQ ID NO: 85 | DNA Heavy | GAAATGCAGGTGTTGGAGTCTGGGGGAGGCCTGGT |
| | Chain | ACAGCCTGGGGGGTCCCTGAGACTCTCCTGTGCAG |
| | | CCTCTGGATTCACCTTTAACAACTATGCCATGAGCT |
| | | GGGTCCGCCAGGTTCCAGGGAAGGGGCTGGAGTGG |
| | | GTCTCAAGTATTAGTGGTAGTGGAGGTAGCACGTA |
| | | CTACGCAGACTCCGTGAAGGGCCAGTTCACCATCT |
| | | CCAGAGACAATTCCAAGAATACGCTGTATCTGCAA |
| | | ATGAACAGCCTGAGAGCCGAGGACACGGCCGTATA |
| | | TTACTGTGCGAAATCCTCTATCTTGAGTGGTGGTCA |
| | | CGCGCGGGTCTACGGCATAGACGTCTGGGGCCAAG |
| | | GGACCACGGTCACCGTCTCCTCAGCCTCCACCAAG |
| | | GGCCCATCGGTCTTCCCCCTGGCACCCTCCTCCAAG |
| | | AGCACCTCTGGGGGCACAGCGGCCCTGGGCTGCCT |
| | | GGTCAAGGACTACTTCCCCGAACCGGTGACGGTGT |
| | | CATGGAACTCAGGCGCCCTGACCAGCGGGCTGCAC |
| | | ACCTTCCCGGCTGTCCTACAGTCCTCAGGACTCTAC |
| | | TCCCTCAGCAGCGTGGTGACCGTGCCCTCCAGCAG |
| | | CTTGGGCACCCAGACCTACATCTGCAACGTGAACC |
| | | ACAAGCCCAGCAACACCAAGGTGGACAAGAGAGT |
| | | GGAGCCCAAGAGCTGCGACAAGACCCACACCTGCC |
| | | CCCCCTGCCCAGCCCCAGAGCTGCTGGGCGGACCC |
| | | TCCGTGTTCCTGTTCCCCCCCAAGCCCAAGGACACC |
| | | CTGATGATCAGCAGGACCCCCGAGGTGACCTGCGT |
| | | GGTGGTGGACGTGAGCCACGAGGACCCAGAGGGTG |
| | | AAGTTCAACTGGTACGTGGACGGCGTGGAGGTGCA |
| | | CAACGCCAAGACCAAGCCCAGAGAGGAGCAGTAC |
| | | AACAGCACCTACAGGGTGGTGTCCGTGCTGACCGT |
| | | GCTGCACCAGGACTGGCTGAACGGCAAGGAATACA |
| | | AGTGCAAGGTCTCCAACAAGGCCCTGCCAGCCCCC |
| | | ATCGAAAAGACCATCAGCAAGGCCAAGGGCCAGC |
| | | CACGGGAGCCCCAGGTGTACACCCTGCCCCCCTCC |
| | | CGGGAGGAGATGACCAAGAACCAGGTGTCCCTGAC |
| | | CTGTCTGGTGAAGGGCTTCTACCCCAGCGACATCG |
| | | CCGTGGAGTGGGAGAGCAACGGCCAGCCCGAGAA |
| | | CAACTACAAGACCACCCCCCCAGTGCTGGACAGCG |
| | | ACGGCAGCTTCTTCCTGTACAGCAAGCTGACCGTG |
| | | GACAAGTCCAGGTGGCAGCAGGGCAACGTGTTCAG |
| | | CTGCAGCGTGATGCACGAGGCCCTGCACAACCACT |
| | | ACACCCAGAAGAGCCTGAGCCTGTCCCCCGGCAAG |
| SEQ ID NO: 86 | LCDR1 | GGNNIGSQSVH |
| (Combined) | | |
| SEQ ID NO: 87 | LCDR2 | DDTDRPS |
| (Combined) | | |
| SEQ ID NO: 88 | LCDR3 | QVWDSSSDHVV |
| (Combined) | | |
| SEQ ID NO: 89 (Kabat) | LCDR1 | GGNNIGSQSVH |
| SEQ ID NO: 90 (Kabat) | LCDR2 | DDTDRPS |
| SEQ ID NO: 91 (Kabat) | LCDR3 | QVWDSSSDHVV |
| SEQ ID NO: 92 | LCDR1 | NNIGSQS |
| (Chothia) | | |
| SEQ ID NO: 93 | LCDR2 | DDT |
| (Chothia) | | |
| SEQ ID NO: 94 | LCDR3 | WDSSSDHV |
| (Chothia) | | |
| SEQ ID NO: 95 (IMGT) | LCDR1 | NIGSQS |
| SEQ ID NO: 96 (IMGT) | LCDR2 | DDT |
| SEQ ID NO: 97 (IMGT) | LCDR3 | QVWDSSSDHVV |
| SEQ ID NO: 98 | VL | QSALTQPPSVSVAPGQTARITCGGNNIGSQSVHWYQQ |
| | | KPGQAPILVVYDDTDRPSGIPARFSGSSSGSTATLTIGR |
| | | VEAGDEADYYCQVWDSSSDHVVFGGGTKLTVL |
| SEQ ID NO: 99 | DNA VL | CAGTCTGCCCTGACTCAGCCACCCTCGGTGTCAGTG |
| | | GCCCCAGGACAGACGGCCAGGATTACCTGTGGGGG |
| | | AAACAACATTGGAAGTCAAAGTGTGCACTGGTACC |
| | | AGCAGAAGCCAGGCCAGGCCCCTATACTGGTCGTC |
| | | TATGATGATACCGACCGGCCCTCAGGGATCCCTGC |
| | | GCGATTCTCTGGCTCCAGCTCTGGGAGCACGGCCA |
| | | CCCTGACCATCGGCAGGGTCGAAGCCGGGGATGAG |
| | | GCCGACTATTACTGTCAGGTGTGGGATAGTAGTAG |
| | | TGATCATGTGGTATTCGGCGGAGGGACCAAGCTGA |
| | | CCGTCTTA |
| SEQ ID NO: 100 | Light Chain | QSALTQPPSVSVAPGQTARITCGGNNIGSQSVHWYQQ |
| | | KPGQAPILVVYDDTDRPSGIPARFSGSSSGSTATLTIGR |
| | | VEAGDEADYYCQVWDSSSDHVVFGGGTKLTVLSQP |
| | | KAAPSVTLFPPSSEELQANKATLVCLISDFYPGAVTVA |
| | | WKADSSPVKAGVETTTPSKQSNNKYAASSYLSLTPEQ |
| | | WHSHRSYSCQVTHEGSTVEKTVAPTECS |
| SEQ ID NO: 101 | DNA Light | CAGTCTGCCCTGACTCAGCCACCCTCGGTGTCAGTG |
| | Chain | GCCCCAGGACAGACGGCCAGGATTACCTGTGGGGG |
| | | AAACAACATTGGAAGTCAAAGTGTGCACTGGTACC |
| | | AGCAGAAGCCAGGCCAGGCCCCTATACTGGTCGTC |
| | | TATGATGATACCGACCGGCCCTCAGGGATCCCTGC |
| | | GCGATTCTCTGGCTCCAGCTCTGGGAGCACGGCCA |
| | | CCCTGACCATCGGCAGGGTCGAAGCCGGGGATGAG |
| | | GCCGACTATTACTGTCAGGTGTGGGATAGTAGTAG |
| | | TGATCATGTGGTATTCGGCGGAGGGACCAAGCTGA |
| | | CCGTCTTAAGTCAGCCCAAGGCTGCCCCCTCGGTCA |
| | | CTCTGTTCCCGCCCTCCTCTGAGGAGCTTCAAGCCA |
| | | ACAAGGCCACACTGGTGTGTCTCATAAGTGACTTCT |
| | | ACCCGGGAGCCGTGACAGTGGCCTGGAAGGCAGAT |
| | | AGCAGCCCCGTCAAGGCGGGAGTGGAGACCACCAC |
| | | ACCCTCCAAACAAAGCAACAACAAGTACGCCGCCA |
| | | GCAGCTACCTGAGCCTGACCCCCGAGCAGTGGAAG |
| | | AGCCACAGAAGCTACAGCTGCCAGGTCACCCACGA |
| | | GGGCAGCACCGTGGAGAAAACCGTGGCCCCCACCG |
| | | AGTGCAGC |
| SEQ ID NO: 102 | HCDR1 | GFTFSPHAMS |
| (Combined) | | |
| SEQ ID NO: 103 | HCDR2 | AISDSGGSTHYADSVKG |
| (Combined) | | |
| SEQ ID NO: 104 | HCDR3 | DDDAWSGYDYWFDY |
| (Combined) | | |
| SEQ ID NO: 105 (Kabat) | HCDR1 | PHAMS |
| SEQ ID NO: 106 (Kabat) | HCDR2 | AISDSGGSTHYADSVKG |
| SEQ ID NO: 107 (Kabat) | HCDR3 | DDDAWSGYDYWFDY |
| SEQ ID NO: 108 | HCDR1 | GFTFSPH |
| (Chothia) | | |
| SEQ ID NO: 109 | HCDR2 | SDSGGS |
| (Chothia) | | |
| SEQ ID NO: 110 | HCDR3 | DDDAWSGYDYWFDY |
| (Chothia) | | |
| SEQ ID NO: 111 (IMGT) | HCDR1 | GFTFSPHA |
| SEQ ID NO: 112 (IMGT) | HCDR2 | ISDSGGST |
| SEQ ID NO: 113 (IMGT) | HCDR3 | ARDDDAWSGYDYWFDY |
| SEQ ID NO: 114 | VH | EVQLLESGGGLVQPGGSLRLSCAASGFTFSPHAMSW |
| | | VRQAPGKGLEWVSAISDSGGSTHYADSVKGRFTISRD |
| | | NSKNTLYLQMNSLRAEDTAVYYCARDDDAWSGYDY |
| | | WFDYWGQGTLVTVSS |
| SEQ ID NO: 115 | DNA VH | GAGGTCCAATTGCTGGAATCTGGCGGAGGACTGGT |
| | | TCAGCCTGGTGGCTCTCTGAGACTGTCTTGTGCCGC |
| | | CAGCGGCTTCACCTTTAGCCCTCATGCCATGTCCTG |
| | | GGTCCGACAGGCTCCTGGAAAAGGACTCGAGTGGGG |
| | | TGTCCGCCATTTCTGATTCTGGCGGCAGCACACACT |
| | | ACGCCGATAGCGTGAAGGGCAGATTCACCATCAGC |
| | | CGGGACAACAGCAAGAACACCCTGTACCTGCAGAT |
| | | GAACAGCCTGAGAGCCGAGGACACAGCCGTGTACT |
| | | ATTGCGCGCGTGACGATGATGCTTGGTCCGGCTAC |
| | | GACTATTGGTTCGATTACTGGGGCCAGGGCACCCT |
| | | GGTCACAGTTAGCTCA |
| SEQ ID NO: 116 | Heavy Chain | EVQLLESGGGLVQPGGSLRLSCAASGFTFSPHAMSW |
| | | VRQAPGKGLEWVSAISDSGGSTHYADSVKGRFTISRD |
| | | NSKNTLYLQMNSLRAEDTAVYYCARDDDAWSGYDY |
| | | WFDYWGQGTLVTVSSASTKGPSVFPLAPSSKSTSGGT |
| | | AALGCLVKDYFPEPVTVSWNSGALTSGVHTPPAVLQ |
| | | SSGLYSLSSVVTVPSSSLGTQTYICNVNHKPSNTKVDK |
| | | RVEPKSCDKTMTCPPCPAPELLGGPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCK |
| | | VSNKALPAPIEKTISKAKGQPREPQVYTLPPSREEMTK |
| | | NQVSLTCLVKGFYPSDIAVEWESNGQPENNYKTTPPV |
| | | LDSDGSFFLYSKLTVDKSRWQQGNVFSCSVMHEALM |
| | | NHYTQKSLSLSPGK |
| SEQ ID NO: 117 | DNA Heavy | GAGGTCCAATTGCTGGAATCTGGCGGAGGACTGGT |
| | Chain | TCAGCCTGGTGGCTCTCTGAGACTGTCTTGTGCCGC |
| | | CAGCGGCTTCACCTTTAGCCCTCATGCCATGTCCTG |
| | | GGTCCGACAGGCTCCTGGAAAAGGACTCGAGTGGGG |
| | | TGTCCGCCATTTCTGATTCTGGCGGCAGCACACACT |
| | | ACGCCGATAGCGTGAAGGGCAGATTCACCATCAGC |
| | | CGGGACAACAGCAAGAACACCCTGTACCTGCAGAT |
| | | GAACAGCCTGAGAGCCGAGGACACAGCCGTGTACT |
| | | ATTGCGCGCGTGACGATGATGCTTGGTCCGGCTAC |
| | | GACTATTGGTTCGATTACTGGGGCCAGGGCACCCT |
| | | GGTCACAGTTAGCTCAGCTAGCACCAAGGGCCCCA |
| | | GCGTGTTCCCCCTGGCCCCCAGCAGCAAGAGCACC |
| | | AGCGGCGGCACAGCCGCCCTGGGCTGCCTGGTGAA |
| | | GGACTACTTCCCCGAGCCCGTGACCGTGTCCTGGA |
| | | ACAGCGGAGCCCTGACCTCCGGCGTGCACACCTTC |
| | | CCCGCCGTGCTGCAGAGCAGCGGCCTGTACAGCCT |
| | | GTCCAGCGTGGTGACAGTGCCCAGCAGCAGCCTGG |
| | | GCACCCAGACCTACATCTGCAACGTGAACCACAAG |
| | | CCCAGCAACACCAAGGTGGACAAGAGAGTGGAGC |
| | | CCAAGAGCTGCGACAAGACCCACACATGCCCCCCC |
| | | TGCCCGGCGCCAGAGCTGCTGGGCGGACCCTCCGT |
| | | GTTCCTGTTCCCCCCCAAGCCCAAGGACACCCTGAT |
| | | GATCAGCAGGACCCCCGAGGTGACCTGCGTGGTGG |
| | | TGGACGTGAGCCACGAGGACCCAGAGGTGAAGTTC |
| | | AACTGGTACGTGGACGGCGTGGAGGTGCACAACGC |
| | | CAAGACCAAGCCCAGAGAGGAGCAGTACAACAGC |
| | | ACCTACAGGGTGGTGTCCGTGCTGACCGTGCTGCA |
| | | CCAGGACTGGCTGAACGGCAAGGAATACAAGTGCA |
| | | AGGTCTCCAACAAGGCCCTGCCAGCCCCCATCGAA |
| | | AAGACCATCAGCAAGGCCAAGGGCCAGCCACGGG |
| | | AGCCCCAGGTGTACACCCTGCCCCCCTCCCGGGAG |
| | | GAGATGACCAAGAACCAGGTGTCCCTGACCTGTCT |
| | | GGTGAAGGGCTTCTACCCCAGCGACATCGCCGTGG |
| | | AGTGGGAGAGCAACGGCCAGCCCGAGAACAACTA |
| | | CAAGACCACCCCCCCAGTGCTGGACAGCGACGGCA |
| | | GCTTCTTCCTGTACAGCAAGCTGACCGTGGACAAG |
| | | TCCAGGTGGCAGCAGGGCAACGTGTTCAGCTGCAG |
| | | CGTGATGCACGAGGCCCTGCACAACCACTACACCC |
| | | AGAAGAGCCTGAGCTTAAGCCCCGGCAAG |
| SEQ ID NO: 118 | LCDR1 | RASQSISPYLN |
| (Combined) | | |
| SEQ ID NO: 119 | LCDR2 | AADSLQS |
| (Combined) | | |
| SEQ ID NO: 120 | LCDR3 | QQSYKIPLT |
| (Combined) | | |
| SEQ ID NO: 121 (Kabat) | LCDR1 | RASQSISPYLN |
| SEQ ID NO: 122 (Kabat) | LCDR2 | AADSLQS |
| SEQ ID NO: 123 (Kabat) | LCDR3 | QQSYKIPLT |
| SEQ ID NO: 124 | LCDR1 | SQSISPY |
| (Chothia) | | |
| SEQ ID NO: 125 | LCDR2 | AAD |
| (Chothia) | | |
| SEQ ID NO: 126 | LCDR3 | SYKIPL |
| (Chothia) | | |
| SEQ ID NO: 127 (IMGT) | LCDR1 | QSISPY |
| SEQ ID NO: 128 (IMGT) | LCDR2 | AAD |
| SEQ ID NO: 129 (IMGT) | LCDR3 | QQSYKIPLT |
| SEQ ID NO: 130 | VL | DIQMTQSPSSLSASVGDRVTITCRASQSISPYLNWYQQ |
| | | KPGKAPKLLIYAADSLQSGVPSRFSGSGSGTDFTLTISS |
| | | LQPEDFATYYCQQSYKIPLTFGQGTKVEIK |
| SEQ ID NO: 131 | DNA VL | GATATCCAGATGACACAGAGCCCTAGCAGCCTGTC |
| | | TGCCTCTGTGGGCGATAGAGTGACCATCACCTGTA |
| | | GAGCCAGCCAGAGCATCAGCCCCTACCTGAATTGG |
| | | TACCAGCAGAAGCCTGGCAAGGCCCCTAAGCTGCT |
| | | GATCTATGCTGCCGACTCTCTGCAGTCTGGCGTGCC |
| | | AAGCAGATTTTCTGGCAGCGGCTCTGGCACCGACT |
| | | TCACCCTGACAATTAGCTCCCTGCAGCCTGAAGACT |
| | | TCGCCACCTACTACTGCCAGCAGAGCTACAAGATC |
| | | CCTCTGACCTTTGGCCAGGGCACCAAGGTGGAAAT |
| | | CAAG |
| SEQ ID NO: 132 | Light Chain | DIQMTQSPSSLSASVGDRVTITCRASQSISPYLNWYQQ |
| | | KPGKAPKLLIYAADSLQSGVPSRFSGSGSGTDFTLTISS |
| | | LQPEDFATYYCQQSYKIPLTFGQGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |
| SEQ ID NO: 133 | DNA Light | GATATCCAGATGACACAGAGCCCTAGCAGCCTGTC |
| | Chain | TGCCTCTGTGGGCGATAGAGTGACCATCACCTGTA |
| | | GAGCCAGCCAGAGCATCAGCCCCTACCTGAATTGG |
| | | TACCAGCAGAAGCCTGGCAAGGCCCCTAAGCTGCT |
| | | GATCTATGCTGCCGACTCTCTGCAGTCTGGCGTGCC |
| | | AAGCAGATTTTCTGGCAGCGGCTCTGGCACCGACT |
| | | TCACCCTGACAATTAGCTCCCTGCAGCCTGAAGACT |
| | | TCGCCACCTACTACTGCCAGCAGAGCTACAAGATC |
| | | CCTCTGACCTTTGGCCAGGGCACCAAGGTGGAAAT |
| | | CAAGCGTACGGTGGCCGCTCCCAGCGTGTTCATCTT |
| | | CCCCCCCAGCGACGAGCAGCTGAAGAGCGGCACCG |
| | | CCAGCGTGGTGTGCCTGCTGAACAACTTCTACCCCC |
| | | GGGAGGCCAAGGTGCAGTGGAAGGTGGACAACGC |
| | | CCTGCAGAGCGGCAACAGCCAGGAGAGCGTCACCG |
| | | AGCAGGACAGCAAGGACTCCACCTACAGCCTGAGC |
| | | AGCACCCTGACCCTGAGCAAGGCCGACTACGAGAA |
| | | GCATAAGGTGTACGCCTGCGAGGTGACCCACCAGG |
| | | GCCTGTCCAGCCCCGTGACCAAGAGCTTCAACAGG |
| | | GGCGAGTGC |
| SEQ ID NO: 134 | HCDR1 | GFTFSPHAMS |
| (Combined) | | |
| SEQ ID NO: 135 | HCDR2 | AISDSGGSTHYADSVKG |
| (Combined) | | |
| SEQ ID NO: 136 | HCDR3 | DDDGWSGYDYWFDY |
| (Combined) | | |
| SEQ ID NO: 137 (Kabat) | HCDR1 | PHAMS |
| SEQ ID NO: 138 (Kabat) | HCDR2 | AISDSGGSTHYADSVKG |
| SEQ ID NO: 139 (Kabat) | HCDR3 | DDDGWSGYDYWFDY |
| SEQ ID NO: 140 | HCDR1 | GFTFSPH |
| (Chothia) | | |
| SEQ ID NO: 141 | HCDR2 | SDSGGS |
| (Chothia) | | |
| SEQ ID NO: 142 | HCDR3 | DDDGWSGYDYWFDY |
| (Chothia) | | |
| SEQ ID NO: 143 (IMGT) | HCDR1 | GTFTSPHA |
| SEQ ID NO: 144 (IMGT) | HCDR2 | ISDSGGST |
| SEQ ID NO: 145 (IMGT) | HCDR3 | ARDDDGWSGYDYWPFY |
| SEQ ID NO: 146 | VH | EVQLLESGGGLVQPGGSLRLSCAASGPTFSPHAMSW |
| | | VRQAPGKGLEWVSAISDSGGSTHYAUSVKGRFTISRD |
| | | NSKNTLYLQMNSLRAEDTAVYYCARDDDGWSGYDY |
| | | WFDYWGQGTLVTVSS |
| SEQ ID NO: 147 | DNA VH | GAGGTCCAATTGCTGGAATCTGGCGGAGGACTGGT |
| | | TCAGCCTGGTGGCTCTCTGAGACTGTCTTGTGCCGC |
| | | CAGCGGCTTCACATTCAGCCCTCATGCCATGTCCTG |
| | | GGTCCGACAGGCTCCTGGAAAAGGACTCGAGTGGG |
| | | TGTCCGCCATTTCTGATTCTGGCGGCAGCACACACT |
| | | ACGCCGATAGCGTGAAGGGCAGATTCACCATCAGC |
| | | CGGGACAACAGCAAGAACACCCTGTACCTGCAGAT |
| | | GAACAGCCTGAGAGCCGAGGACACAGCCGTGTACT |
| | | ATTGCGCGCGTGACGATGATGGATGGTCCGGCTAC |
| | | GACTATTGGTTCGATTACTGGGGCCAGGGCACCCT |
| | | GGTCACAGTTAGCTCA |
| SEQ ID NO: 148 | Heavy Chain | EVQLLESGGGLVQPGGSLRLSCAASGPTFSPHAMSW |
| | | VRQAPGKGLEWVSAISDSGGSTHYAUSVKGRFTISRD |
| | | NSKNTLYLQMNSLRAEDTAVYYCARDDDGWSGYDY |
| | | WFDYWGQGTLVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVDK |
| | | RVEPKSCDKTHTCPPCPAPELLGGPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCK |
| | | VSNKALPAPIEKTISKAKGQPREPQVYTLPPSREEMTK |
| | | NQSDGSFFLYSKLTVDKSRWQGNVFSCSVMHEALH |
| | | NHYTQKSLSLSPGK |
| SEQ ID NO: 149 | DNA Heavy | GAGGTCCAATTGCTGGAATCTGGCGGAGGACTGGT |
| | Chain | TCAGCCTGGTGGCTCTCTGAGACTGTCTTGTGCCGC |
| | | CAGCGGCTTCACATTCAGCCCTCATGCCATGTCCTG |
| | | GGTCCGACAGGCTCCTGGAAAAGGACTCGAGTGGG |
| | | TGTCCGCCATTTCTGATTCTGGCGGCAGCACACACT |
| | | ACGCCGATAGCGTGAAGGGCAGATTCACCATCAGC |
| | | CGGGACAACAGCAAGAACACCCTGTACCTGCAGAT |
| | | GAACAGCCTGAGAGCCGAGGACACAGCCGTGTACT |
| | | ATTGCGCGCGTGACGATGATGGATGGTCCGGCTAC |
| | | GACTATTGGTTCGATTACTGGGGCCAGGGCACCCT |
| | | GGTCACAGTTAGCTCAGCTAGCACCAAGGGCCCCA |
| | | GCGTGTTCCCCCTGGCCCCCAGCAGCAAGAGCACC |
| | | AGCGGCGGCACAGCCGCCCTGGGCTGCCTGGTGAA |
| | | GGACTACTTCCCCGAGCCCGTGACCGTGTCCTGGA |
| | | ACAGCGGAGCCCTGACCTCCGGCGTGCACACCTTC |
| | | CCCGCCGTGCTGCAGAGCAGCGGCCTGTACAGCCT |
| | | GTCCAGCGTGGTGACAGTGCCCAGCAGCAGCCTGG |
| | | GCACCCAGACCTACATCTGCAACGTGAACCACAAG |
| | | CCCAGCAACACCAAGGTGGACAAGAGAGTGGAGC |
| | | CCCAGCAACACCAAGGTGGACAAGAGAGTGGAGC |
| | | TGCCCGGCGCCAGAGCTGCTGGGCGGACCCTCCGT |
| | | GTTCCTGTTCCCCCCCAAGCCCAAGGACACCCTGAT |
| | | GATCAGCAGGACCCCCGAGGTGACCTGCGTGGTGG |
| | | TGGACGTGAGCCACGAGGACCCAGAGGTGAAGTTC |
| | | AACTGGTACGTGGACGGCGTGGAGGTGCACAACGC |
| | | CAAGACCAAGCCCAGAGAGGAGCAGTACAACAGC |
| | | ACCTACAGGGTGGTGTCCGTGCTGACCGTGCTGCA |
| | | CCAGGACTGGCTGAACGGCAAGGAATACAAGTGCA |
| | | AGGTCTCCAACAAGGCCCTGCCAGCCCCCATCGAA |
| | | AAGACCATCAGCAAGGCCAAGGGCCAGCCACGGG |
| | | AGCCCCAGGTGTACACCCTGCCCCCCTCCCGGGAG |
| | | GAGATGACCAAGAACCAGGTGTCCCTGACCTGTCT |
| | | GGTGAAGGGCTTCTACCCCAGCGACATCGCCGTGG |
| | | AGTGGGAGAGCAACGGCCAGCCCGAGAACAACTA |
| | | CAAGACCACCCCCCAGTGCTGGACAGCGACGGCA |
| | | GCTTCTTCCTGTACAGCAAGCTGACCGTGGACAAG |
| | | TCCAGGTGGCAGCAGGGCAACGTGTTCAGCTGCAG |
| | | CGTGATGCACGAGGCCCTGCACAACCACTACACCC |
| | | AGAAGAGCCTGAGCTTAAGCCCCGGCAAG |
| SEQ ID NO: 150 | LCDR1 | RASQSISPYLN |
| (Combined) | | |
| SEQ ID NO: 151 | LCDR2 | AADSLQS |
| (Combined) | | |
| SEQ ID NO: 152 | LCDR3 | QQSYKIPLT |
| (Combined) | | |
| SEQ ID NO: 153 (Kabat) | LCDR1 | RASQSISPYLN |
| SEQ ID NO: 154 (Kabat) | LCDR2 | AADSLQS |
| SEQ ID NO: 155 (Kabat) | LCDR3 | QQSYKIPLT |
| SEQ ID NO: 156 | LCDR1 | SQSISPY |
| (Chothia) | | |
| SEQ ID NO: 157 | LCDR2 | AAD |
| (Chothia) | | |
| SEQ ID NO: 158 | LCDR3 | SYKIPL |
| (Chothia) | | |
| SEQ ID NO: 159 (IMGT) | LCDR1 | QSISPY |
| SEQ ID NO: 160 (IMGT) | LCDR2 | AAD |
| SEQ ID NO: 161 (IMGT) | LCDR3 | QQSYKIPLT |
| SEQ ID NO: 162 | VL | DIQMTQSPSSLSASVGDRVTITCRASQSISPYLNWYQQ |
| | | KPGKAPKLLIYAADSLQSGVPSRFSSGSGTDFTLTISS |
| | | LQPEDFATYYCQQSYKIPLTFGQGTKVEIK |
| SEQ ID NO: 163 | DNA VL | GATATCCAGATGACACAGAGCCCTAGCAGCCTGTC |
| | | TGCCTCTGTGGGCGATAGAGTGACCATCACCTGTA |
| | | GAGCCAGCCAGAGCATCAGCCCCTACCTGAATTGG |
| | | TACCAGCAGAAGCCTGGCAAGGCCCCTAAGCTGCT |
| | | GATCTATGCTGCCGACTCTCTGCAGTCTGGCGTGCC |
| | | AAGCAGATTTTCTGGCAGCGGCTCTGGCACCGACT |
| | | TCACCCTGACAATTAGCTCCCTGCAGCCTGAAGACT |
| | | TCGCCACCTACTACTGCCAGCAGAGCTACAAGATC |
| | | CCTCTGACCTTTGGCCAGGGCACCAAGGTGGAAAT |
| | | CAAG |
| SEQ ID NO: 164 | Light Chain | DIQMTQSPSSLSASVGDRVTITCRASQSISPYLNWYQQ |
| | | KPGKAPKLLIYAADSLQSGVPSRFSSGSGTDFTLTISS |
| | | LQPEDFATYYCQQSYKIPLTFGQGTKVEIKRTVAAPS |
| | | VFTFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |
| SEQ ID NO: 165 | DNA Light | GATATCCAGATGACACAGAGCCCTAGCAGCCTGTC |
| | Chain | TGCCTCTGTGGGCGATAGAGTGACCATCACCTGTA |
| | | GAGCCAGCCAGAGCATCAGCCCCTACCTGAATTGG |
| | | TACCAGCAGAAGCCTGGCAAGGCCCCTAAGCTGCT |
| | | GATCTATGCTGCCGACTCTCTGCAGTCTGGCGTGCC |
| | | AAGCAGATTTTCTGGCAGCGGCTCTGGCACCGACT |
| | | TCACCCTGACAATTAGCTCCCTGCAGCCTGAAGACT |
| | | TCGCCACCTACTACTGCCAGCAGAGCTACAAGATC |
| | | CCTCTGACCTTTGGCCAGGGCACCAAGGTGGAAAT |
| | | CAAGCGTACGGTGGCCGCTCCCAGCGTGTTCATCTT |
| | | CCCCCCCAGCGACGAGCAGCTGAAGAGCGGCACCG |
| | | CCAGCGTGGTGTGCCTGCTGAACAACTTCTACCCCC |
| | | GGGAGGCCAAGGTGCAGTGGAAGGTGGACAACGC |
| | | CCTGCAGAGCGGCAACAGCCAGGAGAGCGTCACCG |
| | | AGCAGGACAGCAAGGACTCCACCTACAGCCTGAGC |
| | | AGCACCCTGACCCTGAGCAAGGCCGACTACGAGAA |
| | | GCATAAGGTGTACGCCTGCGAGGTGACCCACCAGG |
| | | GCCTGTCCAGCCCCGTGACCAAGAGCTTCAACAGG |
| | | GGCGAGTGC |
| SEQ ID NO: 166 | HCDR1 | GFTFNRYGMH |
| (Combined) | | |
| SEQ ID NO: 167 | HCDR2 | GIWHDGSHKYYADSLRG |
| (Combined) | | |
| SEQ ID NO: 168 | HCDR3 | QTNRGRLDDAFDI |
| (Combined) | | |
| SEQ ID NO: 169 (Kabat) | HCDR1 | RYGMH |
| SEQ ID NO: 170 (Kabat) | HCDR2 | GIWHDGSHKYYADSLRG |
| SEQ ID NO: 171 (Kabat) | HCDR3 | QTNRGRLDDAFDI |
| SEQ ID NO: 172 | HCDR1 | GFTFNRY |
| (Chothia) | | |
| SEQ ID NO: 173 | HCDR2 | WHDGSH |
| (Chothia) | | |
| SEQ ID NO: 174 | HCDR3 | QTNRGRLDDAFDI |
| (Chothia) | | |
| SEQ ID NO: 175 (IMGT) | HCDR1 | GFTFNRYG |
| SEQ ID NO: 176 (IMGT) | HCDR2 | IWHDGSHK |
| SEQ ID NO: 177 (IMGT) | HCDR3 | VRQTNRGRLDDAFDI |
| SEQ ID NO: 178 | VH | QVQLVESGGGVVQPGRSLRLSCAASGFTFNRYGMH |
| | | WVRQAPGKGLEWVAGIWHDGSHKYYADSLRGRFTI |
| | | SRDNAKNTLDLQLNRLRAEDTSVYYCVRQTNRGRLD |
| | | DAFDIWGQGTMVTVSS |
| SEQ ID NO: 179 | DNA VH | CAGGTGCAGCTGGTGGAGTCTGGGGGAGGCGTGGT |
| | | CCAGCCTGGGAGGTCCCTGAGACTCTCCTGTGCAG |
| | | CGTCAGGATTCACATTCAATAGATATGGCATGCAC |
| | | TGGGTCCGCCAGGCTCCAGGCAAGGGGTTGGAGTG |
| | | GGTGGCTGGTATATGGCATGATGGAAGTCATAAAT |
| | | ACTATGCAGACTCTCTGAGGGGCCGATTCACCATCT |
| | | CCAGAGACAATGCCAAGAACACGCTGGATCTGCAA |
| | | TTGAACAGGCTGAGAGCCGAAGACACGTCTGTGTA |
| | | TTATTGTGTGAGGCAAACCAACAGGGGACGTCTCG |
| | | ATGATGCTTTTGACATCTGGGGCCAAGGGACAATG |
| | | GTCACCGTTAGCTCA |
| SEQ ID NO: 180 | Heavy Chain | QVQLVESGGGVVQPGRSLRLSCAASGFTFNRYGMH |
| | | WVRQAPGKGLEWVAGIWHDGSHKYYADSLRGRFTI |
| | | SRDNAKNTLDLQLNRLRAEDTSVYYCVRQTNRGRLD |
| | | DAFDIWGQGTMVTVSSASTKGPSVFPLAPSSKSTSGG |
| | | TAALGCLVKDYFPEPVTVSWNSGALTSGVHTFPAVL |
| | | QSSGLYSLSSVVTVPSSSLGTQTYICNVNHKPSNTKVD |
| | | KRVEPKSCDKTHTCPPCPAPELLGGPSVFLPPPKPKDT |
| | | LMISRTPEVTCVVVDVSHEDPEVKFNWYVDGVEVHN |
| | | AKTKPREEQYNSTYRVVSVLTVLHQDWLNGKEYKC |
| | | KVSNKALPAFIEKTISKAKGQPREPQVYTLPPSREEMT |
| | | KNQVSLTCLVKGFYPSDIAVEWESNGQPENNYKTTPP |
| | | VLDSDGSFFLYSKLTVDKSRWQQGNVFSCSVMHEAL |
| | | HNHYTQKSLSLSPGK |
| SEQ ID NO: 181 | DNA Heavy | CAGGTGCAGCTGGTGGAGTCTGGGGGAGGCGTGGT |
| | Chain | CCAGCCTGGGAGGTCCCTGAGACTCTCCTGTGCAG |
| | | CGTCAGGATTCACATTCAATAGATATGGCATGCAC |
| | | TGGGTCCGCCAGGCTCCAGGCAAGGGGTTGGAGTG |
| | | GGTGGCTGGTATATGGCATGATGGAAGTCATAAAT |
| | | ACTATGCAGACTCTCTGAGGGGCCGATTCACCATCT |
| | | CCAGAGACAATGCCAAGAACACGCTGGATCTGCAA |
| | | TTGAACAGGCTGAGAGCCGAAGACACGTCTGTGTA |
| | | TTATTGTGTGAGGCAAACCAACAGGGGACGTCTCG |
| | | ATGATGCTTTTGACATCTGGGGCCAAGGGACAATG |
| | | GTCACCGTTAGCTCAGCTAGCACCAAGGGCCCCAG |
| | | CGTGTTCCCCCTGGCCCCCAGCAGCAAGAGCACCA |
| | | GCGGCGGCACAGCCGCCCTGGGCTGCCTGGTGAAG |
| | | GACTACTCCCCGAGCCCGTGACCGTGTCCTGGAA |
| | | CAGCGGAGCCCTGACCTCCGGCGTGCACACCTTCC |
| | | CCGCCGTGCTGCAGAGCAGCGGCCTGTACAGCCTG |
| | | TCCAGCGTGGTGACAGTGCCCAGCAGCAGCCTGGG |
| | | CACCCAGACCTACATCTGCAACGTGAACCACAAGC |
| | | CCAGCAACACCAAGGTGGACAAGAGAGTGGAGCC |
| | | CAAGAGCTGCGACAAGACCCACACCTGCCCCCCCT |
| | | GCCCAGCCCCAGAGCTGCTGGGCGGACCCTCCGTG |
| | | TTCCTGTTCCCCCCCAAGCCCAAGGACACCCTGATG |
| | | ATCAGCAGGACCCCCGAGGTGACCTGCGTGGTGGT |
| | | GGACGTGAGCCACGAGGACCCAGAGGTGAAGTTCA |
| | | ACTGGTACGTGGACGGCGTGGAGGTGCACAACGCC |
| | | AAGACCAAGCCCAGAGAGGAGCAGTACAACAGCA |
| | | CCTACAGGGTGGTGTCCGTGCTGACCGTGCTGCAC |
| | | CAGGACTGGCTGAACGGCAAGGAATACAAGTGCA |
| | | AGGTCTCCAACAAGGCCCTGCCAGCCCCCATCGAA |
| | | AAGACCATCAGCAAGGCCAAGGGCCAGCCACGGG |
| | | AGCCCCAGGTGTACACCCTGCCCCCCTGACCTGTCT |
| | | GAGATGACCAAGAACCAGGTGTCCCTGACCTGTCT |
| | | GGTGAAGGGCTTCTACCCCAGCGACATCGCCGTGG |
| | | AGTGGGAGAGCAACGGCCAGCCCGAGAACAACTA |
| | | CAAGACCACCCCCCCAGTGCTGGACAGCGACGGCA |
| | | GCTTCTTCCTGTACAGCAAGCTGACCGTGGACAAG |
| | | TCCAGGTGGCAGCAGGGCAACGTGTTCAGCTGCAG |
| | | CGTGATGCACGAGGCCCTGCACAACCACTACACCC |
| | | AGAAGAGCCTGAGCCTGTCCCCCGGCAAG |
| SEQ ID NO: 182 | LCDR1 | RASQTISSYLN |
| (Combined) | | |
| SEQ ID NO: 813 | LCDR2 | AASTLQS |
| (Combined) | | |
| SEQ ID NO: 184 | LCDR3 | QQNYDTLWT |
| (Combined) | | |
| SEQ ID NO: 185 (Kabat) | LCDR1 | RASQTISSYLN |
| SEQ ID NO: 186 (Kabat) | LCDR2 | AASTLQS |
| SEQ ID NO: 187 (Kabat) | LCDR3 | QQNYDTLWT |
| SEQ ID NO: 188 | LCDR1 | SQTISSY |
| (Chothia) | | |
| SEQ ID NO: 189 | LCDR2 | AAS |
| (Chothia) | | |
| SEQ ID NO: 190 | LCDR3 | NYDTLW |
| (Chothia) | | |
| SEQ ID NO: 191 (IMGT) | LCDR1 | QTISSY |
| SEQ ID NO: 192 (IMGT) | LCDR2 | AAS |
| SEQ ID NO: 193 (IMGT) | LCDR3 | QQNYDTLWT |
| SEQ ID NO: 194 | VL | DIQMTQSPSSLSAAVGDRVTISCRASQTISSYLNWYQ |
| | | QKPGEAPKLLIYAASTLQSGVPSRFGGSGSGTDFTLTI |
| | | SSLQPEDSATTYCQQNYDTLWTFGQGTKVEIK |
| SEQ ID NO: 195 | DNA VL | GATATCCAGATGACCCAGTCTCCATCCTCCCTGTCT |
| | | GCAGCTGTAGGAGACAGAGTCACCATCTCTTGCCG |
| | | GGCAAGTCAGACCATTAGTAGTTATTTAAATTGGT |
| | | ATCAGCAGAAACCAGGGGAAGCCCCTAAGCTCCTG |
| | | ATCTATGCTGCCTCCACCTTGCAAAGTGGGGTCCCT |
| | | TCAAGGTTCGGTGGCAGTGGATCTGGGACAGATTT |
| | | CACTCTCACCATCAGCAGTCTGCAACCTGAAGATTC |
| | | TGCAACTTACTACTGTCAACAGAATTACGATACTTT |
| | | GTGGACGTTCGGCCAAGGGACCAAGGTGGAAATCA |
| | | AA |
| SEQ ID NO: 196 | Light Chain | DIQMTQSPSSLSAAVGDRVTISCRASQTISSYLNWYQ |
| | | QKPGEAPKLLIYAASTLQSGVPSRFGGSGSGTDFTLTI |
| | | SSLQPEDSATTYCQQNYDTLWTFGQGTKVEIKRTVA |
| | | APSVFTFPPSDEQLKSGTASVVCLLNNFYPREAKVQW |
| | | KVDNALQSGNSQESVTEQDSKDSTYSLSSTLTLSKAD |
| | | YEKHKVYACEVTHQGLSSPVTKSFNRGEC |
| SEQ ID NO: 197 | DNA Light | GATATCCAGATGACCCAGTCTCCATCCTCCCTGTCT |
| | Chain | GCAGCTGTAGGAGACAGAGTCACCATCTCTTGCCG |
| | | GGCAAGTCAGACCATTAGTAGTTATTTAAATTGGT |
| | | ATCAGCAGAAACCAGGGGAAGCCCCTAAGCTCCTG |
| | | ATCTATGCTGCCTCCACCTTGCAAAGTGGGGTCCCT |
| | | TCAAGGTTCGGTGGCAGTGGATCTGGGACAGATTT |
| | | CACTCTCACCATCAGCAGTCTGCAACCTGAAGATTC |
| | | TGCAACTTACTACTGTCAACAGAATTACGATACTTT |
| | | GTGGACGTTCGGCCAAGGGACCAAGGTGGAAATCA |
| | | AACGTACGGTGGCCGCTCCCAGCGTGTTCATCTTCC |
| | | CCCCCAGCGACGAGCAGCTGAAGAGCGGCACCGCC |
| | | AGCGTGGTGTGCCTGCTGAACAACTTCTACCCCCG |
| | | GGAGGCCAAGGTGCAGTGGAAGGTGGACAACGCC |
| | | CTGCAGAGCGGCAACAGCCAGGAGAGCGTCACCG |
| | | AGCAGGACAGCAAGGACTCCACCTACAGCCTGAGC |
| | | AGCACCCTGACCCTGAGCAAGGCCGACTACGAGAA |
| | | GCATAAGGTGTACGCCTGCGAGGTGACCCACCAGG |
| | | GCTGTCCAGCCCCGTGACCAAGAGCTTCAACAGG |
| | | GGCGAGTGC |
| SEQ ID NO: 198 | HCDR1 | GFTFDRYGMH |
| (Combined) | | |
| SEQ ID NO: 199 | HCDR2 | GIWHEGSHKYYADSLRG |
| (Combined) | | |
| SEQ ID NO: 200 | HCDR3 | QTNRGRLDDAFDI |
| (Combined) | | |
| SEQ ID NO: 201 (Kabat) | HCDR1 | RYGMH |
| SEQ ID NO: 202 (Kabat) | HCDR2 | GIWHEGSHKYYADSLRG |
| SEQ ID NO: 203 (Kabat) | HCDR3 | QTNRGRLDDAFDI |
| SEQ ID NO: 204 | HCDR1 | GFTFDRY |
| (Chothia) | | |
| SEQ ID NO: 205 | HCDR2 | WHEGSM |
| (Chothia) | | |
| SEQ ID NO: 206 | HCDR3 | QTNRGRLDDAFDI |
| (Chothia) | | |
| SEQ ID NO: 207 (IMGT) | HCDR1 | GFTFDRYG |
| SEQ ID NO: 208 (IMGT) | HCDR2 | IWHEGSHK |
| SEQ ID NO: 209 (IMGT) | HCDR3 | VRQTNRGRLDDAFDI |
| SEQ ID NO: 210 | VH | QVQLVESGGGVVQPGRSLRLSCAASGFTFDRYGMH |
| | | WVRQAPGKGLEWVAGIWHEGSHKYYADSLRGRFTIS |
| | | RDNAKNTLDLQLNRLRAEDTSVYYCVRQTNRGRLD |
| | | DAFDIWGQGTMVTVSS |
| SEQ ID NO: 211 | DNA VH | CAGGTGCAGCTGGTTGAATCTGGTGGCGGAGTGGT |
| | | GCAGCCTGGCAGATCTCTGAGACTGTCTTGTGCCGC |
| | | CAGCGGCTTCACCTTCGACAGATATGGCATGCACT |
| | | GGGTCCGACAGGCCCCTGGAAAAGGACTTGAATGG |
| | | GTGGCCGGAATCTGGCACGAAGGCAGCCACAAGTA |
| | | CTACGCCGATAGCCTGAGAGGCCGGTTCACCATC |
| | | GCAGAGACAACGCCAAGAACACCCTGGACCTCCAG |
| | | CTGAACAGACTGAGAGCCGAGGATACCAGCGTGTA |
| | | CTACTGCGTGCGGCAGACCAACAGAGGCAGACTGG |
| | | ACGATGCCTTCGATATCTGGGGCCAAGGGACAATG |
| | | GTCACCGTTAGCTCA |
| SEQ ID NO: 212 | Heavy Chain | QVQLVESGGGVVQPGRSLRLSCAASGFTFDRYGMH |
| | | WVRQAPGKGLEWVAGIWHEGSHKYYADSLRGRFTIS |
| | | RDNAKNTLDLQLNRLRAEDTSVYYCVRQTNRGRLD |
| | | DAFDIWGQGTMVTVSSASTKGPSVFPLAPSSKSTSGG |
| | | TAALGCLVKDYFPEPVTVSWNSGALTSGVHTFPAVL |
| | | QSSGLYSLSSVVTVPSSSLGTQTYICNVNHKPSNTKVD |
| | | KRVEPKSCDKTHTCPPCPAPELLGGPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKC |
| | | KVSNKALPAPIEKTISKAKGQPREPQVYTLPPSREEMT |
| | | KNQVSLTCLVKGFYPSDIAVEWESNGQPENNYKTTPP |
| | | VLDSDGSFFLYSKLTVDKSRWQQGNVFSCSVMHEAL |
| | | HNHYTQKSLSLSPGK |
| SEQ ID NO: 213 | DNA Heavy | CAGGTGCAGCTGGTTGAATCTGGTGGCGGAGTGGT |
| | Chain | GCAGCCTGGCAGATCTCTGAGACTGTCTTGTGCCGC |
| | | CAGCGGCTTCACCTTCGACAGATATGGCATGCACT |
| | | GGGTCCGACAGGCCCCTGGAAAAGGACTTGAATGG |
| | | GTGGCCGGAATCTGGCACGAAGGCAGCCACAAGTA |
| | | CTACGCCGATAGCCTGAGAGGCCGGTTCACCATC |
| | | GCAGAGACAACGCCAAGAACACCCTGGACCTCCAG |
| | | CTGAACAGACTGAGAGCCGAGGATACCAGCGTGTA |
| | | CTACTGCGTGCGGCAGACCAACAGAGGCAGACTGG |
| | | ACGATGCCTTCGATATCTGGGGCCAAGGGACAATG |
| | | GTCACCGTTAGCTCAGCTAGCACCAAGGGCCCCAG |
| | | CGTGTTCCCCCTGGCCCCCAGCAGCAAGAGCACCA |
| | | GCGGCGGCACAGCCGCCCTGGGCTGCCTGGTGAAG |
| | | GACTACTTCCCCGAGCCCGTGACCGTGTCCTGGAA |
| | | CAGCGGAGCCCTGACCTCCGGCGTGCACACCTTCC |
| | | CCGCCGTGCTGCAGAGCAGCGGCCTGTACAGCCTG |
| | | TCCAGCGTGGTGACAGTGCCCAGCAGCAGCCTGGG |
| | | CACCCAGACCTACATCTGCAACGTGAACCACAAGC |
| | | CCAGCAACACCAAGGTGGACAAGAGAGTGGAGCC |
| | | CAAGAGCTGCGACAAGACCCACACCTGCCCCCCCT |
| | | GCCCAGCCCCAGAGCTGCTGGGCGGACCCTCCGTG |
| | | TTCCTGTTCCCCCCCAAGCCCAAGGACACCCTGATG |
| | | ATCAGCAGGACCCCCGAGGTGACCTGCGTGGTGGT |
| | | GGACGTGAGCCACGAGGACCCAGAGGTGAAGTTCA |
| | | ACTGGTACGTGGACGGCGTGGAGGTGCACAACGCC |
| | | AAGACCAAGCCCAGAGAGGAGCAGTACAACAGCA |
| | | CCTACAGGGTGGTGTCCGTGCTGACCGTGCTGCAC |
| | | CAGGACTGGCTGAACGGCAAGGAATACAAGTGCA |
| | | AGGTCTCCAACAAGGCCCTGCCAGCCCCCATCGAA |
| | | AAGACCATCAGCAAGGCCAAGGGCCAGCCACGGG |
| | | AGCCCCAGGTGTACACCCTGCCCCCCTCCCGGGAG |
| | | GAGATGACCAAGAACCAGGTGTCCCTGACCTGTCT |
| | | GGTGAAGGGCTTCTACCCCAGCGACATCGCCGTGG |
| | | AGTGGGAGAGCAACGGCCAGCCCGAGAACAACTA |
| | | CAAGACCACCCCCCCAGTGCTGGACAGCGACGGCA |
| | | GCTTCTTCCTGTACAGCAAGCTGACCGTGGACAAG |
| | | TCCAGGTGGCAGCAGGGCAACGTGTTCAGCTGCAG |
| | | CGTGATGCACGAGGCCCTGCACAACCACTACACCC |
| | | AGAAGAGCCTGAGCCTGTCCCCCGGCAAG |
| SEQ ID NO: 214 | LCDR1 | RASQTISSYLN |
| (Combined) | | |
| SEQ ID NO: 215 | LCDR2 | AASTLQS |
| (Combined) | | |
| SEQ ID NO: 216 | LCDR3 | QQNYDTLWT |
| (Combined) | | |
| SEQ ID NO: 217 (Kabat) | LCDR1 | RASQTISSYLN |
| SEQ ID NO: 218 (Kabat) | LCDR2 | AASTLQS |
| SEQ ID NO: 219 (Kabat) | LCDR3 | QONYDTLWT |
| SEQ ID NO: 220 | LCDR1 | SQTISSY |
| (Chothia) | | |
| SEQ ID NO: 221 | LCDR2 | AAS |
| (Chothia) | | |
| SEQ ID NO: 222 | LCDR3 | NYDTLW |
| (Chothia) | | |
| SEQ ID NO: 223 (IMGT) | LCDR1 | QTISSY |
| SEQ ID NO: 224 (IMGT) | LCDR2 | AAS |
| SEQ ID NO: 225 (IMGT) | LCDR3 | QQNYDTLWT |
| SEQ ID NO: 226 | VL | DIQMTQSPSSLSAAVGDRVTISCRASQTISSYLNWYQ |
| | | QKPGEAPKLLIYAASTLQSGVPSRFGGSGSGTDFTLTI |
| | | SSLQPEDSATYYCQQNYDTLWTFGQGTKVEIK |
| SEQ ID NO: 227 | DNA VL | GATATCCAGATGACCCAGTCTCCATCCTCCCTGTCT |
| | | GCAGCTGTAGGAGACAGAGTCACCATCTCTTGCCG |
| | | GGCAAGTCAGACCATTAGTAGTTATTTAAATTGGT |
| | | ATCAGCAGAAACCAGGGGAAGCCCCTAAGCTCCTG |
| | | ATCTATGCTGCCTCCACCTTGCAAAGTGGGGTCCCT |
| | | TCAAGGTTCGGTGGCAGTGGATCTGGGACAGATTT |
| | | CACTCTCACCATCAGCAGTCTGCAACCTGAAGATTC |
| | | TGCAACTTACTACTGTCAACAGAATTACGATACTTT |
| | | GTGGACGTTCGGCCAAGGGACCAAGGTGGAAATCA |
| | | AA |
| SEQ ID NO: 228 | Light Chain | DIQMTQSPSSLSAAVGDRVTISCRASQTISSYLNWYQ |
| | | QKPGEAPKLLIYAASTLQSGVPSRFGGSGSGTDFTLTI |
| | | SSLQPEDSATYYCQQNYDTLWTFGQGTKVEIKRTVA |
| | | APSVFIFPPSDEQLKSGTASVVCLLNNFYPREAKVQW |
| | | KVDNALQSGNSQESVTEQDSKDSTYSLSSTLTLSKAD |
| | | YEKHKVYACEVTHQGLSSPVTKSFNRGEC |
| SEQ ID NO: 229 | DNA Light | GATATCCAGATGACCCAGTCTCCATCCTCCCTGTCT |
| | Chain | GCAGCTGTAGGAGACAGAGTCACCATCTCTTGCCG |
| | | GGCAAGTCAGACCATTAGTAGTTATTTAAATTGGT |
| | | ATCAGCAGAAACCAGGGGAAGCCCCTAAGCTCCTG |
| | | ATCTATGCTGCCTCCACCTTGCAAAGTGGGGTCCCT |
| | | TCAAGGTTCGGTGGCAGTGGATCTGGGACAGATTT |
| | | CACTCTCACCATCAGCAGTCTGCAACCTGAAGATTC |
| | | TGCAACTTACTACTGTCAACAGAATTACGATACTTT |
| | | GTGGACGTTCGGCCAAGGGACCAAGGTGGAAATCA |
| | | AACGTACGGTGGCCGCTCCCAGCGTGTTCATCTTCC |
| | | CCCCCAGCGACGAGCAGCTGAAGAGCGGCACCGCC |
| | | AGCGTGGTGTGCCTGCTGAACAACTTCTACCCCCG |
| | | GGAGGCCAAGGTGCAGTGGAAGGTGGACAACGCC |
| | | CTGCAGAGCGGCAACAGCCAGGAGAGCGTCACCG |
| | | AGCAGGACAGCAAGGACTCCACCTACAGCCTGAGC |
| | | AGCACCCTGACCCTGAGCAAGGCCGACTACGAGAA |
| | | GCATAAGGTGTACGCCTGCCAGGTGACCCACCAGG |
| | | GCCTGTCCAGCCCCGTGACCAAGAGCTTCAACAGG |
| | | GGCGAGTGC |
| SEQ ID NO: 230 | HCDR1 | GPTPERYGMH |
| (Combined) | | |
| SEQ ID NO: 231 | HCDR2 | GTWHEGSHKYYADSLRG |
| (Combined) | | |
| SEQ ID NO: 232 | HCDR3 | QTNRGRLDDAFDI |
| (Combined) | | |
| SEQ ID NO: 233 (Kabat) | HCDR1 | RYGMH |
| SEQ ID NO: 234 (Kabat) | HCDR2 | GIWHEGSHKYTADSLRG |
| SEQ ID NO: 235 (Kabat) | HCDR3 | QTNRGRLDDAFDI |
| SEQ ID NO: 236 | HCDR1 | GFTFERY |
| (Chothia) | | |
| SEQ ID NO: 237 | HCDR2 | WHEGSH |
| (Chothia) | | |
| SEQ ID NO: 238 | HCDR3 | QTNRGRLDDAFDI |
| (Chothia) | | |
| SEQ ID NO: 239 (IMGT) | HCDR1 | GFTFERYG |
| SEQ ID NO: 240 (IMGT) | HCDR2 | IWHEGSHK |
| SEQ ID NO: 341 (IMGT) | HCDR3 | VRQTNRGRLDDAFDI |
| SEQ ID NO: 242 | VH | QVQLVESGGGVVQPGRSLRLSCAASGFTFERYGMHW |
| | | VRQAPGKGLEWVAGIWHEGSHKYYADSLRGRFTISR |
| | | DNAKNTLDLQLNRLRAEDTSVYYCVRQTNRGRLDD |
| | | AFDIWGQGTMVTVSS |
| SEQ ID NO: 243 | DNA VH | CAGGTGCAGCTGGTTGAATCTGGTGGCGGAGTGGT |
| | | GCAGCCTGGCAGATCTCTGAGACTGTCTTGTGCCGC |
| | | CAGCGGCTTCACCTTCGAGAGATATGGCATGCACT |
| | | GGGTCCGACAGGCCCCTGGAAAAGGACTTGAATGG |
| | | GTGGCCGGAATCTGGCACGAAGGCAGCCACAAGTA |
| | | CTACGCCGATAGCCTGAGAGGCCGGTTCACCATCA |
| | | GCAGAGACAACGCCAAGAACACCCTGGACCTCCAG |
| | | CTGAACAGACTGAGAGCCGAGGATACCAGCGTGTA |
| | | CTACTGCGTGCGGCAGACCAACAGAGGCAGACTGG |
| | | ACGATGCCTTCGATATCTGGGGCCAAGGGACAATG |
| | | GTCACCGTTAGCTCA |
| SEQ ID NO: 244 | Heavy Chain | QVQLVESGGGVVQPGRSLRLSCAASGFTFERYGMHW |
| | | VRQAPGKGLEWVAGIWHEGSHKYYADSLRGRFTISR |
| | | DNAKNTLDLQLNRLRAEDTSVYYCVRQTNRGRLDD |
| | | AFDIWGQGTMVTVSSASTKGPSVFPLAPSSKSTSGGT |
| | | AALGCLVKDYFPEPVTVSWNSGALTSGVHTFPAVLQ |
| | | SSGLYSLSSVVTVPSSLGTQTYINCVNHKPSNTKVDK |
| | | RVEPKSCDKTHTCPPCPAPELLGGPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCK |
| | | VSNKALPAPIEKTISKAKGQPREPQVYTLPPSREEMTK |
| | | NQVSLTCLVKGFYPSDIAVEWESNGQPENNYKTTPPV |
| | | LDSDGSFFLYSKLTVDKSRWQQGNVFSCSVMHEALH |
| | | NHYTQKSLSLSPGK |
| SEQ ID NO: 245 | DNA Heavy | CAGGTGCAGCTGGTTGAATCTGGTGGCGGAGTGGT |
| | Chain | GCAGCCTGGCAGATCTCTGAGACTGTCTTGTGCCGC |
| | | CAGCGGCTTCACCTTCGAGAGATATGGCATGCACT |
| | | GGGTCCGACAGGCCCCTGGAAAAGGACTTGAATGG |
| | | GTGGCCGGAATCTGGCACGAAGGCAGCCACAAGTA |
| | | CTACGCCGATAGCCTGAGAGGCCGGTTCACCATCA |
| | | GCAGAGACAACGCCAAGAACACCCTGGACCTCCAG |
| | | CTGAACAGACTGAGAGCCGAGGATACCAGCGTGTA |
| | | CTACTGCGTGCGGCAGACCAACAGAGGCAGACTGG |
| | | ACGATGCCTTCGATATCTGGGGCCAAGGGACAATG |
| | | GTCACCGTTAGCTCAGCTAGCACCAAGGGCCCCAG |
| | | CGTGTTCCCCCTGGCCCCCAGCAGCAAGAGCACCA |
| | | GCGGCGGCACAGCCGCCCTGGGCTGCCTGGTGAAG |
| | | GACTACTTCCCCGAGCCCGTGACCGTGTCCTGGAA |
| | | CAGCGGAGCCCTGACCTCCGGCGTGCACACCTTCC |
| | | CCGCCGTGCTGCAGAGCAGCGGCCTGTACAGCCTG |
| | | TCCAGCGTGGTGACAGTGCCCAGCAGCAGCCTGGG |
| | | CACCCAGACCTACATCTGCAACGTGAACCACAAGC |
| | | CCAGCAACACCAAGGTGGACAAGAGAGTGGAGCC |
| | | CAAGAGCTGCGACAAGACCCACACCTGCCCCCCCT |
| | | GCCCAGCCCCAGAGCTGCTGGGCGGACCCTCCGTG |
| | | TTCCTGTTCCCCCCCAAGCCCAAGGACACCCTGATG |
| | | ATCAGCAGGACCCCCGAGGTGACCTGCGTGGTGGT |
| | | GGACGTGAGCCACGAGGACCCAGAGGTGAAGTTCA |
| | | ACTGGTACGTGGACGGCGTGGAGGTGCACAACGCC |
| | | AAGACCAAGCCCAGAGAGGAGCAGTACAACAGCA |
| | | CCTACAGGGTGGTGTCCGTGCTGACCGTGCTGCAC |
| | | CAGGACTGGCTGAACGGCAAGGAATACAAGTGCA |
| | | AGGTCTCCAACAAGGCCCTGCCAGCCCCCATCGAA |
| | | AAGACCATCAGCAAGGCCAAGGGCCAGCCACGGG |
| | | AGCCCCAGGTGTACACCCTGCCCCCCTCCCGGGAG |
| | | GAGATGACCAAGAACCAGGTGTCCCTGACCTGTCT |
| | | GGTGAAGGGCTTCTACCCCAGCGACATCGCCGTGG |
| | | AGTGGGAGAGCAACGGCCAGCCCGAGAACAACTA |
| | | CAAGACCACCCCCCCAGTGCTGGACAGCGACGGCA |
| | | GCTTCTTCCTGTACAGCAAGCTGACCGTGGACAAG |
| | | CGTGATGCACGAGGCCCTGCACAACCACTACACCC |
| | | AGAAGAGCCTGAGCCTGTCCCCCGGCAAG |
| SEQ ID NO: 246 | LCDR1 | RASQTISSYLN |
| (Combined) | | |
| SEQ ID NO: 247 | LCDR2 | AASTLQS |
| (Combined) | | |
| SEQ ID NO: 248 | LCDR3 | QQNYDTLWT |
| (Combined) | | |
| SEQ ID NO: 249 (Kabat) | LCDR1 | RASQTISSYLN |
| SEQ ID NO: 250 (Kabat) | LCDR2 | AASTLQS |
| SEQ ID NO: 251 (Kabat) | LCDR3 | QQNYDTLWT |
| SEQ ID NO: 252 | LCDR1 | SQTISSY |
| (Chothia) | | |
| SEQ ID NO: 253 | LCDR2 | AAS |
| (Chothia) | | |
| SEQ ID NO: 254 | LCDR3 | NYDTLW |
| (Chothia) | | |
| SEQ ID NO: 255 (IMGT) | LCDR1 | QTISSY |
| SEQ ID NO: 256 (IMGT) | LCDR2 | AAS |
| SEQ ID NO: 257 (IMGT) | LCDR3 | QQNYDTLWT |
| SEQ ID NO: 258 | VL | DIQMTQSPSSLSAAVGDRVTISCRASQTISSYLNWYQ |
| | | QKPGEAPKLLIYAASTLQSGVPSRFGGSGSGTDFTLTI |
| | | SSLQPEDSATYYCQQNYDTLWTFGQGTKVEIK |
| SEQ ID NO: 259 | DNA VL | GATATCCAGATGACCCAGTCICCATCCTCCCTGTCT |
| | | GCAGCTGTAGGAGACAGAGTCACCATCTCTTGCCG |
| | | GGCAAGTCAGACCATTAGTAGTTATTTAAATTGGT |
| | | ATCAGCAGAAACCAGGGGAAGCCCCTAAGCTCCTG |
| | | ATCTATGCTGCCTCCACCTTGCAAAGTGGGGTCCCT |
| | | TCAAGGTTCGGTGGCAGTGGATCTGGGACAGATTT |
| | | CACTCTCACCATCAGCAGTCTGCAACCTGAAGATTC |
| | | TGCAACTTACTACTGTCAACAGAATTACGATACTTT |
| | | GTGGACGTTCGGCCAAGGGACCAAGGTGGAAATCA |
| | | AA |
| SEQ ID NO: 260 | Light Chain | DIQMTQSPSSLSAAVGDRVTISCRASQTISSYLNWYQ |
| | | QKPGEAPKLLIYAASTLQSGVPSRFGGSGSGTDFTLTI |
| | | SSLQPEDSATYYCQQNYDTLWTFGQGTKVEIKRTVA |
| | | APSVFTFPPSDEQLKSGTASVVCLLNNFYPREAKVQW |
| | | KVDNALQSGNSQESVTEQDSKDSTYSLSSTLTLSKAD |
| | | YEKHKVYACEVTHQGLSSPVTKSFNRGEC |
| SEQ ID NO: 261 | DNA Light | GATATCCAGATGACCCAGTCICCATCCTCCCTGTCT |
| | Chain | GCAGCTGTAGGAGACAGAGTCACCATCTCTTGCCG |
| | | GGCAAGTCAGACCATTAGTAGTTATTTAAATTGGT |
| | | ATCAGCAGAAACCAGGGGAAGCCCCTAAGCTCCTG |
| | | ATCTATGCTGCCTCCACCTTGCAAAGTGGGGTCCCT |
| | | TCAAGGTTCGGTGGCAGTGGATCTGGGACAGATTT |
| | | CACTCTCACCATCAGCAGTCTGCAACCTGAAGATTC |
| | | TGCAACTTACTACTGTCAACAGAATTACGATACTTT |
| | | GTGGACGTTCGGCCAAGGGACCAAGGTGGAAATCA |
| | | AACGTACGGTGGCCGCTCCCAGCGTGTTCATCTTCC |
| | | CCCCCAGCGACGAGCAGCTGAAGAGCGGCACCGCC |
| | | AGCGTGGTGTGCCTGCTGAACAACTTCTACCCCCG |
| | | GGAGGCCAAGGTGCAGTGGAAGGTGGACAACGCC |
| | | CTGCAGAGCGGCAACAGCCAGGAGAGCGTCACCG |
| | | AGCAGGACAGCAAGGACTCCACCTACAGCCTGAGC |
| | | AGCACCCTGACCCTGAGCAAGGCCGACTACGAGAA |
| | | GCATAAGGTGTACGCCTGCGAGGTGACCCACCAGG |
| | | GCCTGTCCAGCCCCGTGACCAAGAGCTTCAACAGG |
| | | GGCGAGTGC |
| SEQ ID NO: 262 | HCDR1 | GFIFTDYYMT |
| (Combined) | | |
| SEQ ID NO: 263 | HCDR2 | FTTSGGETTYYADSVKG |
| (Combined) | | |
| SEQ ID NO: 264 | HCDR3 | AHFDGYQYDTRGDFTYYFDN |
| (Combined) | | |
| SEQ ID NO: 265 (Kabat) | HCDR1 | DYYMT |
| SEQ ID NO: 268 (Kabat) | HCDR2 | FTTSGGETTYYADSVKG |
| SEQ ID NO: 267 (Kabat) | HCDR3 | AHFDGYQYDTRGDFTYYFDN |
| SEQ ID NO: 268 | HCDR1 | GFIFTDY |
| (Chothia) | | |
| SEQ ID NO: 269 | HCDR2 | TSGGET |
| (Chothia) | | |
| SEQ ID NO: 270 | HCDR3 | AHFDGYQYDTRGDFTVYFDN |
| (Chothia) | | |
| SEQ ID NO: 271 (IMGT) | HCDR1 | GFIFTDYY |
| SEQ ID NO: 272 (IMGT) | HCDR2 | ITSGGETT |
| SEQ ID NO: 273 (IMGT) | HCDR3 | VRAHFDGYQYDTRGDFTYYFDN |
| SEQ ID NO: 274 | VH | QVQLQESGGRLVRPGGSLRLSCAASGFIFTDYYMTWI |
| | | RQAPGKGPEWIAFITSGGETTYYADSVKGRFTISRDN |
| | | AKKSLFLQMYSLRADDTAVYYCVRAHFDGYQYDTR |
| | | GDFTYYFDNWGLGTLVSVSS |
| SEQ ID NO: 275 | DNA VH | CAGGTGCAGCTGCAGGAGTCGGGGGGACGCTTGGT |
| | | CAGGCCTGGAGGGTCCCTGAGACTCTCCTGTGCAG |
| | | CCTCCGGATTCATCTTCACTGACTACTACATGACCT |
| | | GGATCCGCCAGGCTCCAGGGAAGGGGCCGGAGTG |
| | | GATTGCATTTATCACAAGTGGGGGCGAGACCACAT |
| | | ACTACGCAGACTCTGTGAAGGGCCGCTTCACCATTT |
| | | CCAGGGACAACGCCAAGAAGTCACTCTTTCTGCAA |
| | | ATGTACAGCCTGAGAGCCGACGACACGGCCGTGTA |
| | | TTATTGTGTGAGAGCCCACTTTGATGGTTATCAGTA |
| | | TGATACTCGTGGTGACTTCACTTATTACTTTGACAA |
| | | CTGGGGCCTGGGAACCCTGGTCAGCGTCTCCTCA |
| SEQ ID NO: 276 | Heavy Chain | QVQLQESGGRLVRPGGSLRLSCAASGFIFTDYYMTWI |
| | | RQAPGKGPEWIAFITSGGETTYYADSVKGRFTISRDN |
| | | AKKSLFLQMYSLRADDTAVYYCVRAHFDGYQYDTR |
| | | GDFTYYFDNWGLGTLVSVSSASTKGPSVFPLAPSSKS |
| | | TSGGTAALGCLVKDYFPEPVTVSWNSGALTSGVHTFP |
| | | AVLQSSGLYSLSSVVTVPSSSLGTQTYICNVNHKPSNT |
| | | KVDKRVEPKSCDKTHTCPPCPAPELLGGPSVFLFPPKP |
| | | KDTLMISRTPEVTCVVVDVSHEDPEVKFNWYVDGVE |
| | | VHNAKTKPREEQYNSTYRVVSVLTVLHQDWLNGKE |
| | | YKCKVSNKALPAPIEKTISKAKGQPREPQVYTLPPSRE |
| | | EMTKNQVSLTCLVKGFYPSDIAVEWESNGQPENNYK |
| | | TTPPVLDSDGSFFLYSKLTVDKSRWQQGNVFSCSVM |
| | | HEALHNHYTQKSLSLSPGK |
| SEQ ID NO: 277 | DNA Heavy | CAGGTGCAGCTGCAGGAGTCGGGGGGACGCTTGGT |
| | Chain | CAGGCCTGGAGGGTCCCTGAGACTCTCCTGTGCAG |
| | | CCTCCGGATTCATCTTCACTGACTACTACATGACCT |
| | | GGATCCGCCAGGCTCCAGGGAAGGGGCCGGAGTG |
| | | GATTGCATTTATCACAAGTGGGGGCGAGACCACAT |
| | | ACTACGCAGACTCTGTGAAGGGCCGCTTCACCATTT |
| | | CCAGGGACAACGCCAAGAAGTCACTCTTTCTGCAA |
| | | ATGTACAGCCTGAGAGCCGACGACACGGCCGTGTA |
| | | TTATTGTGTGAGAGCCCACTTTGATGGTTATCAGTA |
| | | TGATACTCGTGGTGACTTCACTTATTACTTTGACAA |
| | | CTGGGGCCTGGGAACCCTGGTCAGCGTCTCCTCAG |
| | | CTAGCACCAAGGGCCCCAGCGTGTTCCCCCTGGCC |
| | | CCCAGCAGCAAGAGCACCAGCGGCGGCACAGCCG |
| | | CCCTGGGCTGCCTGGTGAAGGACTACTTCCCCGAG |
| | | CCCGTGACCGTGTCCTGGAACAGCGGAGCCCTGAC |
| | | CTCCGGCGTGCACACCTTCCCCGCCGTGCTGCAGA |
| | | GCAGCGGCCTGTACAGCCTGTCCAGCGTGGTGACA |
| | | GTGCCCAGCAGCAGCCTGGGCACCCAGACCTACAT |
| | | CTGCAACGTGAACCACAAGCCCAGCAACACCAAGG |
| | | TGGACAAGAGAGTGGAGCCCAAGAGCTGCGACAA |
| | | GACCCACACCTGCCCCCCCTGCCCAGCCCCAGAGC |
| | | TGCTGGGCGGACCCTCCGTGTTCCTGTTCCCCCCCA |
| | | AGCCCAAGGACACCCTGATGATCAGCAGGACCCCC |
| | | GAGGTGACCTGCGTGGTGGTGGACGTGAGCCACGA |
| | | GGACCCAGAGGTGAAGTTCAACTGGTACGTGGACG |
| | | GCGTGGAGGTGCACAACGCCAAGACCAAGCCCAG |
| | | AGAGGAGCAGTACAACAGCACCTACAGGGTGGTGT |
| | | CCGTGCTGACCGTGCTGCACCAGGACTGGCTGAAC |
| | | GGCAAGGAATACAAGTGCAAGGTCTCCAACAAGGC |
| | | CCTGCCAGCCCCCATCGAAAAGACCATCAGCAAGG |
| | | CCAAGGGCCAGCCACGGGAGCCCCAGGTGTACACC |
| | | CTGCCCCCCTCCCGGGAGGAGATGACCAAGAACCA |
| | | GGTGTCCCTGACCTGTCTGGTGAAGGGCTTCTACC |
| | | CAGCGACATCGCCGTGGAGTGGGAGAGCAACGGCC |
| | | AGCCCGAGAACAACTACAAGACCACCCCCCCAGTG |
| | | CTGGACAGCGACGGCAGCTTCTTCCTGTACAGCAA |
| | | GCTGACCGTGGACAAGTCCAGGTGGCAGCAGGGCA |
| | | ACGTGTTCAGCTGCAGCGTGATGCACGAGGCCCTG |
| | | CACAACCACTACACCCAGAAGAGCCTGAGCCTGTC |
| | | CCCCGGCAAG |
| SEQ ID NO: 278 | LCDR1 | RASQSVSSSLA |
| (Combined) | | |
| SEQ ID NO: 279 | LCDR2 | GASTRAT |
| (Combined) | | |
| SEQ ID NO: 280 | LCDR3 | HQYINWPPGDT |
| (Combined) | | |
| SEQ ID NO: 281 (Kabat) | LCDR1 | RASQSVSSSLA |
| SEQ ID NO: 282 (Kabat) | LCDR2 | GASTRAT |
| SEQ ID NO: 283 (Kabat) | LCDR3 | HQYINWPPGDY |
| SEQ ID NO: 284 | LCDR1 | SQSVSSS |
| (Chothia) | | |
| SEQ ID NO: 285 | LCDR2 | GAS |
| (Chothia) | | |
| SEQ ID NO: 286 | LCDR3 | YINWPPGD |
| (Chothia) | | |
| SEQ ID NO: 287 (IMGT) | LCDR1 | QSVSSS |
| SEQ ID NO: 288 (IMGT) | LCDR2 | GAS |
| SEQ ID NO: 289 (IMGT) | LCDR3 | HQYINWPPGDT |
| SEQ ID NO: 290 | VL | EIVMTQSPATLSVSPGERVTLSCRASQSVSSSLAWYQ |
| | | QKPGRAPRLLIYGASTRATGVPARFSGGGSGTDFTLTI |
| | | SSLQSEDFAVYYCHQYINWPPGDTFGQGTRLDIK |
| SEQ ID NO: 291 | DNA VL | GAAATAGTGATGACTCAGTCTCCAGCCACCCTGTCT |
| | | GTGTCTCCAGGGGAAAGAGTCACCCTCTCCTGCAG |
| | | GGCCAGTCAGAGTGTTAGTAGCAGCTTAGCCTGGT |
| | | ACCAGCAGAAACCTGGCCGGGCTCCCAGGCTCCTC |
| | | ATTTATGGAGCATCCACCAGGGCCACTGGTGTCCC |
| | | AGCCAGGTTCAGTGGCGGTGGGTCTGGGACAGACT |
| | | TCACTCTCACCATCAGCAGCCTGCAGTCTGAAGATT |
| | | TTGCAGTTTATTACTGTCACCAGTATATTAATTGGC |
| | | CTCCGGGGGACACTTTTGGCCAGGGGACGAGGCTG |
| | | GATATCAAA |
| SEQ ID NO: 292 | Light Chain | EIVMTQSPATLSVSPGERVTLSCRASQSVSSSLAWYQ |
| | | QKPGRAPRLLIYGASTRATGVPARFSGGGSGTDFTLTI |
| | | SSLQSEDFAVYYCHQYINWPPGDTFGQGTRLDIKRTV |
| | | AAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| SEQ ID NO: 293 | DNA Light | GAAATAGTGATGACTCAGTCTCCAGCCACCCTGTCT |
| | Chain | GTGTCTCCAGGGGAAAGAGTCACCCTCTCCTGCAG |
| | | GGCCAGTCAGAGTGTTAGTAGCAGCTTAGCCTGGT |
| | | ACCAGCAGAAACCTGGCCGGGCTCCCAGGCTCCTC |
| | | ATTTATGGAGCATCCACCAGGGCCACTGGTGTCCC |
| | | AGCCAGGTTCAGTGGCGGTGGGTCTGGGACAGACT |
| | | TCACTCTCACCATCAGCAGCCTGCAGTCTGAAGATT |
| | | TTGCAGTTTATTACTGTCACCAGTATATTAATTGGC |
| | | CTCCGGGGGACACTTTTGGCCAGGGGACGAGGCTG |
| | | GATATCAAACGTACGGTGGCCGCTCCCAGCGTGTT |
| | | CATCTTCCCCCCCAGCGACGAGCAGCTGAAGAGCG |
| | | GCACCGCCAGCGTGGTGTGCCTGCTGAACAACTTC |
| | | TACCCCCGGGAGGCCAAGGTGCAGTGGAAGGTGGA |
| | | CAACGCCCTGCAGAGCGGCAACAGCCAGGAGAGC |
| | | GTCACCGAGCAGGACAGCAAGGACTCCACCTACAG |
| | | CCTGAGCAGCACCCTGACCCTGAGCAAGGCCGACT |
| | | ACGAGAAGCATAAGGTGTACGCCTGCGAGGTGACC |
| | | CACCAGGGCCTGTCCAGCCCCGTGACCAAGAGCTT |
| | | CAACAGGGGCGAGTGC |
| SEQ ID NO: 294 | HCDR1 | GFIFTDYYMT |
| (Combined) | | |
| SEQ ID NO: 295 | HCDR2 | FTTSGGETTYYADSVKG |
| (Combined) | | |
| SEQ ID NO: 296 | HCDR3 | AHFDLYQYDTRGDFTYYFDN |
| (Combined) | | |
| SEQ ID NO: 297 (Kabat) | HCDR1 | DYYMT |
| SEQ ID NO: 298 (Kabat) | HCDR2 | FTTSGGETTYYADSVKG |
| SEQ ID NO: 299 (Kabat) | HCDR3 | AHFDLYQYDTRGDFTYYFDN |
| SEQ ID NO: 300 | HCDR1 | GFIFTDY |
| (Chothia) | | |
| SEQ ID NO: 301 | HCDR2 | TSGGET |
| {Chothia) | | |
| SEQ ID NO: 302 | HCDR3 | AHFDLYQYDTRGDFTYYFDN |
| (Chothia) | | |
| SEQ ID NO: 303 (IMGT) | HCDR1 | GFIFTDYY |
| SEQ ID NO: 304 (IMGT) | HCDR2 | ITSGGETT |
| SEQ ID NO: 305 (IMGT) | HCDR3 | VRAHFDLYQYDTRGDFTYYFDN |
| SEQ ID NO: 306 | VH | QVQLQESGGRLVRPGGSLRLSCAASGFIFTDYYMTWI |
| | | RQAPGKGPEWIAFTISGGETTYADSVKGRFTISRDN |
| | | AKKSLFLQMYSLRADDTAVYYCVRAHFDLYQYDTR |
| | | GDFTYYFDNWGLGTLVSVSS |
| SEQ ID NO: 307 | DNA VH | CAGGTTCAGCTGCAAGAATCTGGCGGCAGACTCGT |
| | | TAGACCTGGCGGCTCTCTGAGACTGTCTTGTGCCGC |
| | | CAGCGGCTTCATCTTCACCGACTACTACATGACCTG |
| | | GATCAGACAGGCCCCTGGCAAGGGACCTGAGTGGA |
| | | TCGCCTTTATCACAAGCGGCGGAGAGACAACCTAC |
| | | TACGCCGATAGCGTGAAGGGCAGATTCACCATCAG |
| | | CCGGGACAACGCCAAGAAGTCCCTGTTCCTCCAGA |
| | | TGTACAGCCTGAGAGCCGACGATACCGCCGTGTAT |
| | | TATTGCGTGCGGGCCCACTTTGACCTGTACCAGTAC |
| | | GATACCAGAGGCGATTTCACCTACTACTTCGACAA |
| | | CTGGGGCCTGGGAACCCTGGTGTCTGTCTCTTCT |
| SEQ ID NO: 308 | Heavy Chain | QVQLQESGGRLVRPGGSLRLSCAASGFIFTDYYMTWI |
| | | RQAPGKGPEWIAFTISGGETTYADSVKGRFTISRDN |
| | | AKKSLFLQMYSLRADDTAVYYCVRAHFDLYQYDTR |
| | | GDFTYYFDNWGLGTLVSVSSASTKGPSVFPLAPSSKS |
| | | TSGGTAALGCLVKDYFPEPVTVSWNSGALTSGVHTFP |
| | | AVLQSSGLYSLSSVVTVPSSSLGTQTYICNVNHKPSNT |
| | | KVDKRVEPKSCDKTHTCPPCPAPELLGGPSVFLFPKP |
| | | KDTLMISRTPEVTCVVVDVSHEDPEVKFNWYVDGVE |
| | | VHNAKTKPREEQYNSTYRVVSVLTVLHQDWLNGKE |
| | | YKCKVSNKALPAPIEKTISKAKGQPREPQVYTLPPSRE |
| | | EMTKNQVSLTCLVKGFYPSDIAVEWESNGQPENNYK |
| | | TTPPVLDSDGSFFLYSKLTVDKSRWQQGNVPSCSVM |
| | | HEALHNHYTQKSLSLSPGK |
| SEQ ID NO: 309 | DNA Heavy | CAGGTTCAGCTGCAAGAATCTGGCGGCAGACTCGT |
| | Chain | TAGACCTGGCGGCTCTCTGAGACTGTCTTGTGCCGC |
| | | CAGCGGCTTCATCTTCACCGACTACTACATGACCTG |
| | | GATCAGACAGGCCCCTGGCAAGGGACCTGAGTGGA |
| | | TCGCCTTTATCACAAGCGGCGGAGAGACAACCTAC |
| | | TACGCCGATAGCGTGAAGGGCAGATTCACCATCAG |
| | | CCGGGACAACGCCAAGAAGTCCCTGTTCCTCCAGA |
| | | TGTACAGCCTGAGAGCCGACGATACCGCCGTGTAT |
| | | TATTGCGTGCGGGCCCACTTTGACCTGTACCAGTAC |
| | | GATACCAGAGGCGATTTCACCTACTACTTCGACAA |
| | | CTGGGGCCTGGGAACCCTGGTGTCTGTCTCTTCTGC |
| | | TAGCACCAAGGGCCCCAGCGTGTTCCCCCTGGCCC |
| | | CCAGCAGCAAGAGCACCAGCGGCGGCACAGCCGC |
| | | CCTGGGCTGCCTGGTGAAGGACTACTTCCCCGAGC |
| | | CCGTGACCGTGTCCTGGAACAGCGGAGCCCTGACC |
| | | TCCGGCGTGCACACCTTCCCCGCCGTGCTGCAGAG |
| | | CAGCGGCCTGTACAGCCTGTCCAGCGTGGTGACAG |
| | | TGCCCAGCAGCAGCCTGGGCACCCAGACCTACATC |
| | | TGCAACGTGAACCACAAGCCCAGCAACACCAAGGT |
| | | GGACAAGAGAGTGGAGCCCAAGAGCTGCGACAAG |
| | | ACCCACACATGCCCCCCCTGCCCGGCGCCAGAGCT |
| | | GCTGGGCGGACCCTCCGTGTTCCTGTTCCCCCCCAA |
| | | GCCCAAGGACACCCTGATGATCAGCAGGACCCCCG |
| | | AGGTGACCTGCGTGGTGGTGGACGTGAGCCACGAG |
| | | GACCCAGAGGTGAAGTTCAACTGGTACGTGGACGG |
| | | CGTGGAGGTGCACAACGCCAAGACCAAGCCCAGA |
| | | GAGGAGCAGTACAACAGCACCTACAGGGTGGTGTC |
| | | CGTGCTGACCGTGCTGCACCAGGACTGGCTGAACG |
| | | GCAAGGAATACAAGTGCAAGGTCTCCAACAAGGCC |
| | | CTGCCAGCCCCCATCGAAAAGACCATCAGCAAGGC |
| | | CAAGGGCCAGCCACGGGAGCCCCAGGTGTACACCC |
| | | TGCCCCCCTCCCGGGAGGAGATGACCAAGAACCAG |
| | | GTGTCCCTGACCTGTCTGGTGAAGGGCTTCTACCCC |
| | | AGCGACATCGCCGTGGAGTGGGAGAGCAACGGCC |
| | | AGCCCGAGAACAACTACAAGACCACCCCCCCAGTG |
| | | CTGGACAGCGACGGCAGCTTCTTCCTGTACAGCAA |
| | | GCTGACCGTGGACAAGTCCAGGTGGCAGCAGGGCA |
| | | ACGTGTTCAGCTGCAGCGTGATGCACGAGGCCCTG |
| | | CACAACCACTACACCCAGAAGAGCCTGAGCTTAAG |
| | | CCCCGGCAAG |
| SEQ ID NO: 310 | LCDR1 | RASQSVSSSLA |
| (Combined) | | |
| SEQ ID NO: 311 | LCDR2 | GASTRAT |
| (Combined) | | |
| SEQ ID NO: 312 | LCDR3 | HQYINWPPGDT |
| (Combined) | | |
| SEQ ID NO: 313 (Kabat) | LCDR1 | RASQSVSSSLA |
| SEQ ID NO: 314 (Kabat) | LCDR2 | GASTRAT |
| SEQ ID NO: 315 (Kabat) | LCDR3 | HQYINWPPGDT |
| SEQ ID NO: 316 | LCDR1 | SQSVSSS |
| (Chothia) | | |
| SEQ ID NO: 317 | LCDR2 | GAS |
| (Chothia) | | |
| SEQ ID NO: 318 | LCDR3 | YINWPPGD |
| (Chothia) | | |
| SEQ ID NO: 319 (IMGT) | LCDR1 | QSVSSS |
| SEQ ID NO: 320 (IMGT) | LCDR2 | GAS |
| SEQ IO NO: 321 (IMGT) | LCDR3 | HQYINWPPGDT |
| SEQ IO NO: 322 | VL | EIVMTQSPATLSVSPGERVTLSCRASQSVSSSLAWYQ |
| | | QKPGRAPRLLIYGASTRATGVPARFSGGGSGTDFTLTI |
| | | SSLQSEDFAVYYCHQYINWPPGDTFGQGTRLDIK |
| SEQ ID NO: 323 | DNA VL | GAAATAGTGATGACTCAGTCTCCAGCCACCCTGTCT |
| | | GTGTCTCCAGGGGAAAGAGTCACCCTCTCCTGCAG |
| | | GGCCAGTCAGAGTGTTAGTAGCAGCTTAGCCTGGT |
| | | ACCAGCAGAAACCTGGCCGGGCTCCCAGGCTCCTC |
| | | ATTTATGGAGCATCCACCAGGGCCACTGGTGTCCC |
| | | AGCCAGGTTCAGTGGCGGTGGGTCTGGGACAGACT |
| | | TCACTCTCACCATCAGCAGCCTGCAGTCTGAAGATT |
| | | TTGCAGTTTATTACTGTCACCAGTATATTAATTGGC |
| | | CTCCGGGGGACACTTTTGGCCAGGGGACGAGGCTG |
| | | GATATCAAA |
| SEQ ID NO: 324 | Light Chain | EIVMTQSPATLSVSPGERVTLSCRASQSVSSSLAWYQ |
| | | QKPGRAPRLLIYGASTRATGVPARFSGGGSGTDFTLTI |
| | | SSLQSEDFAVYYCHQYINWPPGDTFGQGTRLDIKRTV |
| | | AAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| SEQ ID NO: 325 | DNA Light | GAAATAGTGATGACTCAGTCTCCAGCCACCCTGTCT |
| | Chain | GTGTCTCCAGGGGAAAGAGTCACCCTCTCCTGCAG |
| | | GGCCAGTCAGAGTGTTAGTAGCAGCTTAGCCTGGT |
| | | ACCAGCAGAAACCTGGCCGGGCTCCCAGGCTCCTC |
| | | ATTTATGGAGCATCCACCAGGGCCACTGGTGTCCC |
| | | AGCCAGGTTCAGTGGCGGTGGGTCTGGGACAGACT |
| | | TCACTCTCACCATCAGCAGCCTGCAGTCTGAAGATT |
| | | TTGCAGTTTATTACTGTCACCAGTATATTAATTGGC |
| | | CTCCGGGGGACACTTTTGGCCAGGGGACGAGGCTG |
| | | GATATCAAACGTACGGTGGCCGCTCCCAGCGTGTT |
| | | CATCTTCCCCCCCAGCGACGAGCAGCTGAAGAGCG |
| | | GCACCGCCAGCGTGGTGTGCCTGCTGAACAACTTC |
| | | TACCCCCGGGAGGCCAAGGTGCAGTGGAAGGTGGA |
| | | CAACGCCCTGCAGAGCGGCAACAGCCAGGAGAGC |
| | | GTCACCGAGCAGGACAGCAAGGACTCCACCTACAG |
| | | CCTGAGCAGCACCCTGACCCTGAGCAAGGCCGACT |
| | | ACGAGAAGCATAAGGTGTACGCCTGCGAGGTGACC |
| | | CACCAGGGCCTGTCCAGCCCCGTGACCAAGAGCTT |
| | | CAACAGGGGCGAGTGC |
| SEQ ID NO: 326 | HCDR1 | GFIFTDYYMT |
| (Combined) | | |
| SEQ ID NO: 327 | HCDR2 | FTTSGGETTYYADSVKG |
| (Combined) | | |
| SEQ ID NO: 328 | HCDR3 | AHFDIYQYDTRGDFTYYFDN |
| (Combined) | | |
| SEQ ID NO: 329 (Kabat) | HCDR1 | DYYMT |
| SEQ ID NO: 330 (Kabat) | HCDR2 | FTTSGGETTYYADSVKG |
| SEQ ID NO: 331 (Kabat) | HCDR3 | AHFDIYQYDTRGDFTYYFDN |
| SEQ ID NO: 332 | HCDR1 | GFIFTDY |
| (Chothia) | | |
| SEQ ID NO: 333 | HCDR2 | TSGGET |
| (Chothia) | | |
| SEQ ID NO: 334 | HCDR3 | AHFDIYQYDTRGDFTYYFDN |
| (Chothia) | | |
| SEQ ID NO: 335 (IMGT) | HCDR1 | GFIFTDYY |
| SEQ ID NO: 336 (IMGT) | HCDR2 | TTSGGETT |
| SEQ ID NO: 337 (IMGT) | HCDR3 | VRAHFDIYQYDTRGDFTYYFDN |
| SEQ ID NO: 338 | VH | QVQLQESGGRLVRPGGSLRLSCAASGFIFTDYYMTWI |
| | | RQAPGKGPEWIAFITSGGETTYYADSVKGRFTISRDN |
| | | AKKSLFLQMYSLRADDTAVYYCVRAHFDIYQYDTRG |
| | | DFTYYFDNWGLGTLVSVSS |
| SEQ ID NO: 339 | DNA VH | CAGGTTCAGCTGCAAGAATCTGGCGGCAGACTCGT |
| | | TAGACCTGGCGGCTCTCTGAGACTGTCTTGTGCCGC |
| | | CAGCGGCTTCATCTTCACCGACTACTACATGACCTG |
| | | GATCAGACAGGCCCCTGGCAAGGGACCTGAGTGGA |
| | | TCGCCTTTATCACAAGCGGCGGAGAGACAACCTAC |
| | | TACGCCGATAGCGTGAAGGGCAGATTCACCATCAG |
| | | CCGGGACAACGCCAAGAAGTCCCTGTTCCTCCAGA |
| | | TGTACAGCCTGAGAGCCGACGATACCGCCGTGTAT |
| | | TATTGCGTGCGGGCCCACTTTGACATCTACCAGTAC |
| | | GATACCAGAGGCGATTTCACCTACTACTTCGACAA |
| | | CTGGGGCCTGGGAACCCTGGTGTCTGTCTCTTCT |
| SEQ ID NO: 340 | Heavy Chain | QVQLQESGGRLVRPGGSLRLSCAASGFIFTDYYMTWI |
| | | RQAPGKGPEWIAFITSGGETTYYADSVKGRFTISRDN |
| | | AKKSLFLQMYSLRADDTAVYYCVRAHFDIYQYDTRG |
| | | DFTYYFDNWGLGTLVSVSSASTKGPSVFPLAPSSKSTS |
| | | GGTAALGCLVKDYFPEPVTVSWNSGALTSGVHTFPA |
| | | VLQSSGLYSLSSVVTVPSSSLTQTYICNVNHKPSNTK |
| | | VDKRVEPKSCDKTHTCPPCPAPELLGGPSVFLFPPKPK |
| | | DTLMISRTPEVTCVVVDVSHEDPEVKFNWYVDGVEV |
| | | HNAKTKPREEQYNSTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKT |
| | | TPPVLDSDGSFFLYSKLTVDKSRWQQGNVFSCSVMH |
| | | EALHNHYTQKSLSLSPGK |
| SEQ ID NO: 341 | DNA Heavy | CAGGTTCAGCTGCAAGAATCTGGCGGCAGACTCGT |
| | Chain | TAGACCTGGCGGCTCTCTGAGACTGTCTTGTGCCGC |
| | | CAGCGGCTTCATCTTCACCGACTACTACATGACCTG |
| | | GATCAGACAGGCCCCTGGCAAGGGACCTGAGTGGA |
| | | TCGCCTTTATCACAAGCGGCGGAGAGACAACCTAC |
| | | TACGCCGATAGCGTGAAGGGCAGATTCACCATCAG |
| | | CCGGGACAACGCCAAGAAGTCCCTGTTCCTCCAGA |
| | | TGTACAGCCTGAGAGCCGACGATACCGCCGTGTAT |
| | | TATTGCGTGCGGGCCCACTTTGACATCTACCAGTAC |
| | | GATACCAGAGGCGATTTCACCTACTACTTCGACAA |
| | | CTGGGGCCTGGGAACCCTGGTGTCTGTCTCTTCTGC |
| | | TAGCACCAAGGGCCCCAGCGTGTTCCCCCTGGCCC |
| | | CCAGCAGCAAGAGCACCAGCGGCGGCACAGCCGC |
| | | CCTGGGCTGCCTGGTGAAGGACTACTTCCCCGAGC |
| | | CCGTGACCGTGTCCTGGAACAGCGGAGCCCTGACC |
| | | TCCGGCGTGCACACCTTCCCCGCCGTGCTGCAGAG |
| | | CAGCGGCCTGTACAGCCTGTCCAGCGTGGTGACAG |
| | | TGCCCAGCAGCAGCCTGGGCACCCAGACCTACATC |
| | | TGCAACGTGAACCACAAGCCCAGCAACACCAAGGT |
| | | GGACAAGAGAGTGGAGCCCAAGAGCTGCGACAAG |
| | | ACCCACACATGCCCCCCCTGCCCGGCGCCAGAGCT |
| | | GCTGGGCGGACCCTCCGTGTTCCTGTTCCCCCCCAA |
| | | GCCCAAGGACACCCTGATGATCAGCAGGACCCCCG |
| | | AGGTGACCTGCGTGGTGGTGGACGTGAGCCACGAG |
| | | GACCCAGAGGTGAAGTTCAACTGGTACGTGGACGG |
| | | CGTGGAGGTGCACAACGCCAAGACCAAGCCCAGA |
| | | GAGGAGCAGTACAACAGCACCTACAGGGTGGTGTC |
| | | CGTGCTGACCGTGCTGCACCAGGACTGGCTGAACG |
| | | GCAAGGAATACAAGTGCAAGGTCTCCAACAAGGCC |
| | | CTGCCAGCCCCCATCGAAAAGACCATCAGCAAGGC |
| | | CAAGGGCCAGCCACGGGAGCCCCAGGTGTACACCC |
| | | TGCCCCCCTCCCGGGAGGAGATGACCAAGAACCAG |
| | | GTGTCCCTGACCTGTCTGGTGAAGGGCTTCTACCCC |
| | | AGCGACATCGCCGTGGAGTGGGAGAGCAACGGCC |
| | | AGCCCGAGAACAACTACAAGACCACCCCCCCAGTG |
| | | CTGGACAGCGACGGCAGCTTCTTCCTGTACAGCAA |
| | | GCTGACCGTGGACAAGTCCAGGTGGCAGCAGGGCA |
| | | ACGTGTTCAGCTGCAGCGTGATGCACGAGGCCCTG |
| | | CACAACCACTACACCCAGAAGAGCCTGAGCTTAAG |
| | | CCCCGGCAAG |
| SEQ ID NO: 342 | LCDR1 | RASQSVSSSLA |
| (Combined) | | |
| SEQ ID NO: 343 | LCDR2 | GASTRAT |
| (Combined) | | |
| SEQ ID NO: 344 | LCDR3 | HQYINWPPGDT |
| (Combined) | | |
| SEQ ID NO: 345 (Kabat) | LCDR1 | RASQSVSSSLA |
| SEQ ID NO: 346 (Kabat) | LCDR2 | GASTRAT |
| SEQ ID NO: 347 (Kabat) | LCDR3 | HQYINWPPGDT |
| SEQ ID NO: 348 | LCDR1 | SQSVSSS |
| (Chothia) | | |
| SEQ ID NO: 349 | LCDR2 | GAS |
| (Chothia) | | |
| SEQ ID NO: 350 | LCDR3 | YINWPPGD |
| (Chothia) | | |
| SEQ ID NO: 351 (IMGT) | LCDR1 | QSVSSS |
| SEQ ID NO: 352 (IMGT) | LCDR2 | GAS |
| SEQ ID NO: 353 (IMGT) | LCDR3 | HQYINWPPGDT |
| SEQ ID NO: 354 | VL | EIVMTQSPATLSVSPGERVTLSCRASQSVSSSLAWYQ |
| | | QKPGRAPRLLIYGASTRATGVPARFSGGGSGTDFTLTI |
| | | SSLQSEDFAVYCHQYINWPPGDTFGQGTRLDIK |
| SEQ ID NO: 355 | DNA VL | GAAATAGTGATGACTCAGTCTCCAGCCACCCTGTCT |
| | | GTGTCTCCAGGGGAAAGAGTCACCCTCTCCTGCAG |
| | | GGCCAGTCAGAGTGTTAGTAGCAGCTTAGCCTGGT |
| | | ACCAGCAGAAACCTGGCCGGGCTCCCAGGCTCCTC |
| | | ATTTATGGAGCATCCACCAGGGCCACTGGTGTCCC |
| | | AGCCAGGTTCAGTGGCGGTGGGTCTGGGACAGACT |
| | | TCACTCACACATCAGCAGCCTGCAGTCTGAAGATT |
| | | TTGCAGTTTATTACTGTCACCAGTATATTAATTGGC |
| | | CTCCGGGGGACACTTTTGGCCAGGGGACGAGGCTG |
| | | GATATCAAA |
| SEQ ID NO: 356 | Light Chain | EIVMTQSPATLSVSPGERVTLSCRASQSVSSSLAWYQ |
| | | QKPGRAPRLLIYGASTRATGVPARFSGGGSGTDFTLTI |
| | | SSLQSEDFAVYCHQYINWPPGDTFGQGTRLDIKRTV |
| | | AAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| SEQ ID NO: 357 | DNA Light | GAAATAGTGATGACTCAGTCTCCAGCCACCCTGTCT |
| | Chain | GTGTCTCCAGGGGAAAGAGTCACCCTCTCCTGCAG |
| | | GGCCAGTCAGAGTGTTAGTAGCAGCTTAGCCTGGT |
| | | ACCAGCAGAAACCTGGCCGGGCTCCCAGGCTCCTC |
| | | ATTTATGGAGCATCCACCAGGGCCACTGGTGTCCC |
| | | AGCCAGGTTCAGTGGCGGTGGGTCTGGGACAGACT |
| | | TCACTCACACATCAGCAGCCTGCAGTCTGAAGATT |
| | | TTGCAGTTTATTACTGTCACCAGTATATTAATTGGC |
| | | CTCCGGGGGACACTTTTGGCCAGGGGACGAGGCTG |
| | | GATATCAAACGTACGGTGGCCGCTCCAGCGTGTT |
| | | CATCTTCCCCCCCAGCGACGAGCAGCTGAAGAGCG |
| | | GCACCGCCAGCGTGGTGTGCCTGCTGAACAACTTC |
| | | TACCCCCGGGAGGCCAAGGTGCAGTGGAAGGTGGA |
| | | CAACGCCCTGCAGAGCGGCAACAGCCAGGAGAGC |
| | | GTCACCGAGCAGGACAGCAAGGACTCCACCTACAG |
| | | CCTGAGCAGCACCCTGACCCTGAGCAAGGCCGACT |
| | | ACGAGAAGCATAAGGTGTACGCCTGCGAGGTGACC |
| | | CACCAGGGCCTGTCCAGCCCCGTGACCAAGAGCTT |
| | | CAACAGGGGCGAGTGC |
| SEQ ID NO: 358 | HCDR1 | GFTPSYYGMN |
| (Combined) | | |
| SEQ ID NO: 359 | HCDR2 | GITNSGSTIYYADSVKG |
| (Combined) | | |
| SEQ ID NO: 360 | HCDR3 | VGVRSSSGMWDLDY |
| (Combined) | | |
| SEQ ID NO: 361 (Kabat) | HCDR1 | YYGMN |
| SEQ ID NO: 362 (Kabat) | HCDR2 | GITNSGSTIYYADSVKG |
| SEQ ID NO: 363 (Kabat) | HCDR3 | VGVRSSSGMWDLDY |
| SEQ ID NO: 364 | HCDR1 | GFTFSYY |
| (Chothia) | | |
| SEQ ID NO: 365 | HCDR2 | TNSGSI |
| (Chothia) | | |
| SEQ ID NO: 366 | HCDR3 | VGVRSSSGMWDLDY |
| (Chothia) | | |
| SEQ ID NO: 367 (IMGT) | HCDR1 | GFTFSYYG |
| SEQ ID NO: 368 (IMGT) | HCDR2 | ITNSGSIT |
| SEQ ID NO: 368 (IMGT) | HCDR3 | AKVGVRSSSGMWDLDY |
| SEQ ID NO: 370 | VH | EVQLLESGGGLVQPGGSRRLSCAASGFTFSYYGMNW |
| | | VRQAPGKGLEWVSGITNSGSITYYADSVKGRFSISRD |
| | | NSKNTLFLQMNSLRAEDTAVYYCAKVGVRSSSGMW |
| | | DLDYWGQGTLVTVSS |
| SEQ ID NO: 371 | DNA VH | GAGGTTCAATTGTTGGAGTCTGGGGGAGGCCTGGT |
| | | ACAGCCGGGGGGGTCCCGGAGACTGTCCTGTGCAG |
| | | CCTCTGGATTCACCTTTAGCTACTATGGCATGAACT |
| | | GGGTCCGCCAGGCTCCAGGGAAGGGACTGGAATGG |
| | | GTCTCAGGTATTACTAATAGTGGTAGTATCACATAC |
| | | TACGCAGACTCCGTGAAGGGCCGGTTCAGCATCTC |
| | | CAGAGACAATTCCAAGAACACGTTGTTTCTGCAAA |
| | | TGAACAGCCTGAGAGCCGAGGACACGGCCGTATAT |
| | | TACTGTGCGAAGGTGGGAGTGCGCAGTTCGTCCGG |
| | | GATGTGGGACCTTGACTACTGGGGCCAGGGAACCC |
| | | TGGTCACCGTCAGCTCA |
| SEQ ID NO: 372 | Heavy Chain | EVQLLESGGGLVQPGGSRRLSCAASGFTFSYYGMNW |
| | | VRQAPGKGLEWVSGITNSGSITYYADSVKGRFSISRD |
| | | NSKNTLFLQMNSLRAEDTAVYYCAKVGVRSSSGMW |
| | | DLDYWGQGTLVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNMKPSNTKVDK |
| | | RVEPKSCLDTHTCPPCPAPELLGGPSVFLFPPKPKDTL |
| | | MISRTPEVTCVVVDVSHEDPEVDFNWYVDGVEVHNA |
| | | KTKPREEQYNSTYRVVSVLTVLHQDWLNGKEYKCK |
| | | VSNKALPAPIEKTISKAKGQPREPQVYTLPPSREEMTK |
| | | NQVSLTCLVKGFYPSDIAVEWESNGQPENNYKTTPPV |
| | | LDSDGSFFLYSKLTVDKSRWQQGNVPSCSVMHEALH |
| | | NHYTQKSLSLSPGK |
| SEQ ID NO: 373 | DNA Heavy | GAGGTTCAATTGTTGGAGTCTGGGGGAGGCCTGGT |
| | Chain | ACAGCCGGGGGGGTCCCGGAGACTGTCCTGTGCAG |
| | | CCTCTGGATTCACCTTTAGCTACTATGGCATGAACT |
| | | GGGTCCGCCAGGCTCCAGGGAAGGGACTGGAATGG |
| | | GTCTCAGGTATTACTAATAGTGGTAGTATCACATAC |
| | | TACGCAGACTCCGTGAAGGGCCGGTTCAGCATCTC |
| | | CAGAGACAATTCCAAGAACACGTTGTTTCTGCAAA |
| | | TGAACAGCCTGAGAGCCGAGGACACGGCCGTATAT |
| | | TACTGTGCGAAGGTGGGAGTGCGCAGTTCGTCCGG |
| | | GATGTGGGACCTTGACTACTGGGGCCAGGGAACCC |
| | | TGGTCACCGTCAGCTCAGCTAGCACCAAGGGCCCC |
| | | AGCGTGTTCCCCCTGGCCCCCAGCAGCAAGAGCAC |
| | | CAGCGGCGGCACAGCCGCCCTGGGCTGCCTGGTGA |
| | | AGGACTACTTCCCCGAGCCCGTGACCGTGTCCTGG |
| | | AACAGCGGAGCCCCTGACCTCCGGCGTGCACACCTT |
| | | CCCCGCCGTGCTGCAGAGCAGCGGCCTGTACAGCC |
| | | TGTCCAGCGTGGTGACAGTGCCCAGCAGCAGCCTG |
| | | GGCACCCAGACCTACATCTGCAACGTGAACCACAA |
| | | GCCCAGCAACACCAAGGTGGACAAGAGAGTGGAG |
| | | CCCAAGAGCTGCGACAAGACCCACACCTGCCCCCC |
| | | CTGCCCAGCCCCAGAGCTGCTGGGCGGACCCTCCG |
| | | TGTTCCTGTTCCCCCCCAAGCCCAAGGACACCCTGA |
| | | TGATCAGCAGGACCCCCGAGGTGACCTGCGTGGTG |
| | | GTGGACGTGAGCCACGAGGACCCAGAGGTGAAGTT |
| | | CAACTGGTACGTGGACGGCGTGGAGGTGCACAACG |
| | | CCAAGACCAAGCCCAGAGAGGAGCAGTACAACAG |
| | | CACCTACAGGGTGGTGTCCGTGCTGACCGTGCTGC |
| | | ACCAGGACTGGCTGAACGGCAAGGAATACAAGTGC |
| | | AAGGTCTCCAACAAGGCCCTGCCAGCCCCCATCGA |
| | | AAAGACCATCAGCAAGGCCAAGGGCCAGCCACGG |
| | | GAGCCCCAGGTGTACACCCTGCCCCCCTCCCGGGA |
| | | GGAGATGACCAAGAACCAGGTGTCCCTGACCTGTC |
| | | TGGTGAAGGGCTTCTACCCCAGCGACATCGCCGTG |
| | | GAGTGGGAGAGCAACGGCCAGCCCGAGAACAACT |
| | | ACAAGACCACCCCCCCAGTGCTGGACAGCGACGGC |
| | | AGCTTCTTCCTGTACAGCAAGCTGACCGTGGACAA |
| | | GTCCAGGTGGCAGCAGGGCAACGTGTTCAGCTGCA |
| | | GCGTGATGCACGAGGCCCTGCACAACCACTACACC |
| | | CAGAAGAGCCTGAGCCTGTCCCCCGCAAG |
| SEQ ID NO: 374 | LCDR1 | GGNNIGSKSLQ |
| (Combined) | | |
| SEQ ID NO: 375 | LCDR2 | DDSDRPS |
| (Combined) | | |
| SEQ ID NO: 376 | LCDR3 | QVWDTSSDHVV |
| (Combined) | | |
| SEQ ID NO: 377 (Kabat) | LCDR1 | GGNNIGSKSLQ |
| SEQ ID NO: 378 (Kabat) | LCDR2 | DDSDRPS |
| SEQ ID NO: 379 (Kabat) | LCDR3 | QVWDISSDHVV |
| SEQ ID NO: 380 | LCDR1 | NNIGSKS |
| (Chothia) | | |
| SEQ ID NO: 381 | LCDR2 | DDS |
| (Chothia) | | |
| SEQ ID NO: 382 | LCDR3 | WDTSSDHV |
| (Chothia) | | |
| SEQ ID NO: 383 (IMGT) | LCDR1 | NIGSKS |
| SEQ ID NO: 384 (IMGT) | LCDR2 | DDS |
| SEQ ID NO: 385 (IMGT) | LCDR3 | QVWDTSSDHVV |
| SEQ ID NO: 386 | VL | SYVLTQPPSVSVAPGQTARLSCGGNNIGSKSLQWYQQ |
| | | KPGQAPVLVVNDDSDRPSGIPERFSGSNSGNTATLTIS |
| | | RVEAGDEADYYCQVWDTSSDHVVFGGGTKLTVL |
| SEQ ID NO: 387 | DNA VL | TCCTATGTGCTGACTCAGCCACCCTCGGTGTCAGTG |
| | | GCCCCAGGACAGACGGCCAGACTTTCCTGTGGGGG |
| | | AAACAACATTGGAAGTAAAAGTCTGCAGTGGTACC |
| | | AGCAGAAGCCAGGCCAGGCCCCTGTGCTGGTCGTC |
| | | AATGATGATAGCGACCGGCCCTCAGGGATCCCTGA |
| | | GCGATTCTCTGGCTCCAACTCTGGGAACACGGCCA |
| | | CCCTGACCATCAGCAGGGTCGAAGCCGGGGATGAG |
| | | GCCGACTATTACTGTCAGGTGTGGGATACTAGTAG |
| | | TGATCATGTGGTCTTCGGCGGAGGGACCAAGCTGA |
| | | CCGTCCTA |
| SEQ ID NO: 388 | Light Chain | SYVLTQPPSVSVAPGQTARLSCGGNNIGSKSLQWYQQ |
| | | KPGQAPVLVVNDDSDRPSGIPERFSGSNSGNTATLTIS |
| | | RVEAGDEADYYCQVWDTSSDHVVFGGGTKLTVLGQ |
| | | PKAAPSVTLFPPSSEELQANKATLVCLISDFYPGAVTV |
| | | AWKADSSPVKAGYETTTPSKQSNNKYAASSYLSLTPE |
| | | QWKSHRSYSCQVTHEGSTVEKTVAPTECS |
| SEQ ID NO: 389 | DNA Light | TCCTATGTGCTGACTCAGCCACCCTCGGTGTCAGTG |
| | Chain | GCCCCAGGACAGACGGCCAGACTTTCCTGTGGGGG |
| | | AAACAACATTGGAAGTAAAAGTCTGCAGTGGTACC |
| | | AGCAGAAGCCAGGCCAGGCCCCTGTGCTGGTCGTC |
| | | AATGATGATAGCGACCGGCCCTCAGGGATCCCTGA |
| | | GCGATTCTCTGGCTCCAACTCTGGGAACACGGCCA |
| | | CCCTGACCATCAGCAGGGTCGAAGCCGGGGATGAG |
| | | GCCGACTATTACTGTCAGGTGTGGGATACTAGTAG |
| | | TGATCATGTGGTCTTCGGCGGAGGGACCAAGCTGA |
| | | CCGTCCTAGGCCAGCCTAAGGCCGCTCCCTCCGTG |
| | | ACCCTGTTCCCCCCCAGCTCCGAGGAACTGCAGGC |
| | | CAACAAGGCCACCCTGGTGTGCCTGATCAGCGACT |
| | | TCTACCCTGGCGCCGTGACCGTGGCCTGGAAGGCC |
| | | GACAGCAGCCCCGTGAAGGCCGGCGTGGAGACAA |
| | | CCACCCCAGCAAGCAGAGCAACAACAAGTACGCC |
| | | GCCAGCAGCTACCTGAGCCTGACCCCCGAGCAGTG |
| | | GAAGAGCCACAGAAGCTACAGCTGCCAGGTCACCC |
| | | ACGAGGGCAGCACCGTGGAGAAAACCGTGGCCCCC |
| | | ACCGAGTGCAGG |
| SEQ ID NO: 390 | HCDR1 | GFTFSYYGMN |
| (Combined) | | |
| SEQ ID NO: 391 | HCDR2 | GTTQSGSTTYYADTVKG |
| (Combined) | | |
| SEQ ID NO: 392 | HCDR3 | VGVRSSSGMWDLDY |
| (Combined) | | |
| SEQ ID NO: 393 (Kabat) | HCDR1 | YYGMN |
| SEQ ID NO: 394 (Kabat) | HCDR2 | GTTQSGSTTYTADTVKG |
| SEQ ID NO: 395 (Kabat) | HCDR3 | VGVRSSSGMWDLDY |
| SEQ ID NO: 396 | HCDR1 | FFTFSYY |
| (Chothia) | | |
| SEQ ID NO: 397 | HCDR2 | TQSGSI |
| (Chothia) | | |
| SEQ ID NO: 398 | HCDR3 | VGVRSSSGMWDLDY |
| (Chothia) | | |
| SEQ ID NO: 399 (IMGT) | HCDR1 | GFTFSYYG |
| SEQ ID NO: 400 (IMGT) | HCDR2 | ITQSGSIT |
| SEQ ID NO: 401 (IMGT) | HCDR3 | AKVGVRSSSGMWDLDY |
| SEQ ID NO: 402 | VH | EVQLLESGGGLVQPGGSRRLSCAASGFTFSYYGMNW |
| | | VRQAPGKGLEWVSGTTQSGSITYYADTVKGRFSISRD |
| | | NSKNTLFLQMNSLRAEDTAVYYCAKVGVRSSSGMW |
| | | DLDYWGQGTLVTVSS |
| SEQ ID NO: 403 | DNA VH | GAGGTTCAATTGTTGGAGTCTGGGGGAGGCCTGGT |
| | | ACAGCCGGGGGGGTCCCGGAGACTGTCCTGTGCAG |
| | | CCTCTGGATTCACCTTTAGCTACTATGGCATGAACT |
| | | GGGTCCGCCAGGCTCCAGGGAAGGGACTGGAATGG |
| | | GTCTCAGGTATTACTCAGAGTGGTAGTATCACATAC |
| | | TACGCAGACACCGTGAAGGGCCGGTTCAGCATCTC |
| | | CAGAGACAATTCCAAGAACACGTTGTTTCTGCAAA |
| | | TGAACAGCCTGAGAGCCGAGGACACGGCCGTATAT |
| | | TACTGTGCGAAGGTGGGAGTGCGCAGTTCGTCCGG |
| | | GATGTGGGACCTTGACTACTGGGGCCAGGGAACCC |
| | | TGGTCACCGTCAGCTCA |
| SEQ ID NO: 404 | Heavy Chain | EVQLLESGGGLVQPGGSRRLSCAASGFTFSYYGMNW |
| | | VRQAPGKGLEWVSGTTQSGSITYYADTVKGRFSISRD |
| | | NSKNTLFLQMNSLRAEDTAVYYCAKVGVRSSSGMW |
| | | DLDYWGQGTLVTVSSASTKGPSVFPLAPSSKSTSGGT |
| | | AALGCLVKDYFPEPVTVSWNSGALTSGVHTFPAVLQ |
| | | SSGLYSLSSVTVPSSSLGTQTYICNVNHKPSNTKVDK |
| | | RVEPKSCDKTHTCPPCPAPELLGGPSVFLFPPKPKDTL |
| | | MISRTPEVTCVVVDVSHEDPEVKFNWYVDGVEVHNA |
| | | KTKPREEQYNSTYRVVSVLTVLHQDWLNGKEYKCK |
| | | VSNKALPAPIEKTISKAKGQPREPQVYTLPPSREEMTK |
| | | NQVSLTCLVKGFYPSDIAVEWESNGQPENNYKTTPPV |
| | | LDSDGSFFLYSKLTVDKSRWQQGNVRSCSVMHEALH |
| | | NHYTQKSLSLSPGK |
| SEQ ID NO: 405 | DNA Heavy | GAGGTTCAATTGTTGGAGTCTGGGGGAGGCCTGGT |
| | Chain | ACAGCCGGGGGGGTCCCGGAGACTGTCCTGTGCAG |
| | | CCTCTGGATTCACCTTTAGCTACTATGGCATGAACT |
| | | GGGTCCGCCAGGCTCCAGGGAAGGGACTGGAATGG |
| | | GTCTCAGGTATTACTCAGAGTGGTAGTATCACATAC |
| | | TACGCAGACACCGTGAAGGGCCGGTTCAGCATCTC |
| | | CAGAGACAATTCCAAGAACACGTTGTTTCTGCAAA |
| | | TGAACAGCCTGAGAGCCGAGGACACGGCCGTATAT |
| | | TACTGTGCGAAGGTGGGAGTGCGCAGTTCGTCCGG |
| | | GATGTGGGACCTTGACTACTGGGGCCAGGGAACCC |
| | | TGGTCACCGTCAGCTCAGCTAGCACCAAGGGCCCC |
| | | AGCGTGTTCCCCCTGGCCCCCAGCAGCAAGAGCAC |
| | | CAGCGGCGGCACAGCCGCCCTGGGCTGCCTGGTGA |
| | | AGGACTACTTCCCCGAGCCCGTGACCGTGTCCTGG |
| | | AACAGCGGAGCCCTGACCTCCGGCGTGCACACCTT |
| | | CCCCGCCGTGCTGCAGAGCAGCGGCCTGTACAGCC |
| | | TGTCCAGCGTGGTGACAGTGCCCAGCAGCAGCCTG |
| | | GGCACCCAGACCTACATCTGCAACGTGAACCACAA |
| | | GCCCAGCAACACCAAGGTGGACAAGAGAGTGGAG |
| | | CCCAAGAGCTGCGACAAGACCCACACCTGCCCCCC |
| | | CTGCCCAGCCCCAGAGCTGCTGGGCGGACCCTCCG |
| | | TGTTCCTGTTCCCCCCCAAGCCCAAGGACACCCTGA |
| | | TGATCAGCAGGACCCCCGAGGTGACCTGCGTGGTG |
| | | GTGGACGTGAGCCACGAGGACCCAGAGGTGAAGTT |
| | | CAACTGGTACGTGGACGGCGTGGAGGTGCACAACG |
| | | CCAAGACCAAGCCCAGAGAGGAGCAGTACAACAG |
| | | CACCTACAGGGTGGTGTCCGTGCTGACCGTGCTGC |
| | | ACCAGGACTGGCTGAACGGCAAGGAATACAAGTGC |
| | | AAGGTCTCCAACAAGGCCCTGCCAGCCCCCATCGA |
| | | AAAGACCATCAGCAAGGCCAAGGGCCAGCCACGG |
| | | GAGCCCAGGTGTACACCCTGCCCCCCTCCCGGGA |
| | | GGAGATGACCAAGAACCAGGTGTCCCTGACCTGTC |
| | | TGGTGAAGGGCTTCTACCCCAGCGACATCGCCGTG |
| | | GAGTGGGAGAGCAACGGCCAGCCCGAGAACAACT |
| | | ACAAGACCACCCCCCCAGTGCTGGACAGCGACGGC |
| | | AGCTTCTTCCTGTACAGCAAGCTGACCGTGGACAA |
| | | GTCCAGGTGGCAGCAGGGCAACGTGTTCAGCTGCA |
| | | GCGTGATGCACGAGGCCCTGCACAACCACTACACC |
| | | CAGAAGAGCCTGAGCCTGTCCCCCGGCAAG |
| SEQ ID NO: 406 | LCDR1 | GGNNIGSKSLQ |
| (Combined) | | |
| SEQ ID NO: 407 | LCDR2 | DESDRPS |
| (Combined) | | |
| SEQ ID NO: 408 | LCDR3 | QVWDTSSDHVV |
| (Combined) | | |
| SEQ ID NO: 409 (Kabat) | LCDR1 | GGNNIGSKSLQ |
| SEQ ID NO: 410 (Kabat) | LCDR2 | DESDRPS |
| SEQ ID NO: 411 (Kabat) | LCDR3 | QVWDTSSDNVV |
| SEQ ID NO: 412 | LCDR1 | NNIGSKS |
| (Chothia) | | |
| SEQ ID NO: 413 | LCDR2 | DES |
| (Chothia) | | |
| SEQ ID NO: 414 | LCDR3 | WDTSSDHV |
| (Chothia) | | |
| SEQ ID NO: 415 (IMGT) | LCDR1 | NIGSKS |
| SEQ ID NO: 416 (IMGT) | LCDR2 | DES |
| SEQ ID NO: 417 (IMGT) | LCDR3 | QVWDTSSDHVV |
| SEQ ID NO: 418 | VL | SYVLTQPPSVSVAPGQTARLSCGGNNIGSKSLQWYQQ |
| | | KPGQAPVLVVNDESDRPSGIPERFSGSNSGNTATLTIS |
| | | RVEAGDEADYYCQVWDTSSDHVVFGGGTKLTVL |
| SEQ ID NO: 419 | DNA VL | TCCTATGTGCTGACTCAGCCACCCTCGGTGTCAGTG |
| | | GCCCCAGGACAGACGGCCAGACTTTCCTGTGGGGG |
| | | AAACAACATTGGAAGTAAAAGTCTGCAGTGGTACC |
| | | AGCAGAAGCCAGGCCAGGCCCCTGTGCTGGTCGTC |
| | | AATGATGAGAGCGACCGGCCCTCAGGGATCCCTGA |
| | | GCGATTCTCTGGCTCCAACTCTGGGAACACGGCCA |
| | | CCCTGACCATCAGCAGGGTCGAAGCCGGGGATGAG |
| | | GCCGACTATTACTGTCAGGTGTGGGATACTAGTAG |
| | | TGATCATGTGGTCTTCGGCGGAGGGACCAAGCTGA |
| | | CCGTCCTA |
| SEQ ID NO: 420 | Light Chain | SYVLTQPPSVSVAPGQTARLSCGGNNIGSKSLQWYQQ |
| | | KPGQAPVLVVNDESDRPSGIPERFSGSNSGNTATLTIS |
| | | RVEAGDEADYYCQVWDTSSDHVVFGGGTKLTVLGQ |
| | | PKAAPSVTLFPPSSEELQANKATLVCLISDFYFGAVTV |
| | | AWKADSSPVKAGVETTTPSKQSNNKYAASSYLSLTPE |
| | | QWKSHRSYSCQVTHEGSTVEKTVAPTECS |
| SEQ ID NO: 421 | DNA Light | TCCTATGTGCTGACTCAGCCACCCTCGGTGTCAGTG |
| | Chain | GCCCCAGGACAGACGGCCAGACTTTCCTGTGGGGG |
| | | AAACAACATTGGAAGTAAAAGTCTGCAGTGGTACC |
| | | AGCAGAAGCCAGGCCAGGCCCCTGTGCTGGTCGTC |
| | | AATGATGAGAGCGACCGGCCCTCAGGGATCCCTGA |
| | | GCGATTCTCTGGCTCCAACTCTGGGAACACGGCCA |
| | | CCCTGACCATCAGCAGGGTCGAAGCCGGGGATGAG |
| | | GCCGACTATTACTGTCAGGTGTGGGATACTAGTAG |
| | | TGATCATGTGGTCTTCGGCGGAGGGACCAAGCTGA |
| | | CCGTCCTAGGCCAGCCTAAGGCCGCTCCCTCCGTG |
| | | ACCCTGTTCCCCCCCAGCTCCGAGGAACTGCAGGC |
| | | CAACAAGGCCACCCTGGTGTGCCTGATCAGCGACT |
| | | TCTACCCTGGCGCCGTGACCGTGGCCTGGAAGGCC |
| | | GACAGCAGCCCCGTGAAGGCCGGCGTGGAGACAA |
| | | CCACCCCAGCAAGCAGAGCAAGCAACAAGTACGCC |
| | | GCCAGCAGCTACCTGAGCCTGACCCCCGAGCAGTG |
| | | GAAGAGCCACAGAAGCTACAGCTGCCAGGTCACCC |
| | | ACGAGGGCAGCACCGTGGAGAAAACCGTGGCCCCC |
| | | ACCGAGTGCAGC |
| SEQ ID NO: 422 | HCDR1 | GFTFSYYGMN |
| (Combined) | | |
| SEQ ID NO: 423 | HCDR2 | GTTNVGSITYYADTVKG |
| (Combined) | | |
| SEQ ID NO: 424 | HCDR3 | VGVRSSSGMWDLDY |
| (Combined) | | |
| SEQ ID NO: 425 (Kabat) | HCDR1 | YYGMN |
| SEQ ID NO: 426 (Kabat) | HCDR2 | GTTNVGSITYYADTVKG |
| SEQ ID NO: 427 (Kabat) | HCDR3 | VGVRSSSGMWDLDY |
| SEQ ID NO: 428 | HCDR1 | GFTFSYY |
| (Chothia) | | |
| SEQ ID NO: 429 | HCDR2 | TNVGSI |
| (Chothia) | | |
| SEQ ID NO: 430 | HCDR3 | VGVRSSSGMWDLDY |
| (Chothia) | | |
| SEQ ID NO: 431 (IMGT) | HCDR1 | GFTFSYYG |
| SEQ ID NO: 432 (IMGT) | HCDR2 | ITNVGSIT |
| SEQ ID NO: 433 (IMGT) | HCDR3 | AKVGVRSSSGMWDLDY |
| SEQ ID NO: 434 | VH | EVQLLESGGGLVQPGGSRRLSCAASGFTFSYYGMNW |
| | | VRQAPGKGLEWVSGTTNVGSITYYADTVKGRFSISRD |
| | | NSKNTLFLQMNSLRAEDTAVYYCAKVGVRSSSGMW |
| | | DLDYWGQGTLVTVSS |
| SEQ ID NO: 435 | DNA VH | GAGGTTCAATTGTTGGAGTCTGGGGGAGGCCTGGT |
| | | ACAGCCGGGGGGGTCCCGGAGACTGTCCTGTGCAG |
| | | CCTCTGGATTCACCTTTAGCTACTATGGCATGAACT |
| | | GGGTCCGCCAGGCTCCAGGGAAGGGACTGGAATGG |
| | | GTCTCAGGTATTACTAATGTGGGTAGTATCACATAC |
| | | TACGCAGACACCGTGAAGGGCCGGTTCAGCATCTC |
| | | CAGAGACAATTCCAAGAACACGTTGTTTCTGCAAA |
| | | TGAACAGCCTGAGAGCCGAGGACACGGCCGTATAT |
| | | TACTGTGCGAAGGTGGGAGTGCGCAGTTCGTCCGG |
| | | GATGTGGGACCTTGACTACTGGGGCCAGGGAACCC |
| | | TGGTCACCGTCAGCTCA |
| SEQ ID NO: 436 | Heavy Chain | EVQLLESGGGLVQPGGSRRLSCAASGFTFSYYGMNW |
| | | VRQAPGKGLEWVSGTTNVGSITYYADTVKGRFSISRD |
| | | NSKNTLFLQMNSLRAEDTAVYYCAKVGVRSSSGMW |
| | | DLDYWGQGTLVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVDK |
| | | RVEPKSCDKTHTCPPCPAPELLGGPSVFLFPPKPKDTL |
| | | MISRTPEVTCVVVVDSHEDPEVKFNWYVDGVEVHNA |
| | | KTKPREEQYNSTYRVVSVLTVLHQDWLNGKEYKCK |
| | | VSNKALPAPEKTISKAKGQPREPQVYTLPPSREEMTK |
| | | NQVSLTCLVKGFYPSDIAVEWESNGQPENNYKTTPPV |
| | | LDSDGSFFLYSKLTVDKSRWQQGNVFSCSVMHEALH |
| | | NHYTQKSLSLSPGK |
| SEQ ID NO: 437 | DNA Heavy | GAGGTTCAATTGTTGGAGTCTGGGGGAGGCCTGGT |
| | Chain | ACAGCCGGGGGGGTCCCGGAGACTGTCCTGTGCAG |
| | | CCTCTGGATTCACCTTTAGCTACTATGGCATGAACT |
| | | GGGTCCGCCAGGCTCCAGGGAAGGGACTGGAATGG |
| | | GTCTCAGGTATTACTAATGTGGGTAGTATCACATAC |
| | | TACGCAGACACCGTGAAGGGCCGGTTCAGCATCTC |
| | | CAGAGACAATTCCAAGAACACGTTGTTTCTGCAAA |
| | | TGAACAGCCTGAGAGCCGAGGACACGGCCGTATAT |
| | | TACTGTGCGAAGGTGGGAGTGCGCAGTTCGTCCGG |
| | | GATGTGGGACCTTGACTACTGGGGCCAGGGAACCC |
| | | TGGTCACCGTCAGCTCAGCTAGCACCAAGGGCCCC |
| | | AGCGTGTTCCCCCTGGCCCCCAGCAGCAAGAGCAC |
| | | CAGCGGCGGCACAGCCGCCCTGGGCTGCCTGGTGA |
| | | AGGACTACTTCCCCGAGCCCGTGACCGTGTCCTGG |
| | | AACAGCGGAGCCCTGACCTCCGGCGTGCACACCTT |
| | | CCCCGCCGTGCTGCAGAGCAGCGGCCTGTACAGCC |
| | | TGTCCAGCGTGGTGACAGTGCCCAGCAGCAGCCTG |
| | | GGCACCCAGACCTACATCTGCAACGTGAACCACAA |
| | | GCCCAGCAACACCAAGGTGGACAAGAGAGTGGAG |
| | | CCCAAGAGCTGCGACAAGACCCACACCTGCCCCCC |
| | | CTGCCCAGCCCCAGAGCTGCTGGGCGGACCCTCCG |
| | | TGTTCCTGTTCCCCCCCAAGCCCAAGGACACCCTGA |
| | | TGATCAGCAGGACCCCCGAGGTGACCTGCGTGGTG |
| | | GTGGACGTGAGCCACGAGGACCCAGAGGTGAAGTT |
| | | CAACTGGTACGTGGACGGCGTGGAGGTGCACAACG |
| | | CCAAGACCAAGCCCAGAGAGGAGCAGTACAACAG |
| | | CACCTACAGGGTGGTGTCCGTGCTGACCGTGCTGC |
| | | ACCAGGACTGGCTGAACGGCAAGGAATACAAGTGC |
| | | AAGGTCTCCAACAAGGCCCTGCCAGCCCCCATCGA |
| | | AAAGACCATCAGCAAGGCCAAGGGCCAGCCACGG |
| | | GAGCCCCAGGTGTACACCCTGCCCCCCTCCCGGGA |
| | | GGAGATGACCAAGAACCAGGTGTCCCTGACCTGTC |
| | | TGGTGAAGGGCTTCTACCCCAGCGACATCGCCGTG |
| | | GAGTGGGAGAGCAACGGCCAGCCCGAGAACAACT |
| | | ACAAGACCACCCCCCCAGTGCTGGACAGCGACGGC |
| | | AGCTTCTTCCTGTACAGCAAGCTGACCGTGGACAA |
| | | GTCCAGGTGGCAGCAGGGCAACGTGTTCAGCTGCA |
| | | GCGTGATGCACGAGGCCCTGCACAACCACTACACC |
| | | CAGAAGAGCCTGAGCCTGTCCCCCGGCAAG |
| SEQ ID NO: 438 | LCDR1 | GGNNIGSKSLQ |
| (Combined) | | |
| SEQ ID NO: 439 | LCDR2 | DESDRPS |
| (Combined) | | |
| SEQ ID NO: 440 | LCDR3 | QVWDTSSDHVV |
| (Combined) | | |
| SEQ ID NO: 441 (Kabat) | LCDR1 | GGNNIGSKSL1 |
| SEQ ID NO: 442 (KAbat) | LCDR2 | DESDRPS |
| SEQ ID NO: 443 (Kabat) | LCDR3 | QVWDTSSDHVV |
| SEQ ID NO: 444 | LCDR1 | NNIGSKS |
| (Chothia) | | |
| SEQ ID NO: 445 | LCDR2 | DES |
| (Chothia) | | |
| SEQ ID NO: 446 | LCDR3 | WDTSSDHV |
| (Chothia) | | |
| SEQ ID NO: 447 (IMGT) | LCDR1 | NIGSKS |
| SEQ ID NO: 448 (IMGT) | LCDR2 | DES |
| SEQ ID NO: 449 (IMGT) | LCDR3 | QVWDTSSDHVV |
| SEQ ID NO: 450 | VL | SYVLTQPPSYSVAPGQTARLSCGGNNIGSKSLQWYQQ |
| | | KPGQAPVLVVNDESDRPSGIPERFSGANSGNTATLTIS |
| | | RVEAGDEADYYCQVWDTSSDHVVFGGGTKLTVL |
| SEQ ID NO: 451 | DNA VL | TCCTATGTGCTGACTCAGCCACCCTCGGTGTCAGTG |
| | | GCCCCAGGACAGACGGCCAGACTTTCCTGTGGGGGG |
| | | AAACAACATTGGAAGTAAAAGTCTGCAGTGGTACC |
| | | AGCAGAAGCCAGGCCAGGCCCCTGTGCTGGTCGTC |
| | | AATGATGAGAGCGACCGGCCCTCAGGGATCCCTGA |
| | | GCGATTCTCTGGCTCCAACTCTGGGAACACGGCCA |
| | | CCCTGACCATCAGCAGGGTCGAAGCCGGGGATGAG |
| | | GCCGACTATTACTGTCAGGTGTGGGATACTAGTAG |
| | | TGATCATGTGGTCTTCGGCGGAGGGACCAAGCTGA |
| | | CCGTCCTA |
| SEQ ID NO: 452 | Light Chain | SYVLTQPPSYSVAPGQTARLSCGGNNIGSKSLQWYQQ |
| | | KPGQAPVLVVNDESDRPSGIPERFSGANSGNTATLTIS |
| | | RVEAGDEADYYCQVWDTSSDHVVFGGGTKLTVLGQ |
| | | PKAAPSVTLFPPSSEELQANKATLVCLISDFYFGAVTV |
| | | AWKADSSPVKAGVETTTPSKQSNNKYAASSYLSLTPE |
| | | QWKSHRSYSCQVTHEGSTVEKTVAPTECS |
| SEQ ID NO: 453 | DNA Light | TCCTATGTGCTGACTCAGCCACCCTCGGTGTCAGTG |
| | Chain | GCCCCAGGACAGACGGCCAGACTTTCCTGTGGGGGG |
| | | AAACAACATTGGAAGTAAAAGTCTGCAGTGGTACC |
| | | AGCAGAAGCCAGGCCAGGCCCCTGTGCTGGTCGTC |
| | | AATGATGAGAGCGACCGGCCCTCAGGGATCCCTGA |
| | | GCGATTCTCTGGCTCCAACTCTGGGAACACGGCCA |
| | | CCCTGACCATCAGCAGGGTCGAAGCCGGGGATGAG |
| | | GCCGACTATTACTGTCAGGTGTGGGATACTAGTAG |
| | | TGATCATGTGGTCTTCGGCGGAGGGACCAAGCTGA |
| | | CCGTCCTAGGCCAGCCTAAGGCCGCTCCCTCCGTG |
| | | ACCCTGTTCCCCCCCAGCTCCGAGGAACTGCAGGC |
| | | CAACAAGGCCACCCTGGTGTGCCTGATCAGCGACT |
| | | TCTACCCTGGCGCCGTGACCGTGGCCTGGAAGGCC |
| | | GACAGCAGCCCCGTGAAGGCCGGCGTGGAGACAA |
| | | CCACCCCCAGCAAGCAGAGCAACAACAAGTACGCC |
| | | GCCAGCAGCTACCTGAGCCTGACCCCCGAGCAGTG |
| | | GAAGAGCCACAGAAGCTACAGCTGCCAGGTCACCC |
| | | ACGAGGGCAGCACCGTGGAGAAAACCGTGGCCCCC |
| | | ACCGAGTGCAGC |
| SEQ ID NO: 454 | HCDR1 | GYTFTSYYMH |
| (Combined) | | |
| SEQ ID NO: 455 | HCDR2 | IISPSGGSTSYAQKPQG |
| (Combined) | | |
| SEQ ID NO: 456 | HCDR3 | DWEGGDPYGYYYAFDY |
| (Combined) | | |
| SEQ ID NO: 457 (Kabat) | HCDR1 | SYYMH |
| SEQ ID NO: 458 (Kabat) | HCDR2 | IISPSGGSTSYAQKPQG |
| SEQ ID NO: 459 (Kabat) | HCDR3 | DWEGGDPYGYYYAFDY |
| SEQ ID NO: 460 | HCDR1 | GYTPTSY |
| (Chothia) | | |
| SEQ ID NO: 461 | HCDR2 | SPSGGS |
| (Chothia) | | |
| SEQ ID NO: 462 | HCDR3 | DWEGGDPYGYYYAFDY |
| (Chothia) | | |
| SEQ ID NO: 463 (IMGT) | HCDR1 | GYTFTSYY |
| SED ID NO: 464 (IMGT) | HCDR2 | ISPSGGST |
| SEQ ID NO: 465 (IMGT) | HCDR3 | AKDWEGGDPYGYYYAFDY |
| SEQ ID NO: 466 | VH | QVQLVQSGAEVKKPGASVKVSCKASGYTFTSYYMH |
| | | WVRQAPGQGLEWMGIISPSGGSTSYAQKFQGRFTMT |
| | | RDTSTSTVYMELSSLRSEDTAVYYCARDWEGGDPYG |
| | | YYYAFDYWGQGTLVTVSS |
| SEQ ID NO: 467 | DNA VH | CAGGTGCAATTGGTGCAGAGCGGAGCCGAAGTGAA |
| | | AAAACCTGGGGCCAGCGTGAAAGTGTCCTGCAAAG |
| | | CCTCCGGATACACCTTCACCAGCTACTACATGCACT |
| | | GGGTCCGCCAGGCCCCAGGCCAGGGACTCGAGTGG |
| | | ATGGGCATCATCAGCCCTAGCGGCGGCAGCACCAG |
| | | CTACGCCCAGAAATTCCAGGGCCGGGTGACCATGA |
| | | CCCGCGACACCAGCACCAGCACCGTGTACATGGAA |
| | | CTGAGCAGCCTGCGCAGCGAGGACACCGCCGTGTA |
| | | TTATTGCGCGCGTGACTGGGAAGGTGGTGACCCGT |
| | | ACGGTTACTACTACGCTTTCGACTACTGGGGTCAAG |
| | | GCACCCTGGTTACAGTCAGCTCA |
| SEQ ID NO: 468 | Heavy Chain | QVQLVQSGAEVKKPGASVKVSCKASGYTFTSYYMH |
| | | WVRQAPGQGLEWMGIISPSGGSTSYAQKFQGRFTMT |
| | | RDTSTSTVYMELSSLRSEDTAVYYCARDWEGGDPYG |
| | | YYYAFDYWGQGTLVTVSSASTKGPSVFPLAPSSKSTS |
| | | GGTAALGCLVKDYFPEPVTVSWNSGALTSGVHTFPA |
| | | VLQSSGLYSLSSVVTVPSSSLGTQTYICNVNHKPSNTK |
| | | VDKRVEPKSCDKTHTCPPCPAPELLGGPSVFLFPPKPK |
| | | DTLMISRTPEVTCVVVDVSHEDPEVKFNWYVDGVEV |
| | | HNAKTKPREEQYNSTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKT |
| | | TPPVLDSDGSFFLYSKLTVDKSRWQQGNVFSCSVMH |
| | | EALHNHYTQKSLSLSPGK |
| SEQ ID NO: 469 | DNA Heavy | CAGGTGCAATTGGTGCAGAGCGGAGCCGAAGTGAA |
| | Chain | AAAACCTGGGGCCAGCGTGAAAGTGTCCTGCAAAG |
| | | CCTCCGGATACACCTTCACCAGCTACTACATGCACT |
| | | GGGTCCGCCAGGCCCCAGGCCAGGGACTCGAGTGG |
| | | ATGGGCATCATCAGCCCTAGCGGCGGCAGCACCAG |
| | | CTACGCCCAGAAATTCCAGGGCCGGGTGACCATGA |
| | | CCCGCGACACCAGCACCAGCACCGTGTACATGGAA |
| | | CTGAGCAGCCTGCGCAGCGAGGACACCGCCGTGTA |
| | | TTATTGCGCGCGTGACTGGGAAGGTGGTGACCCGT |
| | | ACGGTTACTACTACGCTTTCGACTACTGGGGTCAAG |
| | | GCACCCTGGTTQACAGTCAGCTCAGCTAGCACCAAG |
| | | GGCCCCAGCGTGTTCCCCCTGGCCCCCAGCAGCAA |
| | | GAGCACCAGCGGCGGCACAGCCGCCCTGGGCTGCC |
| | | TGGTGAAGGACTACTTCCCCGAGCCCGTGACCGTG |
| | | TCCTGGAACAGCGGAGCCCTGACCTCCGGCGTGCA |
| | | CACCTTCCCCGCCGTGCTGCAGAGCAGCGGCCTGT |
| | | ACAGCCTGTCCAGCGTGGTGACAGTGCCCAGCAGC |
| | | AGCCTGGGCACCCAGACCTACATCTGCAACGTGAA |
| | | CCACAAGCCCAGCAACACCAAGGTGGACAAGAGA |
| | | GTGGAGCCCAAGAGCTGCGACAAGACCCACACCTG |
| | | CCCCCCCTGCCCAGCCCCAGAGCTGCTGGGCGGAC |
| | | CCTCCGTGTTCCTGTTCCCCCCCAAGCCCAAGGACA |
| | | CCCTGATGATCAGCAGGACCCCCGAGGTGACCTGC |
| | | GTGGTGGTGGACGTGAGCCACGAGGACCCAGAGGT |
| | | GAAGTTCAACTGGTACGTGGACGGCGTGGAGGTGC |
| | | ACAACGCCAAGACCAAGCCCAGAGAGGAGCAGTA |
| | | CAACAGCACCTACAGGGTGGTGTCCGTGCTGACCG |
| | | TGCTGCACCAGGACTGGCTGAACGGCAAGGAATAC |
| | | AAGTGCAAGGTCTCCAACAAGGCCCTGCCAGCCCC |
| | | CATCGAAAAGACCATCAGCAAGGCCAAGGGCCAG |
| | | CCACGGGAGCCCCAGGTGTACACCCTGCCCCCCTC |
| | | CCGGGAGGAGATGACCAAGAACCAGGTGTCCCTGA |
| | | CCTGTCTGGTGAAGGGCTTCTACCCCAGCGACATC |
| | | GCCGTGGAGTGGGAGAGCAACGGCCAGCCCGAGA |
| | | ACAACTACAAGACCACCCCCCCAGTGCTGGACAGC |
| | | GACGGCAGCTTCTTCCTGTACAGCAAGCTGACCGT |
| | | GGACAAGTCCAGGTGGCAGCAGGGCAACGTGTTCA |
| | | GCTGCAGCGTGATGCACGAGGCCCTGCACAACCAC |
| | | TACACCCAGAAGAGCCTGAGCCTGTCCCCCGGCAA |
| | | G |
| SEQ ID NO: 470 | LCDR1 | RASQSTSSYLN |
| (Combined) | | |
| SEQ ID NO: 471 | LCDR2 | AASSLQS |
| (Combined) | | |
| SEQ ID NO: 472 | LCDR3 | QQSYSTPLT |
| (Combined) | | |
| SEQ ID NO: 473 (Kabat) | LCDR1 | RASQSISSYLN |
| SEQ ID NO: 474 (Kabat) | LCDR2 | AASSLQS |
| SEQ ID NO: 475 (Kabat) | LCDR3 | QQSYSTPLT |
| SEQ ID NO: 476 | LCDR1 | SQSISSY |
| (Chothia) | | |
| SEQ ID NO: 477 | LCDR2 | AAS |
| (Chothia) | | |
| SEQ ID NO: 478 | LCDR3 | SYSTPL |
| (Chothia) | | |
| SEQ ID NO: 479 (IMGT) | LCDR1 | QSISSY |
| SEQ ID NO: 480 (IMGT) | LCDR2 | AAS |
| SEQ ID NO: 481 (IMGT) | LCDR3 | QQSYSTPLT |
| SEQ ID NO: 482 | VL | DIQMTQSPSSLSASVGDRVTITCRASQSISSYLNWYQQ |
| | | KPGKAPKLLIYAASSLQSGVPSRFSGSGSGTDFTLTISS |
| | | LQPEDFATYYCQQSYSTPLTFGQGTKVEIK |
| SEQ ID NO: 483 | DNA VL | GATATCCAGATGACCCAGAGCCCTAGCAGCCTGAG |
| | | CGCCAGCGTGGGCGACCGCGTGACCATTACCTGCA |
| | | GAGCCAGCCAGAGCATCAGCAGCTACCTGAACTGG |
| | | TACCAGCAGAAACCTGGCAAGGCGCCCAAACTATT |
| | | AATCTACGCCGCCAGCAGCCTTCAGAGCGGCGTGC |
| | | CAAGCCGCTTTAGCGGATCCGGCAGCGGCACCGAC |
| | | TTCACCCTGACCATCAGCTCCCTTCAGCCTGAAGAC |
| | | TTCGCCACCTACTACTGCCAGCAGAGCTACAGCAC |
| | | CCCTCTGACCTTTGGCCAGGGCACCAAAGTGGAAA |
| | | TCAAA |
| SEQ ID NO: 484 | Light Chain | DIQMTQSPSSLSASVGDRVTITCRASQSISSYLNWYQQ |
| | | KPGKAPKLLIYAASSLQSGVPSRFSGSGSGTDFTLTISS |
| | | LQPEDFATYYCQQSYSTPLTFGQGTKVEIKRTVAAPS |
| | | VFIFPPSDEQLKSGTASVVCLLNNFYPREAKVQWKVD |
| | | NALQSGNSQESTVEQDSKDSTYSLSSTLTLSKADYEK |
| | | HKVYACEVTHQGLSSPVTKSFNRGEC |
| SEQ ID NO: 485 | DNA Light | GATATCCAGATGACCCAGAGCCCTAGCAGCCTGAG |
| | Chain | CGCCAGCGTGGGCGACCGCGTGACCATTACCTGCA |
| | | GAGCCAGCCAGAGCATCAGCAGCTACCTGAACTGG |
| | | TACCAGCAGAAACCTGGCAAGGCGCCCAAACTATT |
| | | AATCTACGCCGCCAGCAGCCTTCAGAGCGGCGTGC |
| | | CAAGCCGCTTTAGCGGATCCGGCAGCGGCACCGAC |
| | | TTCACCCTGACCATCAGCTCCCTTCAGCCTGAAGAC |
| | | TTCGCCACCTACTACTGCCAGCAGAGCTACAGCAC |
| | | CCCTCTGACCTTTGGCCAGGGCACCAAAGTGGAAA |
| | | TCAAACGTACGGTGGCCGCTCCCAGCGTGTTCATCT |
| | | TCCCCCCCAGCGACGAGCAGCTGAAGAGCGGCACC |
| | | GCCAGCGTGGTGTGCCTGCTGAACAACTTCTACCCC |
| | | CGGGAGGCCAAGGTGCAGTGGAAGGTGGACAACG |
| | | CCCTGCAGAGCGGCAACAGCCAGGAGAGCGTCACC |
| | | GAGCAGGACAGCAAGGACTCCACCTACAGCCTGAG |
| | | CAGCACCCTGACCCTGAGCAAGGCCGACTACGAGA |
| | | AGCATAAGGTGTACGCCTGCGAGGTGACCCACCAG |
| | | GGCCTGTCCAGCCCCGTGACCAAGAGCTTCAACAG |
| | | GGGCGAGTGC |
| SEQ ID NO: 486 | HCDR1 | GYTFTSLEMH |
| (Combined) | | |
| SEQ ID NO: 487 | HCDR2 | HEPSGGSTSYAQKFQG |
| (Combined) | | |
| SEQ ID NO: 488 | HCDR3 | DWEGGDPYGYYYAFDY |
| (Combined) | | |
| SEQ ID NO: 489 (Kabat) | HCDR1 | SLEMH |
| SEQ ID NO: 490 (Kabat) | HCDR2 | HEPSHGSTSYAQKFQG |
| SEQ ID NO: 491 (Kabat) | HCDR3 | DWEGGDPYGYYYAFDY |
| SEQ ID NO: 492 | HCDR1 | GYTFTSL |
| (Chothia) | | |
| SEQ ID NO: 493 | HCDR2 | EPSGGS |
| (Chothia) | | |
| SEQ ID NO: 494 | HCDR3 | DWEGGDPYGYYYAFDY |
| (Chothia) | | |
| SEQ ID NO: 495 (IMGT) | NCDR1 | GYTFTSLE |
| SEQ ID NO: 496 (IMGT) | HCDR1 | IEPSGGST |
| SEQ ID NO: 497 (IMGT) | HCDR3 | ARDWEGGDPYGYYYAFDY |
| SEQ ID NO: 498 | VH | QVQLVQSGAEVKKPGASVKVSCKASGYTFTSLEMH |
| | | WVRQAPGQGLEWMGHEPSGGSTSYAQKFQGRVTMT |
| | | RDTSTSTVYMELSSLRSEDTAVYYCARDWEGGDPYG |
| | | YYYAFDYWGQGTLVTVSS |
| SEQ ID NO: 499 | DNA VH | CAGGTCCAATTGGTGCAGTCTGGCGCCGAAGTGAA |
| | | GAAACCAGGCGCCAGCGTGAAGGTGTCCTGTAAAG |
| | | CCAGCGGCTACACCTTTACCAGCCTGGAAATGCAT |
| | | TGGGTCCGACAGGCTCCAGGACAGGGACTCGAGTG |
| | | GATGGGAATTATCGAGCCTAGCGGCGGCAGCACAA |
| | | GCTACGCCCAGAAATTCCAGGGCAGAGTGACCATG |
| | | ACCAGAGACACCAGCACCTCCACCGTGTACATGGA |
| | | ACTGAGCAGCCTGAGAAGCGAGGACACCGCCGTGT |
| | | ATTATTGCGCGCGTGATTGGGAAGGCGGCGACCCT |
| | | TATGGCTACTACTACGCCTTTGATTACTGGGGCCAG |
| | | GGCACCCTGGTCACAGTTAGCTCA |
| SEQ ID NO: 500 | Heavy Chain | QVQLVQSGAEVKKPGASVKVSCKASGYTFTSLEMH |
| | | WVRQAPGQGLEWMGHEPSGGSTSYAQKFQGRVTMT |
| | | YYYAFDYWGQGTLVTVSSASTKGPSVFPLAPSSKSTS |
| | | GGTAALGCLVKDFPEPVTVSWNSGALTSGVHTFPA |
| | | VLQSSGLYSLSSVVTVPSSSLGTQTYICNVNHKPSNTK |
| | | VDKRVEPKSCDKTHTCPPCPAPELLGGPSVFLFPPKPK |
| | | DTLMISRTPEVTCVVVDVSHEDPEVKFNWYVDGVEV |
| | | HNAKTKPREEQYNSTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKT |
| | | TPPVLDSDGSFFLYSKLTVDKSRWQQGNVFSCSVMH |
| | | EALHNHYTQKSLSLSPGK |
| SEQ ID NO: 501 | DNA Heavy | CAGGTCCAATTGGTGCAGTCTGGCGCCGAAGTGAA |
| | Chain | GAAACCAGGCGCCAGCGTGAAGGTGTCCTGTAAAG |
| | | CCAGCGGCTACACCTTTACCAGCCTGGAAATGCAT |
| | | TGGGTCCGACAGGCTCCAGGACAGGGACTCGAGTG |
| | | GATGGGAATTATCGAGCCTAGCGGCGGCAGCACAA |
| | | GCTACGCCCAGAAATTCCAGGGCAGAGTGACCATG |
| | | ACCAGAGACACCAGCACCTCCACCGTGTACATGGA |
| | | ACTGAGCAGCCTGAGAAGCGAGGACACCGCCGTGT |
| | | ATTATTGCGCGCGTGATTGGGAAGGCGGCGACCCT |
| | | TATGGCTACTACTACGCCTTTGATTACTGGGGCCAG |
| | | GGCACCCTGGTCACAGTTAGCTCAGCTAGCACCAA |
| | | GGGCCCCAGCGTGTTCCCCCTGGCCCCCAGCAGCA |
| | | AGAGCACCAGCGGCGGCACAGCCGCCCTGGGCTGC |
| | | CTGGTGAAGGACTACTTCCCCGAGCCCGTGACCGT |
| | | GTCCTGGAACAGCGGAGCCCTGACCTCCGGCGTGC |
| | | ACACCTTCCCCGCCGTGCTGCAGAGCAGCGGCCTG |
| | | TACAGCCTGTCCAGCGTGGTGACAGTGCCCAGCAG |
| | | CAGCCTGGGCACCCAGACCTACATCTGCAACGTGA |
| | | ACCACAAGCCCAGCAACACCAAGGTGGACAAGAG |
| | | AGTGGAGCCCAAGAGCTGCGACAAGACCCACACAT |
| | | GCCCCCCCTGCCCGGCGCCAGAGCTGCTGGGCGGA |
| | | CCCTCCGTGTTCCTGTTCCCCCCCAAGCCCAAGGAC |
| | | ACCCTGATGATCAGCAGGACCCCCGAGGTGACCTG |
| | | CGTGGTGGTGGACGTGAGCCACGAGGACCCAGAGG |
| | | TGAAGTTCAACTGGTACGTGGACGGCGTGGAGGTG |
| | | CACAACGCCAAGACCAAGCCCAGAGAGGAGCAGT |
| | | ACAACAGCACCTACAGGGTGGTGTCCGTGCTGACC |
| | | GTGCTGCACCAGGACTGGCTGAACGGCAAGGAATA |
| | | CAAGTGCAAGGTCTCCAACAAGGCCCTGCCAGCCC |
| | | CCATCGAAAAGACCATCAGCAAGGCCAAGGGCCA |
| | | GCCACGGGAGCCCCAGGTGTACACCCTGCCCCCCT |
| | | CCCGGGAGGAGATGACCAAGAACCAGGTGTCCCTG |
| | | ACCTGTCTGGTGAAGGGCTTCTACCCCAGCGACAT |
| | | CGCCGTGGAGTGGGAGAGCAACGGCCAGCCCGAG |
| | | AACAACTACAAGACCACCCCCCCAGTGCTGGACAG |
| | | CGACGGCAGCTTCTTCCTGTACAGCAAGCTGACCG |
| | | TGGACAAGTCCAGGTGGCAGCAGGGCAACGTGTTC |
| | | AGCTGCAGCGTGATGCACGAGGCCCTGCACAACCA |
| | | CTACACCCAGAAGAGCCTGAGCTTAAGCCCCGGCA |
| | | AG |
| SEQ ID NO: 502 | LCDR1 | RASQSISSYLN |
| (Combined) | | |
| SEQ ID NO: 503 | LCDR2 | AASSLQS |
| (Combined) | | |
| SEQ ID NO: 504 | LCDR3 | QQSYSTPLT |
| (Combined) | | |
| SEQ ID NO: 505 (Kabat) | LCDR1 | RASQSISSYLN |
| SEQ ID NO: 506 (Kabat) | LCDR2 | AASSLQS |
| SEQ ID NO: 507 (Kabat) | LCDR3 | QQSYSTPLT |
| SEQ ID NO: 508 | LCDR1 | SQSISSY |
| (Chothia) | | |
| SEQ ID NO: 509 | LCDR2 | AAS |
| (Chothia) | | |
| SEQ ID NO: 510 | LCDR3 | SYSTPL |
| (Chothia) | | |
| SEQ ID NO: 511 (IMGT) | LCDR1 | QSISSY |
| SEQ ID NO: 512 (IMGT) | LCDR2 | AAS |
| SEQ ID NO: 513 (IMGT) | LCDR3 | QQSYSTPLT |
| SEQ ID NO: 514 | VL | DIQMTQSPSSLSASVGDRVTITCRASQSISSYLNWYQQ |
| | | KPGKAPKLLIYAASSLQSGVPSRFSGSGSGTDFTLTISS |
| | | LQPEDFATYYCQQSYSTPLTFGQGTKVEIK |
| SEQ ID NO: 515 | DNA VL | GATATCCAGATGACCCAGAGCCCTAGCAGCCTGAG |
| | | CGCCAGCGTGGGCGACCGCGTGACCATTACCTGCA |
| | | GAGCCAGCCAGAGCATCAGCAGCTACCTGAACTGG |
| | | TACCAGCAGAAACCTGGCAAGGCGCCCAAACTATT |
| | | AATCTACGCCGCCAGCAGCCTTCAGAGCGGCGTGC |
| | | CAAGCCGCTTTAGCGGATCCGGCAGCGGCACCGAC |
| | | TTCACCCTGACCATCAGCTCCCTTCAGCCTGAAGAC |
| | | TTCGCCACCTACTACTGCCAGCAGAGCTACAGCAC |
| | | CCCTCTGACCTTTGGCCAGGGCACCAAAGTGGAAA |
| | | TCAAA |
| SEQ ID NO: 516 | Light Chain | DIQMTQSPSSLSASVGDRVTITCRASQSISSYLNWYQQ |
| | | KPGKAPKLLIYAASSLQSGVPSRFSGSGSGTDFTLTISS |
| | | LQPEDFATYYCQQSYSTPLTFGQGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |
| SEQ ID NO: 517 | DNA Light | GATATCCAGATGACCCAGAGCCCTAGCAGCCTGAG |
| | Chain | CGCCAGCGTGGGCGACCGCGTGACCATTACCTGCA |
| | | GAGCCAGCCAGAGCATCAGCAGCTACCTGAACTGG |
| | | TACCAGCAGAAACCTGGCAAGGCGCCCAAACTATT |
| | | AATCTACGCCGCCAGCAGCCTTCAGAGCGGCGTGC |
| | | CAAGCCGCTTTAGCGGATCCGGCAGCGGCACCGAC |
| | | TTCACCCTGACCATCAGCTCCCTTCAGCCTGAAGAC |
| | | TTCGCCACCTACTACTGCCAGCAGAGCTACAGCAC |
| | | CCCTCTGACCTTTGGCCAGGGCACCAAAGTGGAAA |
| | | TCAAACGTACGGTGGCCGCTCCCAGCGTGTTCATCT |
| | | TCCCCCCCAGCGACGAGCAGCTGAAGAGCGGCACC |
| | | GCCAGCGTGGTGTGCCTGCTGAACAACTTCTACCCC |
| | | CGGGAGGCCAAGGTGCAGTGGAAGGTGGACAACG |
| | | CCCTGCAGAGCGGCAACAGCCAGGAGAGCGTCACC |
| | | GAGCAGGACAGCAAGGACTCCACCTACAGCCTGAG |
| | | CAGCACCCTGACCCTGAGCAAGGCCGACTACGAGA |
| | | AGCATAAGGTGTACGCCTGAGAGGTGACCCACCAG |
| | | GGCCTGTCCAGCCCCGTGACCAAGAGCTTCAACAG |
| | | GGGCGAGTGC |

Other antibodies of the present disclosure include those where the amino acids or nucleic acids encoding the amino acids have been mutated; yet have at least 60, 70, 80, 90 or 95 percent identity to the sequences described in Table 1. In some aspects, it includes mutant amino acid sequences wherein no more than 1, 2, 3, 4 or 5 amino acids have been mutated in the variable regions when compared with the variable regions depicted in the sequence described in Table 1, while retaining substantially the same therapeutic activity.

Since these antibodies can bind to HBsAg, the VH, VL, full length light chain, and full length heavy chain sequences (amino acid sequences and the nucleotide sequences encoding the amino acid sequences) can be “mixed and matched” to create other HBsAg-binding antibodies. Such “mixed and matched” HBsAg-binding antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise, a full length light chain sequence from a particular full length heavy chain/full length light chain pairing should be replaced with a structurally similar full length light chain sequence. Accordingly, in one aspect, the disclosure provides for an isolated monoclonal antibody or antigen binding region thereof having: a heavy chain variable region comprising an amino acid sequence selected from the group consisting of SEQ ID NOs: 18, 50, 82, 114, 146, 178, 210, 242, 274, 306, 338, 370, 402, 434, 466 or 498 (Table 1); and a light chain variable region comprising an amino acid sequence selected from the group consisting of SEQ ID NOs: 34, 66, 98, 130, 162, 194, 226, 258, 290, 322, 354, 386, 418, 450, 482 or 514 (Table 1); wherein the antibody specifically binds to HBsAg.

In another aspect, the disclosure provides (i) an isolated monoclonal antibody having: a full length heavy chain comprising an amino acid sequence that has been optimized for expression in the cell of a mammalian selected from the group consisting of SEQ ID NOs: 20, 52, 84, 116, 148, 180, 212, 244, 276, 308, 340, 372, 404, 436, 468 or 500 (Table 1) and a full length light chain comprising an amino acid sequence that has been optimized for expression in the cell of a mammalian selected from the group consisting of SEQ ID NOs: 36, 68, 100, 132, 164, 196, 228, 260, 292, 324, 356, 388, 420, 452, 484, or 516 (Table 1) or (ii) a functional protein comprising an antigen binding portion thereof.

In another aspect, the present disclosure provides HBsAg binding antibodies that comprise the heavy chain and light chain CDR1s, CDR2s and CDR3s as described in Table 1, or combinations thereof. The amino acid sequences of the VH CDR Is of the antibodies are shown in SEQ ID NOs: 9, 41, 73, 105, 137, 169, 201, 233, 265, 297, 329, 361, 393, 425, 457 or 489 The amino acid sequences of the VH CDR2s of the antibodies and are shown in SEQ ID NOs: 10, 42, 74, 106, 138, 170, 202, 234, 266, 298, 330, 362, 394, 426, 458 or 490. The amino acid sequences of the VH CDR3s of the antibodies are shown in SEQ ID NOs: 11, 43, 75, 107, 139, 171, 203, 235, 267, 299, 331, 363, 395, 427, 459 or 491. The amino acid sequences of the VL CDR1s of the antibodies are shown in SEQ ID NOs: 25, 57, 89, 121, 153, 185, 217, 249, 281, 313, 345, 377, 409, 441, 473 or 505. The amino acid sequences of the VL CDR2s of the antibodies are shown in SEQ ID NOs: 26, 58, 90, 122, 154, 186, 218, 250, 282, 314, 346, 378, 410, 442, 474 or 506. The amino acid sequences of the VL CDR3s of the antibodies are shown in SEQ ID NOs: 27, 59, 91, 123, 155, 187, 219, 251, 283, 315, 347, 379, 411, 443, 475 or 507

Given that each of these antibodies can bind to HBsAg and that antigen-binding specificity is provided primarily by the CDR1, 2 and 3 regions, the VH CDR1, 2 and 3 sequences and VL CDR1, 2 and 3 sequences can be “mixed and matched” (i.e., CDRs from different antibodies can be mixed and matched, although each antibody must contain a VH CDR1, 2 and 3 and a VL CDR1, 2 and 3 to create other HBsAg-binding binding molecules. Such “mixed and matched” HBsAg-binding antibodies can be tested using the binding assays known in the art and those described in the Examples (e.g., ELISAs). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for monoclonal antibodies of the present disclosure.

Accordingly, the present disclosure provides an isolated monoclonal antibody or antigen binding region thereof comprising a heavy chain CDR1 comprising an amino acid sequence selected from the group consisting of SEQ ID NOs: 9, 41, 73, 105, 137, 169, 201, 233, 265, 297, 329, 361, 393, 425, 457 or 489; a heavy chain CDR2 comprising an amino acid sequence selected from the group consisting of SEQ ID NOs: 10, 42, 74, 106, 138, 170, 202, 234, 266, 298, 330, 362, 394, 426, 458 or 490; a heavy chain CDR3 comprising an amino acid sequence selected from the group consisting of SEQ ID NOs: 11, 43, 75, 107, 139, 171, 203, 235, 267, 299, 331, 363, 395, 427, 459 or 491; a light chain CDR1 comprising an amino acid sequence selected from the group consisting of SEQ ID NOs: 25, 57, 89, 121, 153, 185, 217, 249, 281, 313, 345, 377, 409, 441, 473 or 505; a light chain CDR2 comprising an amino acid sequence selected from the group consisting of SEQ ID NOs: 26, 58, 90, 122, 154, 186, 218, 250, 282, 314, 346, 378, 410, 442, 474 or 506; and a light chain CDR3 comprising an amino acid sequence selected from the group consisting of SEQ ID NOs: 27, 59, 91, 123, 155, 187, 219, 251, 283, 315, 347, 379, 411, 443, 475 or 507; wherein the antibody specifically binds to HBsAg and neutralizes HDV infections by blocks entry and depletes HBsAg-containing particles

In certain aspects, an antibody that specifically binds to HBsAg is an antibody or antibody fragment (e.g., antigen binding fragment) that is described in Table 1.

### 1. Identification of Antibodies

The present disclosure provides antibodies and antibody fragments (e.g., antigen binding fragments) that bind to HBsAg and neutralizes HDV infections by blocks entry and depletes HBsAg-containing particles. In certain aspects the antibodies and antibody fragments can bind to the same epitope within all four hepatitis serotypes.

The present disclosure also provides antibodies and antibody fragments (e.g., antigen binding fragments) that bind to the same epitope as do the HBsAg antibodies described in Table 1. Additional antibodies and antibody fragments (e.g., antigen binding fragments) can therefore be identified based on their ability to cross-compete (e.g., to competitively inhibit the binding of, in a statistically significant manner) with other antibodies in binding assays. The ability of a test antibody to inhibit the binding of antibodies and antibody fragments (e.g., antigen binding fragments) of the present disclosure to HBsAg demonstrates that the test antibody can compete with that antibody or antibody fragment (e.g., antigen binding fragments) for binding to HBsAg;

such an antibody may, according to non-limiting theory, bind to the same or a related (e.g., a structurally similar or spatially proximal) epitope on HBsAg as the antibody or antibody fragment (e.g., antigen binding fragments) with which it competes. In a certain aspect, the antibody that binds to the same epitope on HBsAg as the antibodies or antibody fragments (e.g., antigen binding fragments) of the present disclosure is a human or humanized monoclonal antibody. Such human or humanized monoclonal antibodies can be prepared and isolated as described herein.

### 2. Further Alteration of the Framework of Fc Region

The present disclosure disclosed specific HBsAg antibodies. These antibodies comprise modified antibodies or antigen binding fragments thereof that further comprise modifications to framework residues within VH and/or VL, e.g. to improve the properties of the antibody. Typically such framework modifications are made to decrease the immunogenicity of the antibody. For example, one approach is to “back-mutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back-mutated” to the germline sequence by, for example, site-directed mutagenesis. Such “back-mutated” antibodies are also intended to be encompassed.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr et al.

In addition or alternative to modifications made within the framework or CDR regions, antibodies can be engineered to include modifications within the Fc region, typically to alter one or more functional properties of the antibody, such as serum half-life, complement fixation, Fc receptor binding, and/or antigen-dependent cellular cytotoxicity. Furthermore, an antibody can be chemically modified (e.g., one or more chemical moieties can be attached to the antibody) or be modified to alter its glycosylation, again to alter one or more functional properties of the antibody. Each of these aspects is described in further detail below.

In one aspect, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another aspect, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acids selected from amino acid residues can be replaced with a different amino acid residue such that the antibody has altered C1q binding and/or reduced or abolished complement dependent cytotoxicity (CDC). This approach is described in, e.g., U.S. Pat. No. 6,194,551 by Idusogie et al.

In another aspect, one or more amino acid residues are altered to thereby alter the ability of the antibody to fix complement. This approach is described in, e.g., the PCT Publication WO 94/29351 by Bodmer et al. In a specific aspect, one or more amino acids of an antibody or antigen binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and

IgG3 subclasses as well as the constant region of the light chain of the kappa isotype as described by Jefferis et al., MAbs. 1:332-338 (2009).

In yet another aspect, the Fc region is modified to increase the ability of the antibody to mediate antibody dependent cellular cytotoxicity (ADCC) and/or to increase the affinity of the antibody for an Fc.gamma. receptor by modifying one or more amino acids. This approach is described in, e.g., the PCT Publication WO 00/42072 by Presta. Moreover, the binding sites on human IgG1 for Fc.gamma.R1, Fc.gamma.RII, Fc.gamma.RIII and FcRn have been mapped and variants with improved binding have been described (see Shields et al., J. Biol. Chem. 276:6591-6604, 2001).

In still another aspect, the glycosylation of an antibody is modified. For example, an aglycosylated antibody can be made (i.e., the antibody lacks glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in, e.g., U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). PCT Publication WO 99/54342 by Umana et al. describes cell lines engineered to express glycoprotein-modifying glycosyl transferases (e.g., beta (1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another aspect, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In order to minimize the ADCC activity of an antibody, specific mutations in the Fc region result in “Fc silent” antibodies that have minimal interaction with effector cells. In general, the “IgG Fc region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody.

Silenced effector functions can be obtained by mutation in the Fc region of the antibodies and have been described in the art: LALA and N297A (Strohl, W., 2009, Curr. Opin. Biotechnol. vol. 20 (6): 685-691); and D265A (Baudino et al., 2008, J. Immunol. 181:6664-69) see also Heusser et al., WO2012065950. Examples of silent Fc lgG1 antibodies are the LALA mutant comprising L234A and L235A mutation in the lgG1 Fc amino acid sequence. Another example of a silent lgG1 antibody is the DAPA (D265A, P329A) mutation (U.S. Pat. No. 6,737,056). Another silent lgG1 antibody comprises the N297A mutation, which results in aglycosylated/non-glycosylated antibodies.

Fc silent antibodies result in no or low ADCC activity, meaning that an Fc silent antibody exhibits an ADCC activity that is below 50% specific cell lysis (low ADCC activity), or that is below 1% specific cell lysis (no ADCC activity).

### 3. Production of the Antibodies

Anti-HBsAg antibodies and antibody fragments (e.g., antigen binding fragments) thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NOs: 19, 51, 83, 115, 147, 179, 211, 243, 275, 307, 339, 371, 403, 435, 467 or 499. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NOs: 35, 67, 99, 131, 163, 195, 227, 259, 291, 323, 355, 387, 419, 451, 483 or 515.

In some aspects, the polynucleotide encoding the heavy chain has at least 85%, 89%, 90%, 91%, 92%, 93%, 94%, 95%, 96%, 97%, 98%, 99%, or 100% nucleic acid sequence identity with a polynucleotide of SEQ ID NO:21, 53, 85, 117, 149, 181, 213, 245, 277, 309, 341, 373, 405, 437, 469 or 501. In some aspects, the polynucleotide encoding the light chain has at least 85%, 89%, 90%, 91%, 92%, 93%, 94%, 95%, 96%, 97%, 98%, 99%, or 100% nucleic acid sequence identity with a polynucleotide of SEQ ID NO: 37, 69, 101, 133, 165, 197, 229, 261, 293, 325, 357, 389, 421, 453, 485 or 517.

The polynucleotides of the present disclosure can encode only the variable region sequence of an anti-HBsAg antibody. They can also encode both a variable region and a constant region of the antibody. Some of the polynucleotide sequences encode a polypeptide that comprises variable regions of both the heavy chain and the light chain of one of an exemplified anti-HBsAg antibody. Some other polynucleotides encode two polypeptide segments that respectively are substantially identical to the variable regions of the heavy chain and the light chain of one of the antibodies.

The polynucleotide sequences can be produced by de novo solid-phase DNA synthesis or by PCR mutagenesis of an existing sequence encoding an anti-HBsAg antibody or its binding fragment. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the present disclosure are expression vectors and host cells for producing the anti-HBsAg antibodies described above. Various expression vectors can be employed to express the polynucleotides encoding the anti-HBsAg antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet 15:345, 1997). For example, nonviral vectors useful for expression of the anti-HBsAg polynucleotides and polypeptides in mammalian (e.g., human) cells include pThioHis A, B & C, pcDNA3.1/His, pEBVHis A, B & C (Invitrogen, San Diego, Calif.), MPSV vectors, and numerous other vectors known in the art for expressing other proteins. Useful viral vectors include vectors based on retroviruses, adenoviruses, adeno-associated viruses, herpes viruses, vectors based on SV40, papilloma virus, HBP Epstein Barr virus, vaccinia virus vectors and Semliki Forest virus (SFV). Sec, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding an anti-HBsAg antibody chain or fragment. In some aspects, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements may also be required or desired for efficient expression of an anti-HBsAg antibody chain or fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted anti-HBsAg antibody sequences. More often, the inserted anti-HBsAg antibody sequences are linked to a signal sequences before inclusion in the vector. Vectors to be used to receive sequences encoding anti-HBsAg antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human.

The host cells for harboring and expressing the anti-HBsAg antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-HBsAg antibodies. Insect cells in combination with baculovirus vectors can also be used.

In other aspects, mammalian host cells are used to express and produce the anti-HBsAg antibodies of the present disclosure. For example, they can be either a hybridoma cell line expressing endogenous immunoglobulin genes (e.g., the myeloma hybridoma clones as described in the Examples) or a mammalian cell line harboring an exogenous expression vector. These include any normal mortal or normal or abnormal immortal animal or human cell. For example, a number of suitable host cell lines capable of secreting intact immunoglobulins have been developed, including the CHO cell lines, various COS cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation: nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express anti-HBsAg antibody chains or binding fragments can be prepared using expression vectors which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### Therapeutic and Diagnostic Uses

The antibodies, antibody fragments (e.g., antigen binding fragments) of the present disclosure are useful in a variety of applications including, but not limited to, hepatitis D viral infection and disease. In certain aspects, the antibodies, antibody fragments (e.g., antigen binding fragments), and are useful for neutralizing hepatitis D infection and the prevention or treatment of liver cirrhosis or liver cancer). The methods of use can be in vitro, ex vivo, or in vivo methods.

In one aspect, the antibodies, antibody fragments (e.g., antigen binding fragments), are useful for detecting the presence of HBsAg in a biological sample. The term “detecting” as used herein encompasses quantitative or qualitative detection. In certain aspects, a biological sample comprises a cell or tissue. In certain aspects, such tissues include normal and/or cancerous tissues that express HBsAg at higher levels relative to other tissues.

In one aspect, the present disclosure provides a method of detecting the presence of HBsAg or hepatitis D in a biological sample. In certain aspects, the method comprises contacting the biological sample with an anti-HBsAg antibody under conditions permissive for binding of the antibody to the antigen, and detecting whether a complex is formed between the antibody and the antigen. The biological sample can include, without limitation, urine or blood samples.

Also included is a method of diagnosing a disorder associated with expression of HBsAg. In certain aspects, the method comprises contacting a test cell with an anti-HBsAg antibody; determining the level of expression (either quantitatively or qualitatively) of HBsAg in the test cell by detecting binding of the antibody to HBsAg; and comparing the level of infection in the test cell with the level of infection of hepatitis D virus in a control cell (e.g., a normal cell of the same tissue origin as the test cell or a non-virus infected cell), wherein a higher level of presence of HBsAg in the test cell as compared to the control cell indicates the presence of a disorder associated with infection with hepatitis D. In certain aspects, the test cell is obtained from an individual suspected of having a hepatitis D virus infection.

In certain aspects, a method of diagnosis or detection, such as those described above, comprises detecting binding of a HBsAg antibody to a hepatitis D virus infected cell. An exemplary assay for detecting binding of an anti-HBsAg antibody to a hepatitis D infected cell is a “FACS” assay.

Certain other methods can be used to detect binding of anti-HBsAg antibodies. Such methods include, but are not limited to, antigen-binding assays that are well known in the art, such as Western blots, radioimmunoassays, ELISA (enzyme linked immunosorbent assay), “sandwich” immunoassays, immunoprecipitation assays, fluorescent immunoassays, protein A immunoassays, and immunohistochemistry (IHC).

In certain aspects, the anti-HBsAg antibodies are labeled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction.

In certain aspects, the anti-HBsAg antibodies are immobilized on an insoluble matrix. Immobilization entails separating the anti-HBsAg antibody from any hepatitis D proteins that remain free in solution. This conventionally is accomplished by either insolubilizing the anti-HBsAg antibody before the assay procedure, as by adsorption to a water-insoluble matrix or surface (Bennich et al, U.S. Pat. No. 3,720,760), or by covalent coupling (for example, using glutaraldehyde cross-linking), or by insolubilizing the anti-HBsAg antibody after formation of a complex between the anti-HBsAg antibody and HBsAg protein, e.g., by immunoprecipitation.

Any of the above aspects of diagnosis or detection can be carried out using an anti-HBsAg antibody of the present disclosure in place of or in addition to another anti-HBsAg antibody.

In one aspect, the disclosure provides for a method of treating, reducing the likelihood of or ameliorating a disease comprising administering the antibodies, antibody fragments (e.g., antigen binding fragments), to a patient, thereby treating the disease. In certain aspects, the disease treated with the antibodies, antibody fragments (e.g., antigen binding fragments), is a hepatitis D viral infection. Examples of hepatitis D diseases which can be treated and/or prevented include, but are not limited to; liver failure, cirrhosis, and hepatocellular carcinoma. In certain aspects, the infection is characterized by HBsAg expressing cells to which the anti-HBsAg antibodies, antibody fragments (e.g., antigen binding fragments) can specifically bind.

The present disclosure provides for methods of treating hepatitis D viral infection and liver failure, cirrhosis, and/or hepatocellular carcinoma comprising administering a therapeutically effective amount of the antibodies, antibody fragments (e.g., antigen binding fragments). In certain aspects, the subject is a human.

In certain aspects, the method of reducing hepatitis D viral infection comprises administering to a subject a therapeutically effective amount of antibodies or antibody fragments (e.g., antigen binding fragments). In certain aspects, the subject is a human. In certain aspects, the subject is immunosuppressed, immunocompromised or has reduced immune function. For immunosuppressed subjects, the amount of immunosuppression can be increased or decreased due to the therapeutic effects of the anti-HBsAg antibodies.

For the treatment of hepatitis D viral infection, the appropriate dosage of the HBsAg antibodies, or antibody fragments (e.g., antigen binding fragments), depend on various factors, such as the type of infection to be treated, the severity and course of the infection, the responsiveness of the infection, the generation of viral resistance to therapy, previous therapy, patient's clinical history, and so on. The antibody can be administered one time or over a series of treatments lasting from several days to several months, or until a cure is effected or a diminution of the infection is achieved (e.g., reduction in viruria or viral damage to the liver). Optimal dosing schedules can be calculated from measurements of drug accumulation in the body of the patient and will vary depending on the relative potency of an individual antibody or antibody fragment (e.g., antigen binding fragment). In certain aspects, dosage is from 0.01 mg to 100 mg (e.g., 0.01 mg, 0.05 mg, 0.1 mg, 0.5 mg, 1 mg, 2 mg, 3 mg, 4 mg, 5 mg, 7 mg, 8 mg, 9 mg, 10 mg, 20 mg, 30 mg, 40 mg, 50 mg, 60 mg, 70 mg, 80 mg. 90 mg or 100 mg) per kg of body weight, and can be given once or more daily, weekly, monthly or yearly. In certain aspects, the antibody or antibody fragment (e.g., antigen binding fragment), of the present disclosure is given once every two weeks or once every three weeks. The treating physician can estimate repetition rates for dosing based on measured half-life and concentrations of the antibody in bodily fluids or tissues.

### Combination Therapy

In certain instances, the antibody or antibody fragment (e.g., antigen binding fragment), of the present disclosure is combined with other therapeutic agents, such as other anti-viral agents, anti-allergic agents, anti-nausea agents (or anti-emetics), pain relievers, cytoprotective agents, immunosuppressants and combinations thereof.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “combination therapy” refers to the administration of two or more therapeutic agents to treat a therapeutic condition or infection described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the individual components separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the individual components are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

In one aspect, the present disclosure provides a method of treating hepatitis D infection by administering to a subject in need thereof anti-HBsAg antibody in together with immunosuppressant therapies. The anti-HBsAg antibodies will reduce the amount of HBsAg in the circulation and allow the immune system to mount a response to the hepatitis D viral infection resulting from the immunosuppressant therapy prior to or post administration. Examples of immunosuppressant therapy include, but are not limited to; a monophosphate dehydrogenase inhibitor, a purine synthesis inhibitor, a calcineurin inhibitor or an mTOR inhibitor. Specific examples of immunosuppressive therapeutics include but are not limited to;

mycophenolate mofetil (MMF), mycophenolate sodium, azathioprine, tacrolimus, sirolimus and cyclosporine.

In one embodiment, an anti-HBsAg antibody combination is used with another antiHBsAg, for HBV and HDV combination therapy.

In one embodiment, an anti-HBsAg antibody combination is used with another antiviral agent, antagonist or agonist for HBV and HDV combination therapy.

The use according to any of the above, wherein the therapeutic agent is an anti-viral agent including siRNA, antisense, gene editing methods directed against hepatitis B or Hepatitis D targets.

The use according to any of the above, wherein the anti-viral agent is: lamivudine, entecavir and tenofovir or alpha-interferon.

The use according to any of the above, wherein the therapeutic agent is an antagonist of immune checkpoint inhibitor.

The use according to any of the above, wherein the antagonist of the immune checkpoint inhibitor is selected from the group consisting of: PD-1, PD-L1, PD-L2, TIM3, CTLA-4, LAG-3, CEACAM-1, CEACAM-5, VISTA, BTLA, TIGIT, LAIR1, CD160, 2B4 and TGFR.

The use according to any of the above, wherein the therapeutic agent is an agonist of immunomodulators, wherein the agonist including but not limited to TLR2, TLR3, TLR4, TLR7, TLR8, TLR9 or combination thereof.

In yet another embodiment, an anti-HBsAg antibody combination is used with a vaccine for prophylactic and therapeutic purpose. When provided prophylactically, the compositions of the invention which are vaccines are provided before any symptom or clinical sign of a pathogen infection becomes manifest. The prophylactic administration of the composition serves to prevent or attenuate any subsequent infection. When provided prophylactically, the compositions of the antibody or its combination of the invention, are provided before any symptom or clinical sign of a disease becomes manifest. The prophylactic administration of the composition serves to prevent or attenuate one or more symptoms or clinical signs associated with the disease.

When provided therapeutically, a vaccine is provided upon the detection of a symptom or clinical sign of actual infection. When provided therapeutically, a composition of the antibody or combination of the invention is provided upon the detection of a symptom or clinical sign of the disease. The therapeutic administration of the compound(s) serves to attenuate a symptom or clinical sign of that disease.

In one embodiment, an anti-HBsAg antibody combination is used with a PD-1 inhibitor, e.g., as described in WO2015/026684 or WO2016/057846. In some embodiments, the PD-1 inhibitor is an anti-PD-1 antibody chosen from Nivolumab, Pembrolizumab or Pidilizumab.

In some embodiments, the anti-PD-1 antibody is Nivolumab. Alternative names for Nivolumab include MDX-1106, MDX-1106-04, ONO-4538, or BMS-936558. In some embodiments, the anti-PD-1 antibody is Nivolumab (CAS Registry Number: 946414-94-4). Nivolumab is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and WO2006/121168. In one embodiment, the inhibitor of PD-1 is Nivolumab, and having a sequence disclosed therein (or a sequence substantially identical or similar thereto, e.g., a sequence at least 85%, 90%, 95% identical or higher to the sequence specified).

In some embodiments, the anti-PD-1 antibody is Pembrolizumab. Pembrolizumab (also referred to as Lambrolizumab, MK-3475, MK03475, SCH-900475 or KEYTRUDA.®.; Merck) is a humanized IgG4 monoclonal antibody that binds to PD-1. Pembrolizumab and other humanized anti-PD-1 antibodies are disclosed in Hamid, O. et al. (2013) New England Journal of Medicine 369 (2): 134-44, U.S. Pat. No. 8,354,509 and WO2009/114335.

In one embodiment, the inhibitor of PD-1 is Pembrolizumab, disclosed in, e.g., U.S. Pat. No. 8,354,509 and WO 2009/114335, and having a sequence disclosed therein (or a sequence substantially identical or similar thereto, e.g., a sequence at least 85%, 90%, 95% identical or higher to the sequence specified).

In some embodiments, the anti-PD-1 antibody is Pidilizumab. Pidilizumab (CT-011; Cure Tech) is a humanized IgGlk monoclonal antibody that binds to PD1. Pidilizumab and other humanized anti-PD-1 monoclonal antibodies are disclosed in WO2009/101611. Other anti-PD1 antibodies include AMP 514 (Amplimmune), among others, e.g., anti-PD1 antibodies disclosed in U.S. Pat. Nos. 8,609,089, 20,100,28330, and/or US20120114649.

In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224 (B7-DCIg; Amplimmune; e.g., disclosed in WO2010/027827 and WO2011/066342), is a PD-L2 Fc fusion soluble receptor that blocks the interaction between PD-1 and B7-H1.

In one embodiment, an anti-HBsAg antibody combination is used with an anti-PD-L1 antibody, e.g., as described in WO2016/061142. In some embodiments, the anti-PD-L1 antibody is Atezolizumab, Avelumab or Durvalumab. In some embodiments, the anti-PD-L1 antibody is Atezolizumab, disclosed in WO2010/077634. In some embodiments, the anti-PD-L1 antibody is Durvalumab, disclosed in WO2011/066389. Other anti-PD-L1 antibodies include BMS-936559 also known as MDX-1105, as disclosed in WO2007/005874 and AMP-224 also known as GSK2661380 disclosed in WO2010/027423.

### Pharmaceutical Compositions

To prepare pharmaceutical or sterile compositions including anti-HBsAg antibodies, the antibodies of the present disclosure are mixed with a pharmaceutically acceptable carrier or excipient. The compositions can additionally contain one or more other therapeutic agents that are suitable for neutralizing hepatitis D infection.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., Goodman and Gilman's The Pharmacological Basis of Therapeutics, McGraw-Hill, New York, N.Y., 2001; Gennaro, Remington: The Science and Practice of Pharmacy, Lippincott, Williams, and Wilkins, New York, N.Y., 2000; Avis, et al. (eds.), Pharmaceutical Dosage Forms: Parenteral Medications, Marcel Dekker, N Y, 1993; Lieberman, et al. (eds.), Pharmaceutical Dosage Forms: Tablets, Marcel Dekker, N Y, 1990; Lieberman, et al. (eds.) Pharmaceutical Dosage Forms: Disperse Systems, Marcel Dekker, N Y, 1990; Weiner and Kotkoskie, Excipient Toxicity and Safety, Marcel Dekker, Inc., New York, N.Y., 2000).

In a specific aspect, the anti-HBsAg antibody is a lyophilisate in a vial containing the antibody. The lyophilisate can be reconstituted with water or a pharmaceutical carrier suitable for injection.

For subsequent intravenous administration, the obtained solution will usually be further diluted into a carrier solution.

The antibodies disclosed herein are useful in the neutralization of hepatitis D in patients suffering from liver failure, cirrhosis, and/or hepatocellular carcinoma, so a pharmaceutical carrier of sucrose and human albumin as used previously in bone marrow transplant patients receiving CytoGam.®. can be used (DeRienzo et al. Pharmacotherapy 2000; 20:1175-8). Alternatively, the anti-HBsAg antibodies can be introduced into transplant patients via a pharmaceutical carrier as described for another anti-viral antibody, Synagis.®., as described in WO2003/105894. In this publication, the pharmaceutical carrier was comprised of histidine and/or glycine, a saccharide (e.g. sucrose) and a polyol (e.g. polysorbate).

Selecting an administration regimen for a therapeutic depends on several factors, including the severity of the infection, the level of symptoms, and the accessibility of the target cells in the biological matrix. In certain aspects, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak, Antibody Therapy, Bios Scientific Pub. Ltd, Oxfordshire, U K, 1996; Kresina (ed.), Monoclonal Antibodies, Cytokines and Arthritis, Marcel Dekker, New York, N.Y., 1991; Bach (ed.), Monoclonal Antibodies and Peptide Therapy in Autoimmune Diseases, Marcel Dekker, New York, N.Y., 1993; Baert et al., New Engl. J. Med. 348:601-608, 2003; Milgrom et al., New Engl. J. Med. 341:1966-1973, 1999; Slamon et al., New Engl. J. Med. 344:783-792, 2001; Beniaminovitz et al., New Engl. J. Med. 342:613-619, 2000; Ghosh et al., New Engl. J. Med. 348:24-32, 2003; Lipsky et al., New Engl. J. Med. 343:1594-1602, 2000).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., infusion reactions.

Actual dosage levels of the active ingredients in the pharmaceutical compositions with the anti-HBsAg antibodies can be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the neutralizing activity of the antibodies, the route of administration, the time of administration, the half-life of the antibody in the patient, the duration of the treatment, other drugs, compounds and/or materials used in combination with the particular compositions employed, the age, sex, weight, condition, general health and prior medical history of the patient being treated, and like factors known in the medical arts.

Compositions comprising antibodies or fragments thereof can be provided by continuous infusion, or by doses at intervals of, e.g., one day, one week, or 1-7 times per week. Doses can be provided intravenously, subcutaneously, topically, orally, nasally, rectally, intramuscular, intracerebrally, or by inhalation. A specific dose protocol is one involving the maximal dose or dose frequency that avoids significant undesirable side effects.

For the antibodies described herein, the dosage administered to a patient may be 0.0001 mg/kg to 50 mg/kg of the patient's body weight. The dosage may be between 0.0001 mg/kg and 40 mg/kg, 0.0001 mg/kg and 30 mg/kg, 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. The dosage of the antibodies or fragments thereof can be calculated using the patient's weight in kilograms (kg) multiplied by the dose to be administered in mg/kg.

Doses of the antibodies then can be repeated and the administrations may be separated by at least 1 day, 2 days, 3 days, 5 days, 10 days, 15 days, 30 days, 45 days, 2 months, 75 days, 3 months, or at least 6 months.

An effective amount for a particular patient may vary depending on factors such as the condition being treated, the overall health of the patient, the method, route and dose of administration and the severity of side effects (see, e.g., Maynard et al., A Handbook of SOPs for Good Clinical Practice, Interpharm Press, Boca Raton, Fla., 1996; Dent, Good Laboratory and Good Clinical Practice, Urch Publ., London, U K, 2001).

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al., Biopolymers 22:547-556, 1983; Langer et al., J. Biomed. Mater. Res. 15:167-277, 1981; Langer, Chem. Tech. 12:98-105, 1982; Epstein et al., Proc. Natl. Acad. Sci. USA 82:3688-3692, 1985; Hwang et al., Proc. Natl. Acad. Sci. USA 77:4030-4034, 1980; U.S. Pat. Nos. 6,350,466 and 6,316,024). Where necessary, the composition may also include a solubilizing agent or a local anesthetic such as lidocaine to case pain at the site of the injection, or both. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

A composition of the present disclosure can also be administered via one or more routes of administration using one or more of a variety of methods known in the art. As will be appreciated by the skilled artisan, the route and/or mode of administration will vary depending upon the desired results. Selected routes of administration for the antibodies include intravenous, intramuscular, intradermal, intraperitoneal, subcutaneous, spinal or other parenteral routes of administration, for example by injection or infusion. Parenteral administration can represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the present disclosure can be administered via a non-parenteral route, such as a topical, epidermal or mucosal route of administration, for example, intranasally, orally, vaginally, rectally, sublingually or topically. In one aspect, the antibodies of the present disclosure are administered by infusion. In another aspect, the antibodies are administered subcutaneously.

If the antibodies of the present disclosure are administered in a controlled release or sustained release system, a pump may be used to achieve controlled or sustained release (see Langer, supra; Sefton, CRC Crit. Ref Biomed. Eng. 14:20, 1987; Buchwald et al., Surgery 88:507, 1980; Saudek et al., N. Engl. J. Med. 321:574, 1989). Polymeric materials can be used to achieve controlled or sustained release of the therapies of the antibodies (see e.g., Medical Applications of Controlled Release, Langer and Wise (eds.), CRC Pres., Boca Raton, Fla., 1974; Controlled Drug Bioavailability, Drug Product Design and Performance, Smolen and Ball (eds.), Wiley, New York, 1984; Ranger and Peppas, J. Macromol. Sci. Rev. Macromol. Chem. 23:61, 1983; see also Levy et al., Science 228:190, 1985; During et al., Ann. Neurol. 25:351, 1989; Howard et al., J. Neurosurg. 7 1:105, 1989; U.S. Pat. Nos. 5,679,377; 5,916,597; 5,912,015; 5,989,463; 5,128,326; PCT Publication No. WO 99/15154; and PCT Publication No. WO 99/20253.

Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one aspect, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138, 1984).

Controlled release systems are discussed in the review by Langer, Science 249:1527-1533, 1990). Any technique known to one of skill in the art can be used to produce sustained release formulations comprising one or more antibodies of the present disclosure. See, e.g., U.S. Pat. No. 4,526,938, PCT publication WO 91/05548, PCT publication WO 96/20698, Ning et al., Radiotherapy & Oncology 39:179-189, 1996; Song et al., PDA Journal of Pharmaceutical Science & Technology 50:372-397, 1995; Cleek et al., Pro. Int'l. Symp. Control. Rd. Bioact. Mater. 24:853-854, 1997; and Lam et al., Proc. Int'l. Symp. Control Rd. Bioact. Mater. 24:759-760, 1997, each of which is incorporated herein by reference in their entirety.

If the antibodies of the disclosure are administered topically, they can be formulated in the form of an ointment, cream, transdermal patch, lotion, gel, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the compositions comprising the antibodies are administered intranasally, it can be formulated in an aerosol form, spray, mist or in the form of drops. In particular, prophylactic or therapeutic agents for use according to the present disclosure can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

Methods for co-administration or treatment with a second therapeutic agent, e.g., an immunosuppressant, a cytokine, steroid, chemotherapeutic agent, antibiotic, or radiation, are known in the art (see, e.g., Hardman et al., (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%; by at least 20%; at least about 30%; at least 40%, or at least 50%.

Additional therapies (e.g., prophylactic or therapeutic agents), which can be administered in combination with the anti-HBsAg antibodies may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the anti-HBsAg antibodies of the present disclosure. The two or more therapies may be administered within one same patient visit.

In certain aspects, anti-HBsAg antibodies can be formulated to ensure proper distribution in vivo. For example, the blood-brain barrier (BBB) excludes many highly hydrophilic compounds. To ensure that the anti-HBsAg antibodies cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade, (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (Bloeman et al., (1995) FEBS Lett. 357:140; Owais et al., (1995) Antimicrob. Agents Chemother. 39:180); surfactant protein A receptor (Briscoe et al., (1995) Am. J. Physiol. 1233:134); p 120 (Schreier et al, (1994) J. Biol. Chem. 269:9090); see also K. Keinanen; M. L. Laukkanen (1994) FEBS Lett. 346:123; J. J. Killion; I. J. Fidler (1994) Immunomethods 4:273.

The present disclosure provides protocols for the administration of pharmaceutical composition comprising antibodies alone or in combination with other therapies to a subject in need thereof. The combination therapies (e.g., prophylactic or therapeutic agents) can be administered concomitantly or sequentially to a subject. The therapy (e.g., prophylactic or therapeutic agents) of the combination therapies can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the disclosure can be administered to a subject concurrently. The term “concurrently” is not limited to the administration of therapies (e.g., prophylactic or therapeutic agents) at exactly the same time, but rather it is meant that a pharmaceutical composition comprising antibodies or fragments thereof are administered to a subject in a sequence and within a time interval such that the antibodies can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various aspects, the therapies (e.g., prophylactic or therapeutic agents) are administered to a subject less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other aspects, two or more therapies (e.g., prophylactic or therapeutic agents) are administered to a within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

### Working Examples

### Example 1: Generation of Test Articles

HBsAgBJ is an HBsAg antibody that was produced by Novartis Institutes for BioMedical Research Biologics Center. Myrcludex B (bulvertide) was provided by WuXi. The detailed information is listed here:

### Compound Information

| Compound | Batch | | Stock | |
|---|---|---|---|---|
| ID | number | M.W | concentration | Source |
| HBsAgBJ | 001-Novartis | ~150,000 | 43 mg/mL | Bluejay |
| | | | | Therapeutics |
| Bulvertide | C5595FF240- | 5,439 | 400 μM | GenScript |
| | 1/PE6884 | | | |

### HDV and HBV

HDV inoculum used in the assays was provided by WuXi, which was produced by co-transfection of plasmids pAAV-1.2×HDV genotype 1-ITR 141 and pAAV-HBsAg-141 ITR into Huh7 cells. HBV inoculum will be provided by WuXi, which is precipitated by PEG8000 from HepG2.2.15 cell culture supernatant.

### Information of the Virus

| Name | Genotype | Source |
|---|---|---|
| HDV | 1 | Huh7 cell culture supernatants |
| HBV | D | HepG2.2.15 culture supernatants |

### Primary Human Hepatocyte (PHHs)

Cryopreserved single donor primary human hepatocytes were provided by WuXi. Other test articles are listed below:

NOV3832, NOV3833, NOV3831, NOV3540, NOV3357, NOV3834, NOV3835, NOV3836, NOV3837, NOV3838, NOV3839, NOV3840, NOV3841, NOV3842, NOV2603, NOV3212 listed in U.S. Pat. No. 21,210,221871A1, WO2019229699

### Example 2 Neutralization Potency of HBsAgBJ Against Hepatitis B and D Viruses

The ability of HBsAgBJ to neutralize HBV and HDV infections was assessed using de novo infection of primary human hepatocytes. HBsAgBJ showed potent neutralization of infectious HBV in a de novo infection assay, with half-maximal effective concentration (EC₅₀) values of 0.09 and 0.11 nM for HBsAg and hepatitis B early antigen (HBeAg), respectively (Table 3). HBsAgBJ also showed potent neutralization of infectious HDV, with an EC₅₀ value of 0.01 nM (Table 4). In contrast, bulevirtide, an entry inhibitor, prevented HBV and HDV infections, with EC₅₀ values ranging from 0.77 to 1.45 nM.

TABLE 2
Neutralization Potency Against HBV

Mean EC₅₀ (nM) ± SD

| Inhibitorb | HBsAg | HBeAg |
|---|---|---|
| HBsAgBJ | 0.09 ± 0.02 | 0.11 ± 0.07 |
| Bulevirtide | 1.09 ± 0.58 | 1.45 ± 0.6 |

EC₅₀ = half-maximal effective concentration; HBeAg = hepatitis B early antigen; HBsAg = hepatitis B surface antigen; HBV = hepatitis B virus; SD = standard deviation.

TABLE 3
Neutralization Potency Against HDV

| | HDV RNA |
|---|---|
| Inhibitor | Mean EC₅₀ (nM) ± SD |
| HBsAgBJ | 0.01 ± 0.003 |
| Bulevirtide | 0.77 ± 0.39 |

EC₅₀ = half-maximal effective concentration; HDV = hepatitis D virus; RNA = ribonucleic acid; SD = standard deviation.

### Example 3. Pharmacologic Assessment of HBsAgBJ in an HBV/HDV Coinfection Mouse Model

The efficacy of HBsAgBJ was investigated in a humanized mouse model of HBV/HDV chronic infection (1). A dose of 20 mg/kg HBsAg demonstrated rapid depletion of HBsAg and HDV-RNA levels from serum. Furthermore, HBsAgBJ was able to maintain depletion of HBsAg and HDV RNA for more than 3 days. The IgG control, had minimal activity against HBsAg and HDV RNA levels in this model. (Marc Lütgehetmann et al. Humanicontroleric uPA mouse model for the study of hepatitis B and D virus interactions and preclinical drug evaluation. Hepatology; 2012 March; 55 (3): 685-94)

### Example 4: Formulation

The anti-HBsAg virus antibodies described herein are monoclonal antibodies, IgG1 isotype with kappa or lambda light chains, and can be lyophilized. For subsequent intravenous administration, the obtained solution will usually be further diluted into a carrier solution to the ready-to-use antibody solution for infusion. Important stability-indicating analytical methods to select the most stable formulation encompassed, amongst others, size-exclusion chromatography to determine aggregation levels, subvisible particulate matter testing, and potency testing.

### Example 5

Preliminary Efficacy of HBsAgBJ in a Clinical Trial for Subjects with Chronic Hepatitis D Infection

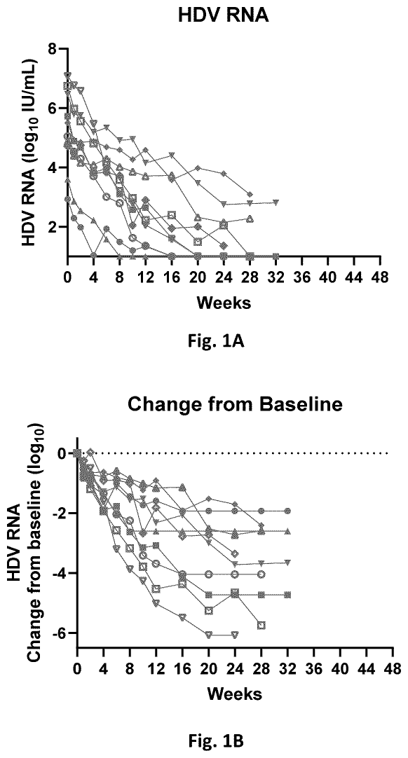

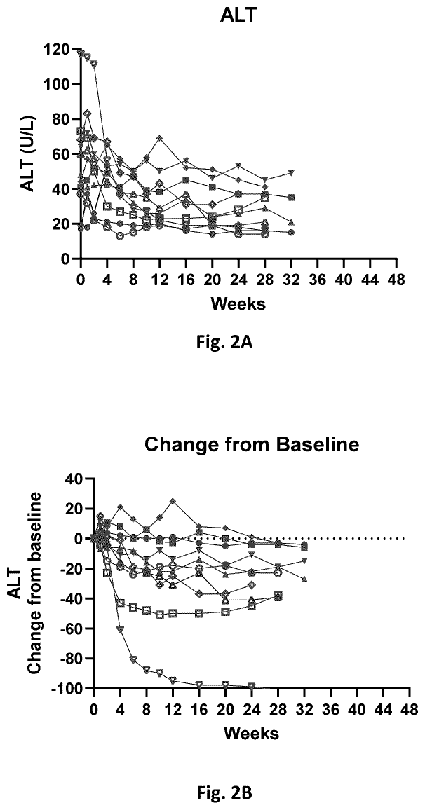

In the study HBsAgBJ-001, ten subjects with chronic HDV are receiving HBsAgBJ 300 mg subcutaneously once weekly for up to 48 weeks. All 10 subjects have HDV RNA data through Week 20 of treatment, and 8 subjects have data up to Week 28. At Week 12 and Week 24, the mean HDV RNA reductions from baseline are 2.7 and 3.6 log₁₀ IU/mL, respectively. To date, all 10 (100%) subjects have achieved a virologic response (>2 log reduction or HDV RNA below the limit of quantification (BLQ, 10 IU/ml in the assay used, Victoria Infectious Disease Reference Laboratory, Melbourne, Australia). Six (60%) have achieved HDV RNA BLQ. Six of 9 (67%) subjects with abnormal ALT at baseline have normalized ALT, while 1 subject had a normal ALT at baseline that has remained normal on treatment. The upper limit of normal of ALT for men in the study is 40 and for women is 30 per the central lab reference ranges. Six of 9 (67%) subjects have achieved the combined endpoint of virologic response and ALT normalization, which is the endpoint that has been used in hepatitis D for approval. The data are shown in FIGS. 1A-1B and FIGS. 2A-2B.

It is understood that the examples and aspects described herein are for illustrative purposes only and that various modifications or changes in light thereof will be suggested to persons skilled in the art and are to be included within the spirit and purview of this application and scope of the appended claims. All references listed in the specification are fully incorporated in the application.

1. A method of neutralizing a hepatitis D virus infection comprising administering via injection or infusion to a patient in need an effective amount of the antibody, wherein said antibody or antigen binding fragment thereof comprises: (i) a heavy chain variable region that comprises (a) a HCDR1 (CDR-Complementarity Determining Region) of SEQ ID NO: 9, (b) a HCDR2 of SEQ ID NO:10, (c) a HCDR3 of SEQ ID NO:11 and a light chain variable region that comprises: (d) a LCDR1 of SEQ ID NO:25, (c) a LCDR2 of SEQ ID NO:26, and (f) a LCDR3 of SEQ ID NO: 27; (ii) a heavy chain variable region that comprises (a) a HCDR1 of SEQ ID NO:41, (b) a HCDR2 of SEQ ID NO:42, (c) a HCDR3 of SEQ ID NO:43; and a light chain variable region that comprises: (d) a LCDR1 of SEQ ID NO:57, (c) a LCDR2 of SEQ ID NO:58, and (f) a LCDR3 of SEQ ID NO:59; (iii) a heavy chain variable region that comprises (a) a HCDR1 of SEQ ID NO:73, (b) a HCDR2 of SEQ ID NO:74, (c) a HCDR3 of SEQ ID NO:75; and a light chain variable region that comprises: (d) a LCDR1 of SEQ ID NO:89, (c) a LCDR2 of SEQ ID NO: 90, and (f) a LCDR3 of SEQ ID NO:91; (iv) a heavy chain variable region that comprises: (a) a HCDR1 of SEQ ID NO:105, (b) a HCDR2 of SEQ ID NO:106, (c) a HCDR3 of SEQ ID NO: 107; and a light chain variable region that comprises: (d) a LCDR1 of SEQ ID NO:121, (c) a LCDR2 of SEQ ID NO:122, and (f) a LCDR3 of SEQ ID NO:123; (v) a heavy chain variable region that comprises: (a) a HCDR1 of SEQ ID NO:137, (b) a HCDR2 of SEQ ID NO:138, (c) a HCDR3 of SEQ ID NO:139; and a light chain variable region that comprises: (d) a LCDR1 of SEQ ID NO:153, (c) a LCDR2 of SEQ ID NO:154, and (f) a LCDR3 of SEQ ID NO:155; (vi) a heavy chain variable region that comprises: (a) a HCDR1 of SEQ ID NO: 169, (b) a HCDR2 of SEQ ID NO:170, (c) a HCDR3 of SEQ ID NO:171; and a light chain variable region that comprises: (d) a LCDR1 of SEQ ID NO:185, (c) a LCDR2 of SEQ ID NO:186, and (f) a LCDR3 of SEQ ID NO:187; (vii) a heavy chain variable region that comprises: (a) a HCDR1 of SEQ ID NO: 201, (b) a HCDR2 of SEQ ID NO: 202, (c) a HCDR3 of SEQ ID NO:203; and a light chain variable region that comprises: (d) a LCDR1 of SEQ ID NO:217, (c) a LCDR2 of SEQ ID NO: 218, and (f) a LCDR3 of SEQ ID NO:219; (viii) a heavy chain variable region that comprises: (a) a HCDR1 of SEQ ID NO:233, (b) a HCDR2 of SEQ ID NO:234, (c) a HCDR3 of SEQ ID NO:235; and a light chain variable region that comprises: (d) a LCDR1 of SEQ ID NO: 249, (c) a LCDR2 of SEQ ID NO:250, and (f) a LCDR3 of SEQ ID NO:251; (ix) a heavy chain variable region that comprises: (a) a HCDR1 of SEQ ID NO:265, (b) a HCDR2 of SEQ ID NO: 266, (c) a HCDR3 of SEQ ID NO:267; and a light chain variable region that comprises: (d) a LCDR1 of SEQ ID NO:281, (c) a LCDR2 of SEQ ID NO:282, and (f) a LCDR3 of SEQ ID NO: 283; (x) a heavy chain variable region that comprises: (a) a HCDR1 of SEQ ID NO:297, (b) a HCDR2 of SEQ ID NO: 298, (c) a HCDR3 of SEQ ID NO:299; and a light chain variable region that comprises: (d) a LCDR1 of SEQ ID NO:313, (e) a LCDR2 of SEQ ID NO:314, and (f) a LCDR3 of SEQ ID NO: 315; (xi) a heavy chain variable region that comprises: (a) a HCDR1 of SEQ ID NO:329, (b) a HCDR2 of SEQ ID NO:330, (c) a HCDR3 of SEQ ID NO: 331; and a light chain variable region that comprises: (d) a LCDR1 of SEQ ID NO:345, (c) a LCDR2 of SEQ ID NO:346, and (f) a LCDR3 of SEQ ID NO: 347; (xii) a heavy chain variable region that comprises: (a) a HCDR1 of SEQ ID NO:361, (b) a HCDR2 of SEQ ID NO:362, (c) a HCDR3 of SEQ ID NO:363; and a light chain variable region that comprises: (d) a LCDR1 of SEQ ID NO:377, (c) a LCDR2 of SEQ ID NO:378, and (f) a LCDR3 of SEQ ID NO: 379; (xiii) a heavy chain variable region that comprises: (a) a HCDR1 of SEQ ID NO:393, (b) a HCDR2 of SEQ ID NO:394, (c) a HCDR3 of SEQ ID NO:395; and a light chain variable region that comprises: (d) a LCDR1 of SEQ ID NO:409, (c) a LCDR2 of SEQ ID NO:410, and (f) a LCDR3 of SEQ ID NO:411; (xiv) a heavy chain variable region that comprises: (a) a HCDR1 of SEQ ID NO: 425, (b) a HCDR2 of SEQ ID NO:426, (c) a HCDR3 of SEQ ID NO:427; and a light chain variable region that comprises: (d) a LCDR1 of SEQ ID NO:441, (c) a LCDR2 of SEQ ID NO: 442, and (f) a LCDR3 of SEQ ID NO: 443; (xv) a heavy chain variable region that comprises: (a) a HCDR1 of SEQ ID NO:457, (b) a HCDR2 of SEQ ID NO:458, (c) a HCDR3 of SEQ ID NO:459; and a light chain variable region that comprises: (d) a LCDR1 of SEQ ID NO: 473, (c) a LCDR2 of SEQ ID NO:474, and (f) a LCDR3 of SEQ ID NO:475; or (xvi) a heavy chain variable region that comprises: (a) a HCDR1 of SEQ ID NO:489, (b) a HCDR2 of SEQ ID NO:490, (c) a HCDR3 of SEQ ID NO:491; and a light chain variable region that comprises: (d) a LCDR1 of SEQ ID NO:505, (e) a LCDR2 of SEQ ID NO:506, and (f) a LCDR3 of SEQ ID NO: 507.

2. The method of claim 1, wherein one or two amino acids of the antibody within a CDR have been modified, deleted or substituted.

3. The method of claim 1, wherein the antibody retains at least 90, 91, 92, 93, 94, 95, 96, 97, 98 or 99% identity over either the variable heavy chain region or the variable light chain region.

4. The method of claim 1, wherein the antibody is a monoclonal antibody, a chimeric antibody, a humanized antibody, a human engineered antibody, a human antibody, a single chain antibody (scFv) or an antibody fragment.

5. A method of neutralizing a hepatitis D virus infection comprising administering via injection or infusion to a patient in need an effective amount of the antibody, wherein said antibody or antigen binding fragment thereof comprises: (i) a heavy chain variable region (vH) that comprises SEQ ID NO: 18, and a light chain variable region (vL) that comprises SEQ ID NO: 34; (ii) a heavy chain variable region (vH) that comprises SEQ ID NO: 50, and a light chain variable region (vL) that comprises SEQ ID NO: 66; (iii) a heavy chain variable region (vH) that comprises SEQ ID NO: 82, and a light chain variable region (vL) that comprises SEQ ID NO:98; (iv) a heavy chain variable region (vH) that comprises SEQ ID NO: 114, and a light chain variable region (vL) that comprises SEQ ID NO:130; (v) a heavy chain variable region (vH) that comprises SEQ ID NO: 146, and a light chain variable region (vL) that comprises SEQ ID NO: 162; (vi) a heavy chain variable region (vH) that comprises SEQ ID NO:178, and a light chain variable region (vL) that comprises SEQ ID NO: 194; (vii) a heavy chain variable region (vH) that comprises SEQ ID NO:210, and a light chain variable region (vL) that comprises SEQ ID NO: 226; (viii) a heavy chain variable region (vH) that comprises SEQ ID NO:242, and a light chain variable region (vL) that comprises SEQ ID NO:258; (ix) a heavy chain variable region (vH) that comprises SEQ ID NO:274, and a light chain variable region (vL) that comprises SEQ ID NO: 290; (x) a heavy chain variable region (vH) that comprises SEQ ID NO:306, and a light chain variable region (vL) that comprises SEQ ID NO:322; (xi) a heavy chain variable region (vH) that comprises SEQ ID NO:338, and a light chain variable region (vL) that comprises SEQ ID NO: 354; (xii) a heavy chain variable region (vH) that comprises SEQ ID NO:370, and a light chain variable region (vL) that comprises SEQ ID NO:386; (xiii) a heavy chain variable region (vH) that comprises SEQ ID NO:402, and a light chain variable region (vL) that comprises SEQ ID NO: 418; (xiv) a heavy chain variable region (vH) that comprises SEQ ID NO:434, and a light chain variable region (vL) that comprises SEQ ID NO:450; (xv) a heavy chain variable region (vH) that comprises SEQ ID NO:466, and a light chain variable region (vL) that comprises SEQ ID NO: 482; or (xvi) a heavy chain variable region (vH) that comprises SEQ ID NO:498, and a light chain variable region (vL) that comprises SEQ ID NO:514.

6. The method of the claim 5, wherein the antibody or fragment thereof retains at least 90, 91, 92, 93, 94, 95, 96, 97, 98 or 99% identity over either the variable light or variable heavy region.

7. The method of claim 5, wherein the antibody with one, two, three, four or five, but less than 10 amino acids within the variable light or variable heavy region have been modified, deleted or substituted.

8. The method of claim 5, wherein the antibody is a monoclonal antibody, a chimeric antibody, a humanized antibody, a human engineered antibody, a human antibody, a single chain antibody (scFv) or an antibody fragment.

9. The method of claim 1 wherein the antibody or fragment thereof has reduced glycosylation or no glycosylation or is hypofucosylated.

10. The method of claim 1 or 5 wherein a pharmaceutical composition comprising the antibody or fragment thereof, of claim 1, further comprising a pharmaceutically acceptable carrier.

11. A pharmaceutical composition comprising a plurality of an antibody or antigen binding fragment of claim 1 wherein at least 0.05%, 0.1%, 0.5%, 1%, 2%, 3%, 5% or more or more of the antibodies in the composition have an .alpha.2,3-linked sialic acid residue.

12. A pharmaceutical composition comprising a plurality of an antibody or antigen binding fragment of claim 1, wherein none of the antibodies comprise a bisecting GlcNAc.

13. The pharmaceutical composition comprising the antibody or fragment thereof of claim 1, wherein the composition is prepared as a lyophilisate.

14. A method of treating or reducing the likelihood of hepatitis D virus associated disorder, comprising administering via injection or infusion to a patient in need an effective amount of the antibody of claim 1, and wherein the disorder is: liver failure, cirrhosis, or hepatocellular carcinoma.

15. A diagnostic reagent comprising the antibody or antigen binding fragment thereof of claim 1 which is labeled.

16. A pharmaceutical composition comprising a plurality of an antibody or antigen binding fragment of claim 5, wherein at least 0.05%, 0.1%, 0.5%, 1%, 2%, 3%, 5% or more or more of the antibodies in the composition have an .alpha.2,3-linked sialic acid residue.

17. A pharmaceutical composition comprising a plurality of an antibody or antigen binding fragment of claim 5, wherein none of the antibodies comprise a bisecting GlcNAc.

18. The pharmaceutical composition comprising the antibody or fragment thereof of claim 5, wherein the composition is prepared as a lyophilisate.

19. A method of treating or reducing the likelihood of hepatitis D virus associated disorder, comprising administering via injection or infusion to a patient in need an effective amount of the antibody of claim 5, and wherein the disorder is: liver failure, cirrhosis, or hepatocellular carcinoma.

20. A diagnostic reagent comprising the antibody or antigen binding fragment thereof of claim 5 which is labeled.

21. A method of neutralizing a hepatitis D virus infection comprising administering via injection or infusion to a patient in need an effective amount of the antibody, wherein said antibody or antigen binding fragment thereof comprises sequences in Table 1.

22. A method of treating or reducing the likelihood of hepatitis D virus associated disorder, comprising administering via injection or infusion to a patient in need an effective amount of the antibody of Table 1, and wherein the disorder is: liver failure, cirrhosis, or hepatocellular carcinoma.


SEQUENCE LISTING (517 sequences)

SEQ ID NO: 1-5 - skipped (5 entries)

SEQ ID NO: 6 - protein, 10 aa, synthetic construct
  REGION 1..10: note = source = /note="Description of Artificial Sequence: Synthetic peptide"
GFTFDNYAMS

SEQ ID NO: 7 - protein, 17 aa, synthetic construct
  REGION 1..17: note = source = /note="Description of Artificial Sequence: Synthetic peptide"
SISGSGGSTYYADSVKG

SEQ ID NO: 8 - protein, 16 aa, synthetic construct
  REGION 1..16: note = source = /note="Description of Artificial Sequence: Synthetic peptide"
SSILSGGHARVYGIDV

SEQ ID NO: 9 - protein, 5 aa, synthetic construct
  REGION 1..5: note = source = /note="Description of Artificial Sequence: Synthetic peptide"
NYAMS

SEQ ID NO: 10 - protein, 17 aa, synthetic construct
  REGION 1..17: note = source = /note="Description of Artificial Sequence: Synthetic peptide"
SISGSGGSTYYADSVKG

SEQ ID NO: 11 - protein, 16 aa, synthetic construct
  REGION 1..16: note = source = /note="Description of Artificial Sequence: Synthetic peptide"
SSILSGGHARVYGIDV

SEQ ID NO: 12 - protein, 7 aa, synthetic construct
  REGION 1..7: note = source = /note="Description of Artificial Sequence: Synthetic peptide"
GFTFDNY

SEQ ID NO: 13 - protein, 6 aa, synthetic construct
  REGION 1..6: note = source = /note="Description of Artificial Sequence: Synthetic peptide"
SGSGGS

SEQ ID NO: 14 - protein, 16 aa, synthetic construct
  REGION 1..16: note = source = /note="Description of Artificial Sequence: Synthetic peptide"
SSILSGGHARVYGIDV

SEQ ID NO: 15 - protein, 8 aa, synthetic construct
  REGION 1..8: note = source = /note="Description of Artificial Sequence: Synthetic peptide"
GFTFDNYA

SEQ ID NO: 16 - protein, 8 aa, synthetic construct
  REGION 1..8: note = source = /note="Description of Artificial Sequence: Synthetic peptide"
ISGSGGST

SEQ ID NO: 17 - protein, 18 aa, synthetic construct
  REGION 1..18: note = source = /note="Description of Artificial Sequence: Synthetic peptide"
AKSSILSGGHARVYGIDV

SEQ ID NO: 18 - protein, 125 aa, synthetic construct
  REGION 1..125: note = source = /note="Description of Artificial Sequence: Synthetic polypeptide"
EMQVLESGGGLVQPGGSLRLSCAASGFTFDNYAMSWVRQVPGKGLEWVSSISGSGGSTYYADSVKGQFTISRDNSKNTLYLQMNSLRAEDTAVYYCAKSSILSGGHARVYGIDVWGQGTTVTVSS

SEQ ID NO: 19 - DNA, 375 bp, synthetic construct
  misc_feature 1..375: note = source = /note="Description of Artificial Sequence: Synthetic polynucleotide"
  source 1..375: mol_type = other DNA
gagatgcaggtcttggaatctggcggaggactggttcaacctggcggctctctgagactgtcttgtgccgccagcggcttcaccttcgataactacgccatgtcctgggtccgacaggtgccaggcaaaggactggaatgggtgtcctctatcagcggctctggcggcagcacatattacgccgatagcgtgaagggccagttcaccatcagccgggacaacagcaagaacaccctgtacctccagatgaacagcctgagagccgaggataccgccgtgtactactgtgccaagagcagcattctgtctggcggccacgccagagtgtatggcattgatgtttggggccagggaaccaccgtgaccgttagttct

SEQ ID NO: 20 - protein, 455 aa, synthetic construct
  REGION 1..455: note = source = /note="Description of Artificial Sequence: Synthetic polypeptide"
EMQVLESGGGLVQPGGSLRLSCAASGFTFDNYAMSWVRQVPGKGLEWVSSISGSGGSTYYADSVKGQFTISRDNSKNTLYLQMNSLRAEDTAVYYCAKSSILSGGHARVYGIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1365 bp, synthetic construct
  misc_feature 1..1365: note = source = /note="Description of Artificial Sequence: Synthetic polynucleotide"
  source 1..1365: mol_type = other DNA
gagatgcaggtcttggaatctggcggaggactggttcaacctggcggctctctgagactgtcttgtgccgccagcggcttcaccttcgataactacgccatgtcctgggtccgacaggtgccaggcaaaggactggaatgggtgtcctctatcagcggctctggcggcagcacatattacgccgatagcgtgaagggccagttcaccatcagccgggacaacagcaagaacaccctgtacctccagatgaacagcctgagagccgaggataccgccgtgtactactgtgccaagagcagcattctgtctggcggccacgccagagtgtatggcattgatgtttggggccagggaaccaccgtgaccgttagttctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacatgccccccctgcccggcgccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcttaagccccggcaag

SEQ ID NO: 22 - protein, 11 aa, synthetic construct
  REGION 1..11: note = source = /note="Description of Artificial Sequence: Synthetic peptide"
GGNNIGSQSVH

SEQ ID NO: 23 - protein, 7 aa, synthetic construct
  REGION 1..7: note = source = /note="Description of Artificial Sequence: Synthetic peptide"
DDTDRPS

SEQ ID NO: 24 - protein, 11 aa, synthetic construct
  REGION 1..11: note = source = /note="Description of Artificial Sequence: Synthetic peptide"
QVWDSSSDHVV

SEQ ID NO: 25 - protein, 11 aa, synthetic construct
  REGION 1..11: note = source = /note="Description of Artificial Sequence: Synthetic peptide"
GGNNIGSQSVH

SEQ ID NO: 26 - protein, 7 aa, synthetic construct
  REGION 1..7: note = source = /note="Description of Artificial Sequence: Synthetic peptide"
DDTDRPS

SEQ ID NO: 27 - protein, 11 aa, synthetic construct
  REGION 1..11: note = source = /note="Description of Artificial Sequence: Synthetic peptide"
QVWDSSSDHVV

SEQ ID NO: 28 - protein, 7 aa, synthetic construct
  REGION 1..7: note = source = /note="Description of Artificial Sequence: Synthetic peptide"
NNIGSQS

SEQ ID NO: 29 - skipped

SEQ ID NO: 30 - protein, 8 aa, synthetic construct
  REGION 1..8: note = source = /note="Description of Artificial Sequence: Synthetic peptide"
WDSSSDHV

SEQ ID NO: 31 - protein, 6 aa, synthetic construct
  REGION 1..6: note = source = /note="Description of Artificial Sequence: Synthetic peptide"
NIGSQS

SEQ ID NO: 32 - skipped

SEQ ID NO: 33 - protein, 11 aa, synthetic construct
  REGION 1..11: note = source = /note="Description of Artificial Sequence: Synthetic peptide"
QVWDSSSDHVV

SEQ ID NO: 34 - protein, 108 aa, synthetic construct
  REGION 1..108: note = source = /note="Description of Artificial Sequence: Synthetic polypeptide"
QSALTQPPSVSVAPGQTARITCGGNNIGSQSVHWYQQKPGQAPILVVYDDTDRPSGIPARFSGSSSGSTATLTIGRVEAGDEADYYCQVWDSSSDHVVFGGGTKLTVL

SEQ ID NO: 35 - DNA, 324 bp, synthetic construct
  misc_feature 1..324: note = source = /note="Description of Artificial Sequence: Synthetic polynucleotide"
  source 1..324: mol_type = other DNA
cagtctgccctgactcagccaccctcggtgtcagtggccccaggacagacggccaggattacctgtgggggaaacaacattggaagtcaaagtgtgcactggtaccagcagaagccaggccaggcccctatactggtcgtctatgatgataccgaccggccctcagggatccctgcgcgattctctggctccagctctgggagcacggccaccctgaccatcggcagggtcgaagccggggatgaggccgactattactgtcaggtgtgggatagtagtagtgatcatgtggtattcggcggagggaccaagctgaccgtctta

SEQ ID NO: 36 - protein, 214 aa, synthetic construct
  REGION 1..214: note = source = /note="Description of Artificial Sequence: Synthetic polypeptide"
QSALTQPPSVSVAPGQTARITCGGNNIGSQSVHWYQQKPGQAPILVVYDDTDRPSGIPARFSGSSSGSTATLTIGRVEAGDEADYYCQVWDSSSDHVVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - DNA, 642 bp, synthetic construct
  misc_feature 1..642: note = source = /note="Description of Artificial Sequence: Synthetic polynucleotide"
  source 1..642: mol_type = other DNA
cagtctgccctgactcagccaccctcggtgtcagtggccccaggacagacggccaggattacctgtgggggaaacaacattggaagtcaaagtgtgcactggtaccagcagaagccaggccaggcccctatactggtcgtctatgatgataccgaccggccctcagggatccctgcgcgattctctggctccagctctgggagcacggccaccctgaccatcggcagggtcgaagccggggatgaggccgactattactgtcaggtgtgggatagtagtagtgatcatgtggtattcggcggagggaccaagctgaccgtcttaagtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgccgccagcagctacctgagcctgacccccgagcagtggaagagccacagaagctacagctgccaggtcacccacgagggcagcaccgtggagaaaaccgtggcccccaccgagtgcagc

SEQ ID NO: 38 - protein, 10 aa, synthetic construct
  REGION 1..10: note = source = /note="Description of Artificial Sequence: Synthetic peptide"
GFTFHNYAMS

SEQ ID NO: 39 - protein, 17 aa, synthetic construct
  REGION 1..17: note = source = /note="Description of Artificial Sequence: Synthetic peptide"
SISGSGGSTYYADSVKG

SEQ ID NO: 40 - protein, 16 aa, synthetic construct
  REGION 1..16: note = source = /note="Description of Artificial Sequence: Synthetic peptide"
SSILSGGHARVYGIDV

SEQ ID NO: 41 - protein, 5 aa, synthetic construct
  REGION 1..5: note = source = /note="Description of Artificial Sequence: Synthetic peptide"
NYAMS

SEQ ID NO: 42 - protein, 17 aa, synthetic construct
  REGION 1..17: note = source = /note="Description of Artificial Sequence: Synthetic peptide"
SISGSGGSTYYADSVKG

SEQ ID NO: 43 - protein, 16 aa, synthetic construct
  REGION 1..16: note = source = /note="Description of Artificial Sequence: Synthetic peptide"
SSILSGGHARVYGIDV

SEQ ID NO: 44 - protein, 7 aa, synthetic construct
  REGION 1..7: note = source = /note="Description of Artificial Sequence: Synthetic peptide"
GFTFHNY

SEQ ID NO: 45 - protein, 6 aa, synthetic construct
  REGION 1..6: note = source = /note="Description of Artificial Sequence: Synthetic peptide"
SGSGGS

SEQ ID NO: 46 - protein, 16 aa, synthetic construct
  REGION 1..16: note = source = /note="Description of Artificial Sequence: Synthetic peptide"
SSILSGGHARVYGIDV

SEQ ID NO: 47 - protein, 8 aa, synthetic construct
  REGION 1..8: note = source = /note="Description of Artificial Sequence: Synthetic peptide"
GFTFHNYA

SEQ ID NO: 48 - protein, 8 aa, synthetic construct
  REGION 1..8: note = source = /note="Description of Artificial Sequence: Synthetic peptide"
ISGSGGST

SEQ ID NO: 49 - protein, 18 aa, synthetic construct
  REGION 1..18: note = source = /note="Description of Artificial Sequence: Synthetic peptide"
AKSSILSGGHARVYGIDV

SEQ ID NO: 50 - protein, 125 aa, synthetic construct
  REGION 1..125: note = source = /note="Description of Artificial Sequence: Synthetic polypeptide"
EMQVLESGGGLVQPGGSLRLSCAASGFTFHNYAMSWVRQVPGKGLEWVSSISGSGGSTYYADSVKGQFTISRDNSKNTLYLQMNSLRAEDTAVYYCAKSSILSGGHARVYGIDVWGQGTTVTVSS

SEQ ID NO: 51 - DNA, 375 bp, synthetic construct
  misc_feature 1..375: note = source = /note="Description of Artificial Sequence: Synthetic polynucleotide"
  source 1..375: mol_type = other DNA
gagatgcaggtcttggaatctggcggaggactggttcaacctggcggctctctgagactgtcttgtgccgccagcggcttcaccttccataactacgccatgtcctgggtccgacaggtgccaggcaaaggactggaatgggtgtcctctatcagcggctctggcggcagcacatattacgccgatagcgtgaagggccagttcaccatcagccgggacaacagcaagaacaccctgtacctccagatgaacagcctgagagccgaggataccgccgtgtactactgtgccaagagcagcattctgtctggcggccacgccagagtgtatggcattgatgtttggggccagggaaccaccgtgaccgttagttct

SEQ ID NO: 52 - protein, 455 aa, synthetic construct
  REGION 1..455: note = source = /note="Description of Artificial Sequence: Synthetic polypeptide"
EMQVLESGGGLVQPGGSLRLSCAASGFTFHNYAMSWVRQVPGKGLEWVSSISGSGGSTYYADSVKGQFTISRDNSKNTLYLQMNSLRAEDTAVYYCAKSSILSGGHARVYGIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - DNA, 1365 bp, synthetic construct
  misc_feature 1..1365: note = source = /note="Description of Artificial Sequence: Synthetic polynucleotide"
  source 1..1365: mol_type = other DNA
gagatgcaggtcttggaatctggcggaggactggttcaacctggcggctctctgagactgtcttgtgccgccagcggcttcaccttccataactacgccatgtcctgggtccgacaggtgccaggcaaaggactggaatgggtgtcctctatcagcggctctggcggcagcacatattacgccgatagcgtgaagggccagttcaccatcagccgggacaacagcaagaacaccctgtacctccagatgaacagcctgagagccgaggataccgccgtgtactactgtgccaagagcagcattctgtctggcggccacgccagagtgtatggcattgatgtttggggccagggaaccaccgtgaccgttagttctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacatgccccccctgcccggcgccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcttaagccccggcaag

SEQ ID NO: 54 - protein, 11 aa, synthetic construct
  REGION 1..11: note = source = /note="Description of Artificial Sequence: Synthetic peptide"
GGNNIGSQSVH

SEQ ID NO: 55 - protein, 7 aa, synthetic construct
  REGION 1..7: note = source = /note="Description of Artificial Sequence: Synthetic peptide"
DDTDRPS

SEQ ID NO: 56 - protein, 11 aa, synthetic construct
  REGION 1..11: note = source = /note="Description of Artificial Sequence: Synthetic peptide"
QVWDSSSDHVV

SEQ ID NO: 57 - protein, 11 aa, synthetic construct
  REGION 1..11: note = source = /note="Description of Artificial Sequence: Synthetic peptide"
GGNNIGSQSVH

SEQ ID NO: 58 - protein, 7 aa, synthetic construct
  REGION 1..7: note = source = /note="Description of Artificial Sequence: Synthetic peptide"
DDTDRPS

SEQ ID NO: 59 - protein, 11 aa, synthetic construct
  REGION 1..11: note = source = /note="Description of Artificial Sequence: Synthetic peptide"
QVWDSSSDHVV

SEQ ID NO: 60 - protein, 7 aa, synthetic construct
  REGION 1..7: note = source = /note="Description of Artificial Sequence: Synthetic peptide"
NNIGSQS

SEQ ID NO: 61 - skipped

SEQ ID NO: 62 - protein, 8 aa, synthetic construct
  REGION 1..8: note = source = /note="Description of Artificial Sequence: Synthetic peptide"
WDSSSDHV

SEQ ID NO: 63 - protein, 6 aa, synthetic construct
  REGION 1..6: note = source = /note="Description of Artificial Sequence: Synthetic peptide"
NIGSQS

SEQ ID NO: 64 - skipped

SEQ ID NO: 65 - protein, 11 aa, synthetic construct
  REGION 1..11: note = source = /note="Description of Artificial Sequence: Synthetic peptide"
QVWDSSSDHVV

SEQ ID NO: 66 - protein, 108 aa, synthetic construct; identical to SEQ ID NO: 34
  REGION 1..108: note = source = /note="Description of Artificial Sequence: Synthetic polypeptide"

SEQ ID NO: 67 - DNA, 324 bp, synthetic construct; identical to SEQ ID NO: 35
  misc_feature 1..324: note = source = /note="Description of Artificial Sequence: Synthetic polynucleotide"
  source 1..324: mol_type = other DNA

SEQ ID NO: 68 - protein, 214 aa, synthetic construct; identical to SEQ ID NO: 36
  REGION 1..214: note = source = /note="Description of Artificial Sequence: Synthetic polypeptide"

SEQ ID NO: 69 - DNA, 642 bp, synthetic construct; identical to SEQ ID NO: 37
  misc_feature 1..642: note = source = /note="Description of Artificial Sequence: Synthetic polynucleotide"
  source 1..642: mol_type = other DNA

SEQ ID NO: 70 - protein, 10 aa, synthetic construct
  REGION 1..10: note = source = /note="Description of Artificial Sequence: Synthetic peptide"
GFTFNNYAMS

SEQ ID NO: 71 - protein, 17 aa, synthetic construct
  REGION 1..17: note = source = /note="Description of Artificial Sequence: Synthetic peptide"
SISGSGGSTYYADSVKG

SEQ ID NO: 72 - protein, 16 aa, synthetic construct
  REGION 1..16: note = source = /note="Description of Artificial Sequence: Synthetic peptide"
SSILSGGHARVYGIDV

SEQ ID NO: 73 - protein, 5 aa, synthetic construct
  REGION 1..5: note = source = /note="Description of Artificial Sequence: Synthetic peptide"
NYAMS

SEQ ID NO: 74 - protein, 17 aa, synthetic construct
  REGION 1..17: note = source = /note="Description of Artificial Sequence: Synthetic peptide"
SISGSGGSTYYADSVKG

SEQ ID NO: 75 - protein, 16 aa, synthetic construct
  REGION 1..16: note = source = /note="Description of Artificial Sequence: Synthetic peptide"
SSILSGGHARVYGIDV

SEQ ID NO: 76 - protein, 7 aa, synthetic construct
  REGION 1..7: note = source = /note="Description of Artificial Sequence: Synthetic peptide"
GFTFNNY

SEQ ID NO: 77 - protein, 6 aa, synthetic construct
  REGION 1..6: note = source = /note="Description of Artificial Sequence: Synthetic peptide"
SGSGGS

SEQ ID NO: 78 - protein, 16 aa, synthetic construct
  REGION 1..16: note = source = /note="Description of Artificial Sequence: Synthetic peptide"
SSILSGGHARVYGIDV

SEQ ID NO: 79 - protein, 8 aa, synthetic construct
  REGION 1..8: note = source = /note="Description of Artificial Sequence: Synthetic peptide"
GFTFNNYA

SEQ ID NO: 80 - protein, 8 aa, synthetic construct
  REGION 1..8: note = source = /note="Description of Artificial Sequence: Synthetic peptide"
ISGSGGST

SEQ ID NO: 81 - protein, 18 aa, synthetic construct
  REGION 1..18: note = source = /note="Description of Artificial Sequence: Synthetic peptide"
AKSSILSGGHARVYGIDV

SEQ ID NO: 82 - protein, 125 aa, synthetic construct
  REGION 1..125: note = source = /note="Description of Artificial Sequence: Synthetic polypeptide"
EMQVLESGGGLVQPGGSLRLSCAASGFTFNNYAMSWVRQVPGKGLEWVSSISGSGGSTYYADSVKGQFTISRDNSKNTLYLQMNSLRAEDTAVYYCAKSSILSGGHARVYGIDVWGQGTTVTVSS

SEQ ID NO: 83 - DNA, 375 bp, synthetic construct
  misc_feature 1..375: note = source = /note="Description of Artificial Sequence: Synthetic polynucleotide"
  source 1..375: mol_type = other DNA
gaaatgcaggtgttggagtctgggggaggcctggtacagcctggggggtccctgagactctcctgtgcagcctctggattcacctttaacaactatgccatgagctgggtccgccaggttccagggaaggggctggagtgggtctcaagtattagtggtagtggaggtagcacgtactacgcagactccgtgaagggccagttcaccatctccagagacaattccaagaatacgctgtatctgcaaatgaacagcctgagagccgaggacacggccgtatattactgtgcgaaatcctctatcttgagtggtggtcacgcgcgggtctacggcatagacgtctggggccaagggaccacggtcaccgtctcctca

SEQ ID NO: 84 - protein, 455 aa, synthetic construct
  REGION 1..455: note = source = /note="Description of Artificial Sequence: Synthetic polypeptide"
EMQVLESGGGLVQPGGSLRLSCAASGFTFNNYAMSWVRQVPGKGLEWVSSISGSGGSTYYADSVKGQFTISRDNSKNTLYLQMNSLRAEDTAVYYCAKSSILSGGHARVYGIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 1365 bp, synthetic construct
  misc_feature 1..1365: note = source = /note="Description of Artificial Sequence: Synthetic polynucleotide"
  source 1..1365: mol_type = other DNA
gaaatgcaggtgttggagtctgggggaggcctggtacagcctggggggtccctgagactctcctgtgcagcctctggattcacctttaacaactatgccatgagctgggtccgccaggttccagggaaggggctggagtgggtctcaagtattagtggtagtggaggtagcacgtactacgcagactccgtgaagggccagttcaccatctccagagacaattccaagaatacgctgtatctgcaaatgaacagcctgagagccgaggacacggccgtatattactgtgcgaaatcctctatcttgagtggtggtcacgcgcgggtctacggcatagacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcatggaactcaggcgccctgaccagcggcgtgcacaccttcccggctgtcctacagtcctcaggactctactccctcagcagcgtggtgaccgtgccctccagcagcttgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 86 - protein, 11 aa, synthetic construct
  REGION 1..11: note = source = /note="Description of Artificial Sequence: Synthetic peptide"
GGNNIGSQSVH

SEQ ID NO: 87 - protein, 7 aa, synthetic construct
  REGION 1..7: note = source = /note="Description of Artificial Sequence: Synthetic peptide"
DDTDRPS

SEQ ID NO: 88 - protein, 11 aa, synthetic construct
  REGION 1..11: note = source = /note="Description of Artificial Sequence: Synthetic peptide"
QVWDSSSDHVV

SEQ ID NO: 89 - protein, 11 aa, synthetic construct
  REGION 1..11: note = source = /note="Description of Artificial Sequence: Synthetic peptide"
GGNNIGSQSVH

SEQ ID NO: 90 - protein, 7 aa, synthetic construct
  REGION 1..7: note = source = /note="Description of Artificial Sequence: Synthetic peptide"
DDTDRPS

SEQ ID NO: 91 - protein, 11 aa, synthetic construct
  REGION 1..11: note = source = /note="Description of Artificial Sequence: Synthetic peptide"
QVWDSSSDHVV

SEQ ID NO: 92 - protein, 7 aa, synthetic construct
  REGION 1..7: note = source = /note="Description of Artificial Sequence: Synthetic peptide"
NNIGSQS

SEQ ID NO: 93 - skipped

SEQ ID NO: 94 - protein, 8 aa, synthetic construct
  REGION 1..8: note = source = /note="Description of Artificial Sequence: Synthetic peptide"
WDSSSDHV

SEQ ID NO: 95 - protein, 6 aa, synthetic construct
  REGION 1..6: note = source = /note="Description of Artificial Sequence: Synthetic peptide"
NIGSQS

SEQ ID NO: 96 - skipped

SEQ ID NO: 97 - protein, 11 aa, synthetic construct
  REGION 1..11: note = source = /note="Description of Artificial Sequence: Synthetic peptide"
QVWDSSSDHVV

SEQ ID NO: 98 - protein, 108 aa, synthetic construct; identical to SEQ ID NO: 34
  REGION 1..108: note = source = /note="Description of Artificial Sequence: Synthetic polypeptide"

SEQ ID NO: 99 - DNA, 324 bp, synthetic construct; identical to SEQ ID NO: 35
  misc_feature 1..324: note = source = /note="Description of Artificial Sequence: Synthetic polynucleotide"
  source 1..324: mol_type = other DNA

SEQ ID NO: 100 - protein, 214 aa, synthetic construct; identical to SEQ ID NO: 36
  REGION 1..214: note = source = /note="Description of Artificial Sequence: Synthetic polypeptide"

SEQ ID NO: 101 - DNA, 642 bp, synthetic construct; identical to SEQ ID NO: 37
  misc_feature 1..642: note = source = /note="Description of Artificial Sequence: Synthetic polynucleotide"
  source 1..642: mol_type = other DNA

SEQ ID NO: 102 - protein, 10 aa, synthetic construct
  REGION 1..10: note = source = /note="Description of Artificial Sequence: Synthetic peptide"
GFTFSPHAMS

SEQ ID NO: 103 - protein, 17 aa, synthetic construct
  REGION 1..17: note = source = /note="Description of Artificial Sequence: Synthetic peptide"
AISDSGGSTHYADSVKG

SEQ ID NO: 104 - protein, 14 aa, synthetic construct
  REGION 1..14: note = source = /note="Description of Artificial Sequence: Synthetic peptide"
DDDAWSGYDYWFDY

SEQ ID NO: 105 - protein, 5 aa, synthetic construct
  REGION 1..5: note = source = /note="Description of Artificial Sequence: Synthetic peptide"
PHAMS

SEQ ID NO: 106 - protein, 17 aa, synthetic construct
  REGION 1..17: note = source = /note="Description of Artificial Sequence: Synthetic peptide"
AISDSGGSTHYADSVKG

SEQ ID NO: 107 - protein, 14 aa, synthetic construct
  REGION 1..14: note = source = /note="Description of Artificial Sequence: Synthetic peptide"
DDDAWSGYDYWFDY

SEQ ID NO: 108 - protein, 7 aa, synthetic construct
  REGION 1..7: note = source = /note="Description of Artificial Sequence: Synthetic peptide"
GFTFSPH

SEQ ID NO: 109 - protein, 6 aa, synthetic construct
  REGION 1..6: note = source = /note="Description of Artificial Sequence: Synthetic peptide"
SDSGGS

SEQ ID NO: 110 - protein, 14 aa, synthetic construct
  REGION 1..14: note = source = /note="Description of Artificial Sequence: Synthetic peptide"
DDDAWSGYDYWFDY

SEQ ID NO: 111 - protein, 8 aa, synthetic construct
  REGION 1..8: note = source = /note="Description of Artificial Sequence: Synthetic peptide"
GFTFSPHA

SEQ ID NO: 112 - protein, 8 aa, synthetic construct
  REGION 1..8: note = source = /note="Description of Artificial Sequence: Synthetic peptide"
ISDSGGST

SEQ ID NO: 113 - protein, 16 aa, synthetic construct
  REGION 1..16: note = source = /note="Description of Artificial Sequence: Synthetic peptide"
ARDDDAWSGYDYWFDY

SEQ ID NO: 114 - protein, 123 aa, synthetic construct
  REGION 1..123: note = source = /note="Description of Artificial Sequence: Synthetic polypeptide"
EVQLLESGGGLVQPGGSLRLSCAASGFTFSPHAMSWVRQAPGKGLEWVSAISDSGGSTHYADSVKGRFTISRDNSKNTLYLQMNSLRAEDTAVYYCARDDDAWSGYDYWFDYWGQGTLVTVSS

SEQ ID NO: 115 - DNA, 369 bp, synthetic construct
  misc_feature 1..369: note = source = /note="Description of Artificial Sequence: Synthetic polynucleotide"
  source 1..369: mol_type = other DNA
gaggtccaattgctggaatctggcggaggactggttcagcctggtggctctctgagactgtcttgtgccgccagcggcttcacctttagccctcatgccatgtcctgggtccgacaggctcctggaaaaggactcgagtgggtgtccgccatttctgattctggcggcagcacacactacgccgatagcgtgaagggcagattcaccatcagccgggacaacagcaagaacaccctgtacctgcagatgaacagcctgagagccgaggacacagccgtgtactattgcgcgcgtgacgatgatgcttggtccggctacgactattggttcgattactggggccagggcaccctggtcacagttagctca

SEQ ID NO: 116 - protein, 453 aa, synthetic construct
  REGION 1..453: note = source = /note="Description of Artificial Sequence: Synthetic polypeptide"
EVQLLESGGGLVQPGGSLRLSCAASGFTFSPHAMSWVRQAPGKGLEWVSAISDSGGSTHYADSVKGRFTISRDNSKNTLYLQMNSLRAEDTAVYYCARDDDAWSGYDYWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - DNA, 1359 bp, synthetic construct
  misc_feature 1..1359: note = source = /note="Description of Artificial Sequence: Synthetic polynucleotide"
  source 1..1359: mol_type = other DNA
gaggtccaattgctggaatctggcggaggactggttcagcctggtggctctctgagactgtcttgtgccgccagcggcttcacctttagccctcatgccatgtcctgggtccgacaggctcctggaaaaggactcgagtgggtgtccgccatttctgattctggcggcagcacacactacgccgatagcgtgaagggcagattcaccatcagccgggacaacagcaagaacaccctgtacctgcagatgaacagcctgagagccgaggacacagccgtgtactattgcgcgcgtgacgatgatgcttggtccggctacgactattggttcgattactggggccagggcaccctggtcacagttagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacatgccccccctgcccggcgccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcttaagccccggcaag

SEQ ID NO: 118 - protein, 11 aa, synthetic construct
  REGION 1..11: note = source = /note="Description of Artificial Sequence: Synthetic peptide"
RASQSISPYLN

SEQ ID NO: 119 - protein, 7 aa, synthetic construct
  REGION 1..7: note = source = /note="Description of Artificial Sequence: Synthetic peptide"
AADSLQS

SEQ ID NO: 120 - protein, 9 aa, synthetic construct
  REGION 1..9: note = source = /note="Description of Artificial Sequence: Synthetic peptide"
QQSYKIPLT

SEQ ID NO: 121 - protein, 11 aa, synthetic construct
  REGION 1..11: note = source = /note="Description of Artificial Sequence: Synthetic peptide"
RASQSISPYLN

SEQ ID NO: 122 - protein, 7 aa, synthetic construct
  REGION 1..7: note = source = /note="Description of Artificial Sequence: Synthetic peptide"
AADSLQS

SEQ ID NO: 123 - protein, 9 aa, synthetic construct
  REGION 1..9: note = source = /note="Description of Artificial Sequence: Synthetic peptide"
QQSYKIPLT

SEQ ID NO: 124 - protein, 7 aa, synthetic construct
  REGION 1..7: note = source = /note="Description of Artificial Sequence: Synthetic peptide"
SQSISPY

SEQ ID NO: 125 - skipped

SEQ ID NO: 126 - protein, 6 aa, synthetic construct
  REGION 1..6: note = source = /note="Description of Artificial Sequence: Synthetic peptide"
SYKIPL

SEQ ID NO: 127 - protein, 6 aa, synthetic construct
  REGION 1..6: note = source = /note="Description of Artificial Sequence: Synthetic peptide"
QSISPY

SEQ ID NO: 128 - skipped

SEQ ID NO: 129 - protein, 9 aa, synthetic construct
  REGION 1..9: note = source = /note="Description of Artificial Sequence: Synthetic peptide"
QQSYKIPLT

SEQ ID NO: 130 - protein, 107 aa, synthetic construct
  REGION 1..107: note = source = /note="Description of Artificial Sequence: Synthetic polypeptide"
DIQMTQSPSSLSASVGDRVTITCRASQSISPYLNWYQQKPGKAPKLLIYAADSLQSGVPSRFSGSGSGTDFTLTISSLQPEDFATYYCQQSYKIPLTFGQGTKVEIK

SEQ ID NO: 131 - DNA, 321 bp, synthetic construct
  misc_feature 1..321: note = source = /note="Description of Artificial Sequence: Synthetic polynucleotide"
  source 1..321: mol_type = other DNA
gatatccagatgacacagagccctagcagcctgtctgcctctgtgggcgatagagtgaccatcacctgtagagccagccagagcatcagcccctacctgaattggtaccagcagaagcctggcaaggcccctaagctgctgatctatgctgccgactctctgcagtctggcgtgccaagcagattttctggcagcggctctggcaccgacttcaccctgacaattagctccctgcagcctgaagacttcgccacctactactgccagcagagctacaagatccctctgacctttggccagggcaccaaggtggaaatcaag

SEQ ID NO: 132 - protein, 214 aa, synthetic construct
  REGION 1..214: note = source = /note="Description of Artificial Sequence: Synthetic polypeptide"
DIQMTQSPSSLSASVGDRVTITCRASQSISPYLNWYQQKPGKAPKLLIYAADSLQSGVPSRFSGSGSGTDFTLTISSLQPEDFATYYCQQSYKIPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - DNA, 642 bp, synthetic construct
  misc_feature 1..642: note = source = /note="Description of Artificial Sequence: Synthetic polynucleotide"
  source 1..642: mol_type = other DNA
gatatccagatgacacagagccctagcagcctgtctgcctctgtgggcgatagagtgaccatcacctgtagagccagccagagcatcagcccctacctgaattggtaccagcagaagcctggcaaggcccctaagctgctgatctatgctgccgactctctgcagtctggcgtgccaagcagattttctggcagcggctctggcaccgacttcaccctgacaattagctccctgcagcctgaagacttcgccacctactactgccagcagagctacaagatccctctgacctttggccagggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 134 - protein, 10 aa, synthetic construct
  REGION 1..10: note = source = /note="Description of Artificial Sequence: Synthetic peptide"
GFTFSPHAMS

SEQ ID NO: 135 - protein, 17 aa, synthetic construct
  REGION 1..17: note = source = /note="Description of Artificial Sequence: Synthetic peptide"
AISDSGGSTHYADSVKG

SEQ ID NO: 136 - protein, 14 aa, synthetic construct
  REGION 1..14: note = source = /note="Description of Artificial Sequence: Synthetic peptide"
DDDGWSGYDYWFDY

SEQ ID NO: 137 - protein, 5 aa, synthetic construct
  REGION 1..5: note = source = /note="Description of Artificial Sequence: Synthetic peptide"
PHAMS

SEQ ID NO: 138 - protein, 17 aa, synthetic construct
  REGION 1..17: note = source = /note="Description of Artificial Sequence: Synthetic peptide"
AISDSGGSTHYADSVKG

SEQ ID NO: 139 - protein, 14 aa, synthetic construct
  REGION 1..14: note = source = /note="Description of Artificial Sequence: Synthetic peptide"
DDDGWSGYDYWFDY

SEQ ID NO: 140 - protein, 7 aa, synthetic construct
  REGION 1..7: note = source = /note="Description of Artificial Sequence: Synthetic peptide"
GFTFSPH

SEQ ID NO: 141 - protein, 6 aa, synthetic construct
  REGION 1..6: note = source = /note="Description of Artificial Sequence: Synthetic peptide"
SDSGGS

SEQ ID NO: 142 - protein, 14 aa, synthetic construct
  REGION 1..14: note = source = /note="Description of Artificial Sequence: Synthetic peptide"
DDDGWSGYDYWFDY

SEQ ID NO: 143 - protein, 8 aa, synthetic construct
  REGION 1..8: note = source = /note="Description of Artificial Sequence: Synthetic peptide"
GFTFSPHA

SEQ ID NO: 144 - protein, 8 aa, synthetic construct
  REGION 1..8: note = source = /note="Description of Artificial Sequence: Synthetic peptide"
ISDSGGST

SEQ ID NO: 145 - protein, 16 aa, synthetic construct
  REGION 1..16: note = source = /note="Description of Artificial Sequence: Synthetic peptide"
ARDDDGWSGYDYWFDY

SEQ ID NO: 146 - protein, 123 aa, synthetic construct
  REGION 1..123: note = source = /note="Description of Artificial Sequence: Synthetic polypeptide"
EVQLLESGGGLVQPGGSLRLSCAASGFTFSPHAMSWVRQAPGKGLEWVSAISDSGGSTHYADSVKGRFTISRDNSKNTLYLQMNSLRAEDTAVYYCARDDDGWSGYDYWFDYWGQGTLVTVSS

SEQ ID NO: 147 - DNA, 369 bp, synthetic construct
  misc_feature 1..369: note = source = /note="Description of Artificial Sequence: Synthetic polynucleotide"
  source 1..369: mol_type = other DNA
gaggtccaattgctggaatctggcggaggactggttcagcctggtggctctctgagactgtcttgtgccgccagcggcttcacattcagccctcatgccatgtcctgggtccgacaggctcctggaaaaggactcgagtgggtgtccgccatttctgattctggcggcagcacacactacgccgatagcgtgaagggcagattcaccatcagccgggacaacagcaagaacaccctgtacctgcagatgaacagcctgagagccgaggacacagccgtgtactattgcgcgcgtgacgatgatggatggtccggctacgactattggttcgattactggggccagggcaccctggtcacagttagctca

SEQ ID NO: 148 - protein, 453 aa, synthetic construct
  REGION 1..453: note = source = /note="Description of Artificial Sequence: Synthetic polypeptide"
EVQLLESGGGLVQPGGSLRLSCAASGFTFSPHAMSWVRQAPGKGLEWVSAISDSGGSTHYADSVKGRFTISRDNSKNTLYLQMNSLRAEDTAVYYCARDDDGWSGYDYWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - DNA, 1359 bp, synthetic construct
  misc_feature 1..1359: note = source = /note="Description of Artificial Sequence: Synthetic polynucleotide"
  source 1..1359: mol_type = other DNA
gaggtccaattgctggaatctggcggaggactggttcagcctggtggctctctgagactgtcttgtgccgccagcggcttcacattcagccctcatgccatgtcctgggtccgacaggctcctggaaaaggactcgagtgggtgtccgccatttctgattctggcggcagcacacactacgccgatagcgtgaagggcagattcaccatcagccgggacaacagcaagaacaccctgtacctgcagatgaacagcctgagagccgaggacacagccgtgtactattgcgcgcgtgacgatgatggatggtccggctacgactattggttcgattactggggccagggcaccctggtcacagttagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacatgccccccctgcccggcgccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcttaagccccggcaag

SEQ ID NO: 150 - protein, 11 aa, synthetic construct
  REGION 1..11: note = source = /note="Description of Artificial Sequence: Synthetic peptide"
RASQSISPYLN

SEQ ID NO: 151 - protein, 7 aa, synthetic construct
  REGION 1..7: note = source = /note="Description of Artificial Sequence: Synthetic peptide"
AADSLQS

SEQ ID NO: 152 - protein, 9 aa, synthetic construct
  REGION 1..9: note = source = /note="Description of Artificial Sequence: Synthetic peptide"
QQSYKIPLT

SEQ ID NO: 153 - protein, 11 aa, synthetic construct
  REGION 1..11: note = source = /note="Description of Artificial Sequence: Synthetic peptide"
RASQSISPYLN

SEQ ID NO: 154 - protein, 7 aa, synthetic construct
  REGION 1..7: note = source = /note="Description of Artificial Sequence: Synthetic peptide"
AADSLQS

SEQ ID NO: 155 - protein, 9 aa, synthetic construct
  REGION 1..9: note = source = /note="Description of Artificial Sequence: Synthetic peptide"
QQSYKIPLT

SEQ ID NO: 156 - protein, 7 aa, synthetic construct
  REGION 1..7: note = source = /note="Description of Artificial Sequence: Synthetic peptide"
SQSISPY

SEQ ID NO: 157 - skipped

SEQ ID NO: 158 - protein, 6 aa, synthetic construct
  REGION 1..6: note = source = /note="Description of Artificial Sequence: Synthetic peptide"
SYKIPL

SEQ ID NO: 159 - protein, 6 aa, synthetic construct
  REGION 1..6: note = source = /note="Description of Artificial Sequence: Synthetic peptide"
QSISPY

SEQ ID NO: 160 - skipped

SEQ ID NO: 161 - protein, 9 aa, synthetic construct
  REGION 1..9: note = source = /note="Description of Artificial Sequence: Synthetic peptide"
QQSYKIPLT

SEQ ID NO: 162 - protein, 107 aa, synthetic construct; identical to SEQ ID NO: 130
  REGION 1..107: note = source = /note="Description of Artificial Sequence: Synthetic polypeptide"

SEQ ID NO: 163 - DNA, 321 bp, synthetic construct; identical to SEQ ID NO: 131
  misc_feature 1..321: note = source = /note="Description of Artificial Sequence: Synthetic polynucleotide"
  source 1..321: mol_type = other DNA

SEQ ID NO: 164 - protein, 214 aa, synthetic construct; identical to SEQ ID NO: 132
  REGION 1..214: note = source = /note="Description of Artificial Sequence: Synthetic polypeptide"

SEQ ID NO: 165 - DNA, 642 bp, synthetic construct; identical to SEQ ID NO: 133
  misc_feature 1..642: note = source = /note="Description of Artificial Sequence: Synthetic polynucleotide"
  source 1..642: mol_type = other DNA

SEQ ID NO: 166 - protein, 10 aa, synthetic construct
  REGION 1..10: note = source = /note="Description of Artificial Sequence: Synthetic peptide"
GFTFNRYGMH

SEQ ID NO: 167 - protein, 17 aa, synthetic construct
  REGION 1..17: note = source = /note="Description of Artificial Sequence: Synthetic peptide"
GIWHDGSHKYYADSLRG

SEQ ID NO: 168 - protein, 13 aa, synthetic construct
  REGION 1..13: note = source = /note="Description of Artificial Sequence: Synthetic peptide"
QTNRGRLDDAFDI

SEQ ID NO: 169 - protein, 5 aa, synthetic construct
  REGION 1..5: note = source = /note="Description of Artificial Sequence: Synthetic peptide"
RYGMH

SEQ ID NO: 170 - protein, 17 aa, synthetic construct
  REGION 1..17: note = source = /note="Description of Artificial Sequence: Synthetic peptide"
GIWHDGSHKYYADSLRG

SEQ ID NO: 171 - protein, 13 aa, synthetic construct
  REGION 1..13: note = source = /note="Description of Artificial Sequence: Synthetic peptide"
QTNRGRLDDAFDI

SEQ ID NO: 172 - protein, 7 aa, synthetic construct
  REGION 1..7: note = source = /note="Description of Artificial Sequence: Synthetic peptide"
GFTFNRY

SEQ ID NO: 173 - protein, 6 aa, synthetic construct
  REGION 1..6: note = source = /note="Description of Artificial Sequence: Synthetic peptide"
WHDGSH

SEQ ID NO: 174 - protein, 13 aa, synthetic construct
  REGION 1..13: note = source = /note="Description of Artificial Sequence: Synthetic peptide"
QTNRGRLDDAFDI

SEQ ID NO: 175 - protein, 8 aa, synthetic construct
  REGION 1..8: note = source = /note="Description of Artificial Sequence: Synthetic peptide"
GFTFNRYG

SEQ ID NO: 176 - protein, 8 aa, synthetic construct
  REGION 1..8: note = source = /note="Description of Artificial Sequence: Synthetic peptide"
IWHDGSHK

SEQ ID NO: 177 - protein, 15 aa, synthetic construct
  REGION 1..15: note = source = /note="Description of Artificial Sequence: Synthetic peptide"
VRQTNRGRLDDAFDI

SEQ ID NO: 178 - protein, 122 aa, synthetic construct
  REGION 1..122: note = source = /note="Description of Artificial Sequence: Synthetic polypeptide"
QVQLVESGGGVVQPGRSLRLSCAASGFTFNRYGMHWVRQAPGKGLEWVAGIWHDGSHKYYADSLRGRFTISRDNAKNTLDLQLNRLRAEDTSVYYCVRQTNRGRLDDAFDIWGQGTMVTVSS

SEQ ID NO: 179 - DNA, 366 bp, synthetic construct
  misc_feature 1..366: note = source = /note="Description of Artificial Sequence: Synthetic polynucleotide"
  source 1..366: mol_type = other DNA
caggtgcagctggtggagtctgggggaggcgtggtccagcctgggaggtccctgagactctcctgtgcagcgtcaggattcacattcaatagatatggcatgcactgggtccgccaggctccaggcaaggggttggagtgggtggctggtatatggcatgatggaagtcataaatactatgcagactctctgaggggccgattcaccatctccagagacaatgccaagaacacgctggatctgcaattgaacaggctgagagccgaagacacgtctgtgtattattgtgtgaggcaaaccaacaggggacgtctcgatgatgcttttgacatctggggccaagggacaatggtcaccgttagctca

SEQ ID NO: 180 - protein, 452 aa, synthetic construct
  REGION 1..452: note = source = /note="Description of Artificial Sequence: Synthetic polypeptide"
QVQLVESGGGVVQPGRSLRLSCAASGFTFNRYGMHWVRQAPGKGLEWVAGIWHDGSHKYYADSLRGRFTISRDNAKNTLDLQLNRLRAEDTSVYYCVRQTNRGRLDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - DNA, 1356 bp, synthetic construct
  misc_feature 1..1356: note = source = /note="Description of Artificial Sequence: Synthetic polynucleotide"
  source 1..1356: mol_type = other DNA
caggtgcagctggtggagtctgggggaggcgtggtccagcctgggaggtccctgagactctcctgtgcagcgtcaggattcacattcaatagatatggcatgcactgggtccgccaggctccaggcaaggggttggagtgggtggctggtatatggcatgatggaagtcataaatactatgcagactctctgaggggccgattcaccatctccagagacaatgccaagaacacgctggatctgcaattgaacaggctgagagccgaagacacgtctgtgtattattgtgtgaggcaaaccaacaggggacgtctcgatgatgcttttgacatctggggccaagggacaatggtcaccgttagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 182 - protein, 11 aa, synthetic construct
  REGION 1..11: note = source = /note="Description of Artificial Sequence: Synthetic peptide"
RASQTISSYLN

SEQ ID NO: 183 - protein, 7 aa, synthetic construct
  REGION 1..7: note = source = /note="Description of Artificial Sequence: Synthetic peptide"
AASTLQS

SEQ ID NO: 184 - protein, 9 aa, synthetic construct
  REGION 1..9: note = source = /note="Description of Artificial Sequence: Synthetic peptide"
QQNYDTLWT

SEQ ID NO: 185 - protein, 11 aa, synthetic construct
  REGION 1..11: note = source = /note="Description of Artificial Sequence: Synthetic peptide"
RASQTISSYLN

SEQ ID NO: 186 - protein, 7 aa, synthetic construct
  REGION 1..7: note = source = /note="Description of Artificial Sequence: Synthetic peptide"
AASTLQS

SEQ ID NO: 187 - protein, 9 aa, synthetic construct
  REGION 1..9: note = source = /note="Description of Artificial Sequence: Synthetic peptide"
QQNYDTLWT

SEQ ID NO: 188 - protein, 7 aa, synthetic construct
  REGION 1..7: note = source = /note="Description of Artificial Sequence: Synthetic peptide"
SQTISSY

SEQ ID NO: 189 - skipped

SEQ ID NO: 190 - protein, 6 aa, synthetic construct
  REGION 1..6: note = source = /note="Description of Artificial Sequence: Synthetic peptide"
NYDTLW

SEQ ID NO: 191 - protein, 6 aa, synthetic construct
  REGION 1..6: note = source = /note="Description of Artificial Sequence: Synthetic peptide"
QTISSY

SEQ ID NO: 192 - skipped

SEQ ID NO: 193 - protein, 9 aa, synthetic construct
  REGION 1..9: note = source = /note="Description of Artificial Sequence: Synthetic peptide"
QQNYDTLWT

SEQ ID NO: 194 - protein, 107 aa, synthetic construct
  REGION 1..107: note = source = /note="Description of Artificial Sequence: Synthetic polypeptide"
DIQMTQSPSSLSAAVGDRVTISCRASQTISSYLNWYQQKPGEAPKLLIYAASTLQSGVPSRFGGSGSGTDFTLTISSLQPEDSATYYCQQNYDTLWTFGQGTKVEIK

SEQ ID NO: 195 - DNA, 321 bp, synthetic construct
  misc_feature 1..321: note = source = /note="Description of Artificial Sequence: Synthetic polynucleotide"
  source 1..321: mol_type = other DNA
gatatccagatgacccagtctccatcctccctgtctgcagctgtaggagacagagtcaccatctcttgccgggcaagtcagaccattagtagttatttaaattggtatcagcagaaaccaggggaagcccctaagctcctgatctatgctgcctccaccttgcaaagtggggtcccttcaaggttcggtggcagtggatctgggacagatttcactctcaccatcagcagtctgcaacctgaagattctgcaacttactactgtcaacagaattacgatactttgtggacgttcggccaagggaccaaggtggaaatcaaa

SEQ ID NO: 196 - protein, 214 aa, synthetic construct
  REGION 1..214: note = source = /note="Description of Artificial Sequence: Synthetic polypeptide"
DIQMTQSPSSLSAAVGDRVTISCRASQTISSYLNWYQQKPGEAPKLLIYAASTLQSGVPSRFGGSGSGTDFTLTISSLQPEDSATYYCQQNYDTLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - DNA, 642 bp, synthetic construct
  misc_feature 1..642: note = source = /note="Description of Artificial Sequence: Synthetic polynucleotide"
  source 1..642: mol_type = other DNA
gatatccagatgacccagtctccatcctccctgtctgcagctgtaggagacagagtcaccatctcttgccgggcaagtcagaccattagtagttatttaaattggtatcagcagaaaccaggggaagcccctaagctcctgatctatgctgcctccaccttgcaaagtggggtcccttcaaggttcggtggcagtggatctgggacagatttcactctcaccatcagcagtctgcaacctgaagattctgcaacttactactgtcaacagaattacgatactttgtggacgttcggccaagggaccaaggtggaaatcaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 198 - protein, 10 aa, synthetic construct
  REGION 1..10: note = source = /note="Description of Artificial Sequence: Synthetic peptide"
GFTFDRYGMH

SEQ ID NO: 199 - protein, 17 aa, synthetic construct
  REGION 1..17: note = source = /note="Description of Artificial Sequence: Synthetic peptide"
GIWHEGSHKYYADSLRG

SEQ ID NO: 200 - protein, 13 aa, synthetic construct
  REGION 1..13: note = source = /note="Description of Artificial Sequence: Synthetic peptide"
QTNRGRLDDAFDI

SEQ ID NO: 201 - protein, 5 aa, synthetic construct
  REGION 1..5: note = source = /note="Description of Artificial Sequence: Synthetic peptide"
RYGMH

SEQ ID NO: 202 - protein, 17 aa, synthetic construct
  REGION 1..17: note = source = /note="Description of Artificial Sequence: Synthetic peptide"
GIWHEGSHKYYADSLRG

SEQ ID NO: 203 - protein, 13 aa, synthetic construct
  REGION 1..13: note = source = /note="Description of Artificial Sequence: Synthetic peptide"
QTNRGRLDDAFDI

SEQ ID NO: 204 - protein, 7 aa, synthetic construct
  REGION 1..7: note = source = /note="Description of Artificial Sequence: Synthetic peptide"
GFTFDRY

SEQ ID NO: 205 - protein, 6 aa, synthetic construct
  REGION 1..6: note = source = /note="Description of Artificial Sequence: Synthetic peptide"
WHEGSH

SEQ ID NO: 206 - protein, 13 aa, synthetic construct
  REGION 1..13: note = source = /note="Description of Artificial Sequence: Synthetic peptide"
QTNRGRLDDAFDI

SEQ ID NO: 207 - protein, 8 aa, synthetic construct
  REGION 1..8: note = source = /note="Description of Artificial Sequence: Synthetic peptide"
GFTFDRYG

SEQ ID NO: 208 - protein, 8 aa, synthetic construct
  REGION 1..8: note = source = /note="Description of Artificial Sequence: Synthetic peptide"
IWHEGSHK

SEQ ID NO: 209 - protein, 15 aa, synthetic construct
  REGION 1..15: note = source = /note="Description of Artificial Sequence: Synthetic peptide"
VRQTNRGRLDDAFDI

SEQ ID NO: 210 - protein, 122 aa, synthetic construct
  REGION 1..122: note = source = /note="Description of Artificial Sequence: Synthetic polypeptide"
QVQLVESGGGVVQPGRSLRLSCAASGFTFDRYGMHWVRQAPGKGLEWVAGIWHEGSHKYYADSLRGRFTISRDNAKNTLDLQLNRLRAEDTSVYYCVRQTNRGRLDDAFDIWGQGTMVTVSS

SEQ ID NO: 211 - DNA, 366 bp, synthetic construct
  misc_feature 1..366: note = source = /note="Description of Artificial Sequence: Synthetic polynucleotide"
  source 1..366: mol_type = other DNA
caggtgcagctggttgaatctggtggcggagtggtgcagcctggcagatctctgagactgtcttgtgccgccagcggcttcaccttcgacagatatggcatgcactgggtccgacaggcccctggaaaaggacttgaatgggtggccggaatctggcacgaaggcagccacaagtactacgccgatagcctgagaggccggttcaccatcagcagagacaacgccaagaacaccctggacctccagctgaacagactgagagccgaggataccagcgtgtactactgcgtgcggcagaccaacagaggcagactggacgatgccttcgatatctggggccaagggacaatggtcaccgttagctca

SEQ ID NO: 212 - protein, 452 aa, synthetic construct
  REGION 1..452: note = source = /note="Description of Artificial Sequence: Synthetic polypeptide"
QVQLVESGGGVVQPGRSLRLSCAASGFTFDRYGMHWVRQAPGKGLEWVAGIWHEGSHKYYADSLRGRFTISRDNAKNTLDLQLNRLRAEDTSVYYCVRQTNRGRLDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - DNA, 1356 bp, synthetic construct
  misc_feature 1..1356: note = source = /note="Description of Artificial Sequence: Synthetic polynucleotide"
  source 1..1356: mol_type = other DNA
caggtgcagctggttgaatctggtggcggagtggtgcagcctggcagatctctgagactgtcttgtgccgccagcggcttcaccttcgacagatatggcatgcactgggtccgacaggcccctggaaaaggacttgaatgggtggccggaatctggcacgaaggcagccacaagtactacgccgatagcctgagaggccggttcaccatcagcagagacaacgccaagaacaccctggacctccagctgaacagactgagagccgaggataccagcgtgtactactgcgtgcggcagaccaacagaggcagactggacgatgccttcgatatctggggccaagggacaatggtcaccgttagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 214 - protein, 11 aa, synthetic construct
  REGION 1..11: note = source = /note="Description of Artificial Sequence: Synthetic peptide"
RASQTISSYLN

SEQ ID NO: 215 - protein, 7 aa, synthetic construct
  REGION 1..7: note = source = /note="Description of Artificial Sequence: Synthetic peptide"
AASTLQS

SEQ ID NO: 216 - protein, 9 aa, synthetic construct
  REGION 1..9: note = source = /note="Description of Artificial Sequence: Synthetic peptide"
QQNYDTLWT

SEQ ID NO: 217 - protein, 11 aa, synthetic construct
  REGION 1..11: note = source = /note="Description of Artificial Sequence: Synthetic peptide"
RASQTISSYLN

SEQ ID NO: 218 - protein, 7 aa, synthetic construct
  REGION 1..7: note = source = /note="Description of Artificial Sequence: Synthetic peptide"
AASTLQS

SEQ ID NO: 219 - protein, 9 aa, synthetic construct
  REGION 1..9: note = source = /note="Description of Artificial Sequence: Synthetic peptide"
QQNYDTLWT

SEQ ID NO: 220 - protein, 7 aa, synthetic construct
  REGION 1..7: note = source = /note="Description of Artificial Sequence: Synthetic peptide"
SQTISSY

SEQ ID NO: 221 - skipped

SEQ ID NO: 222 - protein, 6 aa, synthetic construct
  REGION 1..6: note = source = /note="Description of Artificial Sequence: Synthetic peptide"
NYDTLW

SEQ ID NO: 223 - protein, 6 aa, synthetic construct
  REGION 1..6: note = source = /note="Description of Artificial Sequence: Synthetic peptide"
QTISSY

SEQ ID NO: 224 - skipped

SEQ ID NO: 225 - protein, 9 aa, synthetic construct
  REGION 1..9: note = source = /note="Description of Artificial Sequence: Synthetic peptide"
QQNYDTLWT

SEQ ID NO: 226 - protein, 107 aa, synthetic construct; identical to SEQ ID NO: 194
  REGION 1..107: note = source = /note="Description of Artificial Sequence: Synthetic polypeptide"

SEQ ID NO: 227 - DNA, 321 bp, synthetic construct; identical to SEQ ID NO: 195
  misc_feature 1..321: note = source = /note="Description of Artificial Sequence: Synthetic polynucleotide"
  source 1..321: mol_type = other DNA

SEQ ID NO: 228 - protein, 214 aa, synthetic construct; identical to SEQ ID NO: 196
  REGION 1..214: note = source = /note="Description of Artificial Sequence: Synthetic polypeptide"

SEQ ID NO: 229 - DNA, 642 bp, synthetic construct; identical to SEQ ID NO: 197
  misc_feature 1..642: note = source = /note="Description of Artificial Sequence: Synthetic polynucleotide"
  source 1..642: mol_type = other DNA

SEQ ID NO: 230 - protein, 10 aa, synthetic construct
  REGION 1..10: note = source = /note="Description of Artificial Sequence: Synthetic peptide"
GFTFERYGMH

SEQ ID NO: 231 - protein, 17 aa, synthetic construct
  REGION 1..17: note = source = /note="Description of Artificial Sequence: Synthetic peptide"
GIWHEGSHKYYADSLRG

SEQ ID NO: 232 - protein, 13 aa, synthetic construct
  REGION 1..13: note = source = /note="Description of Artificial Sequence: Synthetic peptide"
QTNRGRLDDAFDI

SEQ ID NO: 233 - protein, 5 aa, synthetic construct
  REGION 1..5: note = source = /note="Description of Artificial Sequence: Synthetic peptide"
RYGMH

SEQ ID NO: 234 - protein, 17 aa, synthetic construct
  REGION 1..17: note = source = /note="Description of Artificial Sequence: Synthetic peptide"
GIWHEGSHKYYADSLRG

SEQ ID NO: 235 - protein, 13 aa, synthetic construct
  REGION 1..13: note = source = /note="Description of Artificial Sequence: Synthetic peptide"
QTNRGRLDDAFDI

SEQ ID NO: 236 - protein, 7 aa, synthetic construct
  REGION 1..7: note = source = /note="Description of Artificial Sequence: Synthetic peptide"
GFTFERY

SEQ ID NO: 237 - protein, 6 aa, synthetic construct
  REGION 1..6: note = source = /note="Description of Artificial Sequence: Synthetic peptide"
WHEGSH

SEQ ID NO: 238 - protein, 13 aa, synthetic construct
  REGION 1..13: note = source = /note="Description of Artificial Sequence: Synthetic peptide"
QTNRGRLDDAFDI

SEQ ID NO: 239 - protein, 8 aa, synthetic construct
  REGION 1..8: note = source = /note="Description of Artificial Sequence: Synthetic peptide"
GFTFERYG

SEQ ID NO: 240 - protein, 8 aa, synthetic construct
  REGION 1..8: note = source = /note="Description of Artificial Sequence: Synthetic peptide"
IWHEGSHK

SEQ ID NO: 241 - protein, 15 aa, synthetic construct
  REGION 1..15: note = source = /note="Description of Artificial Sequence: Synthetic peptide"
VRQTNRGRLDDAFDI

SEQ ID NO: 242 - protein, 122 aa, synthetic construct
  REGION 1..122: note = source = /note="Description of Artificial Sequence: Synthetic polypeptide"
QVQLVESGGGVVQPGRSLRLSCAASGFTFERYGMHWVRQAPGKGLEWVAGIWHEGSHKYYADSLRGRFTISRDNAKNTLDLQLNRLRAEDTSVYYCVRQTNRGRLDDAFDIWGQGTMVTVSS

SEQ ID NO: 243 - DNA, 366 bp, synthetic construct
  misc_feature 1..366: note = source = /note="Description of Artificial Sequence: Synthetic polynucleotide"
  source 1..366: mol_type = other DNA
caggtgcagctggttgaatctggtggcggagtggtgcagcctggcagatctctgagactgtcttgtgccgccagcggcttcaccttcgagagatatggcatgcactgggtccgacaggcccctggaaaaggacttgaatgggtggccggaatctggcacgaaggcagccacaagtactacgccgatagcctgagaggccggttcaccatcagcagagacaacgccaagaacaccctggacctccagctgaacagactgagagccgaggataccagcgtgtactactgcgtgcggcagaccaacagaggcagactggacgatgccttcgatatctggggccaagggacaatggtcaccgttagctca

SEQ ID NO: 244 - protein, 452 aa, synthetic construct
  REGION 1..452: note = source = /note="Description of Artificial Sequence: Synthetic polypeptide"
QVQLVESGGGVVQPGRSLRLSCAASGFTFERYGMHWVRQAPGKGLEWVAGIWHEGSHKYYADSLRGRFTISRDNAKNTLDLQLNRLRAEDTSVYYCVRQTNRGRLDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - DNA, 1356 bp, synthetic construct
  misc_feature 1..1356: note = source = /note="Description of Artificial Sequence: Synthetic polynucleotide"
  source 1..1356: mol_type = other DNA
caggtgcagctggttgaatctggtggcggagtggtgcagcctggcagatctctgagactgtcttgtgccgccagcggcttcaccttcgagagatatggcatgcactgggtccgacaggcccctggaaaaggacttgaatgggtggccggaatctggcacgaaggcagccacaagtactacgccgatagcctgagaggccggttcaccatcagcagagacaacgccaagaacaccctggacctccagctgaacagactgagagccgaggataccagcgtgtactactgcgtgcggcagaccaacagaggcagactggacgatgccttcgatatctggggccaagggacaatggtcaccgttagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 246 - protein, 11 aa, synthetic construct
  REGION 1..11: note = source = /note="Description of Artificial Sequence: Synthetic peptide"
RASQTISSYLN

SEQ ID NO: 247 - protein, 7 aa, synthetic construct
  REGION 1..7: note = source = /note="Description of Artificial Sequence: Synthetic peptide"
AASTLQS

SEQ ID NO: 248 - protein, 9 aa, synthetic construct
  REGION 1..9: note = source = /note="Description of Artificial Sequence: Synthetic peptide"
QQNYDTLWT

SEQ ID NO: 249 - protein, 11 aa, synthetic construct
  REGION 1..11: note = source = /note="Description of Artificial Sequence: Synthetic peptide"
RASQTISSYLN

SEQ ID NO: 250 - protein, 7 aa, synthetic construct
  REGION 1..7: note = source = /note="Description of Artificial Sequence: Synthetic peptide"
AASTLQS

SEQ ID NO: 251 - protein, 9 aa, synthetic construct
  REGION 1..9: note = source = /note="Description of Artificial Sequence: Synthetic peptide"
QQNYDTLWT

SEQ ID NO: 252 - protein, 7 aa, synthetic construct
  REGION 1..7: note = source = /note="Description of Artificial Sequence: Synthetic peptide"
SQTISSY

SEQ ID NO: 253 - skipped

SEQ ID NO: 254 - protein, 6 aa, synthetic construct
  REGION 1..6: note = source = /note="Description of Artificial Sequence: Synthetic peptide"
NYDTLW

SEQ ID NO: 255 - protein, 6 aa, synthetic construct
  REGION 1..6: note = source = /note="Description of Artificial Sequence: Synthetic peptide"
QTISSY

SEQ ID NO: 256 - skipped

SEQ ID NO: 257 - protein, 9 aa, synthetic construct
  REGION 1..9: note = source = /note="Description of Artificial Sequence: Synthetic peptide"
QQNYDTLWT

SEQ ID NO: 258 - protein, 107 aa, synthetic construct; identical to SEQ ID NO: 194
  REGION 1..107: note = source = /note="Description of Artificial Sequence: Synthetic polypeptide"

SEQ ID NO: 259 - DNA, 321 bp, synthetic construct; identical to SEQ ID NO: 195
  misc_feature 1..321: note = source = /note="Description of Artificial Sequence: Synthetic polynucleotide"
  source 1..321: mol_type = other DNA

SEQ ID NO: 260 - protein, 214 aa, synthetic construct; identical to SEQ ID NO: 196
  REGION 1..214: note = source = /note="Description of Artificial Sequence: Synthetic polypeptide"

SEQ ID NO: 261 - DNA, 642 bp, synthetic construct; identical to SEQ ID NO: 197
  misc_feature 1..642: note = source = /note="Description of Artificial Sequence: Synthetic polynucleotide"
  source 1..642: mol_type = other DNA

SEQ ID NO: 262 - protein, 10 aa, synthetic construct
  REGION 1..10: note = source = /note="Description of Artificial Sequence: Synthetic peptide"
GFIFTDYYMT

SEQ ID NO: 263 - protein, 17 aa, synthetic construct
  REGION 1..17: note = source = /note="Description of Artificial Sequence: Synthetic peptide"
FITSGGETTYYADSVKG

SEQ ID NO: 264 - protein, 20 aa, synthetic construct
  REGION 1..20: note = source = /note="Description of Artificial Sequence: Synthetic peptide"
AHFDGYQYDTRGDFTYYFDN

SEQ ID NO: 265 - protein, 5 aa, synthetic construct
  REGION 1..5: note = source = /note="Description of Artificial Sequence: Synthetic peptide"
DYYMT

SEQ ID NO: 266 - protein, 17 aa, synthetic construct
  REGION 1..17: note = source = /note="Description of Artificial Sequence: Synthetic peptide"
FITSGGETTYYADSVKG

SEQ ID NO: 267 - protein, 20 aa, synthetic construct
  REGION 1..20: note = source = /note="Description of Artificial Sequence: Synthetic peptide"
AHFDGYQYDTRGDFTYYFDN

SEQ ID NO: 268 - protein, 7 aa, synthetic construct
  REGION 1..7: note = source = /note="Description of Artificial Sequence: Synthetic peptide"
GFIFTDY

SEQ ID NO: 269 - protein, 6 aa, synthetic construct
  REGION 1..6: note = source = /note="Description of Artificial Sequence: Synthetic peptide"
TSGGET

SEQ ID NO: 270 - protein, 20 aa, synthetic construct
  REGION 1..20: note = source = /note="Description of Artificial Sequence: Synthetic peptide"
AHFDGYQYDTRGDFTYYFDN

SEQ ID NO: 271 - protein, 8 aa, synthetic construct
  REGION 1..8: note = source = /note="Description of Artificial Sequence: Synthetic peptide"
GFIFTDYY

SEQ ID NO: 272 - protein, 8 aa, synthetic construct
  REGION 1..8: note = source = /note="Description of Artificial Sequence: Synthetic peptide"
ITSGGETT

SEQ ID NO: 273 - protein, 22 aa, synthetic construct
  REGION 1..22: note = source = /note="Description of Artificial Sequence: Synthetic peptide"
VRAHFDGYQYDTRGDFTYYFDN

SEQ ID NO: 274 - protein, 129 aa, synthetic construct
  REGION 1..129: note = source = /note="Description of Artificial Sequence: Synthetic polypeptide"
QVQLQESGGRLVRPGGSLRLSCAASGFIFTDYYMTWIRQAPGKGPEWIAFITSGGETTYYADSVKGRFTISRDNAKKSLFLQMYSLRADDTAVYYCVRAHFDGYQYDTRGDFTYYFDNWGLGTLVSVSS

SEQ ID NO: 275 - DNA, 387 bp, synthetic construct
  misc_feature 1..387: note = source = /note="Description of Artificial Sequence: Synthetic polynucleotide"
  source 1..387: mol_type = other DNA
caggtgcagctgcaggagtcggggggacgcttggtcaggcctggagggtccctgagactctcctgtgcagcctccggattcatcttcactgactactacatgacctggatccgccaggctccagggaaggggccggagtggattgcatttatcacaagtgggggcgagaccacatactacgcagactctgtgaagggccgcttcaccatttccagggacaacgccaagaagtcactctttctgcaaatgtacagcctgagagccgacgacacggccgtgtattattgtgtgagagcccactttgatggttatcagtatgatactcgtggtgacttcacttattactttgacaactggggcctgggaaccctggtcagcgtctcctca

SEQ ID NO: 276 - protein, 459 aa, synthetic construct
  REGION 1..459: note = source = /note="Description of Artificial Sequence: Synthetic polypeptide"
QVQLQESGGRLVRPGGSLRLSCAASGFIFTDYYMTWIRQAPGKGPEWIAFITSGGETTYYADSVKGRFTISRDNAKKSLFLQMYSLRADDTAVYYCVRAHFDGYQYDTRGDFTYYFDNWGLGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - DNA, 1377 bp, synthetic construct
  misc_feature 1..1377: note = source = /note="Description of Artificial Sequence: Synthetic polynucleotide"
  source 1..1377: mol_type = other DNA
caggtgcagctgcaggagtcggggggacgcttggtcaggcctggagggtccctgagactctcctgtgcagcctccggattcatcttcactgactactacatgacctggatccgccaggctccagggaaggggccggagtggattgcatttatcacaagtgggggcgagaccacatactacgcagactctgtgaagggccgcttcaccatttccagggacaacgccaagaagtcactctttctgcaaatgtacagcctgagagccgacgacacggccgtgtattattgtgtgagagcccactttgatggttatcagtatgatactcgtggtgacttcacttattactttgacaactggggcctgggaaccctggtcagcgtctcctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 278 - protein, 11 aa, synthetic construct
  REGION 1..11: note = source = /note="Description of Artificial Sequence: Synthetic peptide"
RASQSVSSSLA

SEQ ID NO: 279 - protein, 7 aa, synthetic construct
  REGION 1..7: note = source = /note="Description of Artificial Sequence: Synthetic peptide"
GASTRAT

SEQ ID NO: 280 - protein, 11 aa, synthetic construct
  REGION 1..11: note = source = /note="Description of Artificial Sequence: Synthetic peptide"
HQYINWPPGDT

SEQ ID NO: 281 - protein, 11 aa, synthetic construct
  REGION 1..11: note = source = /note="Description of Artificial Sequence: Synthetic peptide"
RASQSVSSSLA

SEQ ID NO: 282 - protein, 7 aa, synthetic construct
  REGION 1..7: note = source = /note="Description of Artificial Sequence: Synthetic peptide"
GASTRAT

SEQ ID NO: 283 - protein, 11 aa, synthetic construct
  REGION 1..11: note = source = /note="Description of Artificial Sequence: Synthetic peptide"
HQYINWPPGDT

SEQ ID NO: 284 - protein, 7 aa, synthetic construct
  REGION 1..7: note = source = /note="Description of Artificial Sequence: Synthetic peptide"
SQSVSSS

SEQ ID NO: 285 - skipped

SEQ ID NO: 286 - protein, 8 aa, synthetic construct
  REGION 1..8: note = source = /note="Description of Artificial Sequence: Synthetic peptide"
YINWPPGD

SEQ ID NO: 287 - protein, 6 aa, synthetic construct
  REGION 1..6: note = source = /note="Description of Artificial Sequence: Synthetic peptide"
QSVSSS

SEQ ID NO: 288 - skipped

SEQ ID NO: 289 - protein, 11 aa, synthetic construct
  REGION 1..11: note = source = /note="Description of Artificial Sequence: Synthetic peptide"
HQYINWPPGDT

SEQ ID NO: 290 - protein, 109 aa, synthetic construct
  REGION 1..109: note = source = /note="Description of Artificial Sequence: Synthetic polypeptide"
EIVMTQSPATLSVSPGERVTLSCRASQSVSSSLAWYQQKPGRAPRLLIYGASTRATGVPARFSGGGSGTDFTLTISSLQSEDFAVYYCHQYINWPPGDTFGQGTRLDIK

SEQ ID NO: 291 - DNA, 327 bp, synthetic construct
  misc_feature 1..327: note = source = /note="Description of Artificial Sequence: Synthetic polynucleotide"
  source 1..327: mol_type = other DNA
gaaatagtgatgactcagtctccagccaccctgtctgtgtctccaggggaaagagtcaccctctcctgcagggccagtcagagtgttagtagcagcttagcctggtaccagcagaaacctggccgggctcccaggctcctcatttatggagcatccaccagggccactggtgtcccagccaggttcagtggcggtgggtctgggacagacttcactctcaccatcagcagcctgcagtctgaagattttgcagtttattactgtcaccagtatattaattggcctccgggggacacttttggccaggggacgaggctggatatcaaa

SEQ ID NO: 292 - protein, 216 aa, synthetic construct
  REGION 1..216: note = source = /note="Description of Artificial Sequence: Synthetic polypeptide"
EIVMTQSPATLSVSPGERVTLSCRASQSVSSSLAWYQQKPGRAPRLLIYGASTRATGVPARFSGGGSGTDFTLTISSLQSEDFAVYYCHQYINWPPGDTFGQGTRLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 293 - DNA, 648 bp, synthetic construct
  misc_feature 1..648: note = source = /note="Description of Artificial Sequence: Synthetic polynucleotide"
  source 1..648: mol_type = other DNA
gaaatagtgatgactcagtctccagccaccctgtctgtgtctccaggggaaagagtcaccctctcctgcagggccagtcagagtgttagtagcagcttagcctggtaccagcagaaacctggccgggctcccaggctcctcatttatggagcatccaccagggccactggtgtcccagccaggttcagtggcggtgggtctgggacagacttcactctcaccatcagcagcctgcagtctgaagattttgcagtttattactgtcaccagtatattaattggcctccgggggacacttttggccaggggacgaggctggatatcaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 294 - protein, 10 aa, synthetic construct
  REGION 1..10: note = source = /note="Description of Artificial Sequence: Synthetic peptide"
GFIFTDYYMT

SEQ ID NO: 295 - protein, 17 aa, synthetic construct
  REGION 1..17: note = source = /note="Description of Artificial Sequence: Synthetic peptide"
FITSGGETTYYADSVKG

SEQ ID NO: 296 - protein, 20 aa, synthetic construct
  REGION 1..20: note = source = /note="Description of Artificial Sequence: Synthetic peptide"
AHFDLYQYDTRGDFTYYFDN

SEQ ID NO: 297 - protein, 5 aa, synthetic construct
  REGION 1..5: note = source = /note="Description of Artificial Sequence: Synthetic peptide"
DYYMT

SEQ ID NO: 298 - protein, 17 aa, synthetic construct
  REGION 1..17: note = source = /note="Description of Artificial Sequence: Synthetic peptide"
FITSGGETTYYADSVKG

SEQ ID NO: 299 - protein, 20 aa, synthetic construct
  REGION 1..20: note = source = /note="Description of Artificial Sequence: Synthetic peptide"
AHFDLYQYDTRGDFTYYFDN

SEQ ID NO: 300 - protein, 7 aa, synthetic construct
  REGION 1..7: note = source = /note="Description of Artificial Sequence: Synthetic peptide"
GFIFTDY

SEQ ID NO: 301 - protein, 6 aa, synthetic construct
  REGION 1..6: note = source = /note="Description of Artificial Sequence: Synthetic peptide"
TSGGET

SEQ ID NO: 302 - protein, 20 aa, synthetic construct
  REGION 1..20: note = source = /note="Description of Artificial Sequence: Synthetic peptide"
AHFDLYQYDTRGDFTYYFDN

SEQ ID NO: 303 - protein, 8 aa, synthetic construct
  REGION 1..8: note = source = /note="Description of Artificial Sequence: Synthetic peptide"
GFIFTDYY

SEQ ID NO: 304 - protein, 8 aa, synthetic construct
  REGION 1..8: note = source = /note="Description of Artificial Sequence: Synthetic peptide"
ITSGGETT

SEQ ID NO: 305 - protein, 22 aa, synthetic construct
  REGION 1..22: note = source = /note="Description of Artificial Sequence: Synthetic peptide"
VRAHFDLYQYDTRGDFTYYFDN

SEQ ID NO: 306 - protein, 129 aa, synthetic construct
  REGION 1..129: note = source = /note="Description of Artificial Sequence: Synthetic polypeptide"
QVQLQESGGRLVRPGGSLRLSCAASGFIFTDYYMTWIRQAPGKGPEWIAFITSGGETTYYADSVKGRFTISRDNAKKSLFLQMYSLRADDTAVYYCVRAHFDLYQYDTRGDFTYYFDNWGLGTLVSVSS

SEQ ID NO: 307 - DNA, 387 bp, synthetic construct
  misc_feature 1..387: note = source = /note="Description of Artificial Sequence: Synthetic polynucleotide"
  source 1..387: mol_type = other DNA
caggttcagctgcaagaatctggcggcagactcgttagacctggcggctctctgagactgtcttgtgccgccagcggcttcatcttcaccgactactacatgacctggatcagacaggcccctggcaagggacctgagtggatcgcctttatcacaagcggcggagagacaacctactacgccgatagcgtgaagggcagattcaccatcagccgggacaacgccaagaagtccctgttcctccagatgtacagcctgagagccgacgataccgccgtgtattattgcgtgcgggcccactttgacctgtaccagtacgataccagaggcgatttcacctactacttcgacaactggggcctgggaaccctggtgtctgtctcttct

SEQ ID NO: 308 - protein, 459 aa, synthetic construct
  REGION 1..459: note = source = /note="Description of Artificial Sequence: Synthetic polypeptide"
QVQLQESGGRLVRPGGSLRLSCAASGFIFTDYYMTWIRQAPGKGPEWIAFITSGGETTYYADSVKGRFTISRDNAKKSLFLQMYSLRADDTAVYYCVRAHFDLYQYDTRGDFTYYFDNWGLGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - DNA, 1377 bp, synthetic construct
  misc_feature 1..1377: note = source = /note="Description of Artificial Sequence: Synthetic polynucleotide"
  source 1..1377: mol_type = other DNA
caggttcagctgcaagaatctggcggcagactcgttagacctggcggctctctgagactgtcttgtgccgccagcggcttcatcttcaccgactactacatgacctggatcagacaggcccctggcaagggacctgagtggatcgcctttatcacaagcggcggagagacaacctactacgccgatagcgtgaagggcagattcaccatcagccgggacaacgccaagaagtccctgttcctccagatgtacagcctgagagccgacgataccgccgtgtattattgcgtgcgggcccactttgacctgtaccagtacgataccagaggcgatttcacctactacttcgacaactggggcctgggaaccctggtgtctgtctcttctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacatgccccccctgcccggcgccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcttaagccccggcaag

SEQ ID NO: 310 - protein, 11 aa, synthetic construct
  REGION 1..11: note = source = /note="Description of Artificial Sequence: Synthetic peptide"
RASQSVSSSLA

SEQ ID NO: 311 - protein, 7 aa, synthetic construct
  REGION 1..7: note = source = /note="Description of Artificial Sequence: Synthetic peptide"
GASTRAT

SEQ ID NO: 312 - protein, 11 aa, synthetic construct
  REGION 1..11: note = source = /note="Description of Artificial Sequence: Synthetic peptide"
HQYINWPPGDT

SEQ ID NO: 313 - protein, 11 aa, synthetic construct
  REGION 1..11: note = source = /note="Description of Artificial Sequence: Synthetic peptide"
RASQSVSSSLA

SEQ ID NO: 314 - protein, 7 aa, synthetic construct
  REGION 1..7: note = source = /note="Description of Artificial Sequence: Synthetic peptide"
GASTRAT

SEQ ID NO: 315 - protein, 11 aa, synthetic construct
  REGION 1..11: note = source = /note="Description of Artificial Sequence: Synthetic peptide"
HQYINWPPGDT

SEQ ID NO: 316 - protein, 7 aa, synthetic construct
  REGION 1..7: note = source = /note="Description of Artificial Sequence: Synthetic peptide"
SQSVSSS

SEQ ID NO: 317 - skipped

SEQ ID NO: 318 - protein, 8 aa, synthetic construct
  REGION 1..8: note = source = /note="Description of Artificial Sequence: Synthetic peptide"
YINWPPGD

SEQ ID NO: 319 - protein, 6 aa, synthetic construct
  REGION 1..6: note = source = /note="Description of Artificial Sequence: Synthetic peptide"
QSVSSS

SEQ ID NO: 320 - skipped

SEQ ID NO: 321 - protein, 11 aa, synthetic construct
  REGION 1..11: note = source = /note="Description of Artificial Sequence: Synthetic peptide"
HQYINWPPGDT

SEQ ID NO: 322 - protein, 109 aa, synthetic construct; identical to SEQ ID NO: 290
  REGION 1..109: note = source = /note="Description of Artificial Sequence: Synthetic polypeptide"

SEQ ID NO: 323 - DNA, 327 bp, synthetic construct; identical to SEQ ID NO: 291
  misc_feature 1..327: note = source = /note="Description of Artificial Sequence: Synthetic polynucleotide"
  source 1..327: mol_type = other DNA

SEQ ID NO: 324 - protein, 216 aa, synthetic construct; identical to SEQ ID NO: 292
  REGION 1..216: note = source = /note="Description of Artificial Sequence: Synthetic polypeptide"

SEQ ID NO: 325 - DNA, 648 bp, synthetic construct; identical to SEQ ID NO: 293
  misc_feature 1..648: note = source = /note="Description of Artificial Sequence: Synthetic polynucleotide"
  source 1..648: mol_type = other DNA

SEQ ID NO: 326 - protein, 10 aa, synthetic construct
  REGION 1..10: note = source = /note="Description of Artificial Sequence: Synthetic peptide"
GFIFTDYYMT

SEQ ID NO: 327 - protein, 17 aa, synthetic construct
  REGION 1..17: note = source = /note="Description of Artificial Sequence: Synthetic peptide"
FITSGGETTYYADSVKG

SEQ ID NO: 328 - protein, 20 aa, synthetic construct
  REGION 1..20: note = source = /note="Description of Artificial Sequence: Synthetic peptide"
AHFDIYQYDTRGDFTYYFDN

SEQ ID NO: 329 - protein, 5 aa, synthetic construct
  REGION 1..5: note = source = /note="Description of Artificial Sequence: Synthetic peptide"
DYYMT

SEQ ID NO: 330 - protein, 17 aa, synthetic construct
  REGION 1..17: note = source = /note="Description of Artificial Sequence: Synthetic peptide"
FITSGGETTYYADSVKG

SEQ ID NO: 331 - protein, 20 aa, synthetic construct
  REGION 1..20: note = source = /note="Description of Artificial Sequence: Synthetic peptide"
AHFDIYQYDTRGDFTYYFDN

SEQ ID NO: 332 - protein, 7 aa, synthetic construct
  REGION 1..7: note = source = /note="Description of Artificial Sequence: Synthetic peptide"
GFIFTDY

SEQ ID NO: 333 - protein, 6 aa, synthetic construct
  REGION 1..6: note = source = /note="Description of Artificial Sequence: Synthetic peptide"
TSGGET

SEQ ID NO: 334 - protein, 20 aa, synthetic construct
  REGION 1..20: note = source = /note="Description of Artificial Sequence: Synthetic peptide"
AHFDIYQYDTRGDFTYYFDN

SEQ ID NO: 335 - protein, 8 aa, synthetic construct
  REGION 1..8: note = source = /note="Description of Artificial Sequence: Synthetic peptide"
GFIFTDYY

SEQ ID NO: 336 - protein, 8 aa, synthetic construct
  REGION 1..8: note = source = /note="Description of Artificial Sequence: Synthetic peptide"
ITSGGETT

SEQ ID NO: 337 - protein, 22 aa, synthetic construct
  REGION 1..22: note = source = /note="Description of Artificial Sequence: Synthetic peptide"
VRAHFDIYQYDTRGDFTYYFDN

SEQ ID NO: 338 - protein, 129 aa, synthetic construct
  REGION 1..129: note = source = /note="Description of Artificial Sequence: Synthetic polypeptide"
QVQLQESGGRLVRPGGSLRLSCAASGFIFTDYYMTWIRQAPGKGPEWIAFITSGGETTYYADSVKGRFTISRDNAKKSLFLQMYSLRADDTAVYYCVRAHFDIYQYDTRGDFTYYFDNWGLGTLVSVSS

SEQ ID NO: 339 - DNA, 387 bp, synthetic construct
  misc_feature 1..387: note = source = /note="Description of Artificial Sequence: Synthetic polynucleotide"
  source 1..387: mol_type = other DNA
caggttcagctgcaagaatctggcggcagactcgttagacctggcggctctctgagactgtcttgtgccgccagcggcttcatcttcaccgactactacatgacctggatcagacaggcccctggcaagggacctgagtggatcgcctttatcacaagcggcggagagacaacctactacgccgatagcgtgaagggcagattcaccatcagccgggacaacgccaagaagtccctgttcctccagatgtacagcctgagagccgacgataccgccgtgtattattgcgtgcgggcccactttgacatctaccagtacgataccagaggcgatttcacctactacttcgacaactggggcctgggaaccctggtgtctgtctcttct

SEQ ID NO: 340 - protein, 459 aa, synthetic construct
  REGION 1..459: note = source = /note="Description of Artificial Sequence: Synthetic polypeptide"
QVQLQESGGRLVRPGGSLRLSCAASGFIFTDYYMTWIRQAPGKGPEWIAFITSGGETTYYADSVKGRFTISRDNAKKSLFLQMYSLRADDTAVYYCVRAHFDIYQYDTRGDFTYYFDNWGLGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 341 - DNA, 1377 bp, synthetic construct
  misc_feature 1..1377: note = source = /note="Description of Artificial Sequence: Synthetic polynucleotide"
  source 1..1377: mol_type = other DNA
caggttcagctgcaagaatctggcggcagactcgttagacctggcggctctctgagactgtcttgtgccgccagcggcttcatcttcaccgactactacatgacctggatcagacaggcccctggcaagggacctgagtggatcgcctttatcacaagcggcggagagacaacctactacgccgatagcgtgaagggcagattcaccatcagccgggacaacgccaagaagtccctgttcctccagatgtacagcctgagagccgacgataccgccgtgtattattgcgtgcgggcccactttgacatctaccagtacgataccagaggcgatttcacctactacttcgacaactggggcctgggaaccctggtgtctgtctcttctgctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacatgccccccctgcccggcgccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcttaagccccggcaag

SEQ ID NO: 342 - protein, 11 aa, synthetic construct
  REGION 1..11: note = source = /note="Description of Artificial Sequence: Synthetic peptide"
RASQSVSSSLA

SEQ ID NO: 343 - protein, 7 aa, synthetic construct
  REGION 1..7: note = source = /note="Description of Artificial Sequence: Synthetic peptide"
GASTRAT

SEQ ID NO: 344 - protein, 11 aa, synthetic construct
  REGION 1..11: note = source = /note="Description of Artificial Sequence: Synthetic peptide"
HQYINWPPGDT

SEQ ID NO: 345 - protein, 11 aa, synthetic construct
  REGION 1..11: note = source = /note="Description of Artificial Sequence: Synthetic peptide"
RASQSVSSSLA

SEQ ID NO: 346 - protein, 7 aa, synthetic construct
  REGION 1..7: note = source = /note="Description of Artificial Sequence: Synthetic peptide"
GASTRAT

SEQ ID NO: 347 - protein, 11 aa, synthetic construct
  REGION 1..11: note = source = /note="Description of Artificial Sequence: Synthetic peptide"
HQYINWPPGDT

SEQ ID NO: 348 - protein, 7 aa, synthetic construct
  REGION 1..7: note = source = /note="Description of Artificial Sequence: Synthetic peptide"
SQSVSSS

SEQ ID NO: 349 - skipped

SEQ ID NO: 350 - protein, 8 aa, synthetic construct
  REGION 1..8: note = source = /note="Description of Artificial Sequence: Synthetic peptide"
YINWPPGD

SEQ ID NO: 351 - protein, 6 aa, synthetic construct
  REGION 1..6: note = source = /note="Description of Artificial Sequence: Synthetic peptide"
QSVSSS

SEQ ID NO: 352 - skipped

SEQ ID NO: 353 - protein, 11 aa, synthetic construct
  REGION 1..11: note = source = /note="Description of Artificial Sequence: Synthetic peptide"
HQYINWPPGDT

SEQ ID NO: 354 - protein, 109 aa, synthetic construct; identical to SEQ ID NO: 290
  REGION 1..109: note = source = /note="Description of Artificial Sequence: Synthetic polypeptide"

SEQ ID NO: 355 - DNA, 327 bp, synthetic construct; identical to SEQ ID NO: 291
  misc_feature 1..327: note = source = /note="Description of Artificial Sequence: Synthetic polynucleotide"
  source 1..327: mol_type = other DNA

SEQ ID NO: 356 - protein, 216 aa, synthetic construct; identical to SEQ ID NO: 292
  REGION 1..216: note = source = /note="Description of Artificial Sequence: Synthetic polypeptide"

SEQ ID NO: 357 - DNA, 648 bp, synthetic construct; identical to SEQ ID NO: 293
  misc_feature 1..648: note = source = /note="Description of Artificial Sequence: Synthetic polynucleotide"
  source 1..648: mol_type = other DNA

SEQ ID NO: 358 - protein, 10 aa, synthetic construct
  REGION 1..10: note = source = /note="Description of Artificial Sequence: Synthetic peptide"
GFTFSYYGMN

SEQ ID NO: 359 - protein, 17 aa, synthetic construct
  REGION 1..17: note = source = /note="Description of Artificial Sequence: Synthetic peptide"
GITNSGSITYYADSVKG

SEQ ID NO: 360 - protein, 14 aa, synthetic construct
  REGION 1..14: note = source = /note="Description of Artificial Sequence: Synthetic peptide"
VGVRSSSGMWDLDY

SEQ ID NO: 361 - protein, 5 aa, synthetic construct
  REGION 1..5: note = source = /note="Description of Artificial Sequence: Synthetic peptide"
YYGMN

SEQ ID NO: 362 - protein, 17 aa, synthetic construct
  REGION 1..17: note = source = /note="Description of Artificial Sequence: Synthetic peptide"
GITNSGSITYYADSVKG

SEQ ID NO: 363 - protein, 14 aa, synthetic construct
  REGION 1..14: note = source = /note="Description of Artificial Sequence: Synthetic peptide"
VGVRSSSGMWDLDY

SEQ ID NO: 364 - protein, 7 aa, synthetic construct
  REGION 1..7: note = source = /note="Description of Artificial Sequence: Synthetic peptide"
GFTFSYY

SEQ ID NO: 365 - protein, 6 aa, synthetic construct
  REGION 1..6: note = source = /note="Description of Artificial Sequence: Synthetic peptide"
TNSGSI

SEQ ID NO: 366 - protein, 14 aa, synthetic construct
  REGION 1..14: note = source = /note="Description of Artificial Sequence: Synthetic peptide"
VGVRSSSGMWDLDY

SEQ ID NO: 367 - protein, 8 aa, synthetic construct
  REGION 1..8: note = source = /note="Description of Artificial Sequence: Synthetic peptide"
GFTFSYYG

SEQ ID NO: 368 - protein, 8 aa, synthetic construct
  REGION 1..8: note = source = /note="Description of Artificial Sequence: Synthetic peptide"
ITNSGSIT

SEQ ID NO: 369 - protein, 16 aa, synthetic construct
  REGION 1..16: note = source = /note="Description of Artificial Sequence: Synthetic peptide"
AKVGVRSSSGMWDLDY

SEQ ID NO: 370 - protein, 123 aa, synthetic construct
  REGION 1..123: note = source = /note="Description of Artificial Sequence: Synthetic polypeptide"
EVQLLESGGGLVQPGGSRRLSCAASGFTFSYYGMNWVRQAPGKGLEWVSGITNSGSITYYADSVKGRFSISRDNSKNTLFLQMNSLRAEDTAVYYCAKVGVRSSSGMWDLDYWGQGTLVTVSS

SEQ ID NO: 371 - DNA, 369 bp, synthetic construct
  misc_feature 1..369: note = source = /note="Description of Artificial Sequence: Synthetic polynucleotide"
  source 1..369: mol_type = other DNA
gaggttcaattgttggagtctgggggaggcctggtacagccgggggggtcccggagactgtcctgtgcagcctctggattcacctttagctactatggcatgaactgggtccgccaggctccagggaagggactggaatgggtctcaggtattactaatagtggtagtatcacatactacgcagactccgtgaagggccggttcagcatctccagagacaattccaagaacacgttgtttctgcaaatgaacagcctgagagccgaggacacggccgtatattactgtgcgaaggtgggagtgcgcagttcgtccgggatgtgggaccttgactactggggccagggaaccctggtcaccgtcagctca

SEQ ID NO: 372 - protein, 453 aa, synthetic construct
  REGION 1..453: note = source = /note="Description of Artificial Sequence: Synthetic polypeptide"
EVQLLESGGGLVQPGGSRRLSCAASGFTFSYYGMNWVRQAPGKGLEWVSGITNSGSITYYADSVKGRFSISRDNSKNTLFLQMNSLRAEDTAVYYCAKVGVRSSSGMWDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 373 - DNA, 1359 bp, synthetic construct
  misc_feature 1..1359: note = source = /note="Description of Artificial Sequence: Synthetic polynucleotide"
  source 1..1359: mol_type = other DNA
gaggttcaattgttggagtctgggggaggcctggtacagccgggggggtcccggagactgtcctgtgcagcctctggattcacctttagctactatggcatgaactgggtccgccaggctccagggaagggactggaatgggtctcaggtattactaatagtggtagtatcacatactacgcagactccgtgaagggccggttcagcatctccagagacaattccaagaacacgttgtttctgcaaatgaacagcctgagagccgaggacacggccgtatattactgtgcgaaggtgggagtgcgcagttcgtccgggatgtgggaccttgactactggggccagggaaccctggtcaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 374 - protein, 11 aa, synthetic construct
  REGION 1..11: note = source = /note="Description of Artificial Sequence: Synthetic peptide"
GGNNIGSKSLQ

SEQ ID NO: 375 - protein, 7 aa, synthetic construct
  REGION 1..7: note = source = /note="Description of Artificial Sequence: Synthetic peptide"
DDSDRPS

SEQ ID NO: 376 - protein, 11 aa, synthetic construct
  REGION 1..11: note = source = /note="Description of Artificial Sequence: Synthetic peptide"
QVWDTSSDHVV

SEQ ID NO: 377 - protein, 11 aa, synthetic construct
  REGION 1..11: note = source = /note="Description of Artificial Sequence: Synthetic peptide"
GGNNIGSKSLQ

SEQ ID NO: 378 - protein, 7 aa, synthetic construct
  REGION 1..7: note = source = /note="Description of Artificial Sequence: Synthetic peptide"
DDSDRPS

SEQ ID NO: 379 - protein, 11 aa, synthetic construct
  REGION 1..11: note = source = /note="Description of Artificial Sequence: Synthetic peptide"
QVWDTSSDHVV

SEQ ID NO: 380 - protein, 7 aa, synthetic construct
  REGION 1..7: note = source = /note="Description of Artificial Sequence: Synthetic peptide"
NNIGSKS

SEQ ID NO: 381 - skipped

SEQ ID NO: 382 - protein, 8 aa, synthetic construct
  REGION 1..8: note = source = /note="Description of Artificial Sequence: Synthetic peptide"
WDTSSDHV

SEQ ID NO: 383 - protein, 6 aa, synthetic construct
  REGION 1..6: note = source = /note="Description of Artificial Sequence: Synthetic peptide"
NIGSKS

SEQ ID NO: 384 - skipped

SEQ ID NO: 385 - protein, 11 aa, synthetic construct
  REGION 1..11: note = source = /note="Description of Artificial Sequence: Synthetic peptide"
QVWDTSSDHVV

SEQ ID NO: 386 - protein, 108 aa, synthetic construct
  REGION 1..108: note = source = /note="Description of Artificial Sequence: Synthetic polypeptide"
SYVLTQPPSVSVAPGQTARLSCGGNNIGSKSLQWYQQKPGQAPVLVVNDDSDRPSGIPERFSGSNSGNTATLTISRVEAGDEADYYCQVWDTSSDHVVFGGGTKLTVL

SEQ ID NO: 387 - DNA, 324 bp, synthetic construct
  misc_feature 1..324: note = source = /note="Description of Artificial Sequence: Synthetic polynucleotide"
  source 1..324: mol_type = other DNA
tcctatgtgctgactcagccaccctcggtgtcagtggccccaggacagacggccagactttcctgtgggggaaacaacattggaagtaaaagtctgcagtggtaccagcagaagccaggccaggcccctgtgctggtcgtcaatgatgatagcgaccggccctcagggatccctgagcgattctctggctccaactctgggaacacggccaccctgaccatcagcagggtcgaagccggggatgaggccgactattactgtcaggtgtgggatactagtagtgatcatgtggtcttcggcggagggaccaagctgaccgtccta

SEQ ID NO: 388 - protein, 214 aa, synthetic construct
  REGION 1..214: note = source = /note="Description of Artificial Sequence: Synthetic polypeptide"
SYVLTQPPSVSVAPGQTARLSCGGNNIGSKSLQWYQQKPGQAPVLVVNDDSDRPSGIPERFSGSNSGNTATLTISRVEAGDEADYYCQVWDTSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 389 - DNA, 642 bp, synthetic construct
  misc_feature 1..642: note = source = /note="Description of Artificial Sequence: Synthetic polynucleotide"
  source 1..642: mol_type = other DNA
tcctatgtgctgactcagccaccctcggtgtcagtggccccaggacagacggccagactttcctgtgggggaaacaacattggaagtaaaagtctgcagtggtaccagcagaagccaggccaggcccctgtgctggtcgtcaatgatgatagcgaccggccctcagggatccctgagcgattctctggctccaactctgggaacacggccaccctgaccatcagcagggtcgaagccggggatgaggccgactattactgtcaggtgtgggatactagtagtgatcatgtggtcttcggcggagggaccaagctgaccgtcctaggccagcctaaggccgctccctccgtgaccctgttcccccccagctccgaggaactgcaggccaacaaggccaccctggtgtgcctgatcagcgacttctaccctggcgccgtgaccgtggcctggaaggccgacagcagccccgtgaaggccggcgtggagacaaccacccccagcaagcagagcaacaacaagtacgccgccagcagctacctgagcctgacccccgagcagtggaagagccacagaagctacagctgccaggtcacccacgagggcagcaccgtggagaaaaccgtggcccccaccgagtgcagc

SEQ ID NO: 390 - protein, 10 aa, synthetic construct
  REGION 1..10: note = source = /note="Description of Artificial Sequence: Synthetic peptide"
GFTFSYYGMN

SEQ ID NO: 391 - protein, 17 aa, synthetic construct
  REGION 1..17: note = source = /note="Description of Artificial Sequence: Synthetic peptide"
GITQSGSITYYADTVKG

SEQ ID NO: 392 - protein, 14 aa, synthetic construct
  REGION 1..14: note = source = /note="Description of Artificial Sequence: Synthetic peptide"
VGVRSSSGMWDLDY

SEQ ID NO: 393 - protein, 5 aa, synthetic construct
  REGION 1..5: note = source = /note="Description of Artificial Sequence: Synthetic peptide"
YYGMN

SEQ ID NO: 394 - protein, 17 aa, synthetic construct
  REGION 1..17: note = source = /note="Description of Artificial Sequence: Synthetic peptide"
GITQSGSITYYADTVKG

SEQ ID NO: 395 - protein, 14 aa, synthetic construct
  REGION 1..14: note = source = /note="Description of Artificial Sequence: Synthetic peptide"
VGVRSSSGMWDLDY

SEQ ID NO: 396 - protein, 7 aa, synthetic construct
  REGION 1..7: note = source = /note="Description of Artificial Sequence: Synthetic peptide"
GFTFSYY

SEQ ID NO: 397 - protein, 6 aa, synthetic construct
  REGION 1..6: note = source = /note="Description of Artificial Sequence: Synthetic peptide"
TQSGSI

SEQ ID NO: 398 - protein, 14 aa, synthetic construct
  REGION 1..14: note = source = /note="Description of Artificial Sequence: Synthetic peptide"
VGVRSSSGMWDLDY

SEQ ID NO: 399 - protein, 8 aa, synthetic construct
  REGION 1..8: note = source = /note="Description of Artificial Sequence: Synthetic peptide"
GFTFSYYG

SEQ ID NO: 400 - protein, 8 aa, synthetic construct
  REGION 1..8: note = source = /note="Description of Artificial Sequence: Synthetic peptide"
ITQSGSIT

SEQ ID NO: 401 - protein, 16 aa, synthetic construct
  REGION 1..16: note = source = /note="Description of Artificial Sequence: Synthetic peptide"
AKVGVRSSSGMWDLDY

SEQ ID NO: 402 - protein, 123 aa, synthetic construct
  REGION 1..123: note = source = /note="Description of Artificial Sequence: Synthetic polypeptide"
EVQLLESGGGLVQPGGSRRLSCAASGFTFSYYGMNWVRQAPGKGLEWVSGITQSGSITYYADTVKGRFSISRDNSKNTLFLQMNSLRAEDTAVYYCAKVGVRSSSGMWDLDYWGQGTLVTVSS

SEQ ID NO: 403 - DNA, 369 bp, synthetic construct
  misc_feature 1..369: note = source = /note="Description of Artificial Sequence: Synthetic polynucleotide"
  source 1..369: mol_type = other DNA
gaggttcaattgttggagtctgggggaggcctggtacagccgggggggtcccggagactgtcctgtgcagcctctggattcacctttagctactatggcatgaactgggtccgccaggctccagggaagggactggaatgggtctcaggtattactcagagtggtagtatcacatactacgcagacaccgtgaagggccggttcagcatctccagagacaattccaagaacacgttgtttctgcaaatgaacagcctgagagccgaggacacggccgtatattactgtgcgaaggtgggagtgcgcagttcgtccgggatgtgggaccttgactactggggccagggaaccctggtcaccgtcagctca

SEQ ID NO: 404 - protein, 453 aa, synthetic construct
  REGION 1..453: note = source = /note="Description of Artificial Sequence: Synthetic polypeptide"
EVQLLESGGGLVQPGGSRRLSCAASGFTFSYYGMNWVRQAPGKGLEWVSGITQSGSITYYADTVKGRFSISRDNSKNTLFLQMNSLRAEDTAVYYCAKVGVRSSSGMWDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 405 - DNA, 1359 bp, synthetic construct
  misc_feature 1..1359: note = source = /note="Description of Artificial Sequence: Synthetic polynucleotide"
  source 1..1359: mol_type = other DNA
gaggttcaattgttggagtctgggggaggcctggtacagccgggggggtcccggagactgtcctgtgcagcctctggattcacctttagctactatggcatgaactgggtccgccaggctccagggaagggactggaatgggtctcaggtattactcagagtggtagtatcacatactacgcagacaccgtgaagggccggttcagcatctccagagacaattccaagaacacgttgtttctgcaaatgaacagcctgagagccgaggacacggccgtatattactgtgcgaaggtgggagtgcgcagttcgtccgggatgtgggaccttgactactggggccagggaaccctggtcaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 406 - protein, 11 aa, synthetic construct
  REGION 1..11: note = source = /note="Description of Artificial Sequence: Synthetic peptide"
GGNNIGSKSLQ

SEQ ID NO: 407 - protein, 7 aa, synthetic construct
  REGION 1..7: note = source = /note="Description of Artificial Sequence: Synthetic peptide"
DESDRPS

SEQ ID NO: 408 - protein, 11 aa, synthetic construct
  REGION 1..11: note = source = /note="Description of Artificial Sequence: Synthetic peptide"
QVWDTSSDHVV

SEQ ID NO: 409 - protein, 11 aa, synthetic construct
  REGION 1..11: note = source = /note="Description of Artificial Sequence: Synthetic peptide"
GGNNIGSKSLQ

SEQ ID NO: 410 - protein, 7 aa, synthetic construct
  REGION 1..7: note = source = /note="Description of Artificial Sequence: Synthetic peptide"
DESDRPS

SEQ ID NO: 411 - protein, 11 aa, synthetic construct
  REGION 1..11: note = source = /note="Description of Artificial Sequence: Synthetic peptide"
QVWDTSSDHVV

SEQ ID NO: 412 - protein, 7 aa, synthetic construct
  REGION 1..7: note = source = /note="Description of Artificial Sequence: Synthetic peptide"
NNIGSKS

SEQ ID NO: 413 - skipped

SEQ ID NO: 414 - protein, 8 aa, synthetic construct
  REGION 1..8: note = source = /note="Description of Artificial Sequence: Synthetic peptide"
WDTSSDHV

SEQ ID NO: 415 - protein, 6 aa, synthetic construct
  REGION 1..6: note = source = /note="Description of Artificial Sequence: Synthetic peptide"
NIGSKS

SEQ ID NO: 416 - skipped

SEQ ID NO: 417 - protein, 11 aa, synthetic construct
  REGION 1..11: note = source = /note="Description of Artificial Sequence: Synthetic peptide"
QVWDTSSDHVV

SEQ ID NO: 418 - protein, 108 aa, synthetic construct
  REGION 1..108: note = source = /note="Description of Artificial Sequence: Synthetic polypeptide"
SYVLTQPPSVSVAPGQTARLSCGGNNIGSKSLQWYQQKPGQAPVLVVNDESDRPSGIPERFSGSNSGNTATLTISRVEAGDEADYYCQVWDTSSDHVVFGGGTKLTVL

SEQ ID NO: 419 - DNA, 324 bp, synthetic construct
  misc_feature 1..324: note = source = /note="Description of Artificial Sequence: Synthetic polynucleotide"
  source 1..324: mol_type = other DNA
tcctatgtgctgactcagccaccctcggtgtcagtggccccaggacagacggccagactttcctgtgggggaaacaacattggaagtaaaagtctgcagtggtaccagcagaagccaggccaggcccctgtgctggtcgtcaatgatgagagcgaccggccctcagggatccctgagcgattctctggctccaactctgggaacacggccaccctgaccatcagcagggtcgaagccggggatgaggccgactattactgtcaggtgtgggatactagtagtgatcatgtggtcttcggcggagggaccaagctgaccgtccta

SEQ ID NO: 420 - protein, 214 aa, synthetic construct
  REGION 1..214: note = source = /note="Description of Artificial Sequence: Synthetic polypeptide"
SYVLTQPPSVSVAPGQTARLSCGGNNIGSKSLQWYQQKPGQAPVLVVNDESDRPSGIPERFSGSNSGNTATLTISRVEAGDEADYYCQVWDTSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 421 - DNA, 642 bp, synthetic construct
  misc_feature 1..642: note = source = /note="Description of Artificial Sequence: Synthetic polynucleotide"
  source 1..642: mol_type = other DNA
tcctatgtgctgactcagccaccctcggtgtcagtggccccaggacagacggccagactttcctgtgggggaaacaacattggaagtaaaagtctgcagtggtaccagcagaagccaggccaggcccctgtgctggtcgtcaatgatgagagcgaccggccctcagggatccctgagcgattctctggctccaactctgggaacacggccaccctgaccatcagcagggtcgaagccggggatgaggccgactattactgtcaggtgtgggatactagtagtgatcatgtggtcttcggcggagggaccaagctgaccgtcctaggccagcctaaggccgctccctccgtgaccctgttcccccccagctccgaggaactgcaggccaacaaggccaccctggtgtgcctgatcagcgacttctaccctggcgccgtgaccgtggcctggaaggccgacagcagccccgtgaaggccggcgtggagacaaccacccccagcaagcagagcaacaacaagtacgccgccagcagctacctgagcctgacccccgagcagtggaagagccacagaagctacagctgccaggtcacccacgagggcagcaccgtggagaaaaccgtggcccccaccgagtgcagc

SEQ ID NO: 422 - protein, 10 aa, synthetic construct
  REGION 1..10: note = source = /note="Description of Artificial Sequence: Synthetic peptide"
GFTFSYYGMN

SEQ ID NO: 423 - protein, 17 aa, synthetic construct
  REGION 1..17: note = source = /note="Description of Artificial Sequence: Synthetic peptide"
GITNVGSITYYADTVKG

SEQ ID NO: 424 - protein, 14 aa, synthetic construct
  REGION 1..14: note = source = /note="Description of Artificial Sequence: Synthetic peptide"
VGVRSSSGMWDLDY

SEQ ID NO: 425 - protein, 5 aa, synthetic construct
  REGION 1..5: note = source = /note="Description of Artificial Sequence: Synthetic peptide"
YYGMN

SEQ ID NO: 426 - protein, 17 aa, synthetic construct
  REGION 1..17: note = source = /note="Description of Artificial Sequence: Synthetic peptide"
GITNVGSITYYADTVKG

SEQ ID NO: 427 - protein, 14 aa, synthetic construct
  REGION 1..14: note = source = /note="Description of Artificial Sequence: Synthetic peptide"
VGVRSSSGMWDLDY

SEQ ID NO: 428 - protein, 7 aa, synthetic construct
  REGION 1..7: note = source = /note="Description of Artificial Sequence: Synthetic peptide"
GFTFSYY

SEQ ID NO: 429 - protein, 6 aa, synthetic construct
  REGION 1..6: note = source = /note="Description of Artificial Sequence: Synthetic peptide"
TNVGSI

SEQ ID NO: 430 - protein, 14 aa, synthetic construct
  REGION 1..14: note = source = /note="Description of Artificial Sequence: Synthetic peptide"
VGVRSSSGMWDLDY

SEQ ID NO: 431 - protein, 8 aa, synthetic construct
  REGION 1..8: note = source = /note="Description of Artificial Sequence: Synthetic peptide"
GFTFSYYG

SEQ ID NO: 432 - protein, 8 aa, synthetic construct
  REGION 1..8: note = source = /note="Description of Artificial Sequence: Synthetic peptide"
ITNVGSIT

SEQ ID NO: 433 - protein, 16 aa, synthetic construct
  REGION 1..16: note = source = /note="Description of Artificial Sequence: Synthetic peptide"
AKVGVRSSSGMWDLDY

SEQ ID NO: 434 - protein, 123 aa, synthetic construct
  REGION 1..123: note = source = /note="Description of Artificial Sequence: Synthetic polypeptide"
EVQLLESGGGLVQPGGSRRLSCAASGFTFSYYGMNWVRQAPGKGLEWVSGITNVGSITYYADTVKGRFSISRDNSKNTLFLQMNSLRAEDTAVYYCAKVGVRSSSGMWDLDYWGQGTLVTVSS

SEQ ID NO: 435 - DNA, 369 bp, synthetic construct
  misc_feature 1..369: note = source = /note="Description of Artificial Sequence: Synthetic polynucleotide"
  source 1..369: mol_type = other DNA
gaggttcaattgttggagtctgggggaggcctggtacagccgggggggtcccggagactgtcctgtgcagcctctggattcacctttagctactatggcatgaactgggtccgccaggctccagggaagggactggaatgggtctcaggtattactaatgtgggtagtatcacatactacgcagacaccgtgaagggccggttcagcatctccagagacaattccaagaacacgttgtttctgcaaatgaacagcctgagagccgaggacacggccgtatattactgtgcgaaggtgggagtgcgcagttcgtccgggatgtgggaccttgactactggggccagggaaccctggtcaccgtcagctca

SEQ ID NO: 436 - protein, 453 aa, synthetic construct
  REGION 1..453: note = source = /note="Description of Artificial Sequence: Synthetic polypeptide"
EVQLLESGGGLVQPGGSRRLSCAASGFTFSYYGMNWVRQAPGKGLEWVSGITNVGSITYYADTVKGRFSISRDNSKNTLFLQMNSLRAEDTAVYYCAKVGVRSSSGMWDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 437 - DNA, 1359 bp, synthetic construct
  misc_feature 1..1359: note = source = /note="Description of Artificial Sequence: Synthetic polynucleotide"
  source 1..1359: mol_type = other DNA
gaggttcaattgttggagtctgggggaggcctggtacagccgggggggtcccggagactgtcctgtgcagcctctggattcacctttagctactatggcatgaactgggtccgccaggctccagggaagggactggaatgggtctcaggtattactaatgtgggtagtatcacatactacgcagacaccgtgaagggccggttcagcatctccagagacaattccaagaacacgttgtttctgcaaatgaacagcctgagagccgaggacacggccgtatattactgtgcgaaggtgggagtgcgcagttcgtccgggatgtgggaccttgactactggggccagggaaccctggtcaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 438 - protein, 11 aa, synthetic construct
  REGION 1..11: note = source = /note="Description of Artificial Sequence: Synthetic peptide"
GGNNIGSKSLQ

SEQ ID NO: 439 - protein, 7 aa, synthetic construct
  REGION 1..7: note = source = /note="Description of Artificial Sequence: Synthetic peptide"
DESDRPS

SEQ ID NO: 440 - protein, 11 aa, synthetic construct
  REGION 1..11: note = source = /note="Description of Artificial Sequence: Synthetic peptide"
QVWDTSSDHVV

SEQ ID NO: 441 - protein, 11 aa, synthetic construct
  REGION 1..11: note = source = /note="Description of Artificial Sequence: Synthetic peptide"
GGNNIGSKSLQ

SEQ ID NO: 442 - protein, 7 aa, synthetic construct
  REGION 1..7: note = source = /note="Description of Artificial Sequence: Synthetic peptide"
DESDRPS

SEQ ID NO: 443 - protein, 11 aa, synthetic construct
  REGION 1..11: note = source = /note="Description of Artificial Sequence: Synthetic peptide"
QVWDTSSDHVV

SEQ ID NO: 444 - protein, 7 aa, synthetic construct
  REGION 1..7: note = source = /note="Description of Artificial Sequence: Synthetic peptide"
NNIGSKS

SEQ ID NO: 445 - skipped

SEQ ID NO: 446 - protein, 8 aa, synthetic construct
  REGION 1..8: note = source = /note="Description of Artificial Sequence: Synthetic peptide"
WDTSSDHV

SEQ ID NO: 447 - protein, 6 aa, synthetic construct
  REGION 1..6: note = source = /note="Description of Artificial Sequence: Synthetic peptide"
NIGSKS

SEQ ID NO: 448 - skipped

SEQ ID NO: 449 - protein, 11 aa, synthetic construct
  REGION 1..11: note = source = /note="Description of Artificial Sequence: Synthetic peptide"
QVWDTSSDHVV

SEQ ID NO: 450 - protein, 108 aa, synthetic construct; identical to SEQ ID NO: 418
  REGION 1..108: note = source = /note="Description of Artificial Sequence: Synthetic polypeptide"

SEQ ID NO: 451 - DNA, 324 bp, synthetic construct; identical to SEQ ID NO: 419
  misc_feature 1..324: note = source = /note="Description of Artificial Sequence: Synthetic polynucleotide"
  source 1..324: mol_type = other DNA

SEQ ID NO: 452 - protein, 214 aa, synthetic construct; identical to SEQ ID NO: 420
  REGION 1..214: note = source = /note="Description of Artificial Sequence: Synthetic polypeptide"

SEQ ID NO: 453 - DNA, 642 bp, synthetic construct; identical to SEQ ID NO: 421
  misc_feature 1..642: note = source = /note="Description of Artificial Sequence: Synthetic polynucleotide"
  source 1..642: mol_type = other DNA

SEQ ID NO: 454 - protein, 10 aa, synthetic construct
  REGION 1..10: note = source = /note="Description of Artificial Sequence: Synthetic peptide"
GYTFTSYYMH

SEQ ID NO: 455 - protein, 17 aa, synthetic construct
  REGION 1..17: note = source = /note="Description of Artificial Sequence: Synthetic peptide"
IISPSGGSTSYAQKFQG

SEQ ID NO: 456 - protein, 16 aa, synthetic construct
  REGION 1..16: note = source = /note="Description of Artificial Sequence: Synthetic peptide"
DWEGGDPYGYYYAFDY

SEQ ID NO: 457 - protein, 5 aa, synthetic construct
  REGION 1..5: note = source = /note="Description of Artificial Sequence: Synthetic peptide"
SYYMH

SEQ ID NO: 458 - protein, 17 aa, synthetic construct
  REGION 1..17: note = source = /note="Description of Artificial Sequence: Synthetic peptide"
IISPSGGSTSYAQKFQG

SEQ ID NO: 459 - protein, 16 aa, synthetic construct
  REGION 1..16: note = source = /note="Description of Artificial Sequence: Synthetic peptide"
DWEGGDPYGYYYAFDY

SEQ ID NO: 460 - protein, 7 aa, synthetic construct
  REGION 1..7: note = source = /note="Description of Artificial Sequence: Synthetic peptide"
GYTFTSY

SEQ ID NO: 461 - protein, 6 aa, synthetic construct
  REGION 1..6: note = source = /note="Description of Artificial Sequence: Synthetic peptide"
SPSGGS

SEQ ID NO: 462 - protein, 16 aa, synthetic construct
  REGION 1..16: note = source = /note="Description of Artificial Sequence: Synthetic peptide"
DWEGGDPYGYYYAFDY

SEQ ID NO: 463 - protein, 8 aa, synthetic construct
  REGION 1..8: note = source = /note="Description of Artificial Sequence: Synthetic peptide"
GYTFTSYY

SEQ ID NO: 464 - protein, 8 aa, synthetic construct
  REGION 1..8: note = source = /note="Description of Artificial Sequence: Synthetic peptide"
ISPSGGST

SEQ ID NO: 465 - protein, 18 aa, synthetic construct
  REGION 1..18: note = source = /note="Description of Artificial Sequence: Synthetic peptide"
ARDWEGGDPYGYYYAFDY

SEQ ID NO: 466 - protein, 125 aa, synthetic construct
  REGION 1..125: note = source = /note="Description of Artificial Sequence: Synthetic polypeptide"
QVQLVQSGAEVKKPGASVKVSCKASGYTFTSYYMHWVRQAPGQGLEWMGIISPSGGSTSYAQKFQGRVTMTRDTSTSTVYMELSSLRSEDTAVYYCARDWEGGDPYGYYYAFDYWGQGTLVTVSS

SEQ ID NO: 467 - DNA, 375 bp, synthetic construct
  misc_feature 1..375: note = source = /note="Description of Artificial Sequence: Synthetic polynucleotide"
  source 1..375: mol_type = other DNA
caggtgcaattggtgcagagcggagccgaagtgaaaaaacctggggccagcgtgaaagtgtcctgcaaagcctccggatacaccttcaccagctactacatgcactgggtccgccaggccccaggccagggactcgagtggatgggcatcatcagccctagcggcggcagcaccagctacgcccagaaattccagggccgggtgaccatgacccgcgacaccagcaccagcaccgtgtacatggaactgagcagcctgcgcagcgaggacaccgccgtgtattattgcgcgcgtgactgggaaggtggtgacccgtacggttactactacgctttcgactactggggtcaaggcaccctggttacagtcagctca

SEQ ID NO: 468 - protein, 455 aa, synthetic construct
  REGION 1..455: note = source = /note="Description of Artificial Sequence: Synthetic polypeptide"
QVQLVQSGAEVKKPGASVKVSCKASGYTFTSYYMHWVRQAPGQGLEWMGIISPSGGSTSYAQKFQGRVTMTRDTSTSTVYMELSSLRSEDTAVYYCARDWEGGDPYGYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 469 - DNA, 1365 bp, synthetic construct
  misc_feature 1..1365: note = source = /note="Description of Artificial Sequence: Synthetic polynucleotide"
  source 1..1365: mol_type = other DNA
caggtgcaattggtgcagagcggagccgaagtgaaaaaacctggggccagcgtgaaagtgtcctgcaaagcctccggatacaccttcaccagctactacatgcactgggtccgccaggccccaggccagggactcgagtggatgggcatcatcagccctagcggcggcagcaccagctacgcccagaaattccagggccgggtgaccatgacccgcgacaccagcaccagcaccgtgtacatggaactgagcagcctgcgcagcgaggacaccgccgtgtattattgcgcgcgtgactgggaaggtggtgacccgtacggttactactacgctttcgactactggggtcaaggcaccctggttacagtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 470 - protein, 11 aa, synthetic construct
  REGION 1..11: note = source = /note="Description of Artificial Sequence: Synthetic peptide"
RASQSISSYLN

SEQ ID NO: 471 - protein, 7 aa, synthetic construct
  REGION 1..7: note = source = /note="Description of Artificial Sequence: Synthetic peptide"
AASSLQS

SEQ ID NO: 472 - protein, 9 aa, synthetic construct
  REGION 1..9: note = source = /note="Description of Artificial Sequence: Synthetic peptide"
QQSYSTPLT

SEQ ID NO: 473 - protein, 11 aa, synthetic construct
  REGION 1..11: note = source = /note="Description of Artificial Sequence: Synthetic peptide"
RASQSISSYLN

SEQ ID NO: 474 - protein, 7 aa, synthetic construct
  REGION 1..7: note = source = /note="Description of Artificial Sequence: Synthetic peptide"
AASSLQS

SEQ ID NO: 475 - protein, 9 aa, synthetic construct
  REGION 1..9: note = source = /note="Description of Artificial Sequence: Synthetic peptide"
QQSYSTPLT

SEQ ID NO: 476 - protein, 7 aa, synthetic construct
  REGION 1..7: note = source = /note="Description of Artificial Sequence: Synthetic peptide"
SQSISSY

SEQ ID NO: 477 - skipped

SEQ ID NO: 478 - protein, 6 aa, synthetic construct
  REGION 1..6: note = source = /note="Description of Artificial Sequence: Synthetic peptide"
SYSTPL

SEQ ID NO: 479 - protein, 6 aa, synthetic construct
  REGION 1..6: note = source = /note="Description of Artificial Sequence: Synthetic peptide"
QSISSY

SEQ ID NO: 480 - skipped

SEQ ID NO: 481 - protein, 9 aa, synthetic construct
  REGION 1..9: note = source = /note="Description of Artificial Sequence: Synthetic peptide"
QQSYSTPLT

SEQ ID NO: 482 - protein, 107 aa, synthetic construct [sequence omitted]

SEQ ID NO: 483 - DNA, 321 bp, synthetic construct
  misc_feature 1..321: note = source = /note="Description of Artificial Sequence: Synthetic polynucleotide"
  source 1..321: mol_type = other DNA
gatatccagatgacccagagccctagcagcctgagcgccagcgtgggcgaccgcgtgaccattacctgcagagccagccagagcatcagcagctacctgaactggtaccagcagaaacctggcaaggcgcccaaactattaatctacgccgccagcagccttcagagcggcgtgccaagccgctttagcggatccggcagcggcaccgacttcaccctgaccatcagctcccttcagcctgaagacttcgccacctactactgccagcagagctacagcacccctctgacctttggccagggcaccaaagtggaaatcaaa

SEQ ID NO: 484 - protein, 214 aa, synthetic construct
  REGION 1..214: note = source = /note="Description of Artificial Sequence: Synthetic polypeptide"
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 485 - DNA, 642 bp, synthetic construct
  misc_feature 1..642: note = source = /note="Description of Artificial Sequence: Synthetic polynucleotide"
  source 1..642: mol_type = other DNA
gatatccagatgacccagagccctagcagcctgagcgccagcgtgggcgaccgcgtgaccattacctgcagagccagccagagcatcagcagctacctgaactggtaccagcagaaacctggcaaggcgcccaaactattaatctacgccgccagcagccttcagagcggcgtgccaagccgctttagcggatccggcagcggcaccgacttcaccctgaccatcagctcccttcagcctgaagacttcgccacctactactgccagcagagctacagcacccctctgacctttggccagggcaccaaagtggaaatcaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 486 - protein, 10 aa, synthetic construct
  REGION 1..10: note = source = /note="Description of Artificial Sequence: Synthetic peptide"
GYTFTSLEMH

SEQ ID NO: 487 - protein, 17 aa, synthetic construct
  REGION 1..17: note = source = /note="Description of Artificial Sequence: Synthetic peptide"
IIEPSGGSTSYAQKFQG

SEQ ID NO: 488 - protein, 16 aa, synthetic construct
  REGION 1..16: note = source = /note="Description of Artificial Sequence: Synthetic peptide"
DWEGGDPYGYYYAFDY

SEQ ID NO: 489 - protein, 5 aa, synthetic construct
  REGION 1..5: note = source = /note="Description of Artificial Sequence: Synthetic peptide"
SLEMH

SEQ ID NO: 490 - protein, 17 aa, synthetic construct
  REGION 1..17: note = source = /note="Description of Artificial Sequence: Synthetic peptide"
IIEPSGGSTSYAQKFQG

SEQ ID NO: 491 - protein, 16 aa, synthetic construct
  REGION 1..16: note = source = /note="Description of Artificial Sequence: Synthetic peptide"
DWEGGDPYGYYYAFDY

SEQ ID NO: 492 - protein, 7 aa, synthetic construct
  REGION 1..7: note = source = /note="Description of Artificial Sequence: Synthetic peptide"
GYTFTSL

SEQ ID NO: 493 - protein, 6 aa, synthetic construct
  REGION 1..6: note = source = /note="Description of Artificial Sequence: Synthetic peptide"
EPSGGS

SEQ ID NO: 494 - protein, 16 aa, synthetic construct
  REGION 1..16: note = source = /note="Description of Artificial Sequence: Synthetic peptide"
DWEGGDPYGYYYAFDY

SEQ ID NO: 495 - protein, 8 aa, synthetic construct
  REGION 1..8: note = source = /note="Description of Artificial Sequence: Synthetic peptide"
GYTFTSLE

SEQ ID NO: 496 - protein, 8 aa, synthetic construct
  REGION 1..8: note = source = /note="Description of Artificial Sequence: Synthetic peptide"
IEPSGGST

SEQ ID NO: 497 - protein, 18 aa, synthetic construct
  REGION 1..18: note = source = /note="Description of Artificial Sequence: Synthetic peptide"
ARDWEGGDPYGYYYAFDY

SEQ ID NO: 498 - protein, 125 aa, synthetic construct
  REGION 1..125: note = source = /note="Description of Artificial Sequence: Synthetic polypeptide"
QVQLVQSGAEVKKPGASVKVSCKASGYTFTSLEMHWVRQAPGQGLEWMGIIEPSGGSTSYAQKFQGRVTMTRDTSTSTVYMELSSLRSEDTAVYYCARDWEGGDPYGYYYAFDYWGQGTLVTVSS

SEQ ID NO: 499 - DNA, 375 bp, synthetic construct
  misc_feature 1..375: note = source = /note="Description of Artificial Sequence: Synthetic polynucleotide"
  source 1..375: mol_type = other DNA
caggtccaattggtgcagtctggcgccgaagtgaagaaaccaggcgccagcgtgaaggtgtcctgtaaagccagcggctacacctttaccagcctggaaatgcattgggtccgacaggctccaggacagggactcgagtggatgggaattatcgagcctagcggcggcagcacaagctacgcccagaaattccagggcagagtgaccatgaccagagacaccagcacctccaccgtgtacatggaactgagcagcctgagaagcgaggacaccgccgtgtattattgcgcgcgtgattgggaaggcggcgacccttatggctactactacgcctttgattactggggccagggcaccctggtcacagttagctca

SEQ ID NO: 500 - protein, 455 aa, synthetic construct
  REGION 1..455: note = source = /note="Description of Artificial Sequence: Synthetic polypeptide"
QVQLVQSGAEVKKPGASVKVSCKASGYTFTSLEMHWVRQAPGQGLEWMGIIEPSGGSTSYAQKFQGRVTMTRDTSTSTVYMELSSLRSEDTAVYYCARDWEGGDPYGYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 501 - DNA, 1365 bp, synthetic construct
  misc_feature 1..1365: note = source = /note="Description of Artificial Sequence: Synthetic polynucleotide"
  source 1..1365: mol_type = other DNA
caggtccaattggtgcagtctggcgccgaagtgaagaaaccaggcgccagcgtgaaggtgtcctgtaaagccagcggctacacctttaccagcctggaaatgcattgggtccgacaggctccaggacagggactcgagtggatgggaattatcgagcctagcggcggcagcacaagctacgcccagaaattccagggcagagtgaccatgaccagagacaccagcacctccaccgtgtacatggaactgagcagcctgagaagcgaggacaccgccgtgtattattgcgcgcgtgattgggaaggcggcgacccttatggctactactacgcctttgattactggggccagggcaccctggtcacagttagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacatgccccccctgcccggcgccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcttaagccccggcaag

SEQ ID NO: 502 - protein, 11 aa, synthetic construct
  REGION 1..11: note = source = /note="Description of Artificial Sequence: Synthetic peptide"
RASQSISSYLN

SEQ ID NO: 503 - protein, 7 aa, synthetic construct
  REGION 1..7: note = source = /note="Description of Artificial Sequence: Synthetic peptide"
AASSLQS

SEQ ID NO: 504 - protein, 9 aa, synthetic construct
  REGION 1..9: note = source = /note="Description of Artificial Sequence: Synthetic peptide"
QQSYSTPLT

SEQ ID NO: 505 - protein, 11 aa, synthetic construct
  REGION 1..11: note = source = /note="Description of Artificial Sequence: Synthetic peptide"
RASQSISSYLN

SEQ ID NO: 506 - protein, 7 aa, synthetic construct
  REGION 1..7: note = source = /note="Description of Artificial Sequence: Synthetic peptide"
AASSLQS

SEQ ID NO: 507 - protein, 9 aa, synthetic construct
  REGION 1..9: note = source = /note="Description of Artificial Sequence: Synthetic peptide"
QQSYSTPLT

SEQ ID NO: 508 - protein, 7 aa, synthetic construct
  REGION 1..7: note = source = /note="Description of Artificial Sequence: Synthetic peptide"
SQSISSY

SEQ ID NO: 509 - skipped

SEQ ID NO: 510 - protein, 6 aa, synthetic construct
  REGION 1..6: note = source = /note="Description of Artificial Sequence: Synthetic peptide"
SYSTPL

SEQ ID NO: 511 - protein, 6 aa, synthetic construct
  REGION 1..6: note = source = /note="Description of Artificial Sequence: Synthetic peptide"
QSISSY

SEQ ID NO: 512 - skipped

SEQ ID NO: 513 - protein, 9 aa, synthetic construct
  REGION 1..9: note = source = /note="Description of Artificial Sequence: Synthetic peptide"
QQSYSTPLT

SEQ ID NO: 514 - protein, 107 aa, synthetic construct [sequence omitted]

SEQ ID NO: 515 - DNA, 321 bp, synthetic construct; identical to SEQ ID NO: 483
  misc_feature 1..321: note = source = /note="Description of Artificial Sequence: Synthetic polynucleotide"
  source 1..321: mol_type = other DNA

SEQ ID NO: 516 - protein, 214 aa, synthetic construct; identical to SEQ ID NO: 484
  REGION 1..214: note = source = /note="Description of Artificial Sequence: Synthetic polypeptide"

SEQ ID NO: 517 - DNA, 642 bp, synthetic construct; identical to SEQ ID NO: 485
  misc_feature 1..642: note = source = /note="Description of Artificial Sequence: Synthetic polynucleotide"
  source 1..642: mol_type = other DNA